(19) United States
(12) Patent Application Publication
(10) Pub. No.: US 2023/0151095 A1
(43) Pub. Date: May 18, 2023

(54) BISPECIFIC ANTIBODIES THAT BIND TO B7H3 AND NKG2D

(71) Applicant: Xencor, Inc., Monrovia, CA (US)

(72) Inventors: Katrina Bykova, Seattle, WA (US); Matthew Faber, Glendora, CA (US); Matthew Bernett, Monrovia, CA (US); Tian Zhang, Chino Hills, CA (US); Erik Pong, San Gabriel, CA (US); Kendra Avery, Hawthorne, CA (US); John Desjarlais, Pasadena, CA (US)

(21) Appl. No.: 18/053,325

(22) Filed: Nov. 7, 2022

Related U.S. Application Data
(60) Provisional application No. 63/278,999, filed on Nov. 12, 2021.

(51) Int. Cl. C07K 16/28; A61P 35/00

(52) U.S. Cl. CPC C07K 16/2803; A61P 35/00; C07K 2317/622; C07K 2317/52; C07K 2317/565

(57) ABSTRACT

Provided herein are bispecific antibodies that bind to B7H3 and NKG2D (e.g., antibodies having Fab-scFv-Fc, Fab₂-scFv-Fc, mAb-scFv and stackFab₂-scFv-Fc formats) or antigen binding fragments thereof. Also provided herein are polynucleotide sequences encoding a chain and/or a CDR of a bispecific antibody of the disclosure; and vectors and cells comprising such polynucleotide sequences. Also provided herein are methods of treating cancer in a subject with a bispecific antibody of the disclosure.

### CROSS REFERENCE TO RELATED APPLICATIONS

This application claims the benefit of U.S. Provisional Patent Application No. 63/278,999, filed Nov. 12, 2021, which is hereby incorporated by reference in its entirety.

### SEQUENCE LISTING

The instant application contains a Sequence Listing which has been submitted electronically in XML format and is hereby incorporated by reference in its entirety. The XML copy, created on Nov. 7, 2022 is named 51096_4002_US.xml and is 3,532,485 bytes in size.

### BACKGROUND

Antibody-based therapeutics have been used successfully to treat a variety of diseases, including cancer. An increasingly prevalent avenue being explored is the engineering of single immunoglobulin molecules that co-engage two different antigens. Such alternate antibody formats that engage two different antigens are often referred to as bispecific antibodies. Because the considerable diversity of the antibody variable region (Fv) makes it possible to produce an Fv that recognizes virtually any molecule, the typical approach to bispecific antibody generation is the introduction of new variable regions into the antibody.

A number of alternate antibody formats have been explored for bispecific targeting (Chames & Baty, 2009, mAbs 1[6]:1-9; Holliger & Hudson, 2005, Nature Biotechnology 23[9]:1126-1136; and Kontermann, 2012 MAbs 4(2):182, all of which are expressly incorporated herein by reference). Initially, bispecific antibodies were made by fusing two cell lines that each produced a single monoclonal antibody (Milstein et al., 1983, Nature 305:537-540). Although the resulting hybrid hybridoma or quadroma did produce bispecific antibodies, they were only a minor population, and extensive purification was required to isolate the desired antibody. An engineering solution to this was the use of antibody fragments to make bispecifics. Because such fragments lack the complex quaternary structure of a full-length antibody, variable light and heavy chains can be linked in single genetic constructs. Antibody fragments of many different forms have been generated, including diabodies, single chain diabodies, tandem scFv's, and Fab₂ bispecifics (Chames & Baty, 2009, mAbs 1[6]:1-9; Holliger & Hudson, 2005, Nature Biotechnology 23[9]:1126-1136; expressly incorporated herein by reference). While these formats can be expressed at high levels in bacteria and may have favorable penetration benefits due to their small size, they clear rapidly in vivo and can present manufacturing obstacles related to their production and stability. A principal cause of these drawbacks is that antibody fragments typically lack the constant region of the antibody with its associated functional properties, including larger size, high stability, and binding to various Fc receptors and ligands that maintain long half-life in serum (i.e., the neonatal Fc receptor FcRn) or serve as binding sites for purification (i.e., protein A and protein G).

More recent work has attempted to address the shortcomings of fragment-based bispecifics by engineering dual binding into full length antibody-like formats (Wu et al., 2007, Nature Biotechnology 25[11]:1290-1297; U.S. Ser. No. 12/477,711; Michaelson et al., 2009, mAbs 1[2]:128-141; PCT/US2008/074693; Zuo et al., 2000, Protein Engineering 13[5]:361-367; U.S. Ser. No. 09/865,198; Shen et al., 2006, J Biol Chem 281[16]:10706-10714; Lu et al., 2005, J Biol Chem 280[20]:19665-19672; PCT/US2005/025472; and Kontermann, 2012 MAbs 4(2):182, all of which are expressly incorporated herein by reference). These formats overcome some of the obstacles of the antibody fragment bispecifics, principally because they contain an Fc region. One significant drawback of these formats is that, because they build new antigen binding sites on top of the homodimeric constant chains, binding to the new antigen is always bivalent.

For many antigens that are attractive as co-targets in a therapeutic bispecific format, the desired binding is monovalent rather than bivalent. For many immune receptors, cellular activation is accomplished by cross-linking of a monovalent binding interaction. The mechanism of cross-linking is typically mediated by antibody/antigen immune complexes, or via effector cell to target cell engagement. For example, the low affinity Fc gamma receptors (FcγRs) such as FcγRIIa, FcγRIIb, and FcγRIIIa bind monovalently to the antibody Fc region. Monovalent binding does not activate cells expressing these FcγRs; however, upon immune complexation or cell-to-cell contact, receptors are cross-linked and clustered on the cell surface, leading to activation. For receptors responsible for mediating cellular killing, for example FcγRIIIa on natural killer (NK) cells, receptor cross-linking and cellular activation occurs when the effector cell engages the target cell in a highly avid format (Bowles & Weiner, 2005, J Immunol Methods 304:88-99, expressly incorporated by reference). Similarly, on B cells the inhibitory receptor FcγRIIb downregulates B cell activation only when it engages into an immune complex with the cell surface B-cell receptor (BCR), a mechanism that is mediated by immune complexation of soluble IgG's with the same antigen that is recognized by the BCR (Heyman 2003, Immunol Lett 88[2]:157-161; Smith and Clatworthy, 2010, Nature Reviews Immunology 10:328-343; expressly incorporated by reference).

T cell-mediated immune response plays an extremely important role in anti-tumor processes of an organism. However, the activation and proliferation of T cells requires not only an antigen signal recognized by TCR, but also a second signal provided by co-stimulatory molecules. The molecules of the B7 family belong to the co-stimulatory molecule immunoglobulin superfamily. More and more studies have shown that molecules of this family play an important regulatory role in the normal immune function and pathological state in an organism.

B7H3 is a member of B7 family and is a type I transmembrane protein, which contains a signal peptide at the amino terminus, an extracellular immunoglobulin-like variable region (IgV) and constant region (IgC), a transmembrane region, and a cytoplasmic tail region having 45 amino acids (Tissue Antigens. 2007 August; 70(2): 96-104). B7H3 has two kinds of splicing variants, B7H3a and B7H3b. The extracellular domain of B7H3a consists of two immunoglobulin domains of IgV-IgC (also known as 2IgB7H3), and the extracellular domain of B7H3b consists of four immunoglobulin domains of IgV-IgC-IgV-IgC (also known as 4IgB7H3).

B7H3 protein is not expressed or is poorly expressed in normal tissues and cells, but highly expressed in various tumor tissues and is closely correlated with tumor progression, patient survival and prognosis. It has been clinically reported that B7H3 is over-expressed in many types of cancers, especially in non-small cell lung cancer, renal cancer, urinary tract epithelial cancer, colorectal cancer, prostate cancer, glioblastoma multiforme, ovarian cancer and pancreas cancer (Lung Cancer. 2009 November; 66(2): 245-249; Clin Cancer Res. 2008 Aug. 15; 14(16): 5150-5157). In addition, it has also been reported in the literature that, in prostate cancer, the expression level of B7H3 is positively correlated with clinical pathological malignancy (such as tumor volume, extra-prostatic invasion or Gleason score), and is also associated with cancer progression (Cancer Res. 2007 Aug. 15; 67(16):7893-7900). Similarly, in glioblastoma multiforme, the expression of B7H3 is inversely associated with event-free survival, and in pancreatic cancer, the expression of B7H3 is associated with lymph node metastasis and pathological progression. Therefore, B7H3 is considered as a new tumor marker and potential therapeutic target.

Natural killer group 2 member D (NKG2D) is an activating receptor present on the surface of natural killer (NK) cells, some NK T cells, CD8+ cytotoxic T cells, γδ T cells, and CD4+ T cells, under certain conditions. (Champsaur M, Lanier L L. Immunol Rev 2010; 235:267-85; Jamieson A M, Diefenbach A, McMahon C W, et al. Immunity 2002; 17:19-29).

The present disclosure is directed to bispecific B7H3 and NKG2D antibodies and the use of such antibodies for use in therapy (e.g., cancer therapy).

### SUMMARY

In one aspect, provided herein is a heterodimeric antibody comprising: a) a first monomer comprising: i) an anti-NKG2D scFv comprising a first variable heavy VH1 domain, an scFv linker and a first variable light VL1 domain; and ii) a first Fc domain, wherein the scFv is covalently attached to the N-terminus of the first Fc domain using a domain linker; b) a second monomer comprising a VH2-CH1-hinge-CH2-CH3 monomer, wherein VH2 is a second variable heavy domain and CH2-CH3 is a second Fc domain; and c) a light chain comprising a second variable light VL2 domain, wherein the second variable heavy VH2 domain and the second variable light VL2 domain form an B7H3 antigen binding domain, and wherein the first Fc domain and/or the second Fc domain comprise an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering and have enhanced FcγRIIIA (CD16a) binding compared to first and second Fc domains lacking such substitution(s).

In some embodiments, the B7H3 antigen binding domain comprises a set of vhCDR1-3 and vlCDR1-3 from a variable heavy domain and variable light domain pair selected from the group consisting of SEQ ID NOS: 27, 28, and 29 for vhCDR1-3 and SEQ ID NOS: 30, 31, and 32 for vlCDR1-3 of 38E2[B7H3]_H2_L1.1; SEQ ID NOS: 243, 244, and 245 for vhCDR1-3 and SEQ ID NOS: 247, 248, and 249 for vlCDR1-3 of 6A1[B7H3]_H1_L1; SEQ ID NOS: 143, 144, and 22 for vhCDR1-3 and SEQ ID NOS: 23, 24, and 26 for vlCDR1-3 of 2E43.189[B7H3]_H1_L1; and SEQ ID NOS: 20, 21, and 22 for vhCDR1-3 and SEQ ID NOS: 23, 24, and 26 for vlCDR1-3 of 2E43.189[B7H3]_H1.22_L1, as depicted in FIGS. 13 and 14.

In some embodiments, the B7H3 antigen binding domain comprises a variable heavy domain and variable light domain pair selected from the group consisting of SEQ ID NOS: 140 and 141 of 38E2[B7H3]_H2_L1.1; SEQ ID NOS: 242 and 246 of 6A1[B7H3]_H1L1; SEQ ID NOS: 142 and 51 of 2E4A3.189[B7H3] H1_L1; and SEQ ID NOS: 145 and 51 of 2E4A3.189[B7H3] H1.22_L1, as depicted in FIGS. 13 and 14.

In some embodiments, the anti-NKG2D scFv comprises a set of vhCDR1-3 and the vlCDR1-3 from a variable heavy domain and variable light domain pair selected from the group consisting of SEQ ID NOS: 2604-2606 for vhCDR1-3 and SEQ ID NOS: 2608-2610 for vlCDR1-3 of mAb-C[NKG2D]; SEQ ID NOS: 2612-2614 for vhCDR1-3 and SEQ ID NOS: 2616-2618 for vlCDR1-3 of mAb-D[NKG2D]; SEQ ID NOS: 17-18 and 1256 for vhCDR1-3 of 1D7B4[NKG2D]_H1.23 and SEQ ID NOS: 23, 24, and 26 for vlCDR1-3 of 1D7B4[NKG2D]_L1; SEQ ID NOS: 17-18 and 1272 for vhCDR1-3 of 1D7B4[NKG2D]_H1.31 and SEQ ID NOS: 23, 24, and 26 for vlCDR1-3 of 1D7B4[NKG2D]_L1; SEQ ID NOS: 17-19 for vhCDR1-3 and SEQ ID NOS: 23, 24, and 26 for vlCDR1-3 of 1D7B4[NKG2D]_H1_L1; SEQ ID NOS: 33-35 for vhCDR1-3 and SEQ ID NOS: 23, 24, and 26 for vlCDR1-3 of 1D2B4[NKG2D]_H1_L1; SEQ ID NOS: 1212 and 18-19 for vhCDR1-3 of 1D7B4[NKG2D]_H1.1 and SEQ ID NOS: 23, 24, and 26 for vlCDR1-3; SEQ ID NOS: 1214 and 18-19 for vhCDR1-3 of 1D7B4[NKG2D]_H1.2 and SEQ ID NOS: 23, 24, and 26 for vlCDR1-3; SEQ ID NOS: 1216 and 18-19 for vhCDR1-3 of 1D7B4[NKG2D]_H1.3 and SEQ ID NOS: 23, 24, and 26 for vlCDR1-3; SEQ ID NOS: 1218 and 18-19 for vhCDR1-3 of 1D7B4[NKG2D]_H1.4 and SEQ ID NOS: 23, 24, and 26 for vlCDR1-3; SEQ ID NOS: 1220 and 18-19 for vhCDR1-3 of 1D7B4[NKG2D]_H1.5 and SEQ ID NOS: 23, 24, and 26 for vlCDR1-3; SEQ ID NOS: 1222 and 18-19 for vhCDR1-3 of 1D7B4[NKG2D]_H1.6 and SEQ ID NOS: 23, 24, and 26 for vlCDR1-3; SEQ ID NOS: 1224 and 18-19 for vhCDR1-3 of 1D7B4[NKG2D]_H1.7 and SEQ ID NOS: 23, 24, and 26 for vlCDR1-3; SEQ ID NOS: 1226 and 18-19 for vhCDR1-3 of 1D7B4[NKG2D]_H1.8 and SEQ ID NOS: 23, 24, and 26 for vlCDR1-3; SEQ ID NOS: 17-18 and 1228 for vhCDR1-3 of 1D7B4[NKG2D]_H1.9 and SEQ ID NOS: 23, 24, and 26 for vlCDR1-3; SEQ ID NOS: 17, 18 and 1230 for vhCDR1-3 of 1D7B4[NKG2D]_H1.10 and SEQ ID NOS: 23, 24, and 26 for vlCDR1-3; SEQ ID NOS: 17-18 and 1232 for vhCDR1-3 of 1D7B4[NKG2D]_H1.11 and SEQ ID NOS: 23, 24, and 26 for vlCDR1-3; SEQ ID NOS: 12-18 and 1234 for vhCDR1-3 of 1D7B4[NKG2D]_H1.12 and SEQ ID NOS: 23, 24, and 26 for vlCDR1-3; SEQ ID NOS: 12-18 and 1236 for vhCDR1-3 of 1D7B4[NKG2D]_H1.13 and SEQ ID NOS: 23, 24, and 26 for vlCDR1-3; SEQ ID NOS: 17-18 and 1238 for vhCDR1-3 of 1D7B4[NKG2D]_H1.14 and SEQ ID NOS: 23, 24, and 26 for vlCDR1-3; SEQ ID NOS: 17-18 and 1240 for vhCDR1-3 of 1D7B4[NKG2D]_H1.15 and SEQ ID NOS: 23, 24, and 26 for vlCDR1-3; SEQ ID NOS: 17-18 and 1242 for vhCDR1-3 of 1D7B4[NKG2D]_H1.16 and SEQ ID NOS: 23, 24, and 26 for vlCDR1-3; SEQ ID NOS: 17-18 and 1244 for vhCDR1-3 of 1D7B4[NKG2D]_H1.17 and SEQ ID NOS: 23, 24, and 26 for vlCDR1-3; SEQ ID NOS: 17-18 and 1246 for vhCDR1-3 of 1D7B4[NKG2D]_H1.18 and SEQ ID NOS: 23, 24, and 26 for vlCDR1-3; SEQ ID NOS: 17-18 and 1248 for vhCDR1-3 of 1D7B4[NKG2D]_H1.19 and SEQ ID NOS: 23, 24, and 26 for vlCDR1-3; SEQ ID NOS: 17-18 and 1250 for vhCDR1-3 of 1D7B4[NKG2D]_H1.20 and SEQ ID NOS: 23, 24, and 26 for vlCDR1-3; SEQ ID NOS: 17-18 and 1252 for vhCDR1-3 of 1D7B4[NKG2D]_H1.21 and SEQ ID NOS: 23, 24, and 26 for vlCDR1-3; SEQ ID NOS: 17-18 and 1254 for vhCDR1-3 of 1D7B4[NKG2D]_H1.22 and SEQ ID NOS: 23, 24, and 26 for vlCDR1-3; SEQ ID NOS: 17-18 and 1258 for vhCDR1-3 of 1D7B4[NKG2D]_H1.24 and SEQ ID NOS: 23, 24, and 26 for vlCDR1-3; SEQ ID NOS: 17-18 and 1260 for vhCDR1-3 of 1D7B4[NKG2D]_H1.25 and SEQ ID NOS: 23, 24, and 26 for vlCDR1-3; SEQ ID NOS: 17-18 and 1262 for vhCDR1-3 of 1D7B4[NKG2D]_H1.26 and SEQ ID NOS: 23, 24, and 26 for vlCDR1-3; SEQ ID NOS: 17-18 and 1264 for vhCDR1-3 of 1D7B4[NKG2D]_H1.27 and SEQ ID NOS: 23, 24, and 26 for vlCDR1-3; SEQ ID NOS: 17-18 and 1266 for vhCDR1-3 of 1D7B4[NKG2D]_H1.28 and SEQ ID NOS: 23, 24, and 26 for vlCDR1-3; SEQ ID NOS: 17-18 and 1268 for vhCDR1-3 of 1D7B4[NKG2D]_H1.29 and SEQ ID NOS: 23, 24, and 26 for vlCDR1-3; SEQ ID NOS: 17-18 and 1270 for vhCDR1-3 of 1D7B4[NKG2D]_H1.30 and SEQ ID NOS: 23, 24, and 26 for vlCDR1-3; SEQ ID NOS: 17-18 and 1274 for vhCDR1-3 of 1D7B4[NKG2D]_H1.32 and SEQ ID NOS: 23, 24, and 26 for vlCDR1-3; SEQ ID NOS: 17-18 and 1276 for vhCDR1-3 of 1D7B4[NKG2D]_H1.33 and SEQ ID NOS: 23, 24, and 26 for vlCDR1-3; SEQ ID NOS: 17-18 and 1278 for vhCDR1-3 of 1D7B4[NKG2D]_H1.34 and SEQ ID NOS: 23, 24, and 26 for vlCDR1-3; SEQ ID NOS: 17-18 and 1280 for vhCDR1-3 of 1D7B4[NKG2D]_H1.35 and SEQ ID NOS: 23, 24, and 26 for vlCDR1-3; SEQ ID NOS: 17-18 and 1282 for vhCDR1-3 of 1D7B4[NKG2D]_H1.36 and SEQ ID NOS: 23, 24, and 26 for vlCDR1-3; SEQ ID NOS: 17-18 and 1284 for vhCDR1-3 of 1D7B4[NKG2D]_H1.37 and SEQ ID NOS: 23, 24, and 26 for vlCDR1-3; SEQ ID NOS: 17-18 and 1286 for vhCDR1-3 of 1D7B4[NKG2D]_H1.38 and SEQ ID NOS: 23, 24, and 26 for vlCDR1-3; SEQ ID NOS: 17-18 and 1288 for vhCDR1-3 of 1D7B4[NKG2D]_H1.39 and SEQ ID NOS: 23, 24, and 26 for vlCDR1-3; SEQ ID NOS: 17-18 and 1290 for vhCDR1-3 of 1D7B4[NKG2D]_H1.40 and SEQ ID NOS: 23, 24, and 26 for vlCDR1-3; SEQ ID NOS: 17-18 and 1292 for vhCDR1-3 of 1D7B4[NKG2D]_H1.41 and SEQ ID NOS: 23, 24, and 26 for vlCDR1-3; SEQ ID NOS: 17-18 and 1294 for vhCDR1-3 of 1D7B4[NKG2D]_H1.42 and SEQ ID NOS: 23, 24, and 26 for vlCDR1-3; SEQ ID NOS: 17-18 and 1296 for vhCDR1-3 of 1D7B4[NKG2D]_H1.43 and SEQ ID NOS: 23, 24, and 26 for vlCDR1-3; SEQ ID NOS: 17-18 and 1298 for vhCDR1-3 of 1D7B4[NKG2D]_H1.44 and SEQ ID NOS: 23, 24, and 26 for vlCDR1-3; SEQ ID NOS: 17-18 and 1300 for vhCDR1-3 of 1D7B4[NKG2D]_H1.45 and SEQ ID NOS: 23, 24, and 26 for vlCDR1-3; SEQ ID NOS: 17-18 and 1302 for vhCDR1-3 of 1D7B4[NKG2D]_H1.46 and SEQ ID NOS: 23, 24, and 26 for vlCDR1-3; SEQ ID NOS17-18 and 1304 for vhCDR1-3 of 1D7B4[NKG2D]_H1.47 and SEQ ID NOS: 23, 24, and 26 for vlCDR1-3; and SEQ ID NOS: 17-18 and 1306 for vhCDR1-3 of 1D7B4[NKG2D]_H1.48 and SEQ ID NOS: 23, 24, and 26 for vlCDR1-3, as depicted in FIGS. 23 and 58.

In some embodiments, the anti-NKG2D scFv comprises a variable heavy domain and variable light domain pair selected from the group consisting of SEQ ID NOS: 2603 and 2607 of mAb-C[NKG2D]; SEQ ID NOS: 2611 and 2615 of mAb-D[NKG2D]; SEQ ID NOS: 1255 and 51 of 1D7B4[NKG2D]_H1.23_L1; SEQ ID NOS: 1271 and 51 of 1D7B4[NKG2D]_H1.31_L1; SEQ ID NOS: 50 and 51 of 1D7B4[NKG2D]_H1_L1; SEQ ID NOS: 52 and 51 of 1D2B4[NKG2D]; SEQ ID NOS: 1211 and 51 of 1D7B4[NKG2D]_H1.1_L1; SEQ ID NOS: 1213 and 51 of 1D7B4[NKG2D]_H1.2_L1; SEQ ID NOS: 1215 and 51 of 1D7B4[NKG2D]_H1.3_L1; SEQ ID NOS: 1217 and 51 of 1D7B4[NKG2D]_H1.4_L1; SEQ ID NOS: 1219 and 51 of 1D7B4[NKG2D]_H1.5_L1; SEQ ID NOS: 1221 and 51 of 1D7B4[NKG2D]_H1.6_L1; SEQ ID NOS: 1223 and 51 of 1D7B4[NKG2D]_H1.7_L1; SEQ ID NOS: 1225 and 51 of 1D7B4[NKG2D]_H1.8_L1; SEQ ID NOS: 1227 and 51 of 1D7B4[NKG2D]_H1.9_L1; SEQ ID NOS: 1229 and 51 of 1D7B4[NKG2D]_H1.10_L1; SEQ ID NOS: 1231 and 51 of 1D7B4[NKG2D]_H1.11_L1; SEQ ID NOS: 1233 and 51 of 1D7B4[NKG2D]_H1.12_L1; SEQ ID NOS: 1235 and 51 of 1D7B4[NKG2D]_H1.13_L1; SEQ ID NOS: 1237 and 51 of 1D7B4[NKG2D]_H1.14_L1; SEQ ID NOS: 1239 and 51 of 1D7B4[NKG2D]_H1.15_L1; SEQ ID NOS: 1241 and 51 of 1D7B4[NKG2D]_H1.16_L1; SEQ ID NOS: 1243 and 51 of 1D7B4[NKG2D]_H1.17_L1; SEQ ID NOS: 1245 and 51 of 1D7B4[NKG2D]_H1.18_L1; SEQ ID NOS: 1247 and 51 of 1D7B4[NKG2D]_H1.19_L1; SEQ ID NOS: 1249 and 51 of 1D7B4[NKG2D]_H1.20_L1; SEQ ID NOS: 1251 and 51 of 1D7B4[NKG2D]_H1.21_L1; SEQ ID NOS: 1253 and 51 of 1D7B4[NKG2D]_H1.22_L1; SEQ ID NOS: 1257 and 51 of 1D7B4[NKG2D]_H1.24_L1; SEQ ID NOS: 1259 and 51 of 1D7B4[NKG2D]_H1.25_L1; SEQ ID NOS: 1261 and 51 of 1D7B4[NKG2D]_H1.26_L1; SEQ ID NOS: 1263 and 51 of 1D7B4[NKG2D]_H1.27_L1; SEQ ID NOS: 1265 and 51 of 1D7B4[NKG2D]_H1.28_L1; SEQ ID NOS: 1267 and 51 of 1D7B4[NKG2D]_H1.29_L1; SEQ ID NOS: 1269 and 51 of 1D7B4[NKG2D]_H1.30_L1; SEQ ID NOS: 1273 and 51 of 1D7B4[NKG2D]_H1.32_L1; SEQ ID NOS: 1275 and 51 of 1D7B4[NKG2D]_H1.33_L1; SEQ ID NOS: 1277 and 51 of 1D7B4[NKG2D]_H1.34_L1; SEQ ID NOS: 1279 and 51 of 1D7B4[NKG2D]_H1.35_L1; SEQ ID NOS: 1281 and 51 of 1D7B4[NKG2D]_H1.36_L1; SEQ ID NOS: 1283 and 51 of 1D7B4[NKG2D]_H1.37_L1; SEQ ID NOS: 1285 and 51 of 1D7B4[NKG2D]_H1.38_L1; SEQ ID NOS: 1287 and 51 of 1D7B4[NKG2D]_H1.39_L1; SEQ ID NOS: 1289 and 51 of 1D7B4[NKG2D]_H1.40_L1; SEQ ID NOS: 1291 and 51 of 1D7B4[NKG2D]_H1.41_L1; SEQ ID NOS: 1293 and 51 of 1D7B4[NKG2D]_H1.42_L1; SEQ ID NOS: 1295 and 51 of 1D7B4[NKG2D]_H1.43_L1; SEQ ID NOS: 1297 and 51 of 1D7B4[NKG2D]_H1.44_L1; SEQ ID NOS: 1299 and 51 of 1D7B4[NKG2D]_H1.45_L1; SEQ ID NOS: 1301 and 51 of 1D7B4[NKG2D]_H1.46_L1; SEQ ID NOS: 1303 and 51 of 1D7B4[NKG2D]_H1.47_L1; and SEQ ID NOS: 1305 and 51 of 1D7B4[NKG2D]_H1.48_L1, as depicted in FIGS. 23 and 58.

In some embodiments, the first variable light domain of the anti-NKG2D scFv is covalently attached to the N-terminus of the first Fc domain using a domain linker.

In some embodiments, the first variable heavy domain of the anti-NKG2D scFv is covalently attached to the N-terminus of the first Fc domain using a domain linker.

In some embodiments, the scFv linker is a charged scFv linker.

In some embodiments, the scFv linker is a charged scFv linker having the amino acid sequence (GKPGS)4 (SEQ ID NO:96).

In some embodiments, the first domain comprises an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

In some embodiments, the second domain comprises an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

In some embodiments, the first and second Fc domains comprise a set of amino acid substitutions selected from the group consisting of: S239D/I332E:S239D/I332E; S239D:S239D; I332E:I332E; WT:S239D/I332E; WT:S239D; WT:I332E; S239D/I332E:WT; S239D:WT; I332E:WT; S239D/I332E:S239D; S239D/I332E:I332E; S239D:S239D/I332E; I332E:S239D/I332E; S239D:I332E; and I332E:S239D, wherein numbering is according to EU numbering.

In some embodiments, the first or second Fc domain comprises the amino acid substitutions S239D/I332E, wherein numbering is according to EU numbering.

In some embodiments, the first and second Fc domains further comprise a set of heterodimerization variants selected from the group consisting of those depicted in FIGS. 1A-1E, wherein numbering is according to EU numbering.

In some embodiments, the set of heterodimerization variants is selected from the group consisting of S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering.

In some embodiments, the first or second Fc domain further comprises one or more pI variants.

In some embodiments, the one or more pI variants are N208D/Q295E/N384D/Q418E/N421D, wherein numbering is according to EU numbering.

In some embodiments, the first and second monomers each further comprise amino acid substitutions selected from the group consisting of M428L/N434S, M428L/N434A, and M252Y/S254T/T256E, wherein numbering is according to EU numbering.

In some embodiments, the heterodimeric antibody described herein is selected from the group consisting of: the amino acid sequences of SEQ ID NOS:1, 2 and 3 of XENP38597; the amino acid sequences of SEQ ID NOS:4, 5 and 6 of XENP40377; the amino acid sequences of SEQ ID NOS:7, 8 and 3 of XENP38101; the amino acid sequences of SEQ ID NOS:9, 10 and 3 of XENP38108; the amino acid sequences of SEQ ID NOS:11, 2 and 3 of XENP38596; the amino acid sequences of SEQ ID NOS:12, 2, and 13 of XENP38598; the amino acid sequences of SEQ ID NOS:14, 2 and 15 of XENP38599; the amino acid sequences of SEQ ID NOS:4, 16 and 6 of XENP40374; the amino acid sequences of SEQ ID NOS:1307-1308 and 6 of XENP42652; the amino acid sequences of SEQ ID NOS:1309-1310 and 6 of XENP42653; the amino acid sequences of SEQ ID NOS:1311-1312 and 6 of XENP42654; the amino acid sequences of SEQ ID NOS:1313-1314 and 6 of XENP42655; the amino acid sequences of SEQ ID NOS:1315-1316 and 6 of XENP42656, as depicted in FIGS. 19 and 59.

Also provided is a nucleic acid composition comprising nucleic acids encoding the first and second monomers and the light chain of any of the antibodies described.

Also provided is a expression vector comprising the nucleic acids described herein.

Additionally, provided is a host cell transformed with any of the expression vectors described herein.

In some embodiments, provided is a method of making a heterodimeric antibody comprising culturing any one of the host cells described herein under conditions, wherein the heterodimeric antibody is expressed, and recovering the heterodimeric antibody.

In another aspect, described herein is a heterodimeric antibody comprising: a) a first monomer comprising: i) an anti-B7H3 scFv comprising a first variable heavy VH1 domain, an scFv linker and a first variable light VL1 domain; and ii) a first Fc domain, wherein the scFv is covalently attached to the N-terminus of the first Fc domain using a domain linker; b) a second monomer comprising a VH2-CH1-hinge-CH2-CH3 monomer, wherein VH2 is a second variable heavy domain and CH2-CH3 is a second Fc domain; and c) a light chain comprising a second variable light VL2 domain, wherein the second variable heavy domain and the second variable light domain form an NKG2D antigen binding domain, and wherein the first Fc domain and/or the second Fc domain comprise an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering and have enhanced FcγRIIIA (CD16a) binding compared to first and second Fc domains lacking such substitution(s).

In some embodiments, the NKG2D antigen binding domain comprises a set of vhCDR1-3 and the vlCDR1-3 from a variable heavy domain and variable light domain pair selected from the group consisting of SEQ ID NOS: 2604-2606 for vhCDR1-3 and SEQ ID NOS: 2608-2610 for vlCDR1-3 of mAb-C[NKG2D]; SEQ ID NOS: 2612-2614 for vhCDR1-3 and SEQ ID NOS: 2616-2618 for vlCDR1-3 of mAb-D[NKG2D]; SEQ ID NOS: 17-18 and 1256 for vhCDR1-3 of 1D7B4[NKG2D]_H1.23 and SEQ ID NOS: 23, 24, and 26 for vlCDR1-3 of 1D7B4[NKG2D]_L1; SEQ ID NOS: 17-18 and 1272 for vhCDR1-3 of 1D7B4[NKG2D]_H1.31 and SEQ ID NOS: 23, 24, and 26 for vlCDR1-3 of 1D7B4[NKG2D]_L1; SEQ ID NOS: 17-19 for vhCDR1-3 and SEQ ID NOS: 23, 24, and 26 for vlCDR1-3 of 1D7B4[NKG2D]_H1_L1; SEQ ID NOS: 33-35 for vhCDR1-3 and SEQ ID NOS: 23, 24, and 26 for vlCDR1-3 of 1D2B4[NKG2D]_H1_L1; SEQ ID NOS: 1212 and 18-19 for vhCDR1-3 of 1D7B4[NKG2D]_H1.1 and SEQ ID NOS: 23, 24, and 26 for vlCDR1-3; SEQ ID NOS: 1214 and 18-19 for vhCDR1-3 of 1D7B4[NKG2D]_H1.2 and SEQ ID NOS: 23, 24, and 26 for vlCDR1-3; SEQ ID NOS: 1216 and 18-19 for vhCDR1-3 of 1D7B4[NKG2D]_H1.3 and SEQ ID NOS: 23, 24, and 26 for vlCDR1-3; SEQ ID NOS: 1218 and 18-19 for vhCDR1-3 of 1D7B4[NKG2D]_H1.4 and SEQ ID NOS: 23, 24, and 26 for vlCDR1-3; SEQ ID NOS: 1220 and 18-19 for vhCDR1-3 of 1D7B4[NKG2D]_H1.5 and SEQ ID NOS: 23, 24, and 26 for vlCDR1-3; SEQ ID NOS: 1222 and 18-19 for vhCDR1-3 of 1D7B4[NKG2D]_H1.6 and SEQ ID NOS: 23, 24, and 26 for vlCDR1-3; SEQ ID NOS: 1224 and 18-19 for vhCDR1-3 of 1D7B4[NKG2D]_H1.7 and SEQ ID NOS: 23, 24, and 26 for vlCDR1-3; SEQ ID NOS: 1226 and 18-19 for vhCDR1-3 of 1D7B4[NKG2D]_H1.8 and SEQ ID NOS: 23, 24, and 26 for vlCDR1-3; SEQ ID NOS: 17-18 and 1228 for vhCDR1-3 of 1D7B4[NKG2D]_H1.9 and SEQ ID NOS: 23, 24, and 26 for vlCDR1-3; SEQ ID NOS: 17-18 and 1230 for vhCDR1-3 of 1D7B4[NKG2D]_H1.10 and SEQ ID NOS: 23, 24, and 26 for vlCDR1-3; SEQ ID NOS: 17-18 and 1232 for vhCDR1-3 of 1D7B4[NKG2D]_H1.11 and SEQ ID NOS: 23, 24, and 26 for vlCDR1-3; SEQ ID NOS: 12-18 and 1234 for vhCDR1-3 of 1D7B4[NKG2D]_H1.12 and SEQ ID NOS: 23, 24, and 26 for vlCDR1-3; SEQ ID NOS: 12-18 and 1236 for vhCDR1-3 of 1D7B4[NKG2D]_H1.13 and SEQ ID NOS: 23, 24, and 26 for vlCDR1-3; SEQ ID NOS: 17-18 and 1238 for vhCDR1-3 of 1D7B4[NKG2D]_H1.14 and SEQ ID NOS: 23, 24, and 26 for vlCDR1-3; SEQ ID NOS: 17-18 and 1240 for vhCDR1-3 of 1D7B4[NKG2D]_H1.15 and SEQ ID NOS: 23, 24, and 26 for vlCDR1-3; SEQ ID NOS: 17-18 and 1242 for vhCDR1-3 of 1D7B4[NKG2D]_H1.16 and SEQ ID NOS: 23, 24, and 26 for vlCDR1-3; SEQ ID NOS: 17-18 and 1244 for vhCDR1-3 of 1D7B4[NKG2D]_H1.17 and SEQ ID NOS: 23, 24, and 26 for vlCDR1-3; SEQ ID NOS: 17-18 and 1246 for vhCDR1-3 of 1D7B4[NKG2D]_H1.18 and SEQ ID NOS: 23, 24, and 26 for vlCDR1-3; SEQ ID NOS: 17-18 and 1248 for vhCDR1-3 of 1D7B4[NKG2D]_H1.19 and SEQ ID NOS: 23, 24, and 26 for vlCDR1-3; SEQ ID NOS: 17-18 and 1250 for vhCDR1-3 of 1D7B4[NKG2D]_H1.20 and SEQ ID NOS: 23, 24, and 26 for vlCDR1-3; SEQ ID NOS: 17-18 and 1252 for vhCDR1-3 of 1D7B4[NKG2D]_H1.21 and SEQ ID NOS: 23, 24, and 26 for vlCDR1-3; SEQ ID NOS: 17-18 and 1254 for vhCDR1-3 of 1D7B4[NKG2D]_H1.22 and SEQ ID NOS: 23, 24, and 26 for vlCDR1-3; SEQ ID NOS: 17-18 and 1258 for vhCDR1-3 of 1D7B4[NKG2D]_H1.24 and SEQ ID NOS: 23, 24, and 26 for vlCDR1-3; SEQ ID NOS: 17-18 and 1260 for vhCDR1-3 of 1D7B4[NKG2D]_H1.25 and SEQ ID NOS: 23, 24, and 26 for vlCDR1-3; SEQ ID NOS: 17-18 and 1262 for vhCDR1-3 of 1D7B4[NKG2D]_H1.26 and SEQ ID NOS: 23, 24, and 26 for vlCDR1-3; SEQ ID NOS: 17-18 and 1264 for vhCDR1-3 of 1D7B4[NKG2D]_H1.27 and SEQ ID NOS: 23, 24, and 26 for vlCDR1-3; SEQ ID NOS: 17-18 and 1266 for vhCDR1-3 of 1D7B4[NKG2D]_H1.28 and SEQ ID NOS: 23, 24, and 26 for vlCDR1-3; SEQ ID NOS: 17-18 and 1268 for vhCDR1-3 of 1D7B4[NKG2D]_H1.29 and SEQ ID NOS: 23, 24, and 26 for vlCDR1-3; SEQ ID NOS: 17-18 and 1270 for vhCDR1-3 of 1D7B4[NKG2D]_H1.30 and SEQ ID NOS: 23, 24, and 26 for vlCDR1-3; SEQ ID NOS: 17-18 and 1274 for vhCDR1-3 of 1D7B4[NKG2D]_H1.32 and SEQ ID NOS: 23, 24, and 26 for vlCDR1-3; SEQ ID NOS: 17-18 and 1276 for vhCDR1-3 of 1D7B4[NKG2D]_H1.33 and SEQ ID NOS: 23, 24, and 26 for vlCDR1-3; SEQ ID NOS: 17-18 and 1278 for vhCDR1-3 of 1D7B4[NKG2D]_H1.34 and SEQ ID NOS: 23, 24, and 26 for vlCDR1-3; SEQ ID NOS: 17-18 and 1280 for vhCDR1-3 of 1D7B4[NKG2D]_H1.35 and SEQ ID NOS: 23, 24, and 26 for vlCDR1-3; SEQ ID NOS: 17-18 and 1282 for vhCDR1-3 of 1D7B4[NKG2D]_H1.36 and SEQ ID NOS: 23, 24, and 26 for vlCDR1-3; SEQ ID NOS: 17-18 and 1284 for vhCDR1-3 of 1D7B4[NKG2D]_H1.37 and SEQ ID NOS: 23, 24, and 26 for vlCDR1-3; SEQ ID NOS: 17-18 and 1286 for vhCDR1-3 of 1D7B4[NKG2D]_H1.38 and SEQ ID NOS: 23, 24, and 26 for vlCDR1-3; SEQ ID NOS: 17-18 and 1288 for vhCDR1-3 of 1D7B4[NKG2D]_H1.39 and SEQ ID NOS: 23, 24, and 26 for vlCDR1-3; SEQ ID NOS: 17-18 and 1290 for vhCDR1-3 of 1D7B4[NKG2D]_H1.40 and SEQ ID NOS: 23, 24, and 26 for vlCDR1-3; SEQ ID NOS: 17-18 and 1292 for vhCDR1-3 of 1D7B4[NKG2D]_H1.41 and SEQ ID NOS: 23, 24, and 26 for vlCDR1-3; SEQ ID NOS: 17-18 and 1294 for vhCDR1-3 of 1D7B4[NKG2D]_H1.42 and SEQ ID NOS: 23, 24, and 26 for vlCDR1-3; SEQ ID NOS: 17-18 and 1296 for vhCDR1-3 of 1D7B4[NKG2D]_H1.43 and SEQ ID NOS: 23, 24, and 26 for vlCDR1-3; SEQ ID NOS: 17-18 and 1298 for vhCDR1-3 of 1D7B4[NKG2D]_H1.44 and SEQ ID NOS: 23, 24, and 26 for vlCDR1-3; SEQ ID NOS: 17-18 and 1300 for vhCDR1-3 of 1D7B4[NKG2D]_H1.45 and SEQ ID NOS: 23, 24, and 26 for vlCDR1-3; SEQ ID NOS: 17-18 and 1302 for vhCDR1-3 of 1D7B4[NKG2D]_H1.46 and SEQ ID NOS: 23, 24, and 26 for vlCDR1-3; SEQ ID NOS17-18 and 1304 for vhCDR1-3 of 1D7B4[NKG2D]_H1.47 and SEQ ID NOS: 23, 24, and 26 for vlCDR1-3; and SEQ ID NOS: 17-18 and 1306 for vhCDR1-3 of 1D7B4[NKG2D]_H1.48 and SEQ ID NOS: 23, 24, and 26 for vlCDR1-3, as depicted in FIGS. 23 and 58.

In some embodiments, the NKG2D antigen binding domain comprises a variable heavy domain and variable light domain pair selected from the group consisting of SEQ ID NOS: 2603 and 2607 of mAb-C[NKG2D]; SEQ ID NOS: 2611 and 2615 of mAb-D[NKG2D]; SEQ ID NOS: 1255 and 51 of 1D7B4[NKG2D]_H1.23_L1; SEQ ID NOS: 1271 and 51 of 1D7B4[NKG2D]_H1.31_L1; SEQ ID NOS: 50 and 51 of 1D7B4[NKG2D]_H1_L1; SEQ ID NOS: 52 and 51 of 1D2B4[NKG2D]; SEQ ID NOS: 1211 and 51 of 1D7B4[NKG2D]_H1.1_L1; SEQ ID NOS: 1213 and 51 of 1D7B4[NKG2D]_H1.2_L1; SEQ ID NOS: 1215 and 51 of 1D7B4[NKG2D]_H1.3_L1; SEQ ID NOS: 1217 and 51 of 1D7B4[NKG2D]_H1.4_L1; SEQ ID NOS: 1219 and 51 of 1D7B4[NKG2D]_H1.5_L1; SEQ ID NOS: 1221 and 51 of 1D7B4[NKG2D]_H1.6_L1; SEQ ID NOS: 1223 and 51 of 1D7B4[NKG2D]_H1.7_L1; SEQ ID NOS: 1225 and 51 of 1D7B4[NKG2D]_H1.8_L1; SEQ ID NOS: 1227 and 51 of 1D7B4[NKG2D]_H1.9_L1; SEQ ID NOS: 1229 and 51 of 1D7B4[NKG2D]_H1.10_L1; SEQ ID NOS: 1231 and 51 of 1D7B4[NKG2D]_H1.11_L1; SEQ ID NOS: 1233 and 51 of 1D7B4[NKG2D]_H1.12_L1; SEQ ID NOS: 1235 and 51 of 1D7B4[NKG2D]_H1.13_L1; SEQ ID NOS: 1237 and 51 of 1D7B4[NKG2D]_H1.14_L1; SEQ ID NOS: 1239 and 51 of 1D7B4[NKG2D]_H1.15_L1; SEQ ID NOS: 1241 and 51 of 1D7B4[NKG2D]_H1.16_L1; SEQ ID NOS: 1243 and 51 of 1D7B4[NKG2D]_H1.17_L1; SEQ ID NOS: 1245 and 51 of 1D7B4[NKG2D]_H1.18_L1; SEQ ID NOS: 1247 and 51 of 1D7B4[NKG2D]_H1.19_L1; SEQ ID NOS: 1249 and 51 of 1D7B4[NKG2D]_H1.20_L1; SEQ ID NOS: 1251 and 51 of 1D7B4[NKG2D]_H1.21_L1; SEQ ID NOS: 1253 and 51 of 1D7B4[NKG2D]_H1.22_L1; SEQ ID NOS: 1257 and 51 of 1D7B4[NKG2D]_H1.24_L1; SEQ ID NOS: 1259 and 51 of 1D7B4[NKG2D]_H1.25_L1; SEQ ID NOS: 1261 and 51 of 1D7B4[NKG2D]_H1.26_L1; SEQ ID NOS: 1263 and 51 of 1D7B4[NKG2D]_H1.27_L1; SEQ ID NOS: 1265 and 51 of 1D7B4[NKG2D]_H1.28_L1; SEQ ID NOS: 1267 and 51 of 1D7B4[NKG2D]_H1.29_L1; SEQ ID NOS: 1269 and 51 of 1D7B4[NKG2D]_H1.30_L1; SEQ ID NOS: 1273 and 51 of 1D7B4[NKG2D]_H1.32_L1; SEQ ID NOS: 1275 and 51 of 1D7B4[NKG2D]_H1.33_L1; SEQ ID NOS: 1277 and 51 of 1D7B4[NKG2D]_H1.34_L1; SEQ ID NOS: 1279 and 51 of 1D7B4[NKG2D]_H1.35_L1; SEQ ID NOS: 1281 and 51 of 1D7B4[NKG2D]_H1.36_L1; SEQ ID NOS: 1283 and 51 of 1D7B4[NKG2D]_H1.37_L1; SEQ ID NOS: 1285 and 51 of 1D7B4[NKG2D]_H1.38_L1; SEQ ID NOS: 1287 and 51 of 1D7B4[NKG2D]_H1.39_L1; SEQ ID NOS: 1289 and 51 of 1D7B4[NKG2D]_H1.40_L1; SEQ ID NOS: 1291 and 51 of 1D7B4[NKG2D]_H1.41_L1; SEQ ID NOS: 1293 and 51 of 1D7B4[NKG2D]_H1.42_L1; SEQ ID NOS: 1295 and 51 of 1D7B4[NKG2D]_H1.43_L1; SEQ ID NOS: 1297 and 51 of 1D7B4[NKG2D]_H1.44_L1; SEQ ID NOS: 1299 and 51 of 1D7B4[NKG2D]_H1.45_L1; SEQ ID NOS: 1301 and 51 of 1D7B4[NKG2D]_H1.46_L1; SEQ ID NOS: 1303 and 51 of 1D7B4[NKG2D]_H1.47_L1; and SEQ ID NOS: 1305 and 51 of 1D7B4[NKG2D]_H1.48_L1, as depicted in FIGS. 23 and 58.

In some embodiments, the anti-B7H3 scFv comprises a set of vhCDR1-3 and vlCDR1-3 from a variable heavy domain and variable light domain pair, wherein the set of vhCDR1-3 and vlCDR1-3 is selected from the group consisting of SEQ ID NOS: 27, 28, and 29 for vhCDR1-3 and SEQ ID NOS: 30, 31, and 32 for vlCDR1-3 of 38E2[B7H3]_H2_L1.1; SEQ ID NOS: 243, 244, and 245 for vhCDR1-3 and SEQ ID NOS: 247, 248, and 249 for vlCDR1-3 of 6A1[B7H3]_H1_L1; SEQ ID NOS: 143, 144, and 22 for vhCDR1-3 and SEQ ID NOS: 23, 24, and 26 for vlCDR1-3 of 2E43.189[B7H3]_H1_L1, and SEQ ID NOS: 20, 21, and 22 for vhCDR1-3 and SEQ ID NOS: 23, 24, and 26 for vlCDR1-3 of 2E43.189[B7H3]_H1.22_L1, as depicted in FIGS. 13 and 14.

In some embodiments, the anti-B7H3 scFv comprises a variable heavy domain and variable light domain pair selected from the group consisting of SEQ ID NOS: 140 and 141 of 38E2[B7H3]_H2_L1.1; SEQ ID NOS: 242 and 246 of 6A1[B7H3]_H1L1; SEQ ID NOS: 142 and 51 of 2E4A3.189[B7H3] H1_L1; and SEQ ID NOS: 145 and 51 of 2E4A3.189[B7H3]H1.22_L1, as depicted in FIGS. 13 and 14.

In some embodiments, the first variable light domain of the anti-B7H3 scFv is covalently attached to the N-terminus of the first Fc domain using a domain linker.

In some embodiments, the first variable heavy domain of the anti-B7H3 scFv is covalently attached to the N-terminus of the first Fc domain using a domain linker.

In some embodiments, the scFv linker is a charged scFv linker.

In some embodiments, the scFv linker is a charged scFv linker having the amino acid sequence (GKPGS)4 (SEQ ID NO:96).

In some embodiments, the first domain comprises an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

In some embodiments, the second domain comprises an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

In some embodiments, the first and second Fc domains comprise a set of amino acid substitutions selected from the group consisting of: S239D/I332E:S239D/I332E; S239D:S239D; I332E:I332E; WT:S239D/I332E; WT:S239D; WT:I332E; S239D/I332E:WT; S239D:WT; I332E:WT; S239D/I332E:S239D; S239D/I332E:I332E; S239D:S239D/I332E; I332E:S239D/I332E; S239D:I332E; and I332E:S239D, wherein numbering is according to EU numbering.

In some embodiments, the first or second Fc domain comprises the amino acid substitutions S239D/I332E, wherein numbering is according to EU numbering.

In some embodiments, the first and second Fc domains further comprise a set of heterodimerization variants selected from the group consisting of those depicted in FIGS. 1A-1E, wherein numbering is according to EU numbering.

In some embodiments, the set of heterodimerization variants selected is from the group consisting of S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering.

In some embodiments, the first or second Fc domain further comprises one or more pI variants.

In some embodiments, the one or more pI variants are N208D/Q295E/N384D/Q418E/N421D, wherein numbering is according to EU numbering.

In some embodiments, the first and second monomers each further comprise amino acid substitutions selected from the group consisting of M428L/N434S, M428L/N434A, and M252Y/S254T/T256E, wherein numbering is according to EU numbering.

In some embodiments, the heterodimeric antibody described herein is selected from the group consisting of: the amino acid sequences of SEQ ID NOS:1, 2 and 3 of XENP38597; the amino acid sequences of SEQ ID NOS: 4, 5 and 6 of XENP40377; the amino acid sequences of SEQ ID NOS:7, 8 and 3 of XENP38101; the amino acid sequences of SEQ ID NOS: 9, 10 and 3 of XENP38108; the amino acid sequences of SEQ ID NOS: 11, 2 and 3 of XENP38596; the amino acid sequences of SEQ ID NOS: 12, 2, and 13 of XENP38598; the amino acid sequences of SEQ ID NOS:14, 2 and 15 of XENP38599; the amino acid sequences of SEQ ID NOS:4, 16 and 6 of XENP40374; the amino acid sequences of SEQ ID NOS: 1307-1308 and 6 of XENP42652; the amino acid sequences of SEQ ID NOS: 1309-1310 and 6 of XENP42653; the amino acid sequences of SEQ ID NOS: 1311-1312 and 6 of XENP42654; the amino acid sequences of SEQ ID NOS: 1313-1314 and 6 of XENP42655; and the amino acid sequences of SEQ ID NOS: 1315-1316 and 6 of XENP42656; as depicted in FIGS. 19 and 59.

Also provided is a nucleic acid composition comprising nucleic acids encoding the first and second monomers and the light chain of any of the antibodies described.

Also provided is a expression vector comprising the nucleic acids described herein.

Additionally, provided is a host cell transformed with any of the expression vectors described herein.

In some embodiments, provided is a method of making a heterodimeric antibody comprising culturing any one of the host cells described herein under conditions, wherein the heterodimeric antibody is expressed, and recovering the heterodimeric antibody.

In a further aspect, a heterodimeric antibody comprising:

a) a first monomer comprising, from N-terminus to C-terminus, a VH1-CH1-first linker-scFv-second linker-CH2-CH3, wherein the VH1 is a first variable heavy domain, the scFv is an anti-NKG2D scFv, and the CH2-CH3 is a first Fc domain; b) a second monomer comprising, from N-terminus to C-terminus, a VH2-CH1-hinge-CH2-CH3, wherein the VH2 is a second variable heavy domain and the CH2-CH3 is a second Fc domain; and c) a common light chain comprising from N- to C-terminus, VL-CL, wherein the VL is a variable light domain and the CL is a light chain constant domain; wherein the first variable heavy VH1 domain and the variable light VL domain form a first B7H3 antigen binding domain, and the second variable heavy VH2 domain and the variable light VL domain form a second B7H3 antigen binding domain, and wherein the first Fc domain and/or the second Fc domain comprise an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering and have enhanced FcγRIIIA (CD16a) binding compared to first and second Fc domains lacking such substitution(s).

In some embodiments, the anti-NKG2D scFv comprises a variable heavy domain, an scFv linker and a variable light domain.

In some embodiments, the anti-NKG2D scFv comprises a set of vhCDR1-3 and vlCDR1-3 from a variable heavy domain and variable light domain pair selected from the group consisting of SEQ ID NOS: 2604-2606 for vhCDR1-3 and SEQ ID NOS: 2608-2610 for vlCDR1-3 of mAb-C[NKG2D]; SEQ ID NOS: 2612-2614 for vhCDR1-3 and SEQ ID NOS: 2616-2618 for vlCDR1-3 of mAb-D[NKG2D]; SEQ ID NOS: 17-18 and 1256 for vhCDR1-3 of 1D7B4[NKG2D]_H1.23 and SEQ ID NOS: 23, 24, and 26 for vlCDR1-3 of 1D7B4[NKG2D]_L1; SEQ ID NOS: 17-18 and 1272 for vhCDR1-3 of 1D7B4[NKG2D]_H1.31 and SEQ ID NOS: 23, 24, and 26 for vlCDR1-3 of 1D7B4[NKG2D]_L1; SEQ ID NOS: 17-19 for vhCDR1-3 and SEQ ID NOS: 23, 24, and 26 for vlCDR1-3 of 1D7B4[NKG2D]_H1_L1; SEQ ID NOS: 33-35 for vhCDR1-3 and SEQ ID NOS: 23, 24, and 26 for vlCDR1-3 of 1D2B4[NKG2D]_H1_L1, as depicted in FIG. 23; SEQ ID NOS: 1212 and 18-19 for vhCDR1-3 of 1D7B4[NKG2D]_H1.1 and SEQ ID NOS: 23, 24, and 26 for vlCDR1-3; SEQ ID NOS: 1214 and 18-19 for vhCDR1-3 of 1D7B4[NKG2D]_H1.2 and SEQ ID NOS: 23, 24, and 26 for vlCDR1-3; SEQ ID NOS: 1216 and 18-19 for vhCDR1-3 of 1D7B4[NKG2D]_H1.3 and SEQ ID NOS: 23, 24, and 26 for vlCDR1-3; SEQ ID NOS: 1218 and 18-19 for vhCDR1-3 of 1D7B4[NKG2D]_H1.4 and SEQ ID NOS: 23, 24, and 26 for vlCDR1-3; SEQ ID NOS: 1220 and 18-19 for vhCDR1-3 of 1D7B4[NKG2D]_H1.5 and SEQ ID NOS: 23, 24, and 26 for vlCDR1-3; SEQ ID NOS: 1222 and 18-19 for vhCDR1-3 of 1D7B4[NKG2D]_H1.6 and SEQ ID NOS: 23, 24, and 26 for vlCDR1-3; SEQ ID NOS: 1224 and 18-19 for vhCDR1-3 of 1D7B4[NKG2D]_H1.7 and SEQ ID NOS: 23, 24, and 26 for vlCDR1-3; SEQ ID NOS: 1226 and 18-19 for vhCDR1-3 of 1D7B4[NKG2D]_H1.8 and SEQ ID NOS: 23, 24, and 26 for vlCDR1-3; SEQ ID NOS: 17-18 and 1228 for vhCDR1-3 of 1D7B4[NKG2D]_H1.9 and SEQ ID NOS: 23, 24, and 26 for vlCDR1-3; SEQ ID NOS: 17, 18 and 1230 for vhCDR1-3 of 1D7B4[NKG2D]_H1.10 and SEQ ID NOS: 23, 24, and 26 for vlCDR1-3; SEQ ID NOS: 17-18 and 1232 for vhCDR1-3 of 1D7B4[NKG2D]_H1.11 and SEQ ID NOS: 23, 24, and 26 for vlCDR1-3; SEQ ID NOS: 12-18 and 1234 for vhCDR1-3 of 1D7B4[NKG2D]_H1.12 and SEQ ID NOS: 23, 24, and 26 for vlCDR1-3; SEQ ID NOS: 12-18 and 1236 for vhCDR1-3 of 1D7B4[NKG2D]_H1.13 and SEQ ID NOS: 23, 24, and 26 for vlCDR1-3; SEQ ID NOS: 17-18 and 1238 for vhCDR1-3 of 1D7B4[NKG2D]_H1.14 and SEQ ID NOS: 23, 24, and 26 for vlCDR1-3; SEQ ID NOS: 17-18 and 1240 for vhCDR1-3 of 1D7B4[NKG2D]_H1.15 and SEQ ID NOS: 23, 24, and 26 for vlCDR1-3; SEQ ID NOS: 17-18 and 1242 for vhCDR1-3 of 1D7B4[NKG2D]_H1.16 and SEQ ID NOS: 23, 24, and 26 for vlCDR1-3; SEQ ID NOS: 17-18 and 1244 for vhCDR1-3 of 1D7B4[NKG2D]_H1.17 and SEQ ID NOS: 23, 24, and 26 for vlCDR1-3; SEQ ID NOS: 17-18 and 1246 for vhCDR1-3 of 1D7B4[NKG2D]_H1.18 and SEQ ID NOS: 23, 24, and 26 for vlCDR1-3; SEQ ID NOS: 17-18 and 1248 for vhCDR1-3 of 1D7B4[NKG2D]_H1.19 and SEQ ID NOS: 23, 24, and 26 for vlCDR1-3; SEQ ID NOS: 17-18 and 1250 for vhCDR1-3 of 1D7B4[NKG2D]_H1.20 and SEQ ID NOS: 23, 24, and 26 for vlCDR1-3; SEQ ID NOS: 17-18 and 1252 for vhCDR1-3 of 1D7B4[NKG2D]_H1.21 and SEQ ID NOS: 23, 24, and 26 for vlCDR1-3; SEQ ID NOS: 17-18 and 1254 for vhCDR1-3 of 1D7B4[NKG2D]_H1.22 and SEQ ID NOS: 23, 24, and 26 for vlCDR1-3; SEQ ID NOS: 17-18 and 1258 for vhCDR1-3 of 1D7B4[NKG2D]_H1.24 and SEQ ID NOS: 23, 24, and 26 for vlCDR1-3; SEQ ID NOS: 17-18 and 1260 for vhCDR1-3 of 1D7B4[NKG2D]_H1.25 and SEQ ID NOS: 23, 24, and 26 for vlCDR1-3; SEQ ID NOS: 17-18 and 1262 for vhCDR1-3 of 1D7B4[NKG2D]_H1.26 and SEQ ID NOS: 23, 24, and 26 for vlCDR1-3; SEQ ID NOS: 17-18 and 1264 for vhCDR1-3 of 1D7B4[NKG2D]_H1.27 and SEQ ID NOS: 23, 24, and 26 for vlCDR1-3; SEQ ID NOS: 17-18 and 1266 for vhCDR1-3 of 1D7B4[NKG2D]_H1.28 and SEQ ID NOS: 23, 24, and 26 for vlCDR1-3; SEQ ID NOS: 17-18 and 1268 for vhCDR1-3 of 1D7B4[NKG2D]_H1.29 and SEQ ID NOS: 23, 24, and 26 for vlCDR1-3; SEQ ID NOS: 17-18 and 1270 for vhCDR1-3 of 1D7B4[NKG2D]_H1.30 and SEQ ID NOS: 23, 24, and 26 for vlCDR1-3; SEQ ID NOS: 17-18 and 1274 for vhCDR1-3 of 1D7B4[NKG2D]_H1.32 and SEQ ID NOS: 23, 24, and 26 for vlCDR1-3; SEQ ID NOS: 17-18 and 1276 for vhCDR1-3 of 1D7B4[NKG2D]_H1.33 and SEQ ID NOS: 23, 24, and 26 for vlCDR1-3; SEQ ID NOS: 17-18 and 1278 for vhCDR1-3 of 1D7B4[NKG2D]_H1.34 and SEQ ID NOS: 23, 24, and 26 for vlCDR1-3; SEQ ID NOS: 17-18 and 1280 for vhCDR1-3 of 1D7B4[NKG2D]_H1.35 and SEQ ID NOS: 23, 24, and 26 for vlCDR1-3; SEQ ID NOS: 17-18 and 1282 for vhCDR1-3 of 1D7B4[NKG2D]_H1.36 and SEQ ID NOS: 23, 24, and 26 for vlCDR1-3; SEQ ID NOS: 17-18 and 1284 for vhCDR1-3 of 1D7B4[NKG2D]_H1.37 and SEQ ID NOS: 23, 24, and 26 for vlCDR1-3; SEQ ID NOS: 17-18 and 1286 for vhCDR1-3 of 1D7B4[NKG2D]_H1.38 and SEQ ID NOS: 23, 24, and 26 for vlCDR1-3; SEQ ID NOS: 17-18 and 1288 for vhCDR1-3 of 1D7B4[NKG2D]_H1.39 and SEQ ID NOS: 23, 24, and 26 for vlCDR1-3; SEQ ID NOS: 17-18 and 1290 for vhCDR1-3 of 1D7B4[NKG2D]_H1.40 and SEQ ID NOS: 23, 24, and 26 for vlCDR1-3; SEQ ID NOS: 17-18 and 1292 for vhCDR1-3 of 1D7B4[NKG2D]_H1.41 and SEQ ID NOS: 23, 24, and 26 for vlCDR1-3; SEQ ID NOS: 17-18 and 1294 for vhCDR1-3 of 1D7B4[NKG2D]_H1.42 and SEQ ID NOS: 23, 24, and 26 for vlCDR1-3; SEQ ID NOS: 17-18 and 1296 for vhCDR1-3 of 1D7B4[NKG2D]_H1.43 and SEQ ID NOS: 23, 24, and 26 for vlCDR1-3; SEQ ID NOS: 17-18 and 1298 for vhCDR1-3 of 1D7B4[NKG2D]_H1.44 and SEQ ID NOS: 23, 24, and 26 for vlCDR1-3; SEQ ID NOS: 17-18 and 1300 for vhCDR1-3 of 1D7B4[NKG2D]_H1.45 and SEQ ID NOS: 23, 24, and 26 for vlCDR1-3; SEQ ID NOS: 17-18 and 1302 for vhCDR1-3 of 1D7B4[NKG2D]_H1.46 and SEQ ID NOS: 23, 24, and 26 for vlCDR1-3; SEQ ID NOS17-18 and 1304 for vhCDR1-3 of 1D7B4[NKG2D]_H1.47 and SEQ ID NOS: 23, 24, and 26 for vlCDR1-3; and SEQ ID NOS: 17-18 and 1306 for vhCDR1-3 of 1D7B4[NKG2D]_H1.48 and SEQ ID NOS: 23, 24, and 26 for vlCDR1-3, as depicted in FIGS. 23 and 58.

In some embodiments, the anti-NKG2D scFv comprises a variable heavy domain and variable light domain pair selected from the group consisting of SEQ ID NOS: 2603 and 2607 of mAb-C[NKG2D]; SEQ ID NOS: 2611 and 2615 of mAb-D[NKG2D]; SEQ ID NOS: 1255 and 51 of 1D7B4[NKG2D]_H1.23_L1; SEQ ID NOS: 1271 and 51 of 1D7B4[NKG2D]_H1.31_L1; SEQ ID NOS: 50 and 51 of 1D7B4[NKG2D]_H1_L1; SEQ ID NOS: 52 and 51 of 1D2B4[NKG2D]; SEQ ID NOS: 1211 and 51 of 1D7B4[NKG2D]_H1.1_L1; SEQ ID NOS: 1213 and 51 of 1D7B4[NKG2D]_H1.2_L1; SEQ ID NOS: 1215 and 51 of 1D7B4[NKG2D]_H1.3_L1; SEQ ID NOS: 1217 and 51 of 1D7B4[NKG2D]_H1.4_L1; SEQ ID NOS: 1219 and 51 of 1D7B4[NKG2D]_H1.5_L1; SEQ ID NOS: 1221 and 51 of 1D7B4[NKG2D]_H1.6_L1; SEQ ID NOS: 1223 and 51 of 1D7B4[NKG2D]_H1.7_L1; SEQ ID NOS: 1225 and 51 of 1D7B4[NKG2D]_H1.8_L1; SEQ ID NOS: 1227 and 51 of 1D7B4[NKG2D]_H1.9_L1; SEQ ID NOS: 1229 and 51 of 1D7B4[NKG2D]_H1.10_L1; SEQ ID NOS: 1231 and 51 of 1D7B4[NKG2D]_H1.11_L1; SEQ ID NOS: 1233 and 51 of 1D7B4[NKG2D]_H1.12_L1; SEQ ID NOS: 1235 and 51 of 1D7B4[NKG2D]_H1.13_L1; SEQ ID NOS: 1237 and 51 of 1D7B4[NKG2D]_H1.14_L1; SEQ ID NOS: 1239 and 51 of 1D7B4[NKG2D]_H1.15_L1; SEQ ID NOS: 1241 and 51 of 1D7B4[NKG2D]_H1.16_L1; SEQ ID NOS: 1243 and 51 of 1D7B4[NKG2D]_H1.17_L1; SEQ ID NOS: 1245 and 51 of 1D7B4[NKG2D]_H1.18_L1; SEQ ID NOS: 1247 and 51 of 1D7B4[NKG2D]_H1.19_L1; SEQ ID NOS: 1249 and 51 of 1D7B4[NKG2D]_H1.20_L1; SEQ ID NOS: 1251 and 51 of 1D7B4[NKG2D]_H1.21_L1; SEQ ID NOS: 1253 and 51 of 1D7B4[NKG2D]_H1.22_L1; SEQ ID NOS: 1257 and 51 of 1D7B4[NKG2D]_H1.24_L1; SEQ ID NOS: 1259 and 51 of 1D7B4[NKG2D]_H1.25_L1; SEQ ID NOS: 1261 and 51 of 1D7B4[NKG2D]_H1.26_L1; SEQ ID NOS: 1263 and 51 of 1D7B4[NKG2D]_H1.27_L1; SEQ ID NOS: 1265 and 51 of 1D7B4[NKG2D]_H1.28_L1; SEQ ID NOS: 1267 and 51 of 1D7B4[NKG2D]_H1.29_L1; SEQ ID NOS: 1269 and 51 of 1D7B4[NKG2D]_H1.30_L1; SEQ ID NOS: 1273 and 51 of 1D7B4[NKG2D]_H1.32_L1; SEQ ID NOS: 1275 and 51 of 1D7B4[NKG2D]_H1.33_L1; SEQ ID NOS: 1277 and 51 of 1D7B4[NKG2D]_H1.34_L1; SEQ ID NOS: 1279 and 51 of 1D7B4[NKG2D]_H1.35_L1; SEQ ID NOS: 1281 and 51 of 1D7B4[NKG2D]_H1.36_L1; SEQ ID NOS: 1283 and 51 of 1D7B4[NKG2D]_H1.37_L1; SEQ ID NOS: 1285 and 51 of 1D7B4[NKG2D]_H1.38_L1; SEQ ID NOS: 1287 and 51 of 1D7B4[NKG2D]_H1.39_L1; SEQ ID NOS: 1289 and 51 of 1D7B4[NKG2D]_H1.40_L1; SEQ ID NOS: 1291 and 51 of 1D7B4[NKG2D]_H1.41_L1; SEQ ID NOS: 1293 and 51 of 1D7B4[NKG2D]_H1.42_L1; SEQ ID NOS: 1295 and 51 of 1D7B4[NKG2D]_H1.43_L1; SEQ ID NOS: 1297 and 51 of 1D7B4[NKG2D]_H1.44_L1; SEQ ID NOS: 1299 and 51 of 1D7B4[NKG2D]_H1.45_L1; SEQ ID NOS: 1301 and 51 of 1D7B4[NKG2D]_H1.46_L1; SEQ ID NOS: 1303 and 51 of 1D7B4[NKG2D]_H1.47_L1; and SEQ ID NOS: 1305 and 51 of 1D7B4[NKG2D]_H1.48_L1, as depicted in FIGS. 23 and 58.

In some embodiments, the first and/or second B7H3 antigen binding domains comprise a set of vhCDR1-3 and vlCDR1-3 from a variable heavy domain and variable light domain pair, wherein the set of vhCDR1-3 and vlCDR1-3 is selected from the group consisting of SEQ ID NOS: 27, 28, and 29 for vhCDR1-3 and SEQ ID NOS: 30, 31, and 32 for vlCDR1-3 of 38E2[B7H3]_H2_L1.1; SEQ ID NOS:243, 244, and 245 for vhCDR1-3 and SEQ ID NOS:247, 248, and 249 for vlCDR1-3 of 6A1[B7H3]_H1_L1; SEQ ID NOS:143, 144, and 22 for vhCDR1-3 and SEQ ID NOS:23, 24, and 26 for vlCDR1-3 of 2E43.189[B7H3]_H1_L1; and SEQ ID NOS:20, 21, and 22 for vhCDR1-3 and SEQ ID NOS:23, 24, and 26 for vlCDR1-3 of 2E43.189[B7H3]_H1.22_L1, as depicted in FIGS. 13 and 14.

In some embodiments, the first and/or second B7H3 antigen binding domains comprise a variable heavy domain and variable light domain pair selected from the group consisting of SEQ ID NOS:140 and 141 of 38E2[B7H3]_H2_L1.1; SEQ ID NOS:242 and 246 of 6A1[B7H3]_H1L1; SEQ ID NOS:142 and 51 of 2E4A3.189[B7H3] H1_L1; and SEQ ID NOS:145 and 51 of 2E4A3.189[B7H3] H1.22_L1, as depicted in FIGS. 13 and 14.

In some embodiments, the scFv linker is a charged scFv linker.

In some embodiments, the scFv linker is a charged scFv linker having the amino acid sequence (GKPGS)4 (SEQ ID NO:96).

In some embodiments, the first and second linkers are each domain linkers.

In some embodiments, the first domain comprises an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

In some embodiments, the second domain comprises an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

In some embodiments, the first and second Fc domains comprise a set of amino acid substitutions selected from the group consisting of: S239D/I332E:S239D/I332E; S239D:S239D; I332E:I332E; WT:S239D/I332E; WT:S239D; WT:I332E; S239D/I332E:WT; S239D:WT; I332E:WT; S239D/I332E:S239D; S239D/I332E:I332E; S239D:S239D/I332E; I332E:S239D/I332E; S239D:I332E; and I332E:S239D, wherein numbering is according to EU numbering.

In some embodiments, the first or second Fc domain comprises the amino acid substitutions S239D/I332E, wherein numbering is according to EU numbering.

In some embodiments, the first and second Fc domains further comprise a set of heterodimerization variants selected from the group consisting of those depicted in FIGS. 1A-1E, wherein numbering is according to EU numbering.

In some embodiments, the set of heterodimerization variants selected is from the group consisting of S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering.

In some embodiments, the first or second Fc domain further comprises one or more pI variants.

In some embodiments, the one or more pI variants are N208D/Q295E/N384D/Q418E/N421D, wherein numbering is according to EU numbering.

In some embodiments, the first and second monomers each further comprise amino acid substitutions selected from the group consisting of M428L/N434S, M428L/N434A, and M252Y/S254T/T256E, wherein numbering is according to EU numbering.

The heterodimeric antibody according any one of claims 47-64 comprising the amino acid sequences of SEQ ID NOS:4, 48 and 6 of XENP40591, as depicted in FIG. 20.

Also provided is a nucleic acid composition comprising nucleic acids encoding the first and second monomers and the light chain of any of the antibodies described.

Also provided is a expression vector comprising the nucleic acids described herein.

Additionally, provided is a host cell transformed with any of the expression vectors described herein.

In some embodiments, provided is a method of making a heterodimeric antibody comprising culturing any one of the host cells described herein under conditions, wherein the heterodimeric antibody is expressed, and recovering the heterodimeric antibody.

Provided herein is a method of treating cancer in a subject comprising administering any of the heterodimeric antibodies described herein to a subject in need thereof.

In yet another aspect, provided herein is a heterodimeric antibody comprising: a) a first monomer comprising, from N-terminal to C-terminal, a VH1-CH1-hinge-CH2-CH3-domain linker-scFv, wherein VH1 is a first variable heavy domain, scFv is an anti-NKG2D scFv, and CH2-CH3 is a first Fc domain; b) a second monomer comprising, from N-terminal to C-terminal, a VH2-CH1-hinge-CH2-CH3, wherein CH2-CH3 is a second Fc domain; and c) a light chain comprising, from N-terminus to C-terminus, a VL1-CL, wherein VL1 is a first variable light domain and CL is a constant light domain, wherein the VH1 and the VL1 form a first B7H3 antigen binding domain, and the VH2 domain and the VL1 form a second B7H3 antigen binding domain, and wherein the anti-NKG2D scFv comprises a VH3 domain, a scFv linker, and a VL2 domain, and wherein the first Fc domain and/or the second Fc domain comprise an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering and have enhanced FcγRIIIA (CD16a) binding compared to first and second Fc domains lacking such substitution(s).

In some embodiments, the B7H3 antigen binding domain comprises a set of vhCDR1-3 and vlCDR1-3 from a variable heavy domain and variable light domain pair, wherein the set of vhCDR1-3 and vlCDR1-3 is selected from the group consisting of SEQ ID NOS: 27, 28, and 29 for vhCDR1-3 and SEQ ID NOS: 30, 31, and 32 for vlCDR1-3 of 38E2[B7H3]_H2_L1.1; SEQ ID NOS: 243, 244, and 245 for vhCDR1-3 and SEQ ID NOS: 247, 248, and 249 for vlCDR1-3 of 6A1[B7H3]_H1_L1; SEQ ID NOS: 143, 144, and 22 for vhCDR1-3 and SEQ ID NOS: 23, 24, and 26 for vlCDR1-3 of 2E43.189[B7H3]_H1_L1, and SEQ ID NOS: 20, 21, and 22 for vhCDR1-3 and SEQ ID NOS: 23, 24, and 26 for vlCDR1-3 of 2E43.189[B7H3]_H1.22_L1, as depicted in FIGS. 13 and 14.

In some embodiments, the B7H3 antigen binding domain comprises a variable heavy domain and variable light domain pair selected from the group consisting of SEQ ID NOS: 140 and 141 of 38E2[B7H3]_H2_L1.1; SEQ ID NOS: 242 and 246 of 6A1[B7H3]_H1_L1; SEQ ID NOS: 142 and 51 of 2E4A3.189[B7H3]_H1_L1; and SEQ ID NOS: 145 and 51 of 2E4A3.189[B7H3]_H1.22_L1, as depicted in FIGS. 13 and 14.

In some embodiments, the anti-NKG2D scFv comprises a set of vhCDR1-3 and vlCDR1-3 from a variable heavy domain and variable light domain pair selected from the group consisting of SEQ ID NOS: 2604-2606 for vhCDR1-3 and SEQ ID NOS: 2608-2610 for vlCDR1-3 of mAb-C[NKG2D]; SEQ ID NOS: 2612-2614 for vhCDR1-3 and SEQ ID NOS: 2616-2618 for vlCDR1-3 of mAb-D[NKG2D]; SEQ ID NOS: 17-18 and 1256 for vhCDR1-3 of 1D7B4[NKG2D]_H1.23 and SEQ ID NOS: 23, 24, and 26 for vlCDR1-3 of 1D7B4[NKG2D]_L1; SEQ ID NOS: 17-18 and 1272 for vhCDR1-3 of 1D7B4[NKG2D]_H1.31 and SEQ ID NOS: 23, 24, and 26 for vlCDR1-3 of 1D7B4[NKG2D]_L1; SEQ ID NOS: 17-19 for vhCDR1-3 and SEQ ID NOS: 23, 24, and 26 for vlCDR1-3 of 1D7B4[NKG2D]_H1_L1; SEQ ID NOS: 33-35 for vhCDR1-3 and SEQ ID NOS: 23, 24, and 26 for vlCDR1-3 of 1D2B4[NKG2D]_H1_L1; SEQ ID NOS: 1212 and 18-19 for vhCDR1-3 of 1D7B4[NKG2D]_H1.1 and SEQ ID NOS: 23, 24, and 26 for vlCDR1-3; SEQ ID NOS: 1214 and 18-19 for vhCDR1-3 of 1D7B4[NKG2D]_H1.2 and SEQ ID NOS: 23, 24, and 26 for vlCDR1-3; SEQ ID NOS: 1216 and 18-19 for vhCDR1-3 of 1D7B4[NKG2D]_H1.3 and SEQ ID NOS: 23, 24, and 26 for vlCDR1-3; SEQ ID NOS: 1218 and 18-19 for vhCDR1-3 of 1D7B4[NKG2D]_H1.4 and SEQ ID NOS: 23, 24, and 26 for vlCDR1-3; SEQ ID NOS: 1220 and 18-19 for vhCDR1-3 of 1D7B4[NKG2D]_H1.5 and SEQ ID NOS: 23, 24, and 26 for vlCDR1-3; SEQ ID NOS: 1222 and 18-19 for vhCDR1-3 of 1D7B4[NKG2D]_H1.6 and SEQ ID NOS: 23, 24, and 26 for vlCDR1-3; SEQ ID NOS: 1224 and 18-19 for vhCDR1-3 of 1D7B4[NKG2D]_H1.7 and SEQ ID NOS: 23, 24, and 26 for vlCDR1-3; SEQ ID NOS: 1226 and 18-19 for vhCDR1-3 of 1D7B4[NKG2D]_H1.8 and SEQ ID NOS: 23, 24, and 26 for vlCDR1-3; SEQ ID NOS: 17-18 and 1228 for vhCDR1-3 of 1D7B4[NKG2D]_H1.9 and SEQ ID NOS: 23, 24, and 26 for vlCDR1-3; SEQ ID NOS: 14-18 and 1230 for vhCDR1-3 of 1D7B4[NKG2D]_H1.10 and SEQ ID NOS: 23, 24, and 26 for vlCDR1-3; SEQ ID NOS: 17-18 and 1232 for vhCDR1-3 of 1D7B4[NKG2D]_H1.11 and SEQ ID NOS: 23, 24, and 26 for vlCDR1-3; SEQ ID NOS: 12-18 and 1234 for vhCDR1-3 of 1D7B4[NKG2D]_H1.12 and SEQ ID NOS: 23, 24, and 26 for vlCDR1-3; SEQ ID NOS: 12-18 and 1236 for vhCDR1-3 of 1D7B4[NKG2D]_H1.13 and SEQ ID NOS: 23, 24, and 26 for vlCDR1-3; SEQ ID NOS: 17-18 and 1238 for vhCDR1-3 of 1D7B4[NKG2D]_H1.14 and SEQ ID NOS: 23, 24, and 26 for vlCDR1-3; SEQ ID NOS: 17-18 and 1240 for vhCDR1-3 of 1D7B4[NKG2D]_H1.15 and SEQ ID NOS: 23, 24, and 26 for vlCDR1-3; SEQ ID NOS: 17-18 and 1242 for vhCDR1-3 of 1D7B4[NKG2D]_H1.16 and SEQ ID NOS: 23, 24, and 26 for vlCDR1-3; SEQ ID NOS: 17-18 and 1244 for vhCDR1-3 of 1D7B4[NKG2D]_H1.17 and SEQ ID NOS: 23, 24, and 26 for vlCDR1-3; SEQ ID NOS: 17-18 and 1246 for vhCDR1-3 of 1D7B4[NKG2D]_H1.18 and SEQ ID NOS: 23, 24, and 26 for vlCDR1-3; SEQ ID NOS: 17-18 and 1248 for vhCDR1-3 of 1D7B4[NKG2D]_H1.19 and SEQ ID NOS: 23, 24, and 26 for vlCDR1-3; SEQ ID NOS: 17-18 and 1250 for vhCDR1-3 of 1D7B4[NKG2D]_H1.20 and SEQ ID NOS: 23, 24, and 26 for vlCDR1-3; SEQ ID NOS: 17-18 and 1252 for vhCDR1-3 of 1D7B4[NKG2D]_H1.21 and SEQ ID NOS: 23, 24, and 26 for vlCDR1-3; SEQ ID NOS: 17-18 and 1254 for vhCDR1-3 of 1D7B4[NKG2D]_H1.22 and SEQ ID NOS: 23, 24, and 26 for vlCDR1-3; SEQ ID NOS: 17-18 and 1258 for vhCDR1-3 of 1D7B4[NKG2D]_H1.24 and SEQ ID NOS: 23, 24, and 26 for vlCDR1-3; SEQ ID NOS: 17-18 and 1260 for vhCDR1-3 of 1D7B4[NKG2D]_H1.25 and SEQ ID NOS: 23, 24, and 26 for vlCDR1-3; SEQ ID NOS: 17-18 and 1262 for vhCDR1-3 of 1D7B4[NKG2D]_H1.26 and SEQ ID NOS: 23, 24, and 26 for vlCDR1-3; SEQ ID NOS: 17-18 and 1264 for vhCDR1-3 of 1D7B4[NKG2D]_H1.27 and SEQ ID NOS: 23, 24, and 26 for vlCDR1-3; SEQ ID NOS: 17-18 and 1266 for vhCDR1-3 of 1D7B4[NKG2D]_H1.28 and SEQ ID NOS: 23, 24, and 26 for vlCDR1-3; SEQ ID NOS: 17-18 and 1268 for vhCDR1-3 of 1D7B4[NKG2D]_H1.29 and SEQ ID NOS: 23, 24, and 26 for vlCDR1-3; SEQ ID NOS: 17-18 and 1270 for vhCDR1-3 of 1D7B4[NKG2D]_H1.30 and SEQ ID NOS: 23, 24, and 26 for vlCDR1-3; SEQ ID NOS: 17-18 and 1274 for vhCDR1-3 of 1D7B4[NKG2D]_H1.32 and SEQ ID NOS: 23, 24, and 26 for vlCDR1-3; SEQ ID NOS: 17-18 and 1276 for vhCDR1-3 of 1D7B4[NKG2D]_H1.33 and SEQ ID NOS: 23, 24, and 26 for vlCDR1-3; SEQ ID NOS: 17-18 and 1278 for vhCDR1-3 of 1D7B4[NKG2D]_H1.34 and SEQ ID NOS: 23, 24, and 26 for vlCDR1-3; SEQ ID NOS: 17-18 and 1280 for vhCDR1-3 of 1D7B4[NKG2D]_H1.35 and SEQ ID NOS: 23, 24, and 26 for vlCDR1-3; SEQ ID NOS: 17-18 and 1282 for vhCDR1-3 of 1D7B4[NKG2D]_H1.36 and SEQ ID NOS: 23, 24, and 26 for vlCDR1-3; SEQ ID NOS: 17-18 and 1284 for vhCDR1-3 of 1D7B4[NKG2D]_H1.37 and SEQ ID NOS: 23, 24, and 26 for vlCDR1-3; SEQ ID NOS: 17-18 and 1286 for vhCDR1-3 of 1D7B4[NKG2D]_H1.38 and SEQ ID NOS: 23, 24, and 26 for vlCDR1-3; SEQ ID NOS: 17-18 and 1288 for vhCDR1-3 of 1D7B4[NKG2D]_H1.39 and SEQ ID NOS: 23, 24, and 26 for vlCDR1-3; SEQ ID NOS: 17-18 and 1290 for vhCDR1-3 of 1D7B4[NKG2D]_H1.40 and SEQ ID NOS: 23, 24, and 26 for vlCDR1-3; SEQ ID NOS: 17-18 and 1292 for vhCDR1-3 of 1D7B4[NKG2D]_H1.41 and SEQ ID NOS: 23, 24, and 26 for vlCDR1-3; SEQ ID NOS: 17-18 and 1294 for vhCDR1-3 of 1D7B4[NKG2D]_H1.42 and SEQ ID NOS: 23, 24, and 26 for vlCDR1-3; SEQ ID NOS: 17-18 and 1296 for vhCDR1-3 of 1D7B4[NKG2D]_H1.43 and SEQ ID NOS: 23, 24, and 26 for vlCDR1-3; SEQ ID NOS: 17-18 and 1298 for vhCDR1-3 of 1D7B4[NKG2D]_H1.44 and SEQ ID NOS: 23, 24, and 26 for vlCDR1-3; SEQ ID NOS: 17-18 and 1300 for vhCDR1-3 of 1D7B4[NKG2D]_H1.45 and SEQ ID NOS: 23, 24, and 26 for vlCDR1-3; SEQ ID NOS: 17-18 and 1302 for vhCDR1-3 of 1D7B4[NKG2D]_H1.46 and SEQ ID NOS: 23, 24, and 26 for vlCDR1-3; SEQ ID NOS17-18 and 1304 for vhCDR1-3 of 1D7B4[NKG2D]_H1.47 and SEQ ID NOS: 23, 24, and 26 for vlCDR1-3; and SEQ ID NOS: 17-18 and 1306 for vhCDR1-3 of 1D7B4[NKG2D]_H1.48 and SEQ ID NOS: 23, 24, and 26 for vlCDR1-3, as depicted in FIGS. 23 and 58.

In some embodiments, the anti-NKG2D scFv comprises a variable heavy domain and variable light domain pair selected from the group consisting of SEQ ID NOS: 2603 and 2607 of mAb-C[NKG2D]; SEQ ID NOS: 2611 and 2615 of mAb-D[NKG2D]; SEQ ID NOS: 1255 and 51 of 1D7B4[NKG2D]_H1.23_L1; SEQ ID NOS: 1271 and 51 of 1D7B4[NKG2D]_H1.31_L1; SEQ ID NOS: 50 and 51 of 1D7B4[NKG2D]_H1_L1; SEQ ID NOS: 52 and 51 of 1D2B4[NKG2D]; SEQ ID NOS: 1211 and 51 of 1D7B4[NKG2D]_H1.1_L1; SEQ ID NOS: 1213 and 51 of 1D7B4[NKG2D]_H1.2_L1; SEQ ID NOS: 1215 and 51 of 1D7B4[NKG2D]_H1.3_L1; SEQ ID NOS: 1217 and 51 of 1D7B4[NKG2D]_H1.4_L1; SEQ ID NOS: 1219 and 51 of 1D7B4[NKG2D]_H1.5_L1; SEQ ID NOS: 1221 and 51 of 1D7B4[NKG2D]_H1.6_L1; SEQ ID NOS: 1223 and 51 of 1D7B4[NKG2D]_H1.7_L1; SEQ ID NOS: 1225 and 51 of 1D7B4[NKG2D]_H1.8_L1; SEQ ID NOS: 1227 and 51 of 1D7B4[NKG2D]_H1.9_L1; SEQ ID NOS: 1229 and 51 of 1D7B4[NKG2D]_H1.10_L1; SEQ ID NOS: 1231 and 51 of 1D7B4[NKG2D]_H1.11_L1; SEQ ID NOS: 1233 and 51 of 1D7B4[NKG2D]_H1.12_L1; SEQ ID NOS: 1235 and 51 of 1D7B4[NKG2D]_H1.13_L1; SEQ ID NOS: 1237 and 51 of 1D7B4[NKG2D]_H1.14_L1; SEQ ID NOS: 1239 and 51 of 1D7B4[NKG2D]_H1.15_L1; SEQ ID NOS: 1241 and 51 of 1D7B4[NKG2D]_H1.16_L1; SEQ ID NOS: 1243 and 51 of 1D7B4[NKG2D]_H1.17_L1; SEQ ID NOS: 1245 and 51 of 1D7B4[NKG2D]_H1.18_L1; SEQ ID NOS: 1247 and 51 of 1D7B4[NKG2D]_H1.19_L1; SEQ ID NOS: 1249 and 51 of 1D7B4[NKG2D]_H1.20_L1; SEQ ID NOS: 1251 and 51 of 1D7B4[NKG2D]_H1.21_L1; SEQ ID NOS: 1253 and 51 of 1D7B4[NKG2D]_H1.22_L1; SEQ ID NOS: 1257 and 51 of 1D7B4[NKG2D]_H1.24_L1; SEQ ID NOS: 1259 and 51 of 1D7B4[NKG2D]_H1.25_L1; SEQ ID NOS: 1261 and 51 of 1D7B4[NKG2D]_H1.26_L1; SEQ ID NOS: 1263 and 51 of 1D7B4[NKG2D]_H1.27_L1; SEQ ID NOS: 1265 and 51 of 1D7B4[NKG2D]_H1.28_L1; SEQ ID NOS: 1267 and 51 of 1D7B4[NKG2D]_H1.29_L1; SEQ ID NOS: 1269 and 51 of 1D7B4[NKG2D]_H1.30_L1; SEQ ID NOS: 1273 and 51 of 1D7B4[NKG2D]_H1.32_L1; SEQ ID NOS: 1275 and 51 of 1D7B4[NKG2D]_H1.33_L1; SEQ ID NOS: 1277 and 51 of 1D7B4[NKG2D]_H1.34_L1; SEQ ID NOS: 1279 and 51 of 1D7B4[NKG2D]_H1.35_L1; SEQ ID NOS: 1281 and 51 of 1D7B4[NKG2D]_H1.36_L1; SEQ ID NOS: 1283 and 51 of 1D7B4[NKG2D]_H1.37_L1; SEQ ID NOS: 1285 and 51 of 1D7B4[NKG2D]_H1.38_L1; SEQ ID NOS: 1287 and 51 of 1D7B4[NKG2D]_H1.39_L1; SEQ ID NOS: 1289 and 51 of 1D7B4[NKG2D]_H1.40_L1; SEQ ID NOS: 1291 and 51 of 1D7B4[NKG2D]_H1.41_L1; SEQ ID NOS: 1293 and 51 of 1D7B4[NKG2D]_H1.42_L1; SEQ ID NOS: 1295 and 51 of 1D7B4[NKG2D]_H1.43_L1; SEQ ID NOS: 1297 and 51 of 1D7B4[NKG2D]_H1.44_L1; SEQ ID NOS: 1299 and 51 of 1D7B4[NKG2D]_H1.45_L1; SEQ ID NOS: 1301 and 51 of 1D7B4[NKG2D]_H1.46_L1; SEQ ID NOS: 1303 and 51 of 1D7B4[NKG2D]_H1.47_L1; and SEQ ID NOS: 1305 and 51 of 1D7B4[NKG2D]_H1.48_L1, as depicted in FIGS. 23 and 58.

In some embodiments, the scFv linker is a charged scFv linker.

In some embodiments, the scFv linker is a charged scFv linker having the amino acid sequence (GKPGS)4 (SEQ ID NO:96).

In some embodiments, the first domain comprises an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

In some embodiments, the second domain comprises an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

In some embodiments, the first and second Fc domains comprise a set of amino acid substitutions selected from the group consisting of: S239D/I332E:S239D/I332E; S239D:S239D; I332E:I332E; WT:S239D/I332E; WT:S239D; WT:I332E; S239D/I332E:WT; S239D:WT; I332E:WT; S239D/I332E:S239D; S239D/I332E:I332E; S239D:S239D/I332E; I332E:S239D/I332E; S239D:I332E; and I332E:S239D, wherein numbering is according to EU numbering.

In some embodiments, the first or second Fc domain comprises the amino acid substitutions S239D/I332E, wherein numbering is according to EU numbering.

In some embodiments, the first and second Fc domains further comprise a set of heterodimerization variants selected from the group consisting of those depicted in FIGS. 1A-1E, wherein numbering is according to EU numbering.

In some embodiments, the set of heterodimerization variants is selected from the group consisting of S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering.

In some embodiments, the first or second Fc domain comprises one or more pI variants.

In some embodiments, the one or more pI variants are N208D/Q295E/N384D/Q418E/N421D, wherein numbering is according to EU numbering.

In some embodiments, the first and second monomers each further comprise amino acid substitutions selected from the group consisting of M428L/N434S, M428L/N434A, and M252Y/S254T/T256E, wherein numbering is according to EU numbering.

In many embodiments, the heterodimeric antibody comprises the amino acid sequences of SEQ ID NOS:4, 49 and 6 of XENP40556.

In one aspect, provided herein is a heterodimeric antibody comprising: a) a first monomer comprising, from N-terminal to C-terminal, a VH1-CH1-hinge-first linker-VH1-CH1-hinge-CH2-CH3, wherein VH1 is a first variable heavy domain and CH2-CH3 is a first Fc domain; b) a light chain comprising, from N-terminus to C-terminus, a VL1-CL, wherein VL1 is a first variable light domain and CL is a constant light domain, and wherein the VH1 and the VL1 form B7H3 antigen binding domains; and c) a second monomer comprising, from N-terminal to C-terminal, an anti-NKG2D scFv and a second Fc domain, wherein the scFv is covalently attached to the N-terminus of the second Fc domain using a domain linker, and wherein the first Fc domain and/or the second Fc domain comprise an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering and have enhanced FcγRIIIA (CD16a) binding compared to first and second Fc domains lacking such substitution(s).

In some embodiments, the anti-NKG2D scFv comprising a second variable heavy VH2 domain, an scFv linker and a second variable light VL2 domain.

In some embodiments, the anti-NKG2D scFv comprises a set of vhCDR1-3 and vlCDR1-3 from a variable heavy domain and variable light domain pair selected from the group consisting of SEQ ID NOS: 2604-2606 for vhCDR1-3 and SEQ ID NOS: 2608-2610 for vlCDR1-3 of mAb-C[NKG2D]; SEQ ID NOS: 2612-2614 for vhCDR1-3 and SEQ ID NOS: 2616-2618 for vlCDR1-3 of mAb-D[NKG2D]; SEQ ID NOS: 17-18 and 1256 for vhCDR1-3 of 1D7B4[NKG2D]_H1.23 and SEQ ID NOS: 23, 24, and 26 for vlCDR1-3 of 1D7B4[NKG2D]_L1; SEQ ID NOS: 17-18 and 1272 for vhCDR1-3 of 1D7B4[NKG2D]_H1.31 and SEQ ID NOS: 23, 24, and 26 for vlCDR1-3 of 1D7B4[NKG2D]_L1; SEQ ID NOS: 17-19 for vhCDR1-3 and SEQ ID NOS: 23, 24, and 26 for vlCDR1-3 of 1D7B4[NKG2D]_H1_L1; SEQ ID NOS: 33-35 for vhCDR1-3 and SEQ ID NOS: 23, 24, and 26 for vlCDR1-3 of 1D2B4[NKG2D]_H1_L1; SEQ ID NOS: 1212 and 18-19 for vhCDR1-3 of 1D7B4[NKG2D]_H1.1 and SEQ ID NOS: 23, 24, and 26 for vlCDR1-3; SEQ ID NOS: 1214 and 18-19 for vhCDR1-3 of 1D7B4[NKG2D]_H1.2 and SEQ ID NOS: 23, 24, and 26 for vlCDR1-3; SEQ ID NOS: 1216 and 18-19 for vhCDR1-3 of 1D7B4[NKG2D]_H1.3 and SEQ ID NOS: 23, 24, and 26 for vlCDR1-3; SEQ ID NOS: 1218 and 18-19 for vhCDR1-3 of 1D7B4[NKG2D]_H1.4 and SEQ ID NOS: 23, 24, and 26 for vlCDR1-3; SEQ ID NOS: 1220 and 18-19 for vhCDR1-3 of 1D7B4[NKG2D]_H1.5 and SEQ ID NOS: 23, 24, and 26 for vlCDR1-3; SEQ ID NOS: 1222 and 18-19 for vhCDR1-3 of 1D7B4[NKG2D]_H1.6 and SEQ ID NOS: 23, 24, and 26 for vlCDR1-3; SEQ ID NOS: 1224 and 18-19 for vhCDR1-3 of 1D7B4[NKG2D]_H1.7 and SEQ ID NOS: 23, 24, and 26 for vlCDR1-3; SEQ ID NOS: 1226 and 18-19 for vhCDR1-3 of 1D7B4[NKG2D]_H1.8 and SEQ ID NOS: 23, 24, and 26 for vlCDR1-3; SEQ ID NOS: 17-18 and 1228 for vhCDR1-3 of 1D7B4[NKG2D]_H1.9 and SEQ ID NOS: 23, 24, and 26 for vlCDR1-3; SEQ ID NOS: 17-18 and 1230 for vhCDR1-3 of 1D7B4[NKG2D]_H1.10 and SEQ ID NOS: 23, 24, and 26 for vlCDR1-3; SEQ ID NOS: 17-18 and 1232 for vhCDR1-3 of 1D7B4[NKG2D]_H1.11 and SEQ ID NOS: 23, 24, and 26 for vlCDR1-3; SEQ ID NOS: 12-18 and 1234 for vhCDR1-3 of 1D7B4[NKG2D]_H1.12 and SEQ ID NOS: 23, 24, and 26 for vlCDR1-3; SEQ ID NOS: 17-18 and 1236 for vhCDR1-3 of 1D7B4[NKG2D]_H1.13 and SEQ ID NOS: 23, 24, and 26 for vlCDR1-3; SEQ ID NOS: 17-18 and 1238 for vhCDR1-3 of 1D7B4[NKG2D]_H1.14 and SEQ ID NOS: 23, 24, and 26 for vlCDR1-3; SEQ ID NOS: 17-18 and 1240 for vhCDR1-3 of 1D7B4[NKG2D]_H1.15 and SEQ ID NOS: 23, 24, and 26 for vlCDR1-3; SEQ ID NOS: 17-18 and 1242 for vhCDR1-3 of 1D7B4[NKG2D]_H1.16 and SEQ ID NOS: 23, 24, and 26 for vlCDR1-3; SEQ ID NOS: 17-18 and 1244 for vhCDR1-3 of 1D7B4[NKG2D]_H1.17 and SEQ ID NOS: 23, 24, and 26 for vlCDR1-3; SEQ ID NOS: 17-18 and 1246 for vhCDR1-3 of 1D7B4[NKG2D]_H1.18 and SEQ ID NOS: 23, 24, and 26 for vlCDR1-3; SEQ ID NOS: 17-18 and 1248 for vhCDR1-3 of 1D7B4[NKG2D]_H1.19 and SEQ ID NOS: 23, 24, and 26 for vlCDR1-3; SEQ ID NOS: 17-18 and 1250 for vhCDR1-3 of 1D7B4[NKG2D]_H1.20 and SEQ ID NOS: 23, 24, and 26 for vlCDR1-3; SEQ ID NOS: 17-18 and 1252 for vhCDR1-3 of 1D7B4[NKG2D]_H1.21 and SEQ ID NOS: 23, 24, and 26 for vlCDR1-3; SEQ ID NOS: 17-18 and 1254 for vhCDR1-3 of 1D7B4[NKG2D]_H1.22 and SEQ ID NOS: 23, 24, and 26 for vlCDR1-3; SEQ ID NOS: 17-18 and 1258 for vhCDR1-3 of 1D7B4[NKG2D]_H1.24 and SEQ ID NOS: 23, 24, and 26 for vlCDR1-3; SEQ ID NOS: 17-18 and 1260 for vhCDR1-3 of 1D7B4[NKG2D]_H1.25 and SEQ ID NOS: 23, 24, and 26 for vlCDR1-3; SEQ ID NOS: 17-18 and 1262 for vhCDR1-3 of 1D7B4[NKG2D]_H1.26 and SEQ ID NOS: 23, 24, and 26 for vlCDR1-3; SEQ ID NOS: 17-18 and 1264 for vhCDR1-3 of 1D7B4[NKG2D]_H1.27 and SEQ ID NOS: 23, 24, and 26 for vlCDR1-3; SEQ ID NOS: 17-18 and 1266 for vhCDR1-3 of 1D7B4[NKG2D]_H1.28 and SEQ ID NOS: 23, 24, and 26 for vlCDR1-3; SEQ ID NOS: 17-18 and 1268 for vhCDR1-3 of 1D7B4[NKG2D]_H1.29 and SEQ ID NOS: 23, 24, and 26 for vlCDR1-3; SEQ ID NOS: 17-18 and 1270 for vhCDR1-3 of 1D7B4[NKG2D]_H1.30 and SEQ ID NOS: 23, 24, and 26 for vlCDR1-3; SEQ ID NOS: 17-18 and 1274 for vhCDR1-3 of 1D7B4[NKG2D]_H1.32 and SEQ ID NOS: 23, 24, and 26 for vlCDR1-3; SEQ ID NOS: 17-18 and 1276 for vhCDR1-3 of 1D7B4[NKG2D]_H1.33 and SEQ ID NOS: 23, 24, and 26 for vlCDR1-3; SEQ ID NOS: 17-18 and 1278 for vhCDR1-3 of 1D7B4[NKG2D]_H1.34 and SEQ ID NOS: 23, 24, and 26 for vlCDR1-3; SEQ ID NOS: 17-18 and 1280 for vhCDR1-3 of 1D7B4[NKG2D]_H1.35 and SEQ ID NOS: 23, 24, and 26 for vlCDR1-3; SEQ ID NOS: 17-18 and 1282 for vhCDR1-3 of 1D7B4[NKG2D]_H1.36 and SEQ ID NOS: 23, 24, and 26 for vlCDR1-3; SEQ ID NOS: 17-18 and 1284 for vhCDR1-3 of 1D7B4[NKG2D]_H1.37 and SEQ ID NOS: 23, 24, and 26 for vlCDR1-3; SEQ ID NOS: 17-18 and 1286 for vhCDR1-3 of 1D7B4[NKG2D]_H1.38 and SEQ ID NOS: 23, 24, and 26 for vlCDR1-3; SEQ ID NOS: 17-18 and 1288 for vhCDR1-3 of 1D7B4[NKG2D]_H1.39 and SEQ ID NOS: 23, 24, and 26 for vlCDR1-3; SEQ ID NOS: 17-18 and 1290 for vhCDR1-3 of 1D7B4[NKG2D]_H1.40 and SEQ ID NOS: 23, 24, and 26 for vlCDR1-3; SEQ ID NOS: 17-18 and 1292 for vhCDR1-3 of 1D7B4[NKG2D]_H1.41 and SEQ ID NOS: 23, 24, and 26 for vlCDR1-3; SEQ ID NOS: 17-18 and 1294 for vhCDR1-3 of 1D7B4[NKG2D]_H1.42 and SEQ ID NOS: 23, 24, and 26 for vlCDR1-3; SEQ ID NOS: 17-18 and 1296 for vhCDR1-3 of 1D7B4[NKG2D]_H1.43 and SEQ ID NOS: 23, 24, and 26 for vlCDR1-3; SEQ ID NOS: 17-18 and 1298 for vhCDR1-3 of 1D7B4[NKG2D]_H1.44 and SEQ ID NOS: 23, 24, and 26 for vlCDR1-3; SEQ ID NOS: 17-18 and 1300 for vhCDR1-3 of 1D7B4[NKG2D]_H1.45 and SEQ ID NOS: 23, 24, and 26 for vlCDR1-3; SEQ ID NOS: 17-18 and 1302 for vhCDR1-3 of 1D7B4[NKG2D]_H1.46 and SEQ ID NOS: 23, 24, and 26 for vlCDR1-3; SEQ ID NOS17-18 and 1304 for vhCDR1-3 of 1D7B4[NKG2D]_H1.47 and SEQ ID NOS: 23, 24, and 26 for vlCDR1-3; and SEQ ID NOS: 17-18 and 1306 for vhCDR1-3 of 1D7B4[NKG2D]_H1.48 and SEQ ID NOS: 23, 24, and 26 for vlCDR1-3, as depicted in FIGS. 23 and 58.

In some embodiments, the anti-NKG2D scFv comprises a variable heavy domain and variable light domain pair selected from the group consisting of SEQ ID NOS: 2603 and 2607 of mAb-C[NKG2D]; SEQ ID NOS: 2611 and 2615 of mAb-D[NKG2D]; SEQ ID NOS: 1255 and 51 of 1D7B4[NKG2D]_H1.23_L1; SEQ ID NOS: 1271 and 51 of 1D7B4[NKG2D]_H1.31_L1; SEQ ID NOS: 50 and 51 of 1D7B4[NKG2D]_H1_L1; SEQ ID NOS: 52 and 51 of 1D2B4[NKG2D]; SEQ ID NOS: 1211 and 51 of 1D7B4[NKG2D]_H1.1_L1; SEQ ID NOS: 1213 and 51 of 1D7B4[NKG2D]_H1.2_L1; SEQ ID NOS: 1215 and 51 of 1D7B4[NKG2D]_H1.3_L1; SEQ ID NOS: 1217 and 51 of 1D7B4[NKG2D]_H1.4_L1; SEQ ID NOS: 1219 and 51 of 1D7B4[NKG2D]_H1.5_L1; SEQ ID NOS:1221 and 51 of 1D7B4[NKG2D]_H1.6_L1; SEQ ID NOS:1223 and 51 of 1D7B4[NKG2D]_H1.7_L1; SEQ ID NOS: 1225 and 51 of 1D7B4[NKG2D]_H1.8_L1; SEQ ID NOS: 1227 and 51 of 1D7B4[NKG2D]_H1.9_L1; SEQ ID NOS: 1229 and 51 of 1D7B4[NKG2D]_H1.10_L1; SEQ ID NOS: 1231 and 51 of 1D7B4[NKG2D]_H1.11_L1; SEQ ID NOS: 1233 and 51 of 1D7B4[NKG2D]_H1.12_L1; SEQ ID NOS: 1235 and 51 of 1D7B4[NKG2D]_H1.13_L1; SEQ ID NOS: 1237 and 51 of 1D7B4[NKG2D]_H1.14_L1; SEQ ID NOS: 1239 and 51 of 1D7B4[NKG2D]_H1.15_L1; SEQ ID NOS: 1241 and 51 of 1D7B4[NKG2D]_H1.16_L1; SEQ ID NOS: 1243 and 51 of 1D7B4[NKG2D]_H1.17_L1; SEQ ID NOS: 1245 and 51 of 1D7B4[NKG2D]_H1.18_L1; SEQ ID NOS: 1247 and 51 of 1D7B4[NKG2D]_H1.19_L1; SEQ ID NOS: 1249 and 51 of 1D7B4[NKG2D]_H1.20_L1; SEQ ID NOS: 1251 and 51 of 1D7B4[NKG2D]_H1.21_L1; SEQ ID NOS: 1253 and 51 of 1D7B4[NKG2D]_H1.22_L1; SEQ ID NOS: 1257 and 51 of 1D7B4[NKG2D]_H1.24_L1; SEQ ID NOS: 1259 and 51 of 1D7B4[NKG2D]_H1.25_L1; SEQ ID NOS: 1261 and 51 of 1D7B4[NKG2D]_H1.26_L1; SEQ ID NOS: 1263 and 51 of 1D7B4[NKG2D]_H1.27_L1; SEQ ID NOS: 1265 and 51 of 1D7B4[NKG2D]_H1.28_L1; SEQ ID NOS: 1267 and 51 of 1D7B4[NKG2D]_H1.29_L1; SEQ ID NOS: 1269 and 51 of 1D7B4[NKG2D]_H1.30_L1; SEQ ID NOS: 1273 and 51 of 1D7B4[NKG2D]_H1.32_L1; SEQ ID NOS: 1275 and 51 of 1D7B4[NKG2D]_H1.33_L1; SEQ ID NOS: 1277 and 51 of 1D7B4[NKG2D]_H1.34_L1; SEQ ID NOS: 1279 and 51 of 1D7B4[NKG2D]_H1.35_L1; SEQ ID NOS: 1281 and 51 of 1D7B4[NKG2D]_H1.36_L1; SEQ ID NOS: 1283 and 51 of 1D7B4[NKG2D]_H1.37_L1; SEQ ID NOS: 1285 and 51 of 1D7B4[NKG2D]_H1.38_L1; SEQ ID NOS: 1287 and 51 of 1D7B4[NKG2D]_H1.39_L1; SEQ ID NOS: 1289 and 51 of 1D7B4[NKG2D]_H1.40_L1; SEQ ID NOS: 1291 and 51 of 1D7B4[NKG2D]_H1.41_L1; SEQ ID NOS: 1293 and 51 of 1D7B4[NKG2D]_H1.42_L1; SEQ ID NOS: 1295 and 51 of 1D7B4[NKG2D]_H1.43_L1; SEQ ID NOS: 1297 and 51 of 1D7B4[NKG2D]_H1.44_L1; SEQ ID NOS: 1299 and 51 of 1D7B4[NKG2D]_H1.45_L1; SEQ ID NOS: 1301 and 51 of 1D7B4[NKG2D]_H1.46_L1; SEQ ID NOS: 1303 and 51 of 1D7B4[NKG2D]_H1.47_L1; and SEQ ID NOS: 1305 and 51 of 1D7B4[NKG2D]_H1.48_L1, as depicted in FIGS. 23 and 58.

In some embodiments, each of the B7H3 antigen binding domains comprise a set of vhCDR1-3 and vlCDR1-3 from a variable heavy domain and variable light domain pair, wherein the set of vhCDR1-3 and vlCDR1-3 is selected from the group consisting of SEQ ID NOS: 27, 28, and 29 for vhCDR1-3 and SEQ ID NOS: 30, 31, and 32 for vlCDR1-3 of 38E2[B7H3]_H2_L1.1; SEQ ID NOS: 243, 244, and 245 for vhCDR1-3 and SEQ ID NOS: 247, 248, and 249 for vlCDR1-3 of 6A1[B7H3]_H1_L1; SEQ ID NOS: 143, 144, and 22 for vhCDR1-3 and SEQ ID NOS: 23, 24, and 26 for vlCDR1-3 of 2E43.189[B7H3]_H1_L1; and SEQ ID NOS: 20, 21, and 22 for vhCDR1-3 and SEQ ID NOS: 23, 24, and 26 for vlCDR1-3 of 2E43.189[B7H3]_H1.22_L1, as depicted in FIGS. 13 and 14.

In some embodiments, each of the B7H3 antigen binding domains comprise a variable heavy domain and variable light domain pair selected from the group consisting of SEQ ID NOS: 140 and 141 of 38E2[B7H3]_H2_L1.1; SEQ ID NOS: 242 and 246 of 6A1[B7H3]_H1L1; SEQ ID NOS:142 and 51 of 2E4A3.189[B7H3]_H1_L1; and SEQ ID NOS: 145 and 51 of 2E4A3.189[B7H3]_H1.22_L1, as depicted in FIGS. 13 and 14.

In some embodiments, the scFv linker is a charged scFv linker.

In some embodiments, the scFv linker is a charged scFv linker having the amino acid sequence (GKPGS)4 (SEQ ID NO:96).

In some embodiments, the first and second linkers are each domain linkers.

In some embodiments, the first domain comprises an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

In some embodiments, the second domain comprises an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

In some embodiments, the first and second Fc domains comprise a set of amino acid substitutions selected from the group consisting of: S239D/I332E:S239D/I332E; S239D S239D; I332E:I332E; WT:S239D/I332E; WT:S239D; WT:I332E; S239D/I332E:WT; S239D:WT; I332E:WT; S239D/I332E:S239D; S239D/I332E:I332E; S239D:S239D/I332E; I332E:S239D/I332E; S239D:I332E; and I332E:S239D, wherein numbering is according to EU numbering.

In some embodiments, the first or second Fc domain comprises the amino acid substitutions S239D/I332E, wherein numbering is according to EU numbering.

In some embodiments, the first and second Fc domains further comprise a set of heterodimerization variants selected from the group consisting of those depicted in FIGS. 1A-1E, wherein numbering is according to EU numbering.

In some embodiments, the set of heterodimerization variants is selected from the group consisting of S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering.

In some embodiments, the first or second Fc domain further comprises one or more pI variants.

In some embodiments, the one or more pI variants are N208D/Q295E/N384D/Q418E/N421D, wherein numbering is according to EU numbering.

In some embodiments, the first and second monomers each further comprise amino acid substitutions selected from the group consisting of M428L/N434S, M428L/N434A, and M252Y/S254T/T256E, wherein numbering is according to EU numbering.

The heterodimeric antibody according any one of claims 88-105, comprises the amino acid sequences of SEQ ID NOS:1209, 1210 and 6 of XENP42983, as depicted in FIG. 57.

In some aspects, described herein is a composition comprising an NKG2D antigen binding domain, wherein the NKG2D antigen binding domain comprises a set of vhCDR1-3 and vlCDR1-3 from a variable heavy domain and variable light domain pair selected from the group consisting of: SEQ ID NOS: 17-18 and 1256 for vhCDR1-3 of 1D7B4[NKG2D]_H1.23 and SEQ ID NOS: 23, 24, and 26 for vlCDR1-3 of 1D7B4[NKG2D]_L1; SEQ ID NOS: 17-18 and 1272 for vhCDR1-3 of 1D7B4[NKG2D]_H1.31 and SEQ ID NOS: 23, 24, and 26 for vlCDR1-3 of 1D7B4[NKG2D]_L1; SEQ ID NOS: 1212 and 18-19 for vhCDR1-3 of 1D7B4[NKG2D]_H1.1 and SEQ ID NOS: 23, 24, and 26 for vlCDR1-3; SEQ ID NOS: 1214 and 18-19 for vhCDR1-3 of 1D7B4[NKG2D]_H1.2 and SEQ ID NOS: 23, 24, and 26 for vlCDR1-3; SEQ ID NOS: 1216 and 18-19 for vhCDR1-3 of 1D7B4[NKG2D]_H1.3 and SEQ ID NOS: 23, 24, and 26 for vlCDR1-3; SEQ ID NOS: 1218 and 18-19 for vhCDR1-3 of 1D7B4[NKG2D]_H1.4 and SEQ ID NOS: 23, 24, and 26 for vlCDR1-3; SEQ ID NOS: 1220 and 18-19 for vhCDR1-3 of 1D7B4[NKG2D]_H1.5 and SEQ ID NOS: 23, 24, and 26 for vlCDR1-3; SEQ ID NOS: 1222 and 18-19 for vhCDR1-3 of 1D7B4[NKG2D]_H1.6 and SEQ ID NOS: 23, 24, and 26 for vlCDR1-3; SEQ ID NOS: 1224 and 18-19 for vhCDR1-3 of 1D7B4[NKG2D]_H1.7 and SEQ ID NOS: 23, 24, and 26 for vlCDR1-3; SEQ ID NOS: 1226 and 18-19 for vhCDR1-3 of 1D7B4[NKG2D]_H1.8 and SEQ ID NOS: 23, 24, and 26 for vlCDR1-3; SEQ ID NOS: 17-18 and 1228 for vhCDR1-3 of 1D7B4[NKG2D]_H1.9 and SEQ ID NOS: 23, 24, and 26 for vlCDR1-3; SEQ ID NOS: 17, 18 and 1230 for vhCDR1-3 of 1D7B4[NKG2D]_H1.10 and SEQ ID NOS: 23, 24, and 26 for vlCDR1-3; SEQ ID NOS: 17-18 and 1232 for vhCDR1-3 of 1D7B4[NKG2D]_H1.11 and SEQ ID NOS: 23, 24, and 26 for vlCDR1-3; SEQ ID NOS: 12-18 and 1234 for vhCDR1-3 of 1D7B4[NKG2D]_H1.12 and SEQ ID NOS: 23, 24, and 26 for vlCDR1-3; SEQ ID NOS: 12-18 and 1236 for vhCDR1-3 of 1D7B4[NKG2D]_H1.13 and SEQ ID NOS: 23, 24, and 26 for vlCDR1-3; SEQ ID NOS: 17-18 and 1238 for vhCDR1-3 of 1D7B4[NKG2D]_H1.14 and SEQ ID NOS: 23, 24, and 26 for vlCDR1-3; SEQ ID NOS: 17-18 and 1240 for vhCDR1-3 of 1D7B4[NKG2D]_H1.15 and SEQ ID NOS: 23, 24, and 26 for vlCDR1-3; SEQ ID NOS: 17-18 and 1242 for vhCDR1-3 of 1D7B4[NKG2D]_H1.16 and SEQ ID NOS: 23, 24, and 26 for vlCDR1-3; SEQ ID NOS: 17-18 and 1244 for vhCDR1-3 of 1D7B4[NKG2D]_H1.17 and SEQ ID NOS: 23, 24, and 26 for vlCDR1-3; SEQ ID NOS: 17-18 and 1246 for vhCDR1-3 of 1D7B4[NKG2D]_H1.18 and SEQ ID NOS: 23, 24, and 26 for vlCDR1-3; SEQ ID NOS: 17-18 and 1248 for vhCDR1-3 of 1D7B4[NKG2D]_H1.19 and SEQ ID NOS: 23, 24, and 26 for vlCDR1-3; SEQ ID NOS: 17-18 and 1250 for vhCDR1-3 of 1D7B4[NKG2D]_H1.20 and SEQ ID NOS: 23, 24, and 26 for vlCDR1-3; SEQ ID NOS: 17-18 and 1252 for vhCDR1-3 of 1D7B4[NKG2D]_H1.21 and SEQ ID NOS: 23, 24, and 26 for vlCDR1-3; SEQ ID NOS: 17-18 and 1254 for vhCDR1-3 of 1D7B4[NKG2D]_H1.22 and SEQ ID NOS: 23, 24, and 26 for vlCDR1-3; SEQ ID NOS: 17-18 and 1258 for vhCDR1-3 of 1D7B4[NKG2D]_H1.24 and SEQ ID NOS: 23, 24, and 26 for vlCDR1-3; SEQ ID NOS: 17-18 and 1260 for vhCDR1-3 of 1D7B4[NKG2D]_H1.25 and SEQ ID NOS: 23, 24, and 26 for vlCDR1-3; SEQ ID NOS: 17-18 and 1262 for vhCDR1-3 of 1D7B4[NKG2D]_H1.26 and SEQ ID NOS: 23, 24, and 26 for vlCDR1-3; SEQ ID NOS: 17-18 and 1264 for vhCDR1-3 of 1D7B4[NKG2D]_H1.27 and SEQ ID NOS: 23, 24, and 26 for vlCDR1-3; SEQ ID NOS: 17-18 and 1266 for vhCDR1-3 of 1D7B4[NKG2D]_H1.28 and SEQ ID NOS: 23, 24, and 26 for vlCDR1-3; SEQ ID NOS: 17-18 and 1268 for vhCDR1-3 of 1D7B4[NKG2D]_H1.29 and SEQ ID NOS: 23, 24, and 26 for vlCDR1-3; SEQ ID NOS: 17-18 and 1270 for vhCDR1-3 of 1D7B4[NKG2D]_H1.30 and SEQ ID NOS: 23, 24, and 26 for vlCDR1-3; SEQ ID NOS: 17-18 and 1274 for vhCDR1-3 of 1D7B4[NKG2D]_H1.32 and SEQ ID NOS: 23, 24, and 26 for vlCDR1-3; SEQ ID NOS: 17-18 and 1276 for vhCDR1-3 of 1D7B4[NKG2D]_H1.33 and SEQ ID NOS: 23, 24, and 26 for vlCDR1-3; SEQ ID NOS: 17-18 and 1278 for vhCDR1-3 of 1D7B4[NKG2D]_H1.34 and SEQ ID NOS: 23, 24, and 26 for vlCDR1-3; SEQ ID NOS: 17-18 and 1280 for vhCDR1-3 of 1D7B4[NKG2D]_H1.35 and SEQ ID NOS: 23, 24, and 26 for vlCDR1-3; SEQ ID NOS: 17-18 and 1282 for vhCDR1-3 of 1D7B4[NKG2D]_H1.36 and SEQ ID NOS: 23, 24, and 26 for vlCDR1-3; SEQ ID NOS: 17-18 and 1284 for vhCDR1-3 of 1D7B4[NKG2D]_H1.37 and SEQ ID NOS: 23, 24, and 26 for vlCDR1-3; SEQ ID NOS: 17-18 and 1286 for vhCDR1-3 of 1D7B4[NKG2D]_H1.38 and SEQ ID NOS: 23, 24, and 26 for vlCDR1-3; SEQ ID NOS: 17-18 and 1288 for vhCDR1-3 of 1D7B4[NKG2D]_H1.39 and SEQ ID NOS: 23, 24, and 26 for vlCDR1-3; SEQ ID NOS: 17-18 and 1290 for vhCDR1-3 of 1D7B4[NKG2D]_H1.40 and SEQ ID NOS: 23, 24, and 26 for vlCDR1-3; SEQ ID NOS: 17-18 and 1292 for vhCDR1-3 of 1D7B4[NKG2D]_H1.41 and SEQ ID NOS: 23, 24, and 26 for vlCDR1-3; SEQ ID NOS: 17-18 and 1294 for vhCDR1-3 of 1D7B4[NKG2D]_H1.42 and SEQ ID NOS: 23, 24, and 26 for vlCDR1-3; SEQ ID NOS: 17-18 and 1296 for vhCDR1-3 of 1D7B4[NKG2D]_H1.43 and SEQ ID NOS: 23, 24, and 26 for vlCDR1-3; SEQ ID NOS: 17-18 and 1298 for vhCDR1-3 of 1D7B4[NKG2D]_H1.44 and SEQ ID NOS: 23, 24, and 26 for vlCDR1-3; SEQ ID NOS: 17-18 and 1300 for vhCDR1-3 of 1D7B4[NKG2D]_H1.45 and SEQ ID NOS: 23, 24, and 26 for vlCDR1-3; SEQ ID NOS: 17-18 and 1302 for vhCDR1-3 of 1D7B4[NKG2D]_H1.46 and SEQ ID NOS: 23, 24, and 26 for vlCDR1-3; SEQ ID NOS17-18 and 1304 for vhCDR1-3 of 1D7B4[NKG2D]_H1.47 and SEQ ID NOS: 23, 24, and 26 for vlCDR1-3; and SEQ ID NOS: 17-18 and 1306 for vhCDR1-3 of 1D7B4[NKG2D]_H1.48 and SEQ ID NOS: 23, 24, and 26 for vlCDR1-3, as depicted in FIGS. 23 and 58.

In certain aspects, described herein is a composition comprising an NKG2D antigen binding domain, wherein the NKG2D antigen binding domain comprises a variable heavy domain and variable light domain pair selected from the group consisting of SEQ ID NOS: 1255 and 51 of 1D7B4[NKG2D]_H1.23_L1; SEQ ID NOS: 1271 and 51 of 1D7B4[NKG2D]_H1.31_L1; SEQ ID NOS: 1211 and 51 of 1D7B4[NKG2D]_H1.1_L1; SEQ ID NOS: 1213 and 51 of 1D7B4[NKG2D]_H1.2_L1; SEQ ID NOS: 1215 and 51 of 1D7B4[NKG2D]_H1.3_L1; SEQ ID NOS: 1217 and 51 of 1D7B4[NKG2D]_H1.4_L1; SEQ ID NOS: 1219 and 51 of 1D7B4[NKG2D]_H1.5_L1; SEQ ID NOS: 1221 and 51 of 1D7B4[NKG2D]_H1.6_L1; SEQ ID NOS: 1223 and 51 of 1D7B4[NKG2D]_H1.7_L1; SEQ ID NOS: 1225 and 51 of 1D7B4[NKG2D]_H1.8_L1; SEQ ID NOS: 1227 and 51 of 1D7B4[NKG2D]_H1.9_L1; SEQ ID NOS: 1229 and 51 of 1D7B4[NKG2D]_H1.10_L1; SEQ ID NOS: 1231 and 51 of 1D7B4[NKG2D]_H1.11_L1; SEQ ID NOS: 1233 and 51 of 1D7B4[NKG2D]_H1.12_L1; SEQ ID NOS: 1235 and 51 of 1D7B4[NKG2D]_H1.13_L1; SEQ ID NOS: 1237 and 51 of 1D7B4[NKG2D]_H1.14_L1; SEQ ID NOS: 1239 and 51 of 1D7B4[NKG2D]_H1.15_L1; SEQ ID NOS: 1241 and 51 of 1D7B4[NKG2D]_H1.16_L1; SEQ ID NOS: 1243 and 51 of 1D7B4[NKG2D]_H1.17_L1; SEQ ID NOS: 1245 and 51 of 1D7B4[NKG2D]_H1.18_L1; SEQ ID NOS: 1247 and 51 of 1D7B4[NKG2D]_H1.19_L1; SEQ ID NOS: 1249 and 51 of 1D7B4[NKG2D]_H1.20_L1; SEQ ID NOS: 1251 and 51 of 1D7B4[NKG2D]_H1.21_L1; SEQ ID NOS: 1253 and 51 of 1D7B4[NKG2D]_H1.22_L1; SEQ ID NOS: 1257 and 51 of 1D7B4[NKG2D]_H1.24_L1; SEQ ID NOS: 1259 and 51 of 1D7B4[NKG2D]_H1.25_L1; SEQ ID NOS: 1261 and 51 of 1D7B4[NKG2D]_H1.26_L1; SEQ ID NOS: 1263 and 51 of 1D7B4[NKG2D]_H1.27_L1; SEQ ID NOS: 1265 and 51 of 1D7B4[NKG2D]_H1.28_L1; SEQ ID NOS: 1267 and 51 of 1D7B4[NKG2D]_H1.29_L1; SEQ ID NOS: 1269 and 51 of 1D7B4[NKG2D]_H1.30_L1; SEQ ID NOS: 1273 and 51 of 1D7B4[NKG2D]_H1.32_L1; SEQ ID NOS: 1275 and 51 of 1D7B4[NKG2D]_H1.33_L1; SEQ ID NOS: 1277 and 51 of 1D7B4[NKG2D]_H1.34_L1; SEQ ID NOS: 1279 and 51 of 1D7B4[NKG2D]_H1.35_L1; SEQ ID NOS: 1281 and 51 of 1D7B4[NKG2D]_H1.36_L1; SEQ ID NOS: 1283 and 51 of 1D7B4[NKG2D]_H1.37_L1; SEQ ID NOS: 1285 and 51 of 1D7B4[NKG2D]_H1.38_L1; SEQ ID NOS: 1287 and 51 of 1D7B4[NKG2D]_H1.39_L1; SEQ ID NOS: 1289 and 51 of 1D7B4[NKG2D]_H1.40_L1; SEQ ID NOS: 1291 and 51 of 1D7B4[NKG2D]_H1.41_L1; SEQ ID NOS: 1293 and 51 of 1D7B4[NKG2D]_H1.42_L1; SEQ ID NOS: 1295 and 51 of 1D7B4[NKG2D]_H1.43_L1; SEQ ID NOS: 1297 and 51 of 1D7B4[NKG2D]_H1.44_L1; SEQ ID NOS: 1299 and 51 of 1D7B4[NKG2D]_H1.45_L1; SEQ ID NOS: 1301 and 51 of 1D7B4[NKG2D]_H1.46_L1; SEQ ID NOS: 1303 and 51 of 1D7B4[NKG2D]_H1.47_L1; and SEQ ID NOS: 1305 and 51 of 1D7B4[NKG2D]_H1.48_L1, as depicted in FIGS. 23 and 58.

In yet other aspects, provided herein is an antibody comprising any NKG2D antigen binding domain described herein. In some embodiments, the antibody is a monoclonal antibody.

In many embodiments, the antibody is a bispecific antibody.

In some aspects, provided herein is a nucleic acid composition comprising (a) a first nucleic acid encoding the any one of the variable heavy domains described; and (b) a second nucleic acid encoding the any one of the variable light domains described.

In other aspects, provided herein is an expression vector composition comprising (a) a first expression vector comprising any first nucleic acid described; and (b) a second expression vector comprising any second nucleic acid described.

In some embodiments, provided herein is a host cell comprising any one of the expression vector compositions described.

In some embodiments, provided herein is a method of making an NKG2D antigen binding domain or an antibody comprising such comprising culturing a host cell described under conditions, wherein the NKG2D binding domain or an antibody thereof is expressed, and recovering the NKG2D antigen binding domain or the antibody comprising such.

In some aspects, described herein is a method of treating cancer or reducing tumor growth or inhibiting cancer cell proliferation in a subject in need thereof comprising administering to the subject a therapeutically effective amount of any of the compositions described, or any of the antibodies described or an antigen binding fragments thereof to the subject.

In some aspects, described herein is a method of treating cancer or reducing tumor growth or inhibiting cancer cell proliferation in a subject in need thereof comprising administering to the subject a therapeutically effective amount of any of the heterodimeric antibodies described or antigen binding fragments thereof to the subject.

In some embodiments, the method further comprises administering an IL-12-Fc fusion protein and/or an IL-15-Fc fusion protein to the subject. In some embodiments, the IL-12-Fc fusion protein comprises amino acid sequences of SEQ ID NOS:166 and 167 or amino acid sequences of SEQ ID NOS:168 and 169. In some embodiments, the IL-15-Fc fusion protein comprises amino acid sequences of SEQ ID NOS:164 and 165 or amino acid sequences of SEQ ID NOS:1077 and 1078.

In some embodiments, the method further comprises administering a bispecific T-cell engager antibody or an antigen binding fragment thereof to the subject. In some embodiments, the bispecific T-cell engager antibody or antigen binding fragment thereof is a heterodimeric antibody that binds to B7H3 and CD3 or an antigen binding fragment thereof.

In yet another aspect, provided herein is a method of selectively killing cancer cells in a population of cells comprising: a) contacting the population of cells with any one of the heterodimeric antibodies described herein, or an antigen binding fragments thereof, and b) contacting the population of cells with an IL-15-Fc fusion protein and/or an IL-12-Fc fusion protein. In some embodiments, the IL-12-Fc fusion protein comprises amino acid sequences of SEQ ID NOS:166 and 167 or amino acid sequences of SEQ ID NOS:168 and 169. In some embodiments, the IL-15-Fc fusion protein comprises amino acid sequences of SEQ ID NOS:164 and 165 or amino acid sequences of SEQ ID NOS:1077 and 1078.

In some embodiments, the subject is a human subject. In some embodiments, the human subject has cancer, was diagnosed with cancer, or has at least one symptom associated with cancer.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Fig.”, “FIG.,” “Figure,” “Figures,”, “Figs.,” and “FIGs.” herein) of which:

FIGS. 1A-1E depict useful pairs of Fc heterodimerization variant sets (including skew and pI variants). There are variants for which there are no corresponding “monomer 2” variants; these are pI variants which can be used alone on either monomer.

FIG. 2 depicts a list of isosteric variant antibody constant regions and their respective substitutions. pI_(−) indicates lower pI variants, while pI_(+) indicates higher pI variants. These can be optionally and independently combined with other heterodimerization variants of the inventions (and other variant types as well, as outlined herein).

FIG. 3 depicts useful ablation variants that ablate FcγR binding (sometimes referred to as “knock outs” or “KO” variants). Generally, ablation variants are found on both monomers, although in some cases they may be on only one monomer.

FIG. 4 depicts particularly useful embodiments of “non-Fv” components of the invention.

FIG. 5 depicts a number of charged scFv linkers that find use in increasing or decreasing the pI of the subject heterodimeric bsAbs that utilize one or more scFv as a component, as described herein. The (+H) positive linker finds particular use herein, particularly with anti-CD3 V_{L} and V_{H} sequences shown herein. A single prior art scFv linker with a single charge is referenced as “Whitlow”, from Whitlow et al., Protein Engineering 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab₂-scFv-Fc formats).

FIG. 6 depicts a number of exemplary domain linkers. In some embodiments, these linkers find use linking a single-chain Fv to an Fc chain. In some embodiments, these linkers may be combined. For example, a (G)₄S (SEQ ID NO:109) linker may be combined with a “half hinge” linker.

FIGS. 7A-7C depict the sequences of heterodimeric αB7H3×αNKG2D 1+1 Fab-scFv-Fc bispecific antibody format heavy chain backbones with ablated effector function, also referred to as the FcKO variants. Backbone 1 is based on human IgG1 (356E/358M allotype), and includes the S364K/E357Q:L368D/K370S skew variants, C220S on the chain with the S364K/E357Q skew variants, the N208D/Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes S364K:L368D/K370S skew variants, C220S on the chain with the S364K skew variant, the N208D/Q295E/N384D/Q418E/N421D pI variants on the chain with L368D/K370S skew variants, and the E233P/L234V/L235A/G236del/S267K ablation variants on both chains. Backbone 3 is based on human IgG1 (356E/358M allotype), and includes S364K:L368E/K370S skew variants, C220S on the chain with the S364K skew variant, the N208D/Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes D401K:K360E/Q362E/T411E skew variants, C220S on the chain with the D401K skew variant, the N208D/Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes S364K/E357Q:L368D/K370S skew variants, C220S on the chain with the S364K/E357Q skew variants, the N208D/Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes S364K/E357Q:L368D/K370S skew variants, C220S on the chain with the S364K/E357Q skew variants, N208D/Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Backbone 8 is based on human IgG4, and includes the S364K/E357Q:L368D/K370S skew variants, the N208D/Q295E/N384D/Q418E/N421D pI variants on the chain with L368D/K370S skew variants, as well as a S228P (EU numbering, this is S241P in Kabat) variant on both chains that ablates Fab arm exchange as is known in the art. Backbone 9 is based on human IgG2, and includes the S364K/E357Q:L368D/K370S skew variants, the N208D/Q295E/N384D/Q418E/N421D pI variants on the chain with L368D/K370S skew variants. Backbone 10 is based on human IgG2, and includes the S364K/E357Q:L368D/K370S skew variants, the N208D/Q295E/N384D/Q418E/N421D pI variants on the chain with L368D/K370S skew variants as well as a S267K variant on both chains. Backbone 11 is identical to backbone 1, except it includes M428L/N434S Xtend mutations. Backbone 12 is based on human IgG1 (356E/358M allotype), and includes S364K/E357Q:L368D/K370S skew variants, C220S and the P217R/P229R/N276K pI variants on the chain with S364K/E357Q skew variants and the E233P/L234V/L235A/G236del/S267K ablation variants on both chains. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIGS. 8A-8C depict the sequences of several useful 2+1 Fab₂-scFv-Fc bispecific antibody format heavy chain backbones based on human IgG1, without the Fv sequences (e.g., the scFv and the V_{H} for the Fab side). Backbone 1 is based on human IgG1 (356E/358M allotype), and includes the S364K/E357Q:L368D/K370S skew variants, the N208D/Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes S364K:L368D/K370S skew variants, the N208D/Q295E/N384D/Q418E/N421D pI variants on the chain with L368D/K370S skew variants, and the E233P/L234V/L235A/G236del/S267K ablation variants on both chains. Backbone 3 is based on human IgG1 (356E/358M allotype), and includes S364K:L368E/K370S skew variants, the N208D/Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes D401K: K360E/Q362E/T411E skew variants, the N208D/Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes S364K/E357Q:L368D/K370S skew variants, the N208D/Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes S364K/E357Q:L368D/K370S skew variants, N208D/Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Backbone 8 is identical to backbone 1, except it includes M428L/N434S Xtend mutations. Backbone 9 is based on human IgG1 (356E/358M allotype), and includes S364K/E357Q:L368D/K370S skew variants, the P217R/P229R/N276K pI variants on the chain with S364K/E357Q skew variants and the E233P/L234V/L235A/G236del/S267K ablation variants on both chains. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIG. 9 depicts illustrative sequences of heterodimeric αNKG2D×αB7H3 backbone for use in the 2+1 mAb-scFv format. The format depicted here is based on heterodimeric Fc backbone 1 as depicted in the figures including FIG. 35, except further including G446_ on monomer 1 (−) and G446_/K447_ on monomer 2 (+). It should be noted that any of the additional backbones depicted in FIG. 35 may be adapted for use in the 2+1 mAb-scFv format with or without including K447_ on one or both chains. It should be noted that these sequences may further include the M428L/N434S or M428L/N434A variants.

FIG. 10 depicts the sequences of several useful constant light domain backbones based on human IgG1, without the Fv sequences (e.g., the scFv or the Fab). Included herein are constant light backbone sequences that are 90, 95, 98 and 99% identical (as defined herein) to the recited sequences, and/or contain from 1, 2, 3, 4, 5, 6, 7, 8, 9 or 10 additional amino acid modifications.

FIG. 11 depicts sequences for human & cynomolgus NKG2D antigens.

FIGS. 12A-12B depict sequences for human, mouse, and cynomolgus B7H3.

FIG. 13 depicts the variable heavy and variable light chain sequences for humanized 38E2, as well as 6A1, both exemplary rabbit hybridoma-derived B7H3 binding domain. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2 and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 14 depicts the variable heavy and variable light chain sequences for 2E4A3.189, an exemplary phage-derived B7H3 binding domain, as well as the sequences for affinity-optimized variable heavy 2E4A3.189_H1.22, and XENP38571, a bivalent antibody having a 2E4A3.189_H1.22 B7H3 binding domain. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIGS. 15A-15D depict several formats of the present invention as utilized in NK cell engaging antibodies. FIG. 15A depicts the “1+1 Fab-scFv-Fc” format, which comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. In this format, the Fab arm binds Antigen #1 and a second scFv arm binds Antigen #2. In one embodiment Antigen #1 is B7H3 and Antigen #2 is NKG2D. In another embodiment Antigen #1 is NKG2D and Antigen #2 is B7H3. FIG. 15B depicts the “2+1 Fab₂-scFv-Fc” format, with a first Fab arm and a second Fab-scFv arm, wherein the Fab binds B7H3 and the scFv binds NKG2D. The 2+1 Fab₂-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 15C depicts the “2+1 mAb-scFv” format, with a first Fc comprising an N-terminal Fab arm binding B7H3 and a second Fc comprising an N-terminal Fab arm binding B7H3 and a C-terminal scFv binding NKG2D. The 2+1 mAb-scFv format comprises a first monomer comprising VH1-CH1-hinge-CH2-CH3, a second monomer comprising VH1-CH1-hinge-CH2-CH3-scFv, and a third monomer comprising V_{L}-C_{L}. The V_{L} pairs with the first and second VH1 to form binding domains with binding specificity for the tumor-associated antigen. Lastly, FIG. 15D depicts the “stackFab2-scFv-Fc” format which comprises a first monomer comprising from N-terminal to C-terminal VH1-CH1-linker-VH2-CH1-hinge-CH2-CH3 wherein CH2-CH3 is a first heterodimeric Fc domain; a second monomer comprising from N-terminal to C-terminal scFv-linker-CH2-CH3 wherein CH2-CH3 is a second heterodimeric Fc domain complementary to the first heterodimeric Fc domain and wherein the scFv has a first antigen specificity; and a third monomer that is a common light chain comprising from N-terminal to C-terminal V_{L}-C_{L} wherein the V_{L} pairs with VH1 and VH2 of the first monomer to form two antigen binding domains each having a specificity for a second antigen binding domain.

FIG. 16 depicts illustrations of NK engagers inducing both NK cell activation and T cell co-stimulation.

FIG. 17 depicts the monovalent binding affinities (K_{D}) of various B7H3 binding domains in the context of 1+1 bispecific formats.

FIG. 18 depicts an exemplary backbone having the S239D/I332E variants (also referred to as v90 variants) that result in improved binding to FcγRIIIa (CD16a) and increased levels of ADCC activity. It should be noted that the S239D/I332E variants may be used in any of the antibody formats or backbones described herein.

FIGS. 19A-19D depict the sequences for illustrative αNKG2D×αB7H3 bsAbs in the 1+1 Fab-scFv-Fc format. CDRs are underlined and slashes indicate the border(s) between the variable regions, linkers, Fc regions, and constant domains. It should be noted that the αNKG2D×αB7H3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIG. 20 depicts the sequences for illustrative αNKG2D×αB7H3 bsAbs in the 2+1 Fab₂-scFv-Fc format. CDRs are underlined and slashes indicate the border(s) between the variable regions, linkers, Fc regions, and constant domains. The scFv domain has orientation (N- to C-terminus) of V_{H}-scFv linker-V_{L}, although this can be reversed. It should be noted that the scFv domain includes an scFv linker between the variable heavy and variable light region the sequence GKPGSGKPGSGKPGSGKPGS (SEQ ID NO: 96); however, this linker can be replaced with any of the scFv linkers in FIG. 5. It should also be noted that the Chain 2 sequences include as the domain linker between the C-terminus of the scFv and the N-terminus of the CH2 domain the sequence GGGGSGGGGS (SEQ ID NO: 110) which is a “(GGGGS)₂” domain linker and the sequence KTHTCPPCP (SEQ ID NO: 126) which is a “lower half hinge” domain linker (collectively forming a “flex lower half hinge” (SEQ ID NO: 128); however, this linker can be replaced with any of the “useful domain linkers” of FIG. 6. It should be noted that the αNKG2D×αB7H3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIG. 21 depicts the sequence for an illustrative αNKG2D×αB7H3 bsAb in the 2+1 mAb-scFv format. CDRs are underlined and slashes indicate the border(s) between the variable regions, linkers, Fc regions, and constant domains. The scFv domain has orientation (N- to C-terminus) of V_{H}-scFv linker-V_{L}, although this can be reversed. It should be noted that the Chain 2 sequences include as a domain linker the sequence GGGGSGGGGSGGGGS (SEQ ID NO: 87); however, this linker can be replaced with any domain linker including any of the “useful domain linkers” of FIG. 5. It should be noted that the αNKG2D×αB7H3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 22A-22F depict sequences for illustrative IL-15-Fc fusion and IL-12-Fc fusion proteins. FIGS. 22A-22B depict sequences for illustrative IL-15-Fc fusion and IL-12-Fc fusion proteins that may be used in combination with αNKG2D×αB7H3 bsAbs to show a synergistic effect. FIGS. 22C-22D depict human IL-15 and its receptors. FIGS. 22E-22F depict the sequences for IL-12 and its receptors.

FIGS. 23A-23B depict variable heavy and variable light domains as well as CDRS for NKG2D binding clones 1D7B4, 1D2B4, mAb-C and mAb-D. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 24 depicts that Fc engagement of CD16 is crucial for NK cell activation. In this assay, PBMCs were mixed with MCF7 cancer cells at a 40:1 ratio, which corresponds to approximately a 1:1 NK cell to MCF7 ratio. Cells were then treated with the one of the three XENPs at a range of concentrations as indicated in the figure and incubated for 4 hours. The XENPs used in this experiment had either normal binding to FcγRs (WT), ablated binding to FcγRs (KO), or enhanced binding to FcγRIIIa (v90). Flow cytometry was used to measure degranulation marker CD107a and activation marker CD69.

FIG. 25 depicts that NK cell engagers with 1D7B4 and 1D2B4 NKG2D binding domains show strong activation of NK cells. In this assay, PBMCs were mixed with MCF7 cancer cells at a 40:1 ratio, which corresponds to approximately a 1:1 NK cell to MCF7 ratio. Cells were then treated with the one of the three XENPS at a range of concentrations as indicated in the figure and incubated for 4 hours. Flow cytometry was used to measure degranulation marker CD107a and activation marker CD69.

FIGS. 26A-26D depict additional antibodies which may be referred to in the specification.

FIGS. 27A-27B depict the ability of NKG2D×B7H3 bsAbs to effectively kill target cells. In the experiment depicted in FIG. 27A, resting NK cells were co-cultured with MCF7-RFP tumor cells at an E:T ratio of 5:1. Treatments of either isotype control XENP40371 or NKG2D×B7H3 bsAb XENP40377 were added at a concentration of 4.6 ng/ml. MCF7 cell growth was assessed over time with Incucyte. In FIG. 27B, NK cells were also co-cultured with MCF7-RFP tumor cells, and treated with test articles at a range of concentrations. Tumor cell killing was then assessed after 24 hours.

FIG. 28 depicts that NKEs delivered as a single agent showed improved cell killing on MDA-MB-231 B2M knockout cells (right column) compared to parental MDA-MB-231 WT cells (left column). In this experiment, resting NK cells were co-cultured with MDA-MB-231 WT or MDA-MB-231 B2M knockout cells at a 5:1 E:T ratio. NKEs XENP38597 or XENP40377 were added at a range of 0.1 μg/ml to 10 μg/ml, and the target cell count was recorded over time by the Incucyte® system.

FIGS. 29A-29C depict that only XENP38597, and not the comparator molecules, was able to co-stimulate T cells to kill tumor cells. In this experiment, T cells were added to MCF7 target cells at a 5:1 E:T ratio. All cells were dosed at a constant concentration of 10 μg/ml of NKEs, while B7H3×CD3 bispecific XENP31346 was titrated in at doses ranging from 1.52 ng/ml to 10 μg/ml (as indicated at the top of each graph). One NKE used in this experiment was XENP38597, having a 1D7B4 NKG2D binding domain. The other two NKEs used were XENP38600 and XENP38601, which have the same format and B7H3 binding domain as XENP38597, but which use comparator NKG2D binding domains LB1001 and LB1002 instead. Incucyte was used to measure target cell viability every 6 hours over a span of 160 hours. FIG. 29B shows a close-up of the boxed graph in FIG. 29A. FIG. 29C shows a similar experiment with a different test article, XENP40735, having the 38E2 B7H3 arm instead of the 2E4, and comparing it to an RSV isotype control instead of a comparator test article. FIG. 29C depicts the effect over a range of concentrations instead of a range of time.

FIG. 30 depicts the ability of NKEs to synergize with IL-15 to enhance NKE activity. NK cells were added to MCF7 target cells at a 5:1 E:T ratio. A control group of cells was then left alone, while other groups were dosed with either IL15-Fc, NKG2D×B7H3 bsAb, or both. Target cell counts were then measured over time using the Incucyte® system. As seen in the figure, the combination of both IL15-Fc and NKE demonstrated significantly better target cell killing ability than either of the two treatments alone.

FIGS. 31A-31B depict the ability of NKEs to synergize with IL-12 to enhance NKE activity. In the experimental set up for FIG. 32A, NK cells were added to MCF7 target cells at a 5:1 E:T ratio. A control group of cells was then left alone, while other groups were dosed with either 10 μg/ml IL-12-Fc (XENP27201), 4 μg/ml NKG2D×B7H3 bsAb (XENP40377), or both. Target cell counts were then measured over time using the Incucyte® system. As seen in the figure, the combination of both IL-12-Fc and XENP40377 was significantly more effective at killing target cells than either of the two treatments alone. FIG. 31B depicts the results of a similar experimental set-up as described for FIG. 32A, but using 2 μg/ml IL-12-Fc XmAb662 (XENP39662) instead of 10 μg/ml XENP27201.

FIGS. 32A-32B depict the K_{D} values for NKG2D binding domains in the format of a bispecific antibody binding to both human NKG2D (FIG. 32A) and cynomolgus NKG2D (FIG. 32B). In this Octet experiment, HIS1K sensors were used to capture human NKG2D (XENP23311) or cynomolgus NKG2D (XENP23309) antigens at 20 nM for 3 minutes. Antigens were then dipped into a respective test article at concentrations of 300 nM, 150 nM, 75 nM, 37.5 nM, 18.75 nM, 9.38 nM, 4.69 nM, and 0 nM with an association time of 5 minutes and a dissociation time of 10 minutes.

FIG. 33 depicts the ability of NKEs, particularly those with 1D7B4 and 1D2B4 NKG2D binding domains, to synergize with IL-15 for target cell killing and titration response. In this experiment, NK cells were added to OVCAR8 target cells at a 5:1 E:T ratio. Cells were dosed with 10 μg/ml IL-15 Fc (XENP24045) and NKEs were titrated in at a dose range of 1.5 ng/ml to 10,000 μg/ml. Incucyte was used to quantify target cells over time.

FIG. 34 depicts useful Fc variants that increase FcγRIIIA binding and enhance ADCC activity. These variants may be used with or without Xtend variants (M428L/N434S or M428L/N434A).

FIGS. 35A-35D show the sequences of several heterodimeric αNKG2D×αB7H3 backbones with ablated effector function (also referred to as the FcKO variants). Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 14 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure.

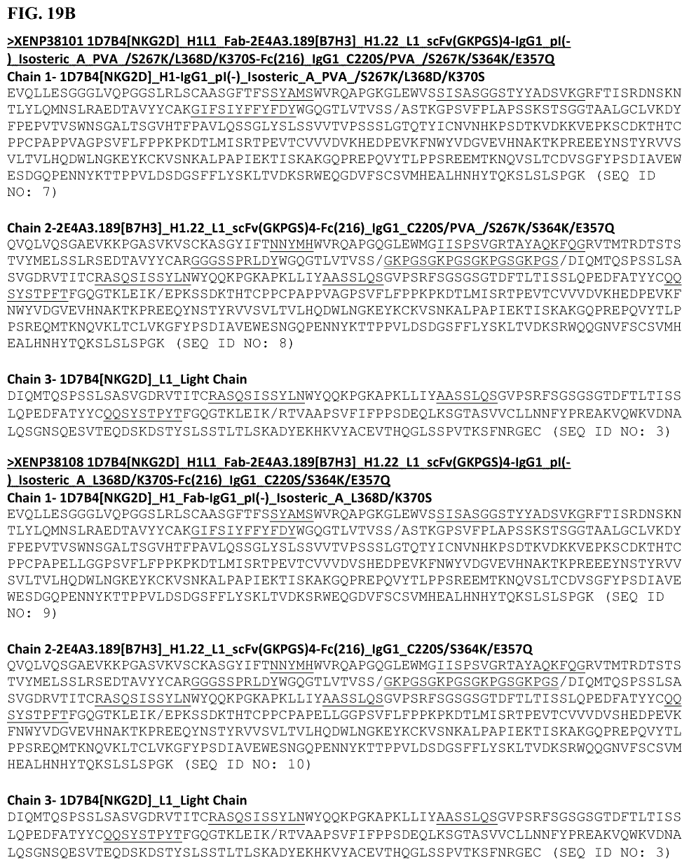

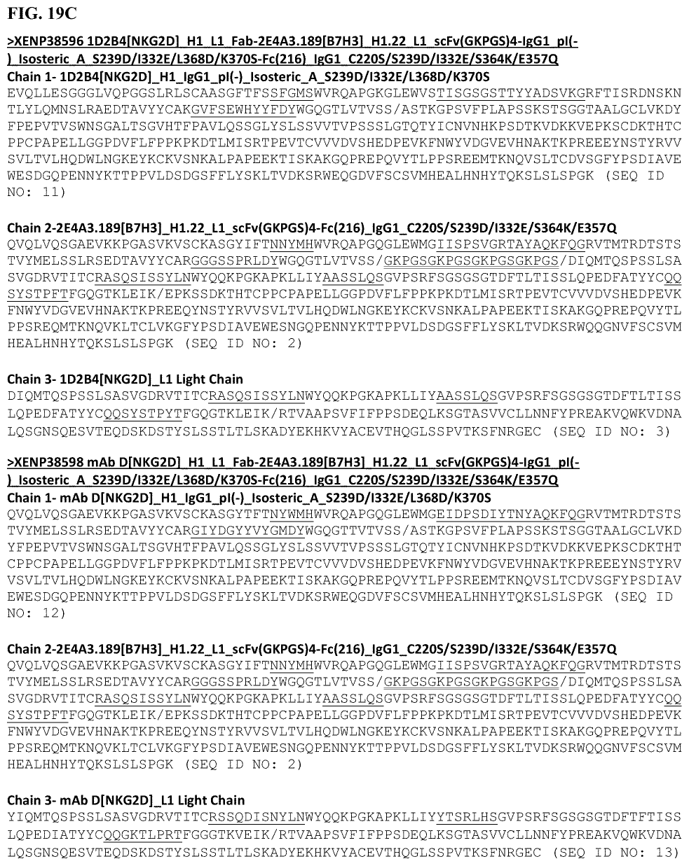

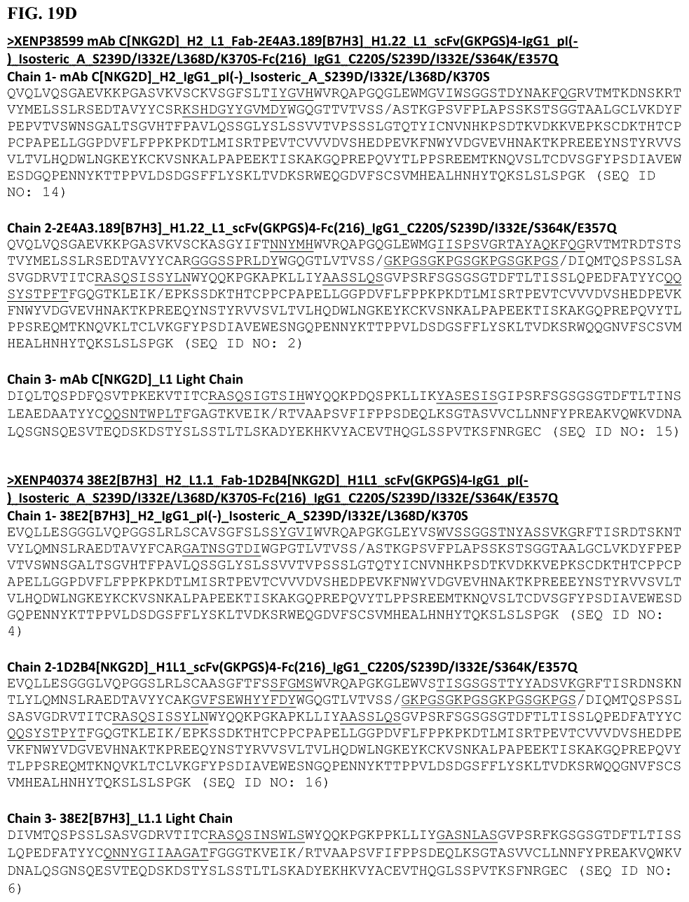

FIG. 36 depicts sequences for “CH1+hinge” that find use in embodiments of αNKG2D×αB7H3 bsAbs that utilize a Fab binding domain. The “CH1+hinge” sequences find use linking the variable heavy domain (V_{H}) to the Fc backbones depicted in FIG. 19. For particular embodiments wherein the Fab is on the (+) side, the “CH1(+)+hinge” sequences may find use. For particular embodiments wherein the Fab is on the (−) side, the “CH1(−)+hinge” sequences may find use.

FIG. 37 depicts sequences for “CH1+half hinge” domain linker that find use in embodiments of αNKG2D×αB7H3 bsAbs in the 2+1 Fab2-scFv-Fc format. In the 2+1 Fab2-scFv-Fc format, the “CH1+half hinge” sequences find use linking the variable heavy domain (V_{H}) to the scFv domain on the Fab-scFv-Fc side of the bispecific antibody. It should be noted that other linkers may be used in place of the “CH1+half hinge”. It should also be noted that although the sequences here are based on the IgG1 sequence, equivalents can be constructed based on the IgG2 or IgG4 sequences.

FIG. 38 depicts sequences for “CH1” that find use in embodiments of αNKG2D×αB7H3 bsAbs.

FIG. 39 depicts sequences for “hinge” that find use in embodiments of αNKG2D×αB7H3 bsAbs.

FIG. 40 depicts a matrix of symmetric and asymmetric v90 Fc variants that have been engineered, as well as the corresponding Tm data, affinity data, production yield, ADCC activity and target cell killing activity. As shown, each Fc monomer (−Fc HC or +Fc-scFv-Fc) has either the S239D and I332E (V90) variants, the S239D variant alone, the I332E variant alone, or is wild-type at the 239 and 332 positions; and each test article has a different combination of these Fc monomers.

FIG. 41 depicts the range of ADCC activity of the various symmetric and asymmetric V90 variants outlined in FIG. 40. The results show a large range in levels of fold change in ADCC activity of each construct compared to wildtype, (“WT”) with V90 having one of the highest fold changes in ADCC activity compared to WT, and the various S239D and I332E combinations showing a broad range of intermediate levels fold changes.

FIG. 42 depicts the affinity data for detuned 1D7B4 Fab variants.

FIG. 43 depicts the affinity data for select detuned 1D7B4 variants in the context of a αB7H3×αNKG2D 1+1 Fab-scFv-Fc.

FIGS. 44A-44B depict the NK cell killing and the NK cell degranulation by 1D7B4 detuned variants. FIG. 44A depicts the NK cell killing and the NK cell degranulation of 1D7B4 detuned variants. As seen, most of the detuned variants, with the exception of 1D7B4_H1.28 (XENP42660), showed a significant decrease in the amount of fratricide when compared to the parental XENP40377. FIG. 44B depicts the same effect with a smaller set of test articles. In this experiment, NK cells were co-cultured with treatments for 12 hours. NK cell lysis was assessed via staining with a viability dye, staining was quantified by flow cytometry.

FIGS. 45A-45C depict the ability αB7H3×αNKG2D 1+1 molecules having the affinity detuned 1D7B4 variants to induce target cell lysis and IFNγ production, with and without IL-15. In this experiment, parental A375 cells were plated, and effector cells were added at a 5:1 E:T ratio. Tumor cell growth was assessed using Incucyte. The IL15-Fc used was XENP24045.

FIG. 46 depicts the reduction of potency of fratricide when the NKE is in the scFv format. It also demonstrates that the extent of this reduction in fratricide is influenced by the level of CD16 expression on the NK cells.

FIG. 47 depicts the impact of different NKE antibody formats on their binding to NK cells and corresponding affinities.

FIG. 48 depicts the affinities of NKEs having NKG2D in either the V_{H}-V_{L} or the V_{L}-V_{H} orientation as well as in different antibody formats.

FIGS. 49A-49D depict the ability of NKEs to induce NK cell and CD8+ T cell activation in vivo. In this study, female huCD34+ NSG mice were inoculated intradermally with 3×106 ppMCF7-GFP cells per mouse on Day −16. Then on Day 0, they were dosed with a 5 mg/kg NKE treatment and a 0.2 mg/kg IL-15-Fc (XENP24045) treatments intraperitoneally. Activation marker CD69 is upregulated in NK cells and CD8+ T cells in groups treated with NKG2D×B7H3 NKEs but not in groups treated with RSV×B7H3 controls.

FIGS. 50A-50C show the sequences of several useful heterodimeric αB7H3×αNKG2D bsAb backbones based on human IgG1 and having WT effector function. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and N297S variant that removes glycosylation on both chains. Heterodimeric Fc backbone 8 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434S Xtend variants on both chains. Heterodimeric Fc backbone 9 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 10 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434A Xtend variants on both chains. Heterodimeric Fc backbone 11 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIGS. 51A-51C depict the sequences of several useful heterodimeric αB7H3×αNKG2D bsAb backbones based on human IgG1 and having enhanced ADCC function. The sequences here are based on heterodimeric Fc backbone 1 in FIG. 50, although the ADCC variants in FIG. 51 may also be included in any of the other heterodimeric Fc backbones in FIG. 50. ADCC-enhanced Heterodimeric Backbone 1 includes S239D/I332E on both the first and the second heterodimeric Fc chain. ADCC-enhanced Heterodimeric Backbone 2 includes S239D on both the first and the second heterodimeric Fc chain. ADCC-enhanced Heterodimeric Backbone 3 includes I332E on both the first and the second heterodimeric Fc chain. ADCC-enhanced Heterodimeric Backbone 4 includes S239D/I332E on the second heterodimeric Fc chain. ADCC-enhanced Heterodimeric Backbone 5 includes S239D on the second heterodimeric Fc chain. ADCC-enhanced Heterodimeric Backbone 6 includes I332E on the second heterodimeric Fc chain. ADCC-enhanced Heterodimeric Backbone 7 includes S239D/I332E on the first heterodimeric Fc chain. ADCC-enhanced Heterodimeric Backbone 8 includes S239D on the first heterodimeric Fc chain. ADCC-enhanced Heterodimeric Backbone 9 includes I332E on the first heterodimeric Fc chain. ADCC-enhanced Heterodimeric Backbone 10 includes S239D/I332E on the first heterodimeric Fc chain and S239D on the second heterodimeric Fc chain. ADCC-enhanced Heterodimeric Backbone 11 includes S239D/I332E on the first heterodimeric Fc chain and I332E on the second heterodimeric Fc chain. ADCC-enhanced Heterodimeric Backbone 12 includes S239D on the first heterodimeric Fc chain and S239D/I332E on the second heterodimeric Fc chain. ADCC-enhanced Heterodimeric Backbone 13 includes I332E on the first heterodimeric Fc chain and S239D/I332E on the second heterodimeric Fc chain. ADCC-enhanced Heterodimeric Backbone 14 includes S239D on the first heterodimeric Fc chain and I332E on the second heterodimeric Fc chain. ADCC-enhanced Heterodimeric Backbone 15 includes I332E on the first heterodimeric Fc chain and S239D on the second heterodimeric Fc chain.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1. That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

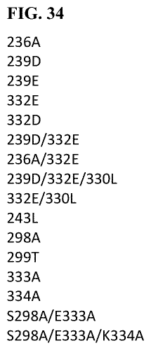

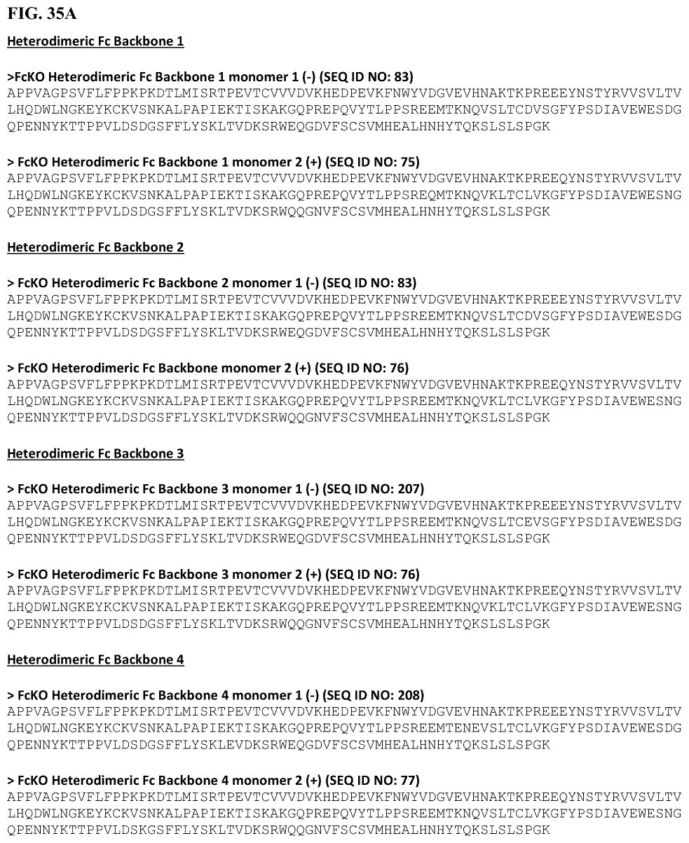

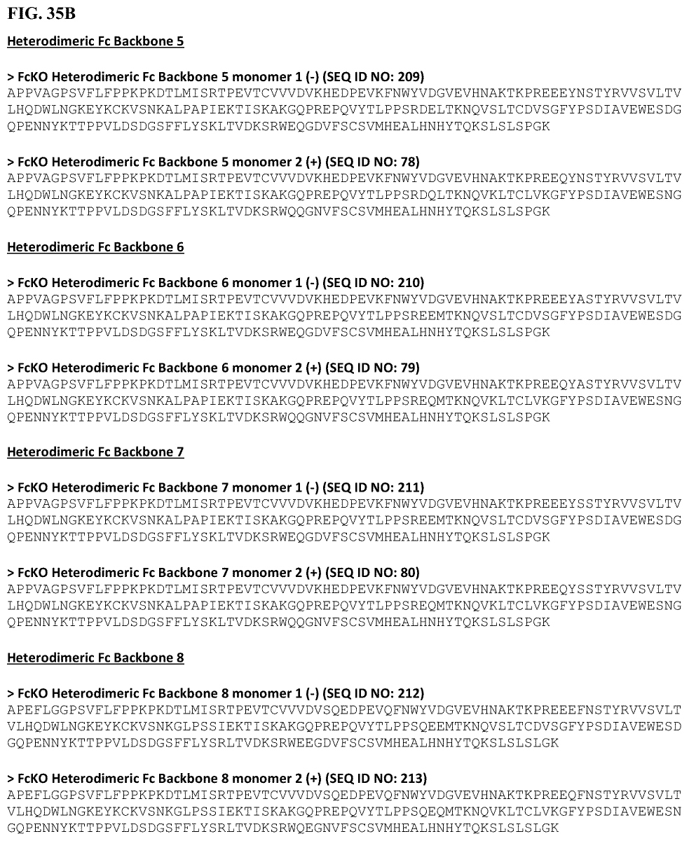

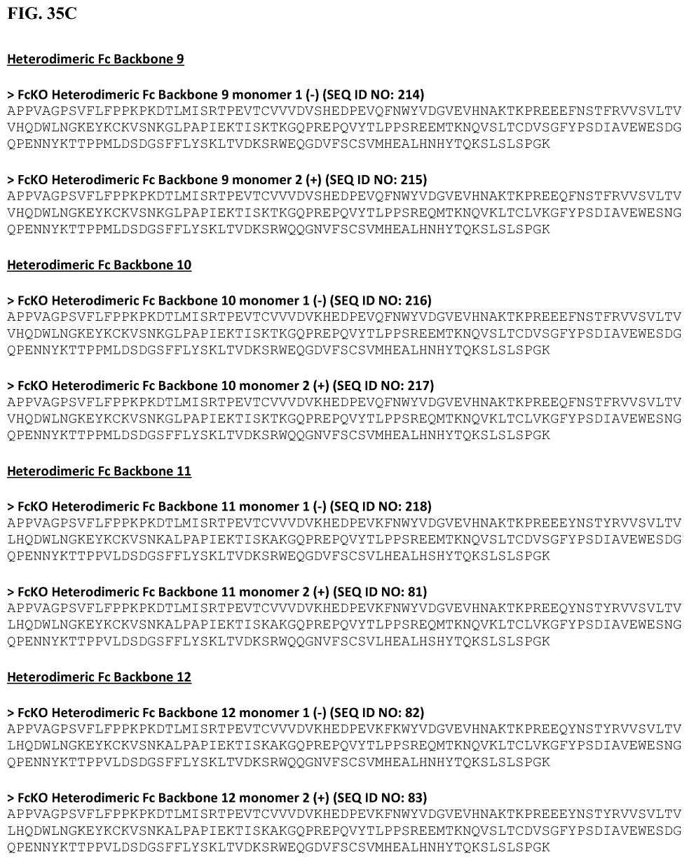

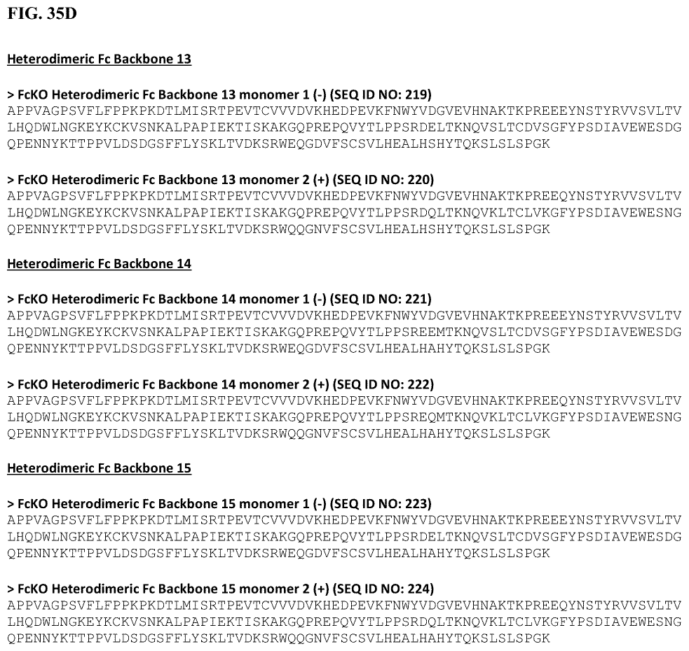

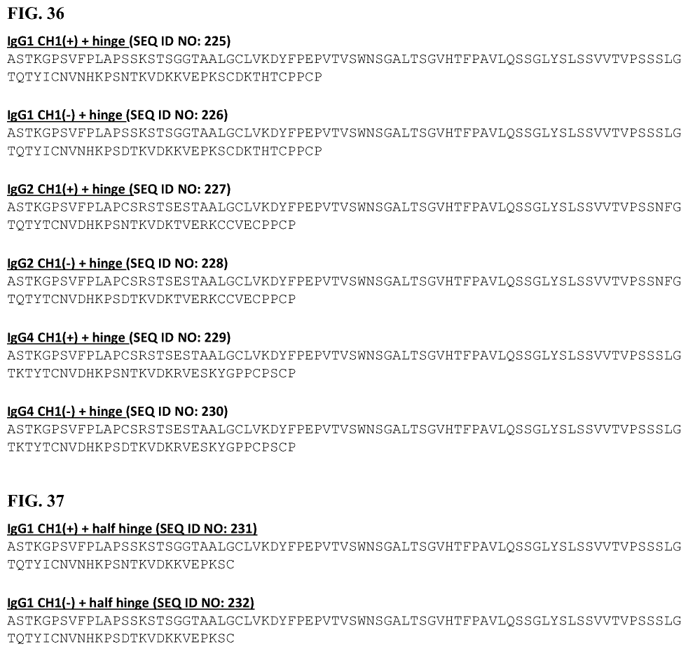

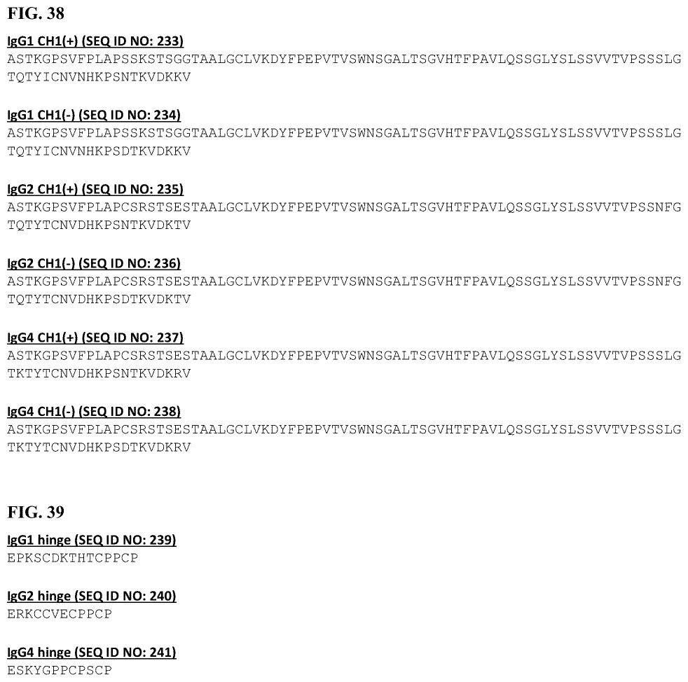

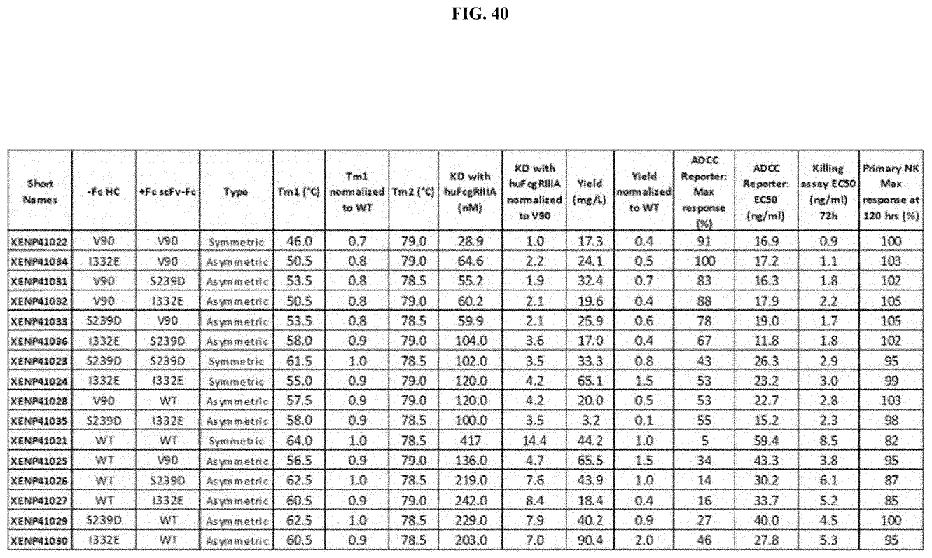

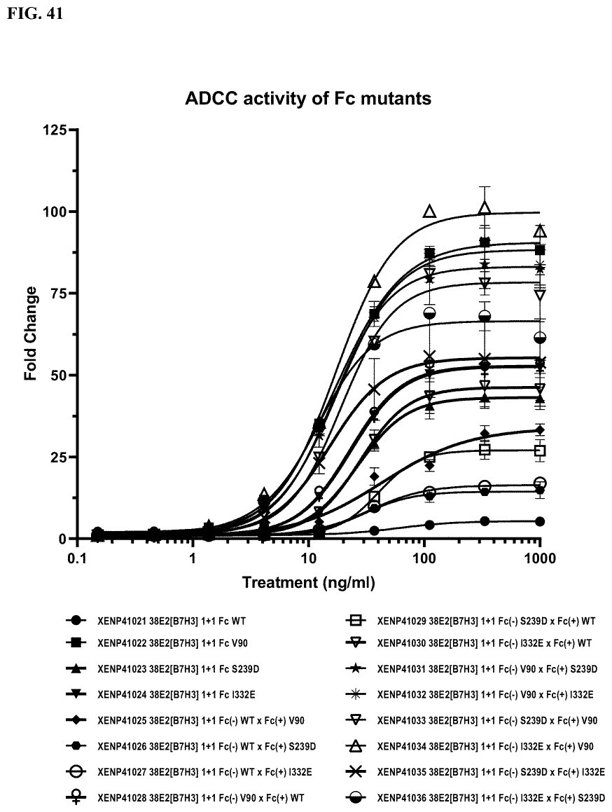

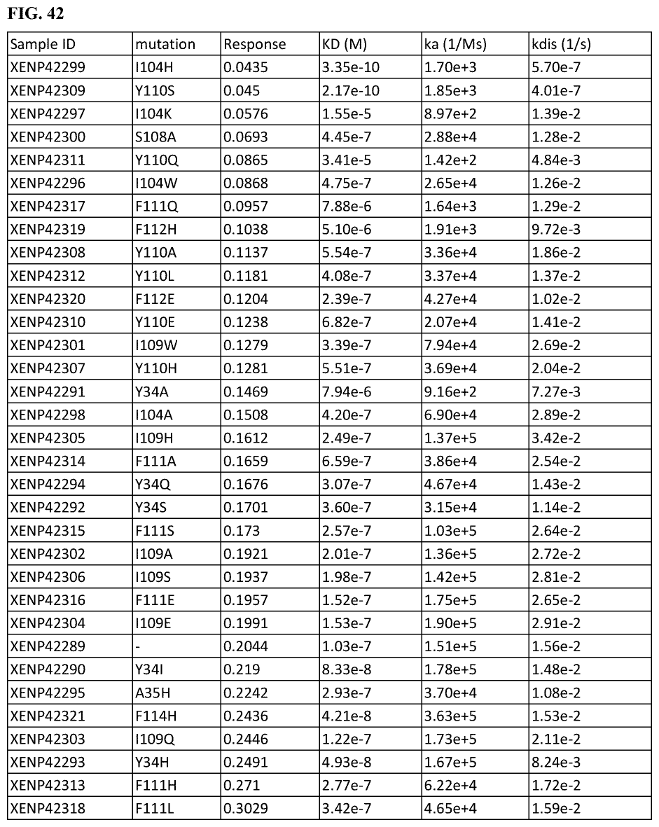

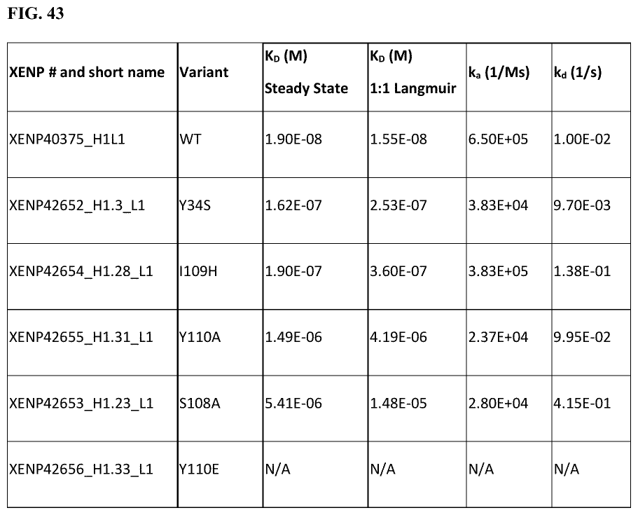

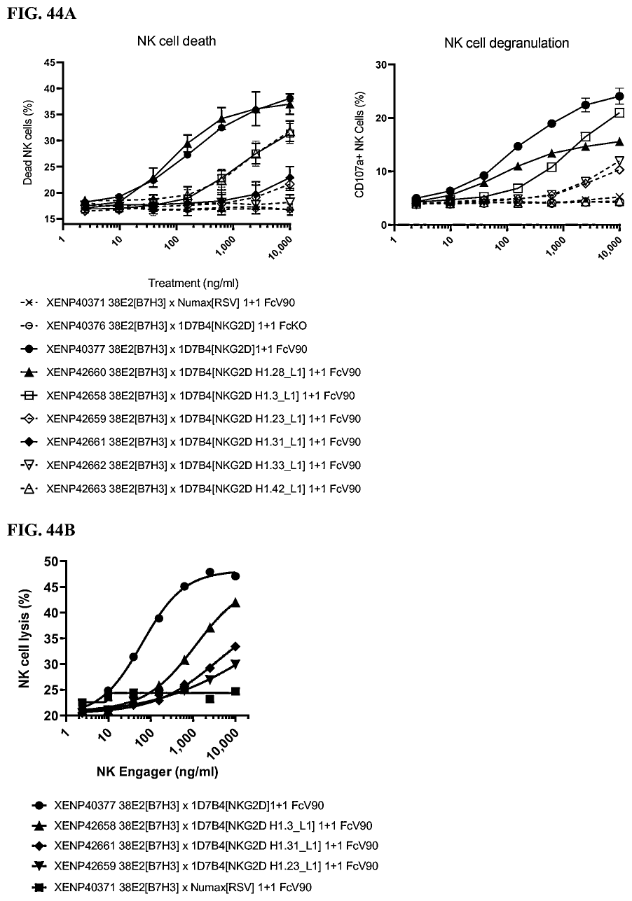

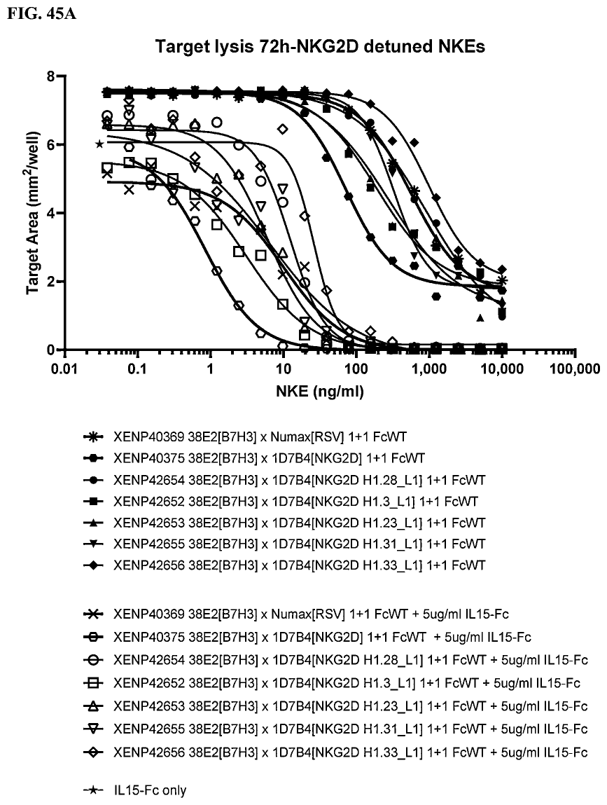

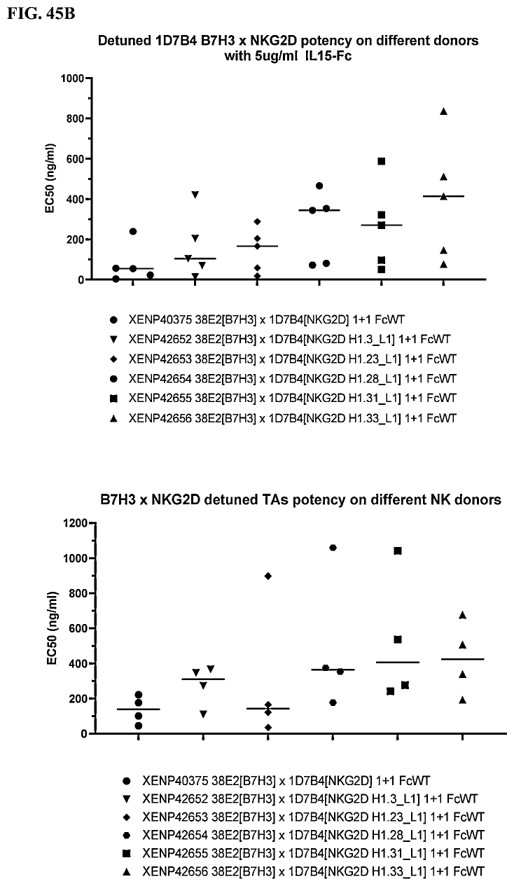

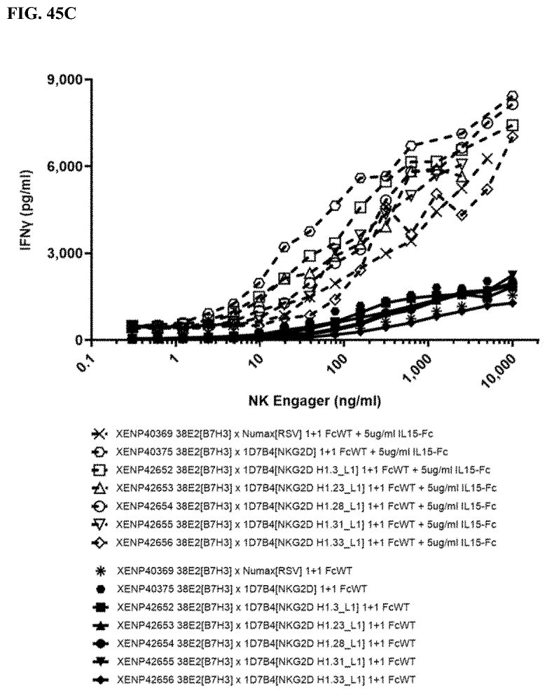

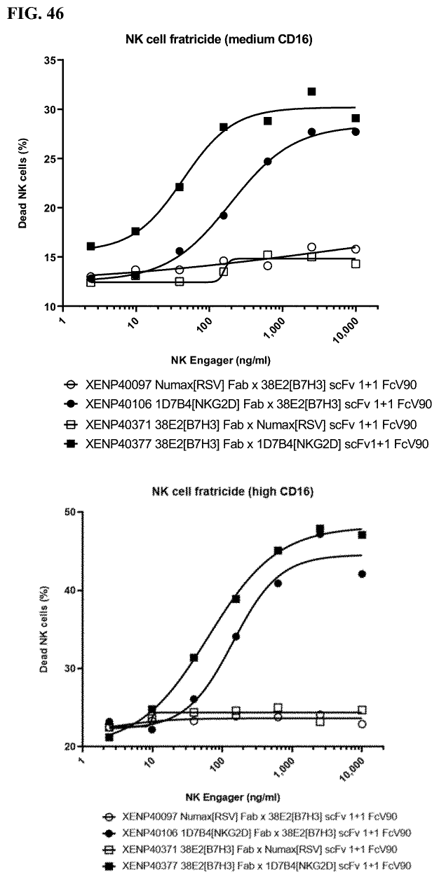

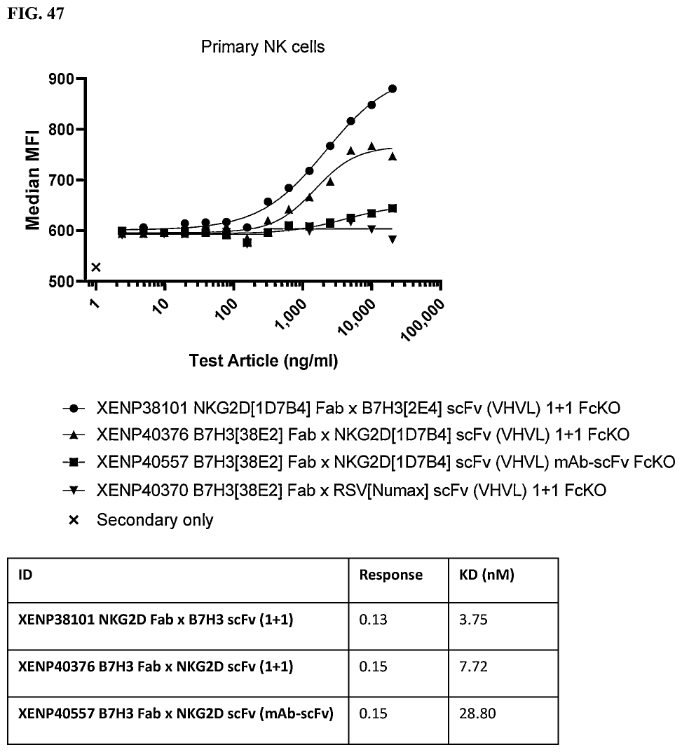

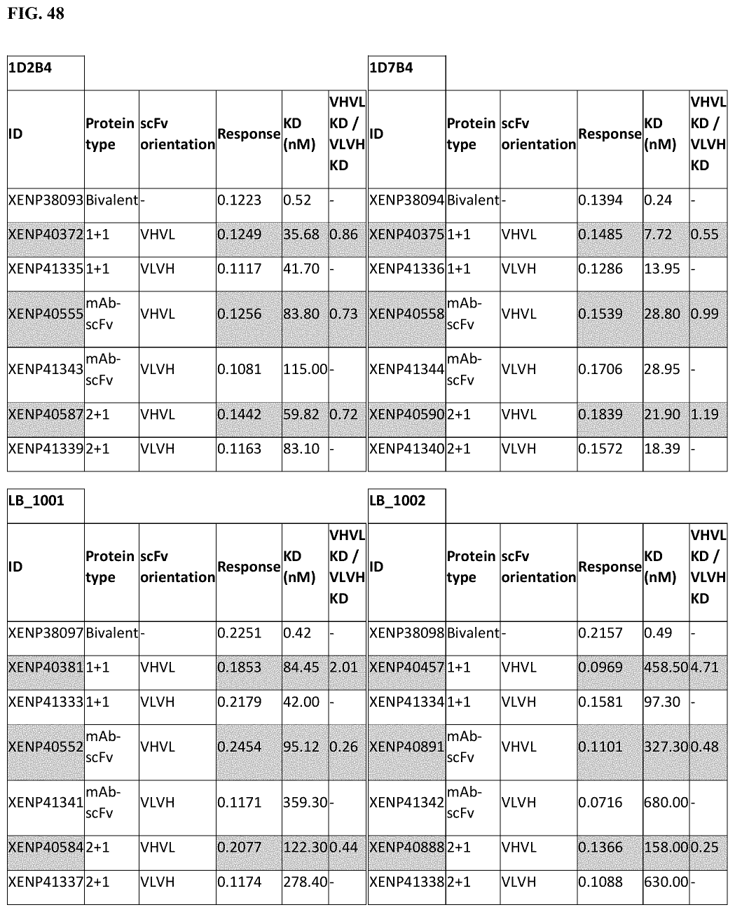

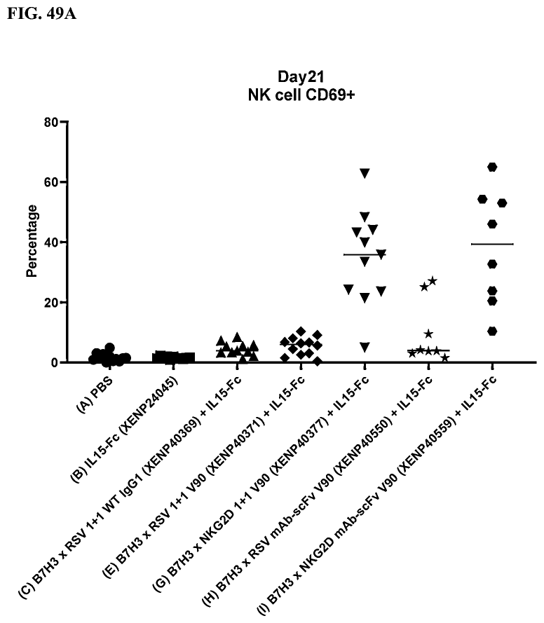

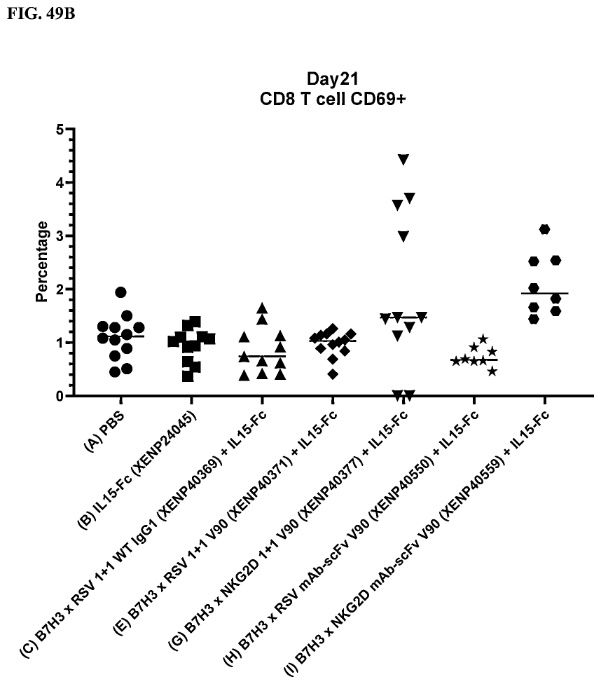

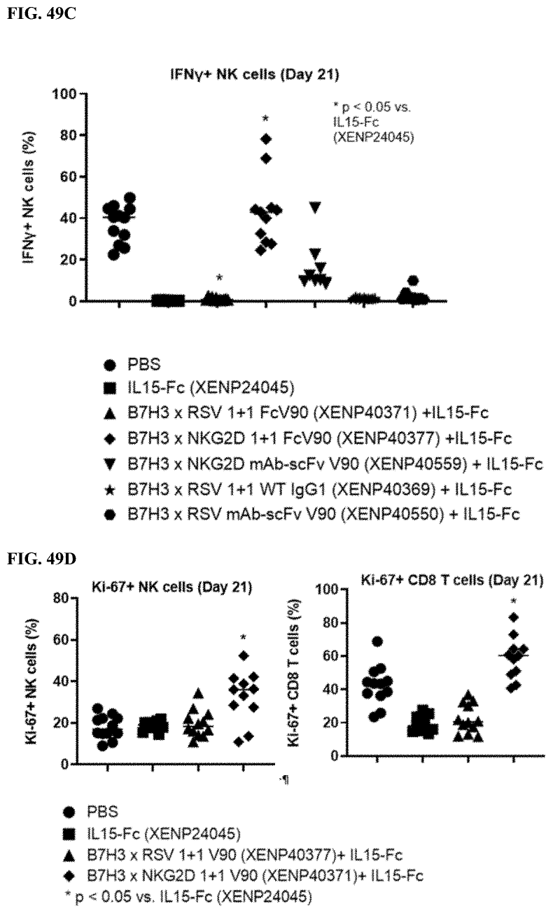

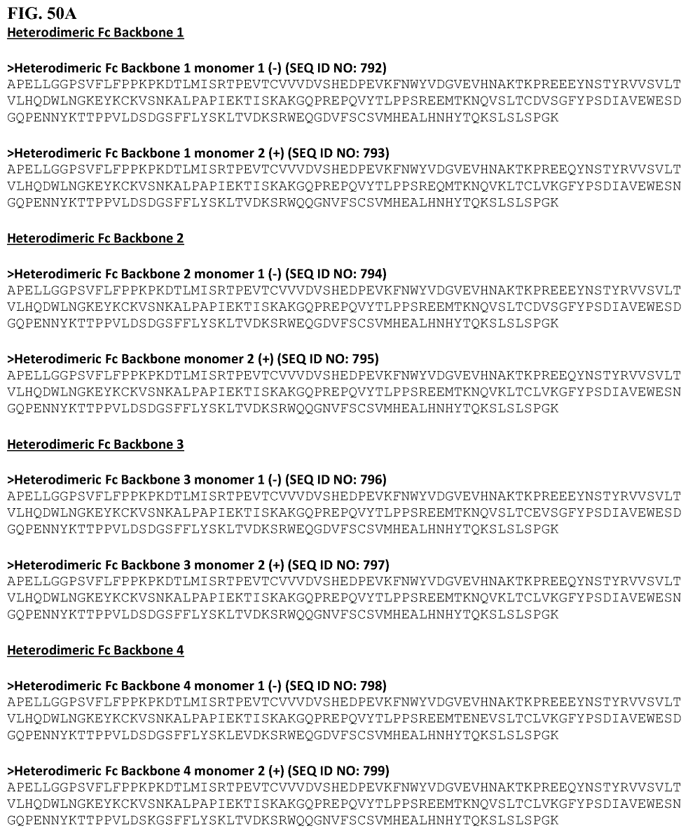

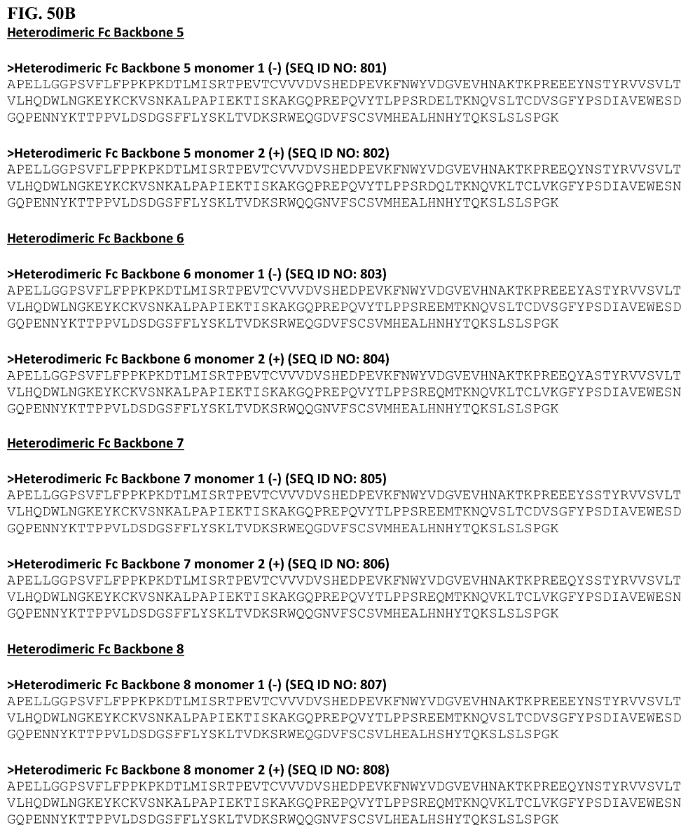

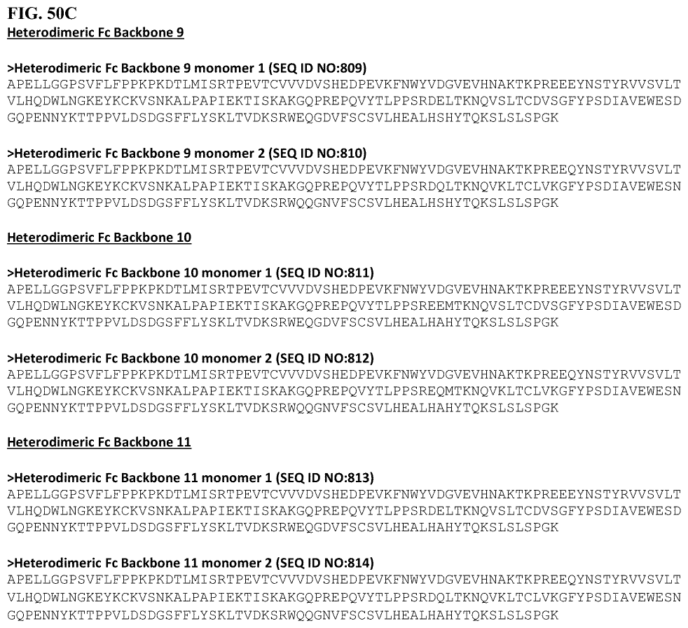

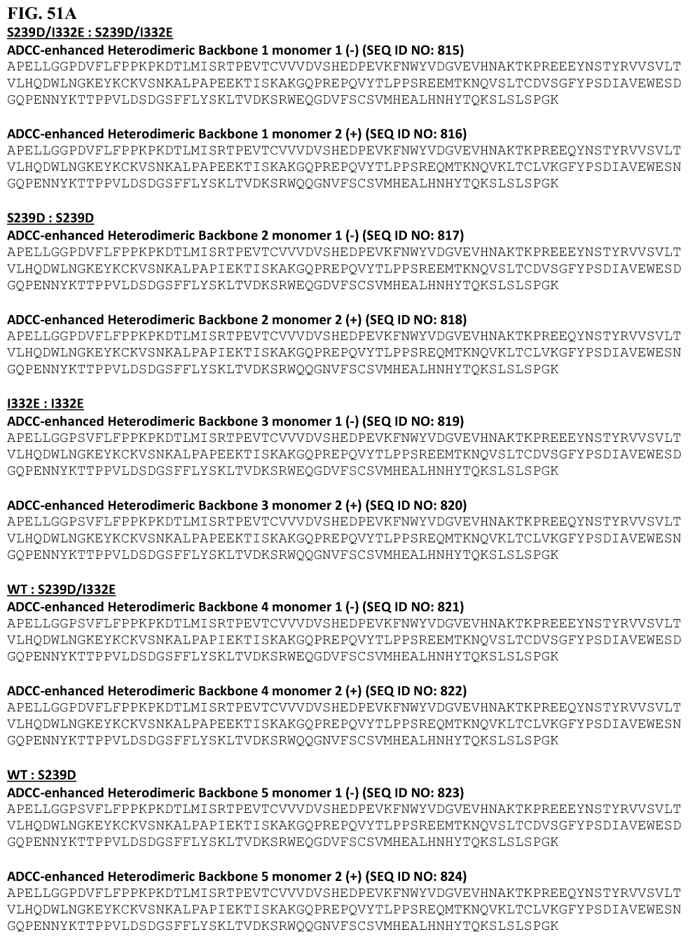

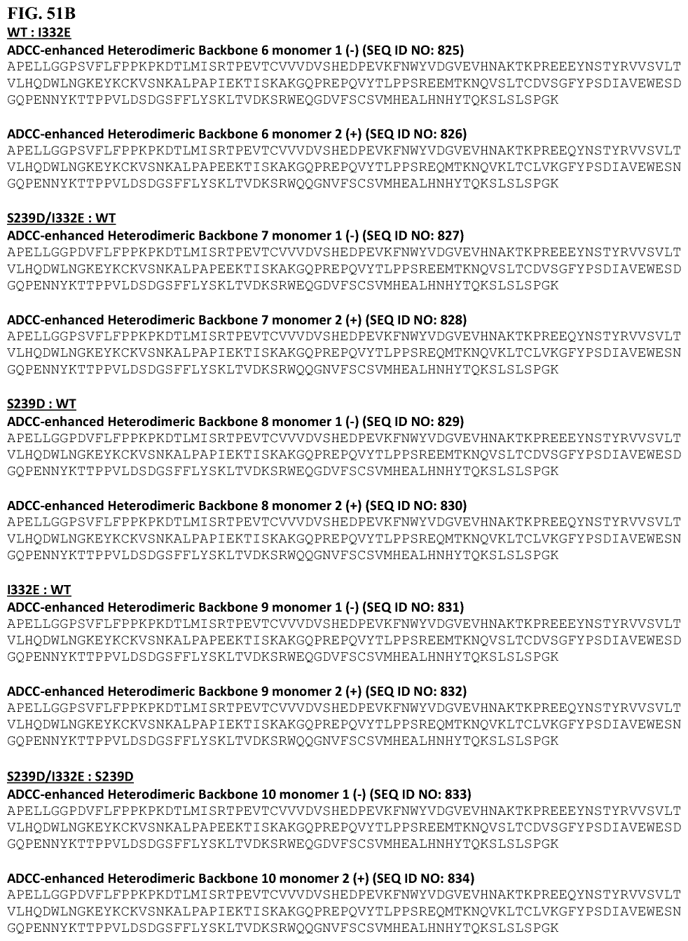

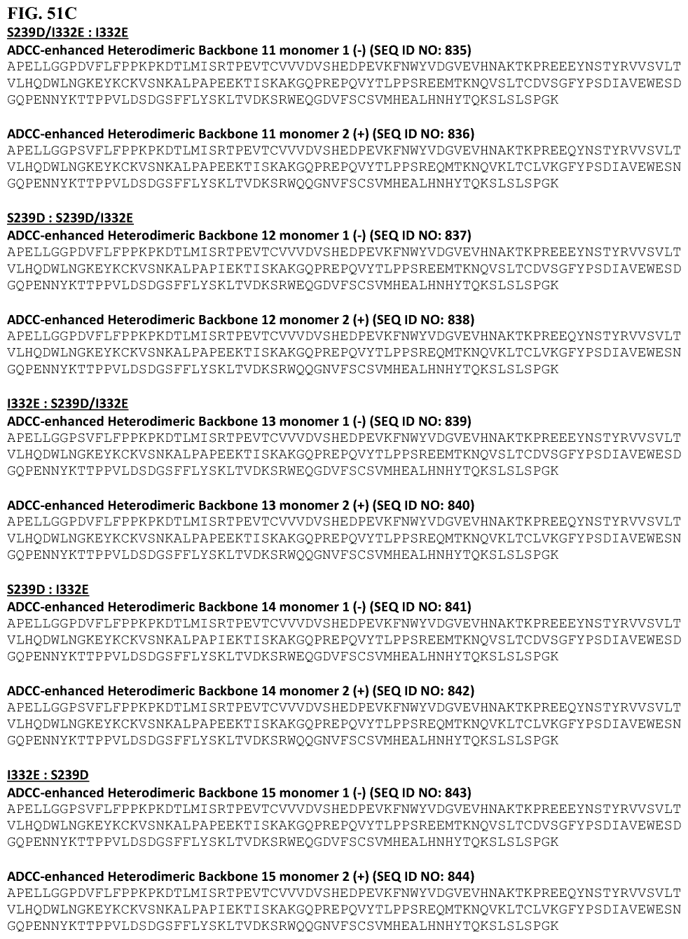

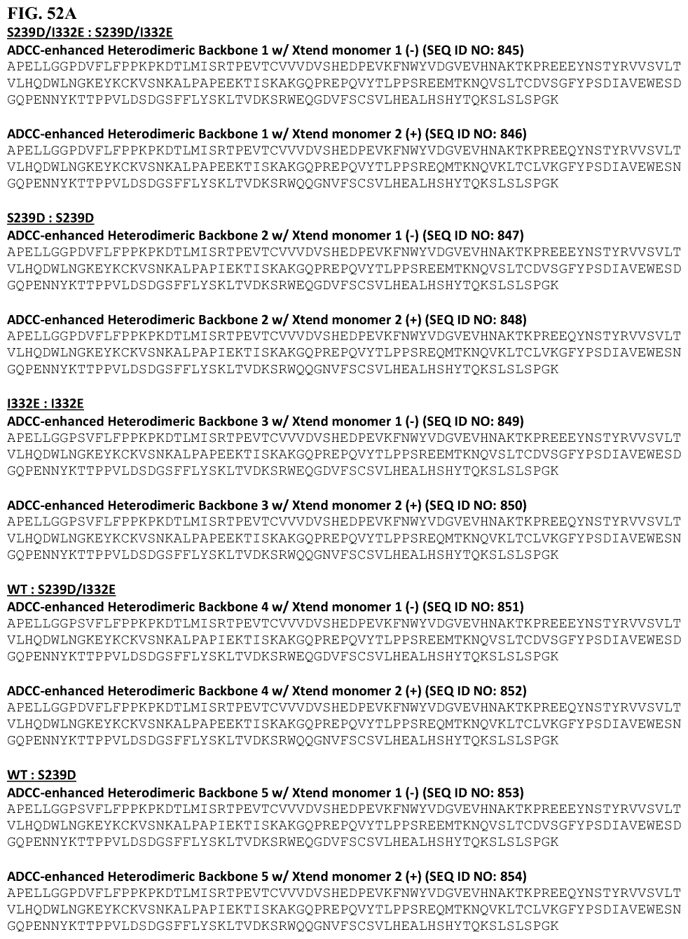

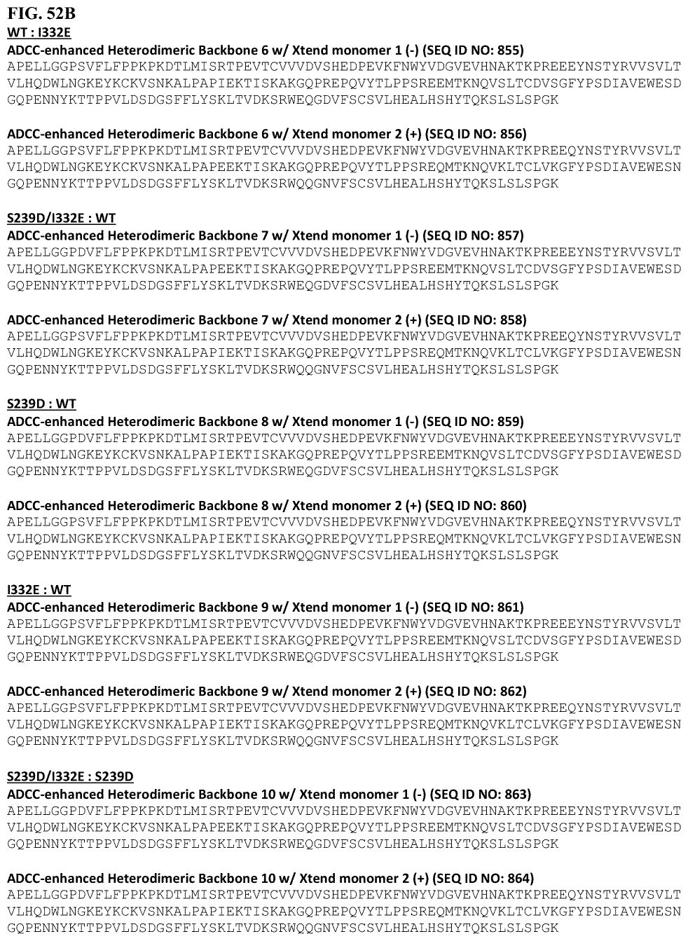

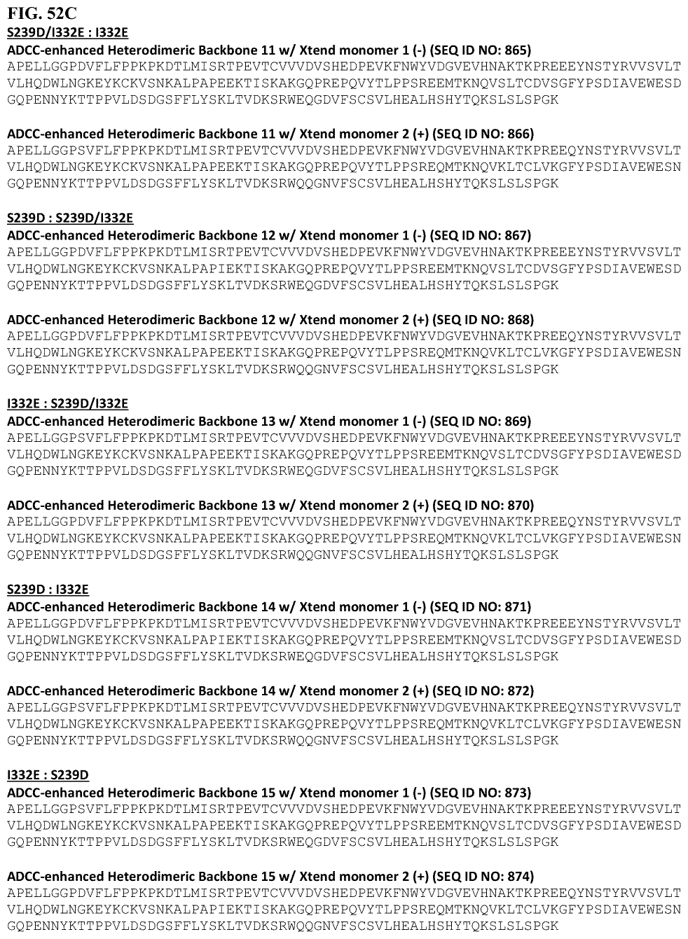

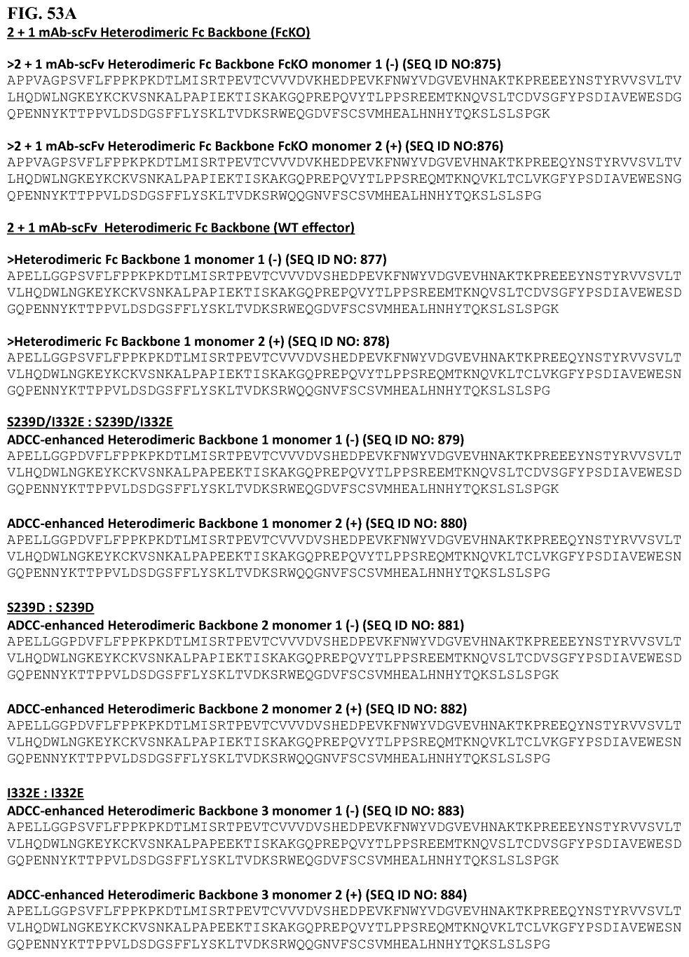

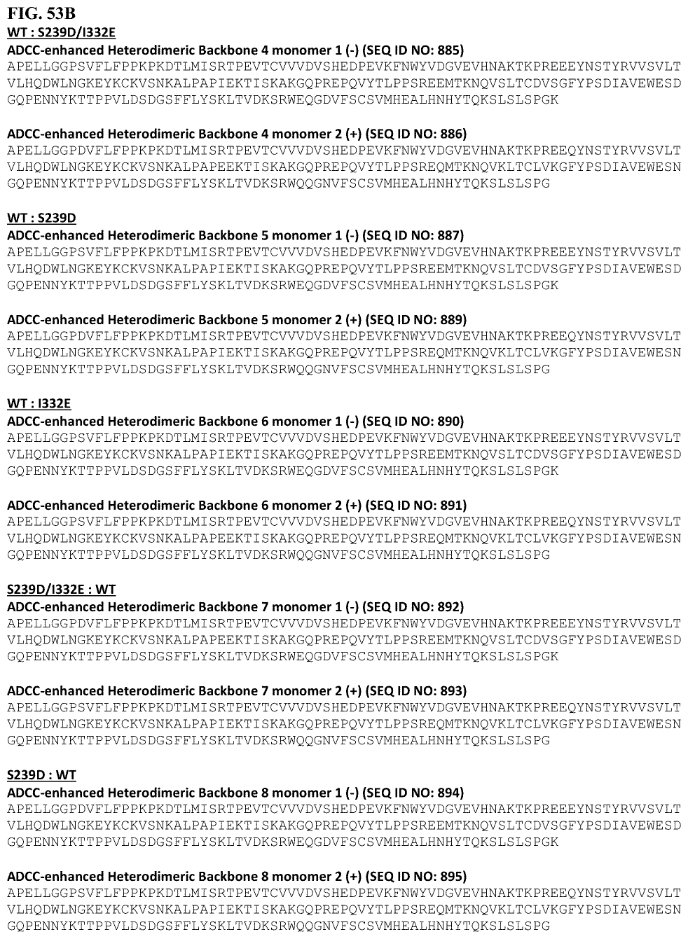

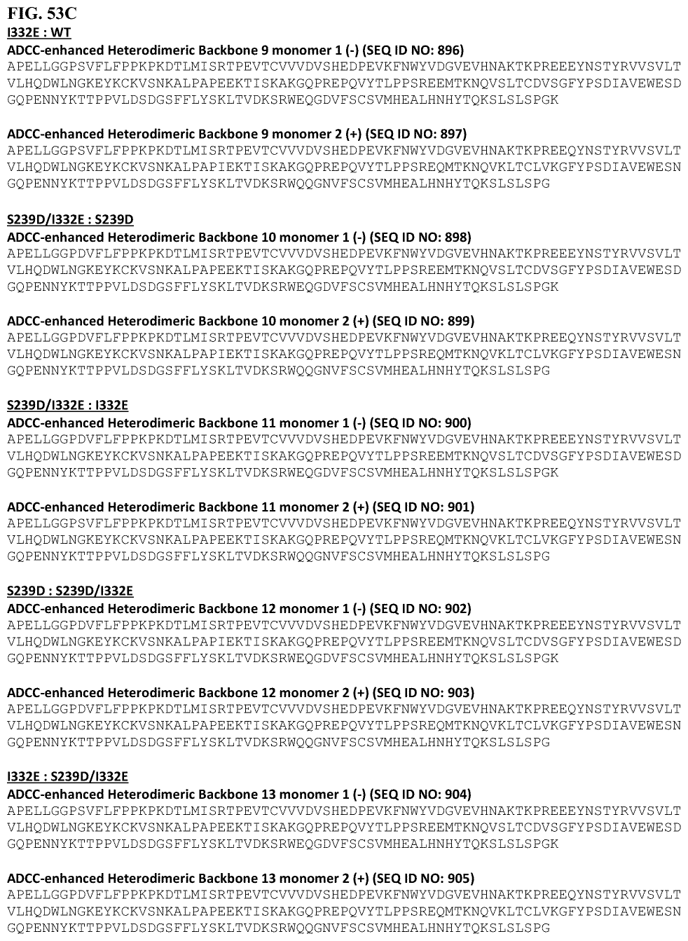

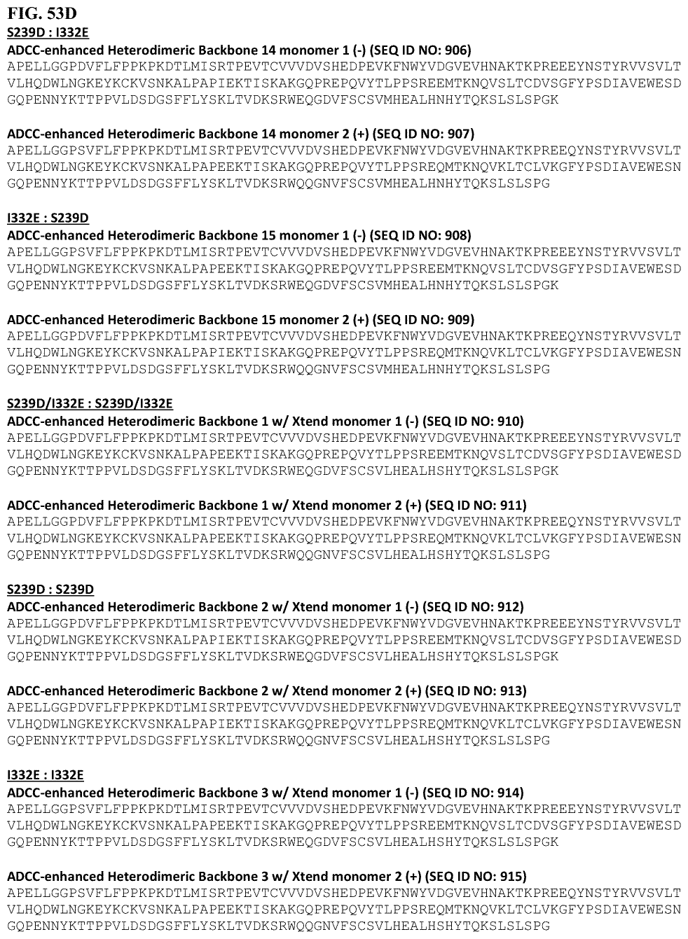

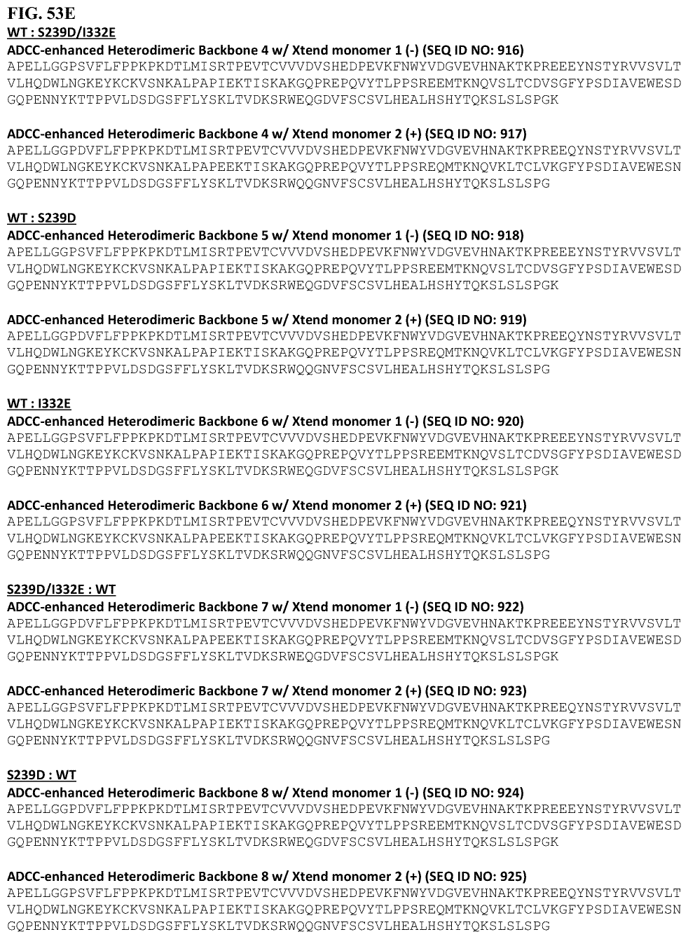

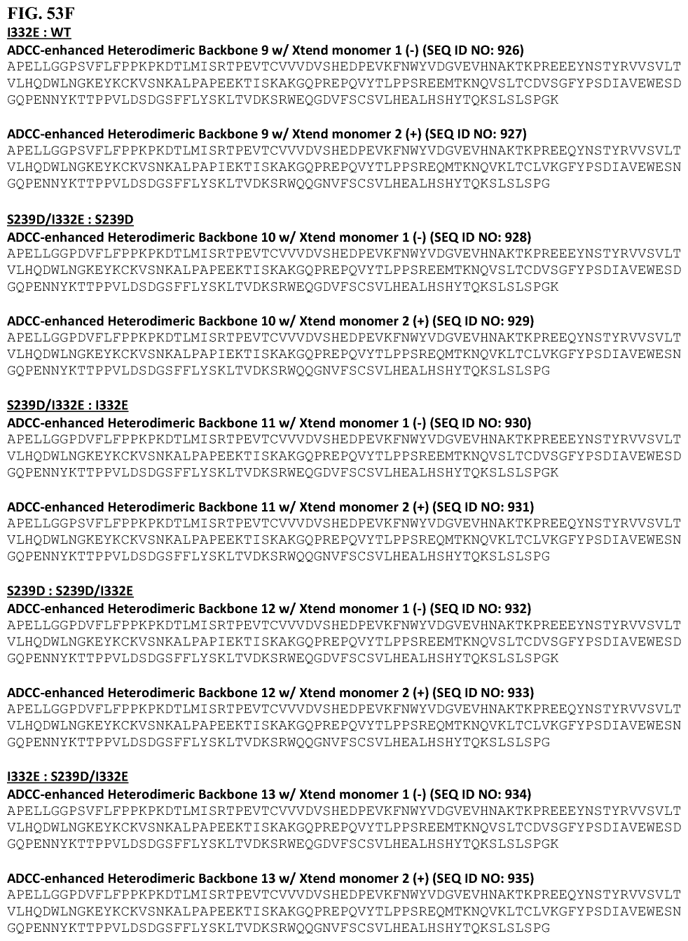

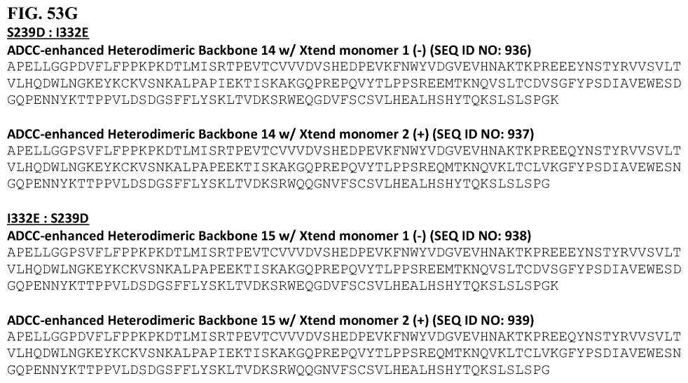

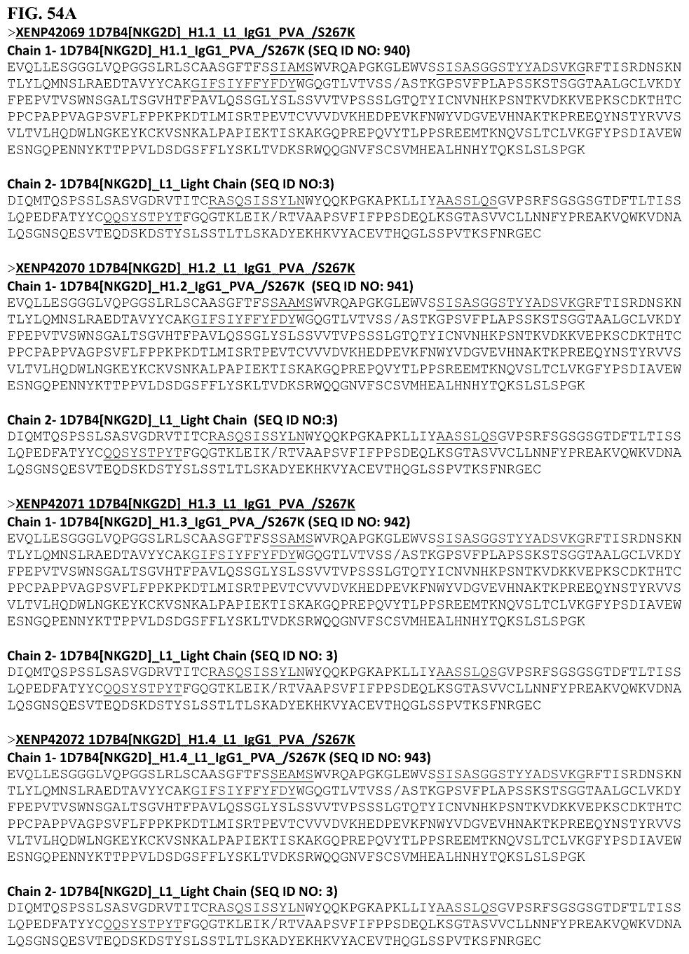

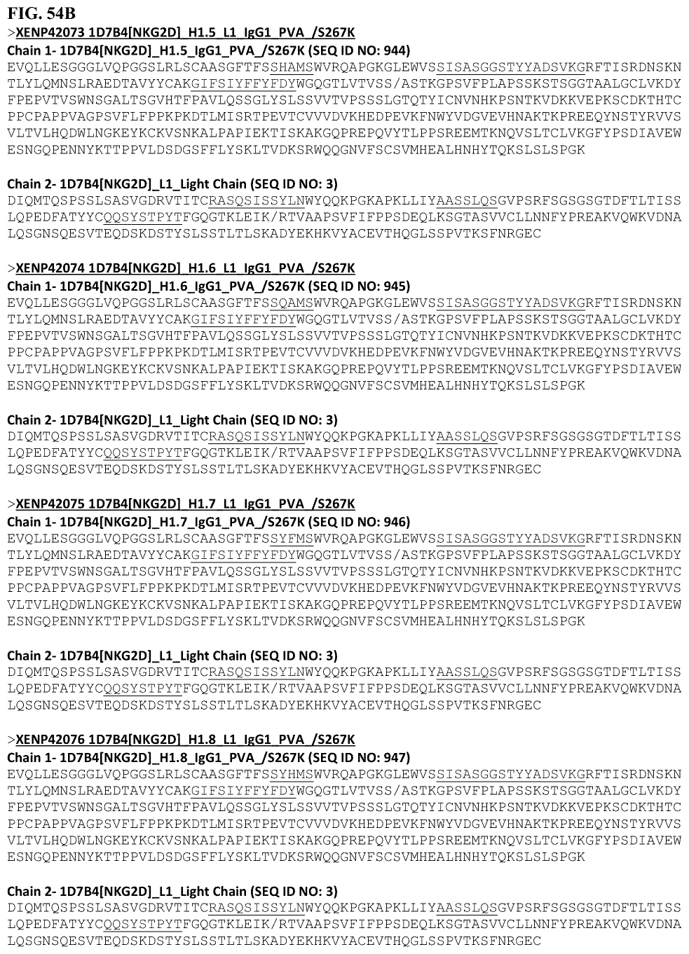

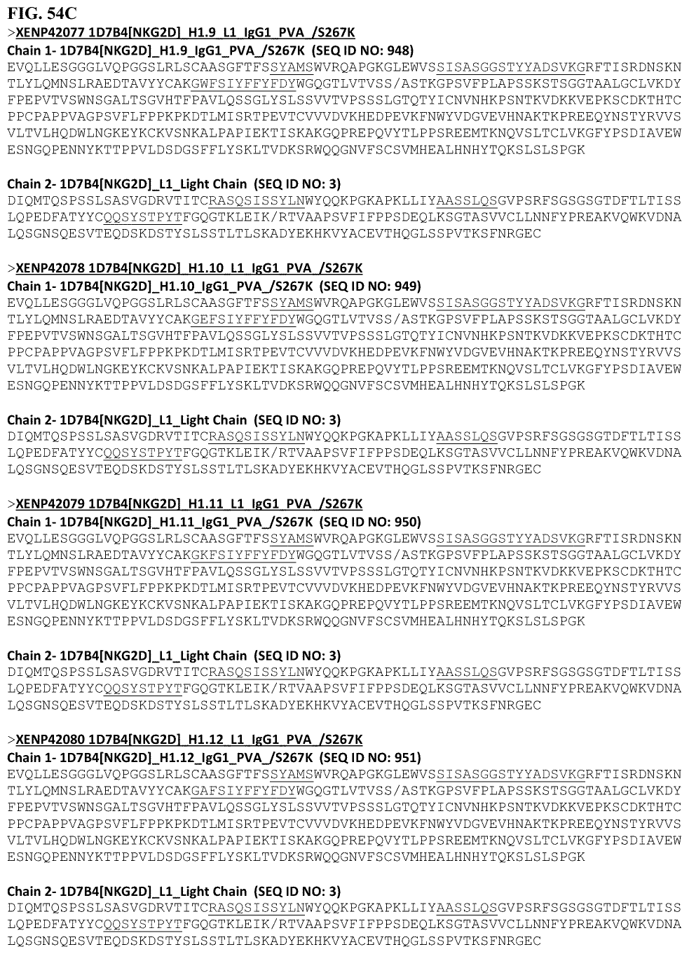

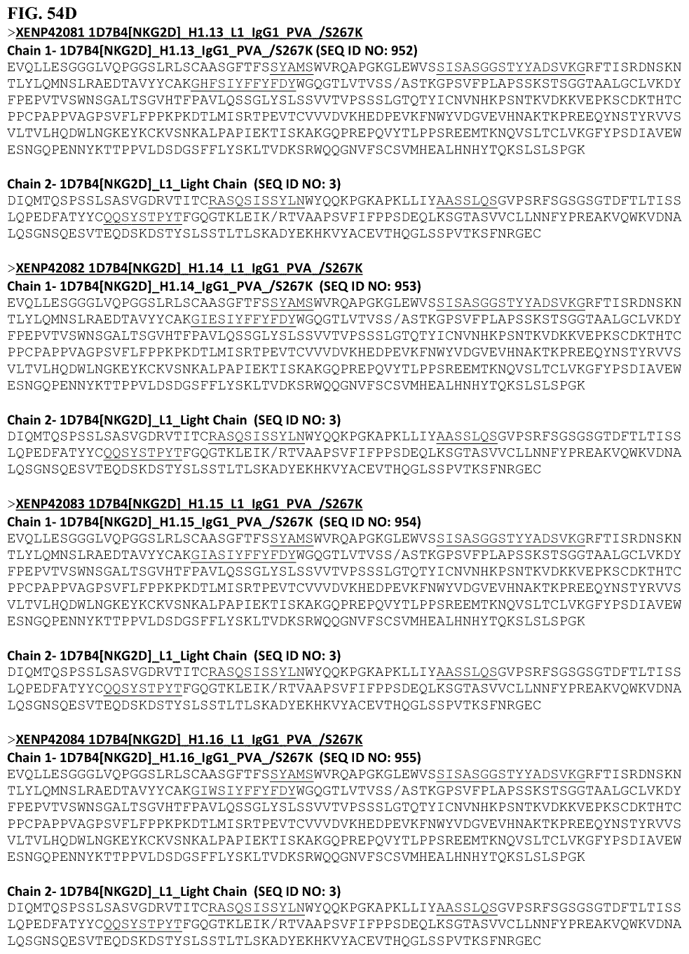

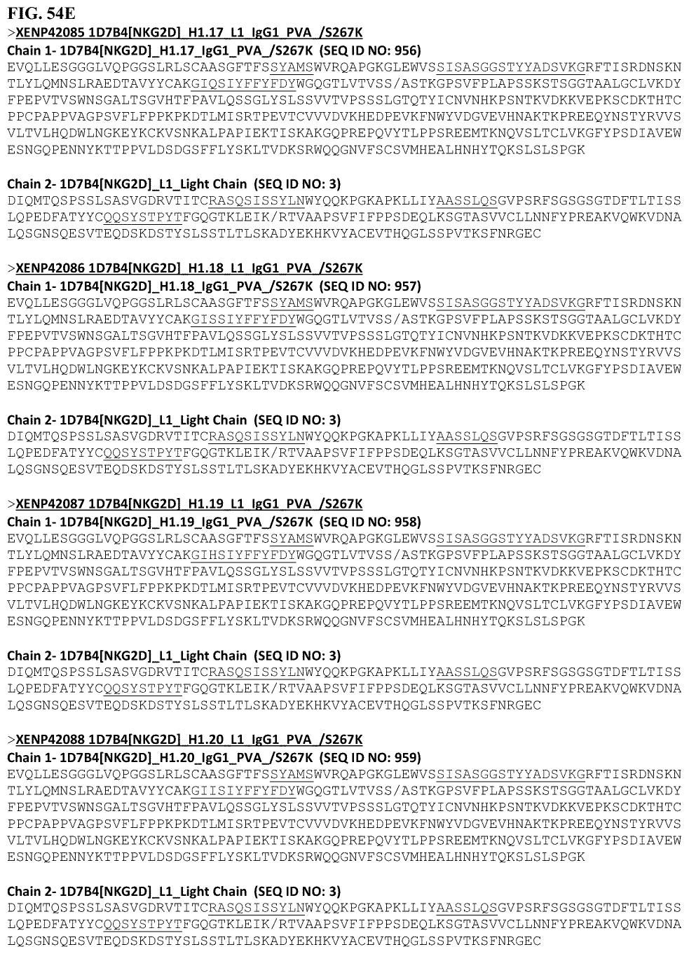

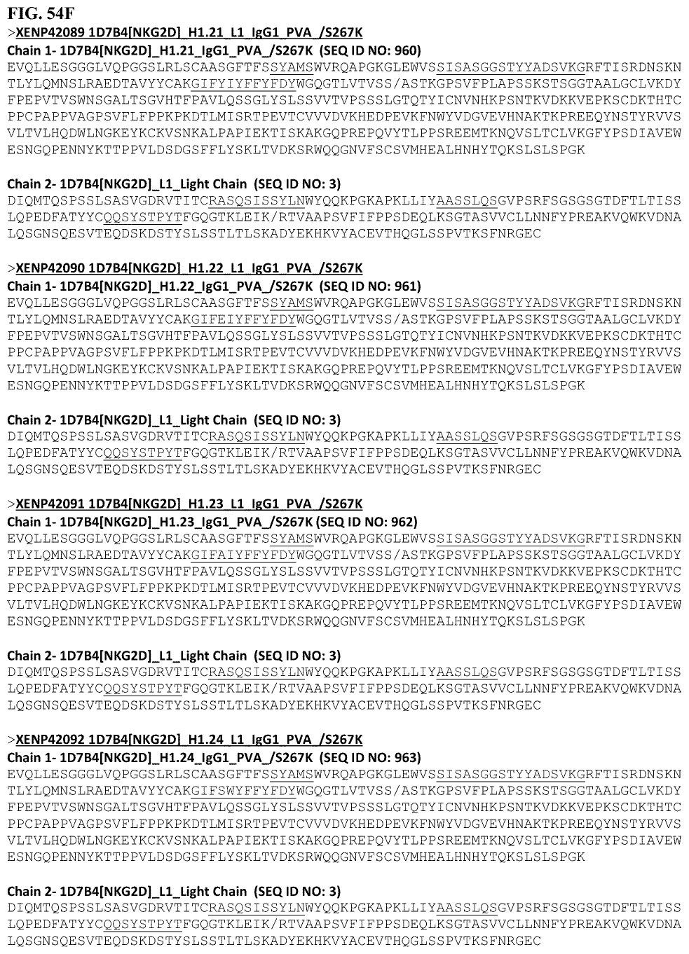

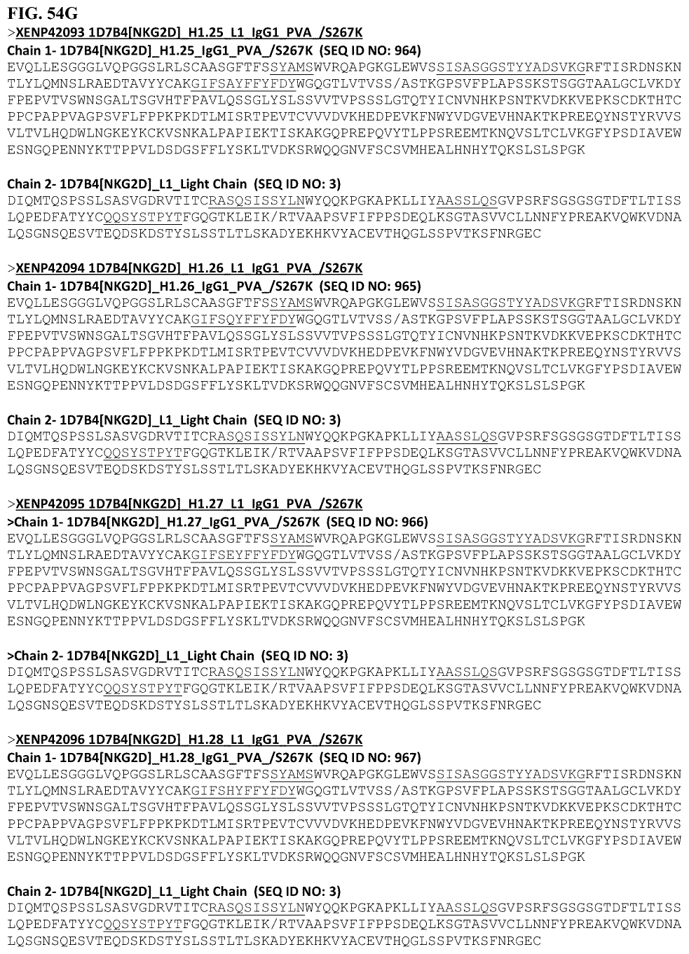

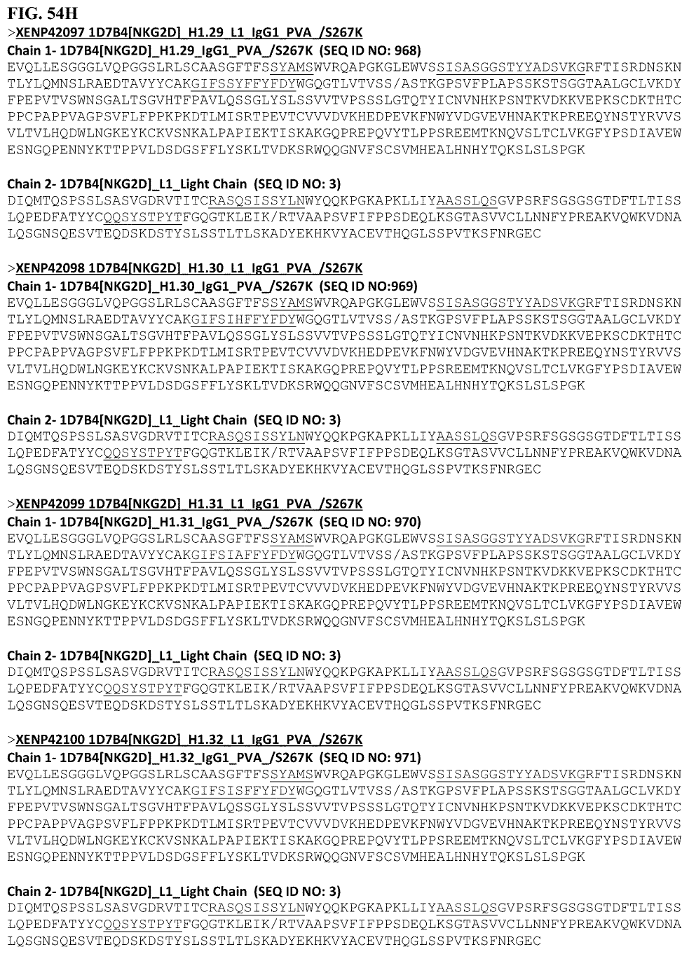

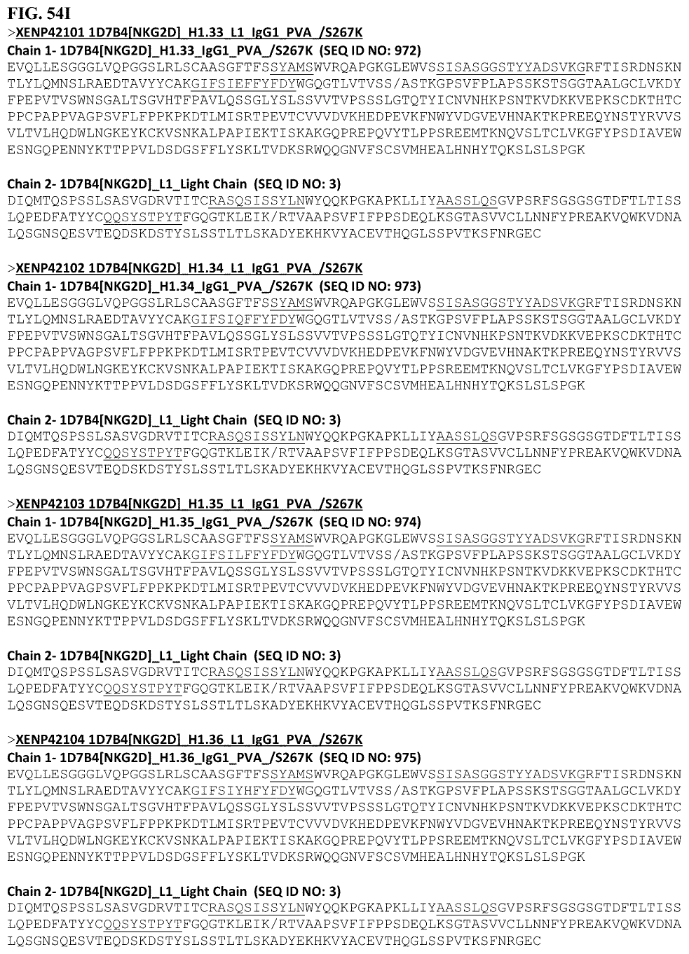

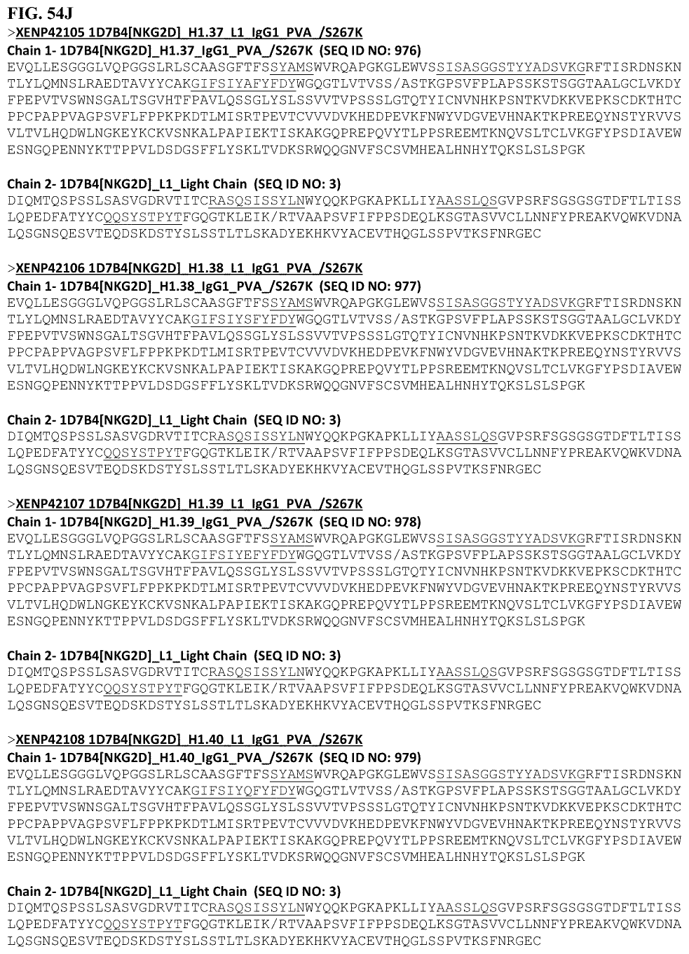

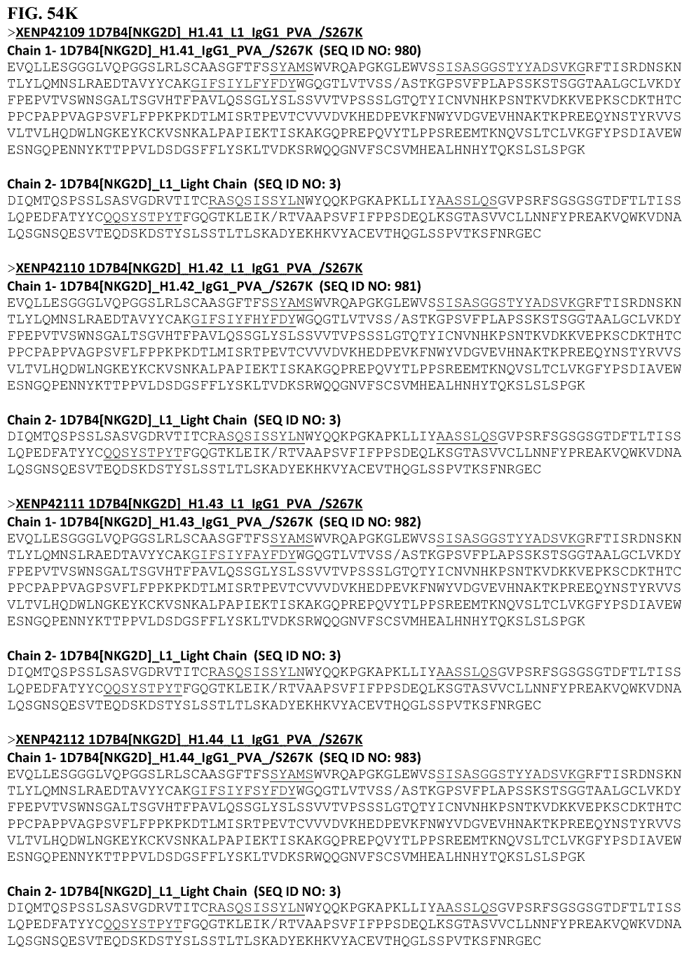

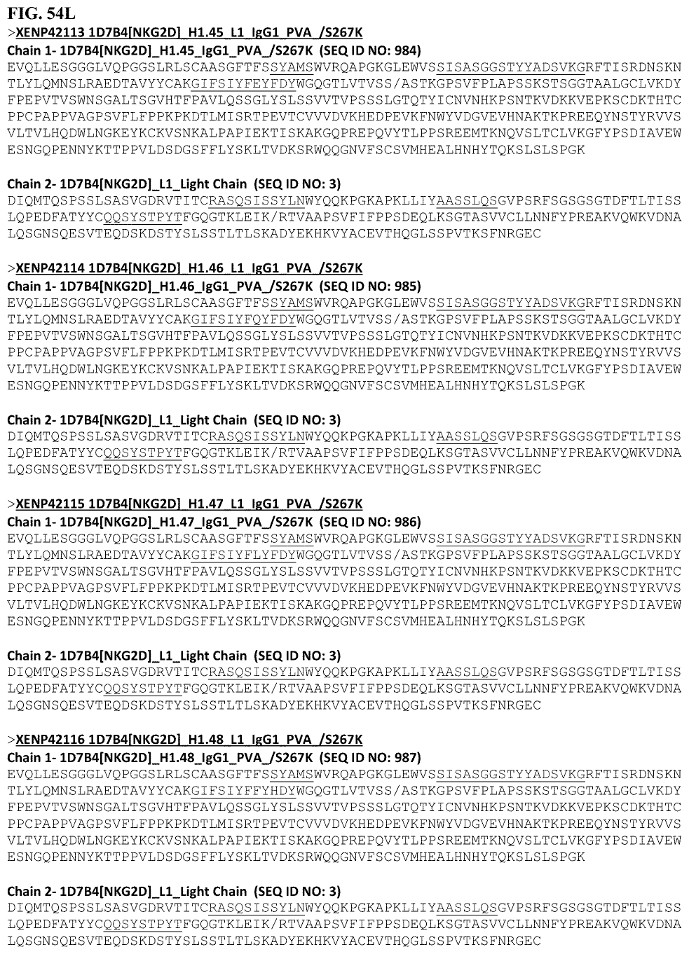

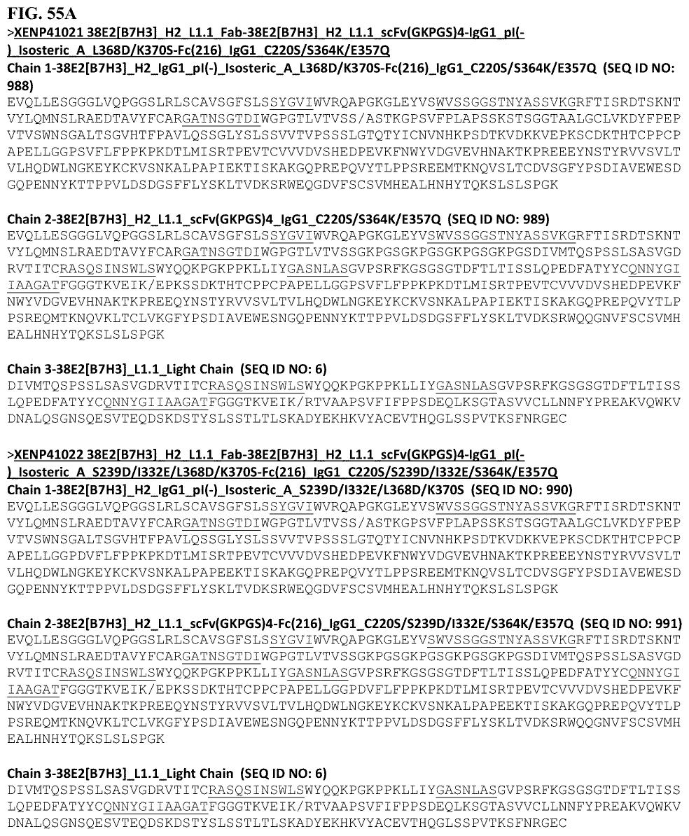

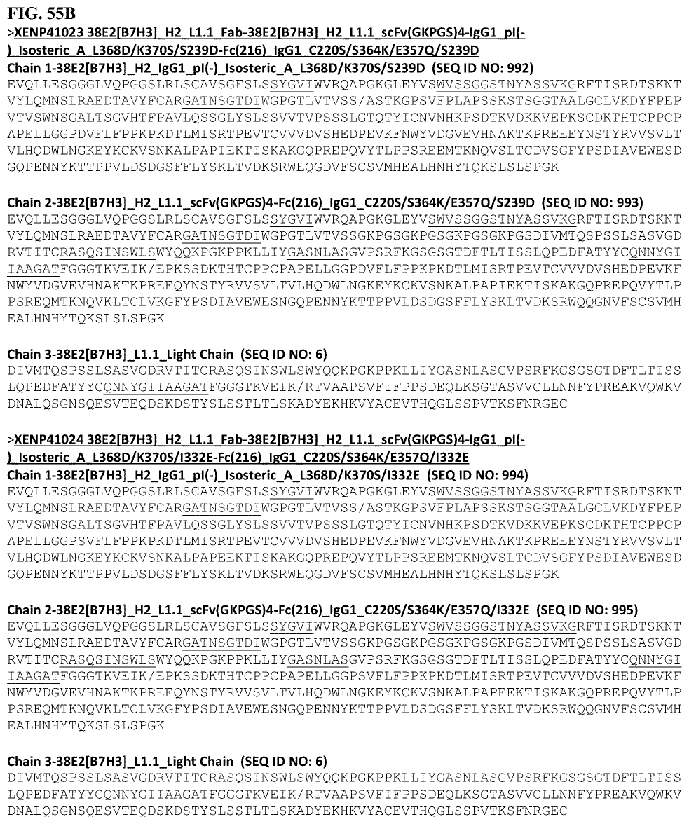

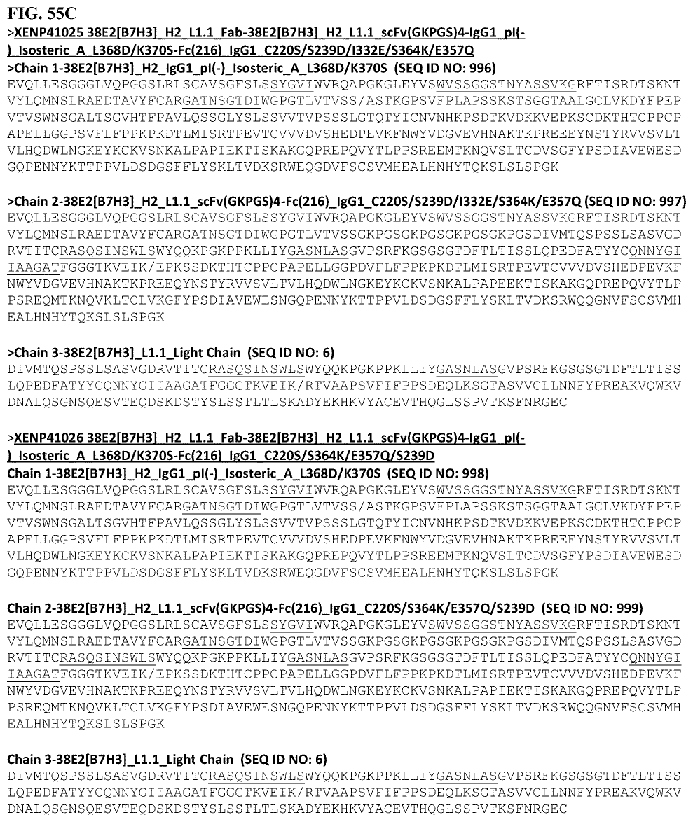

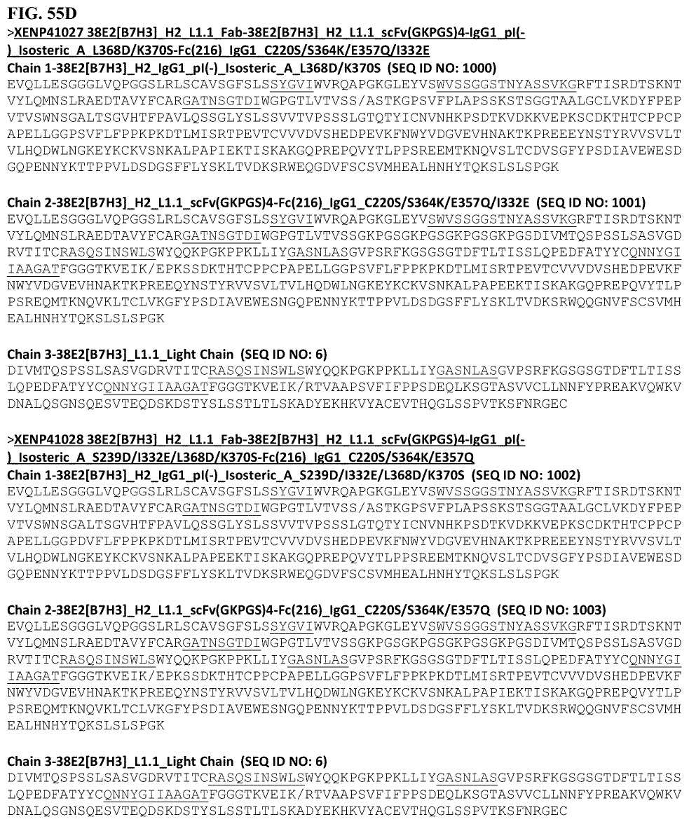

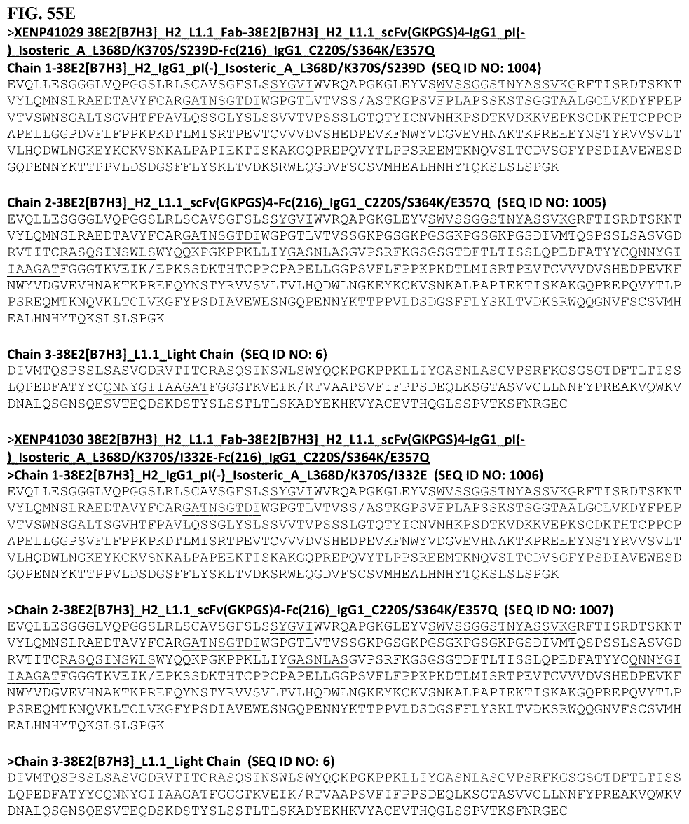

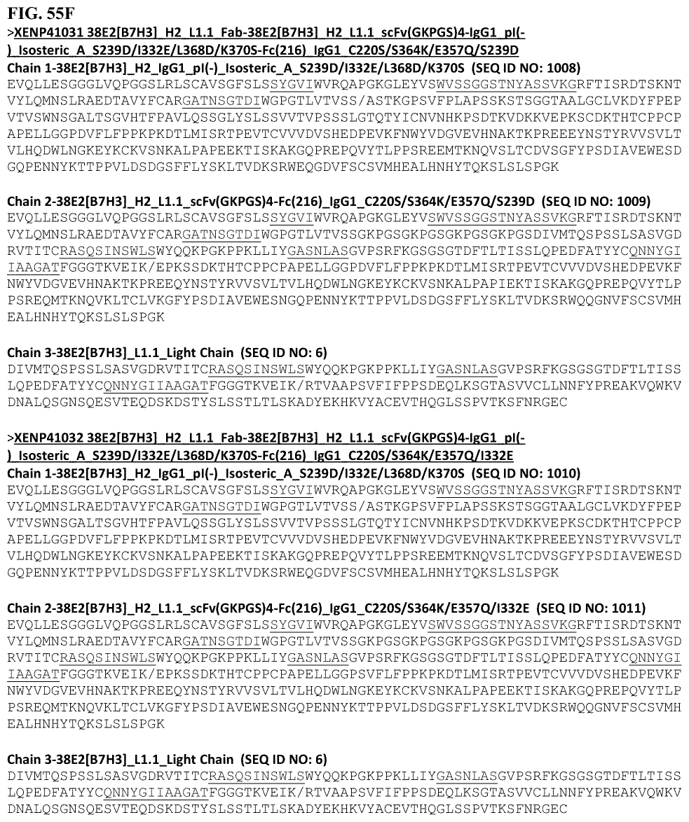

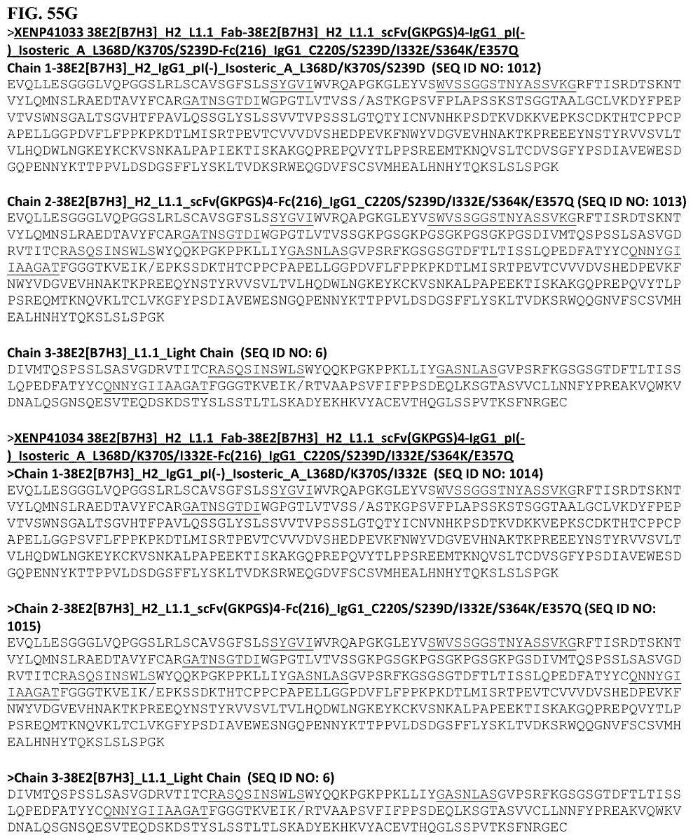

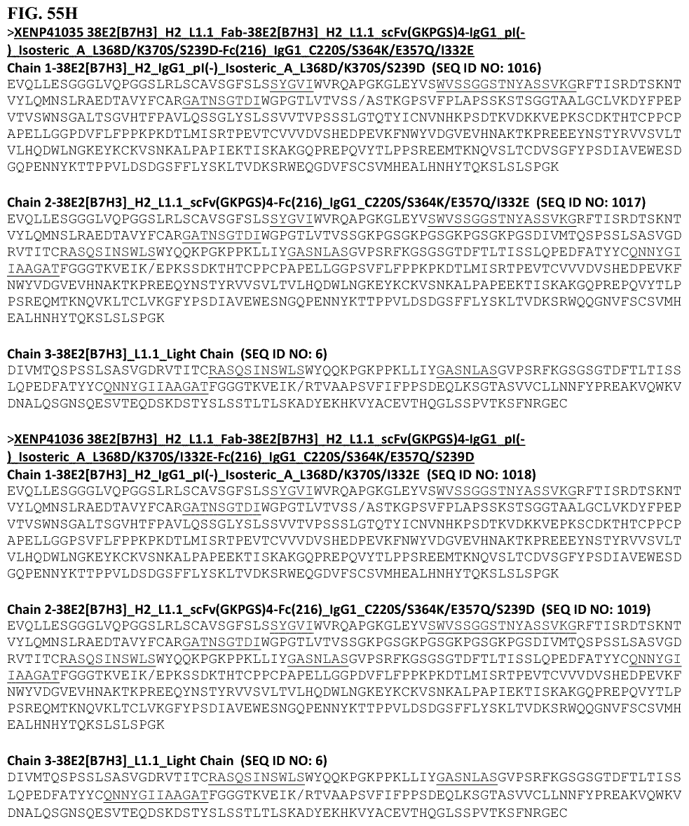

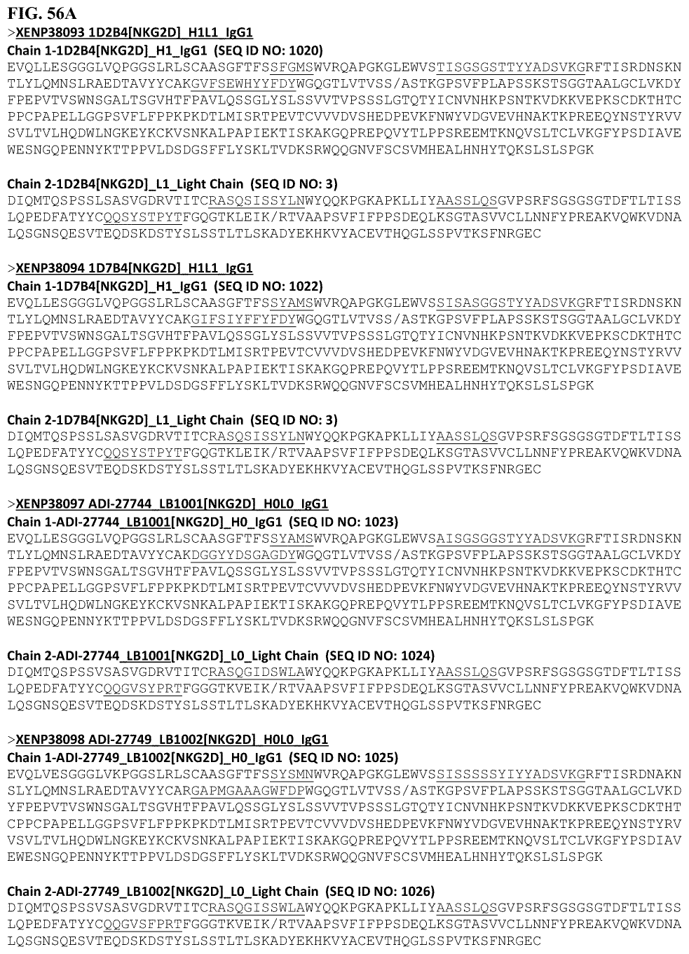

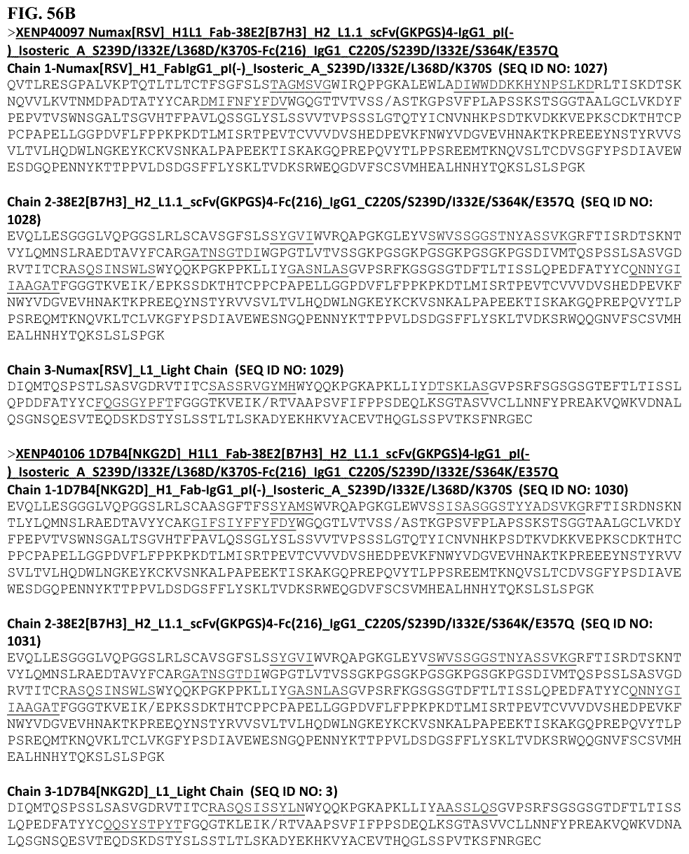

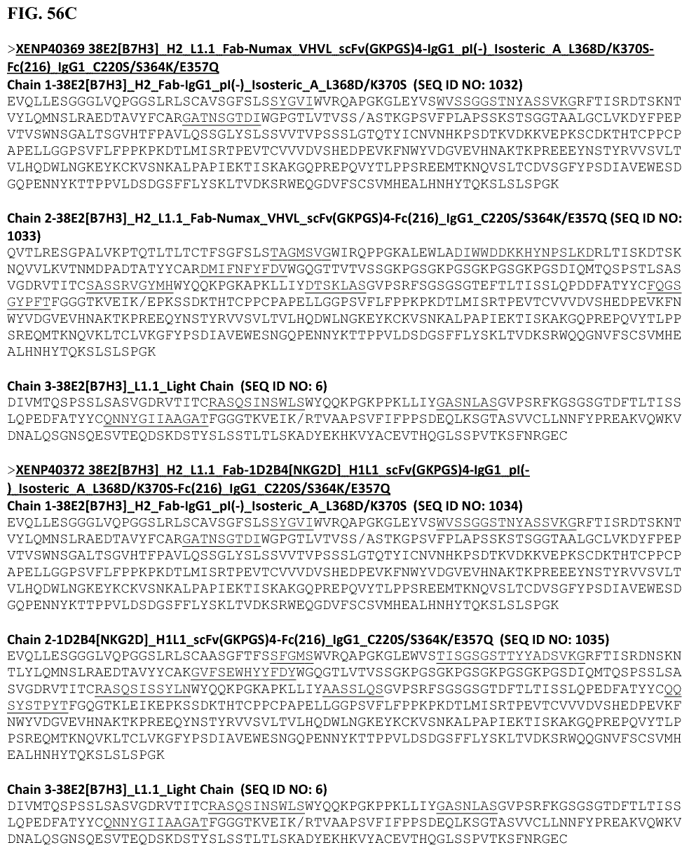

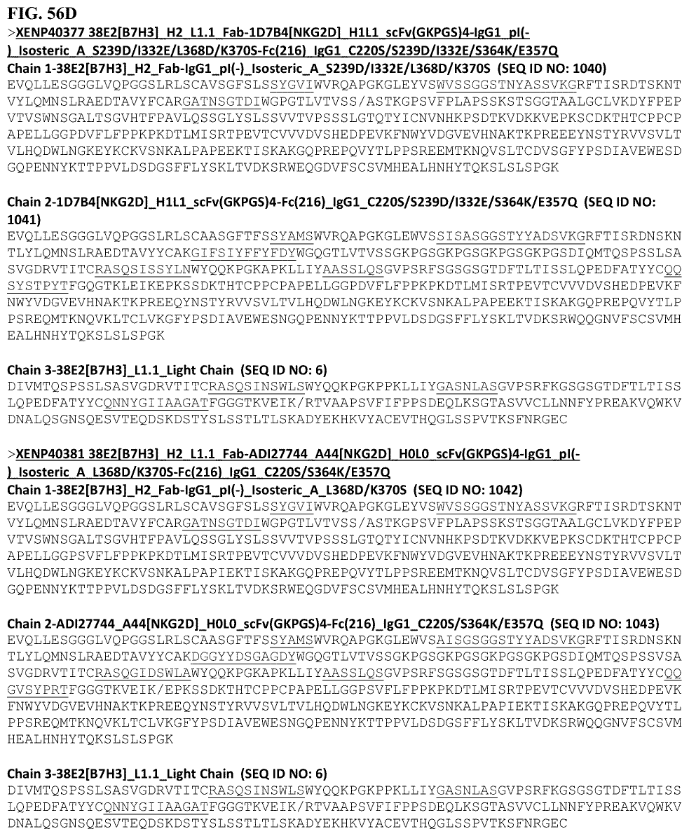

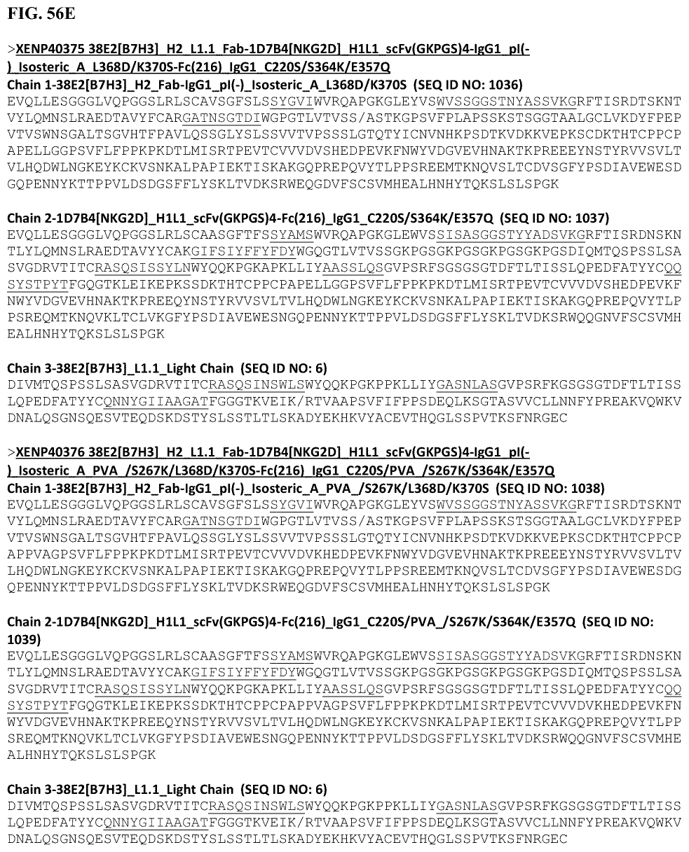

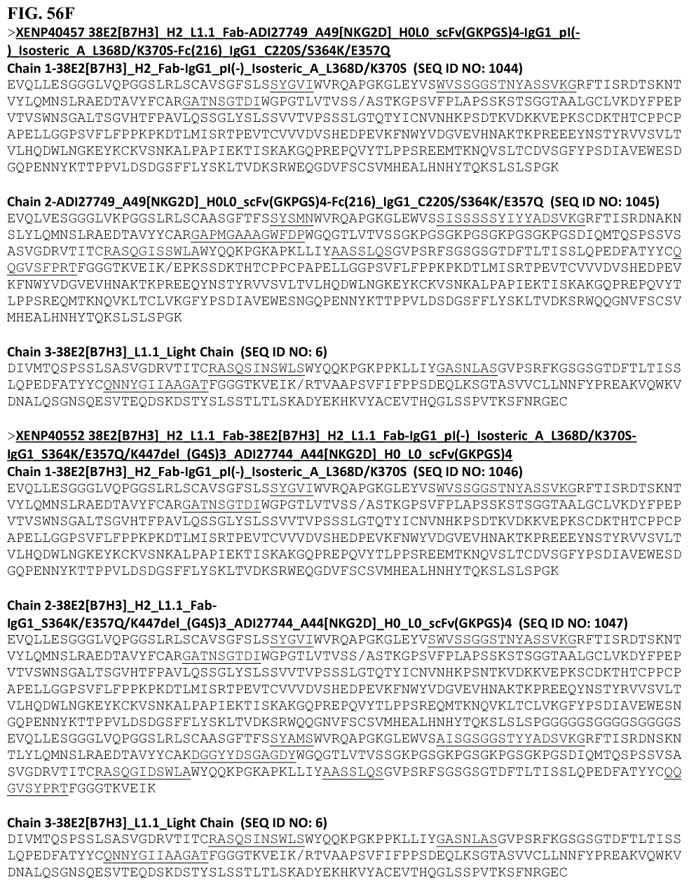

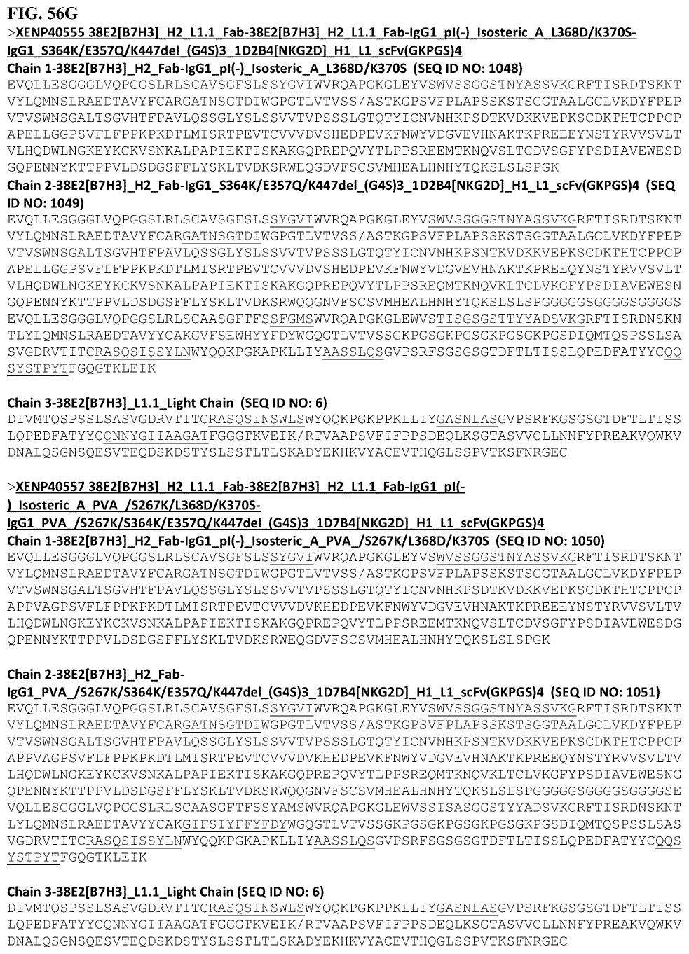

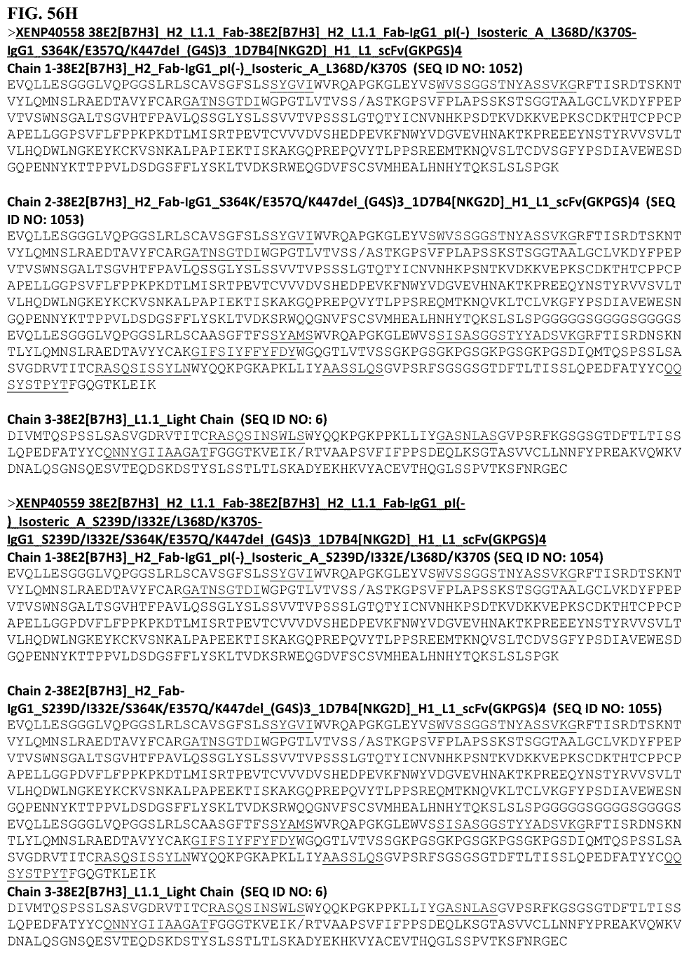

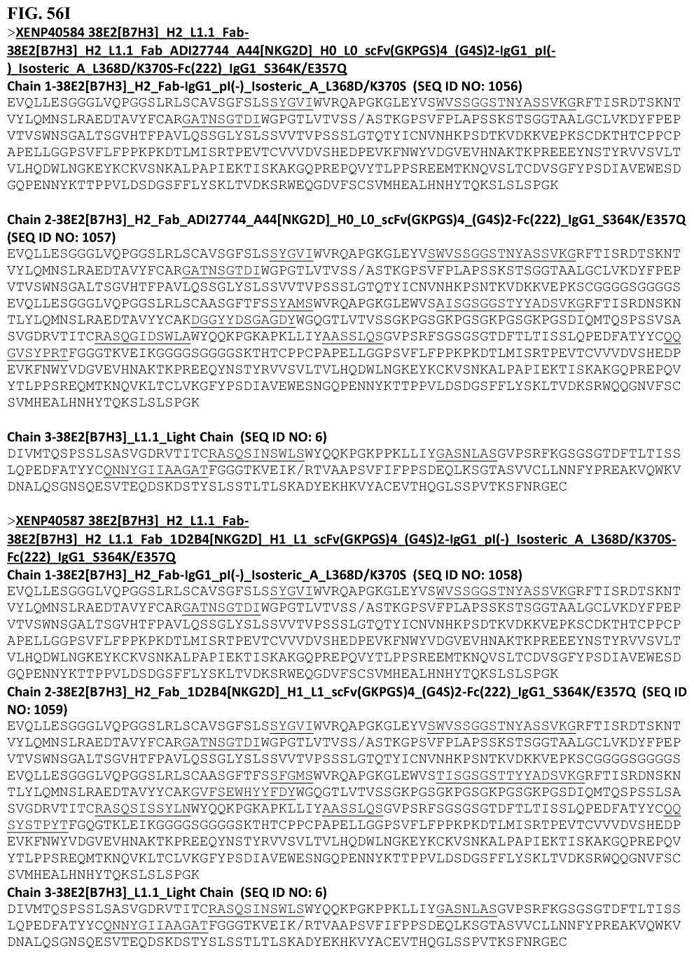

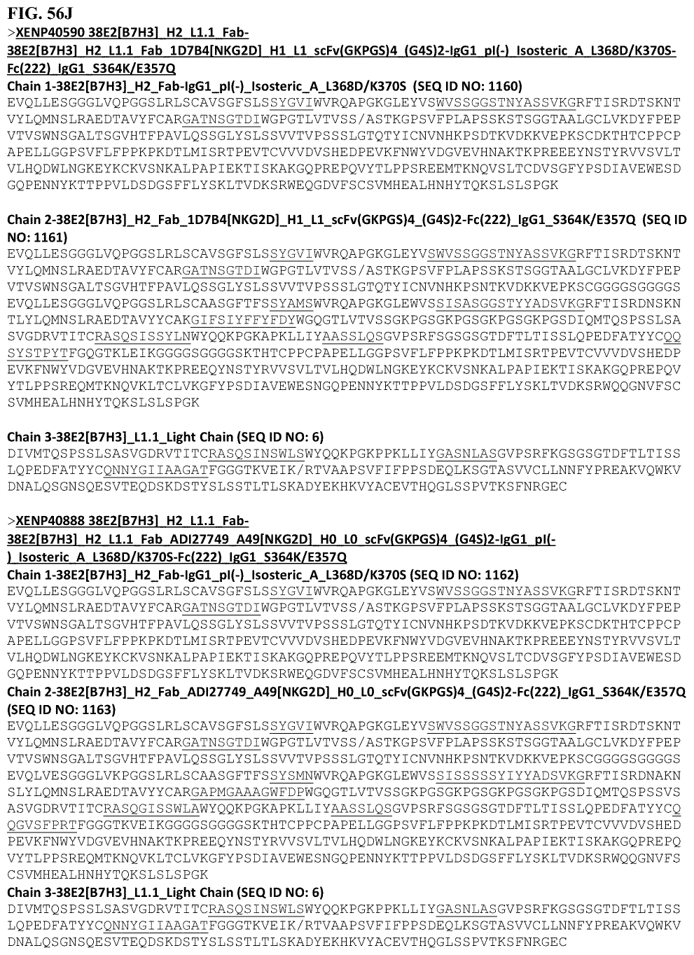

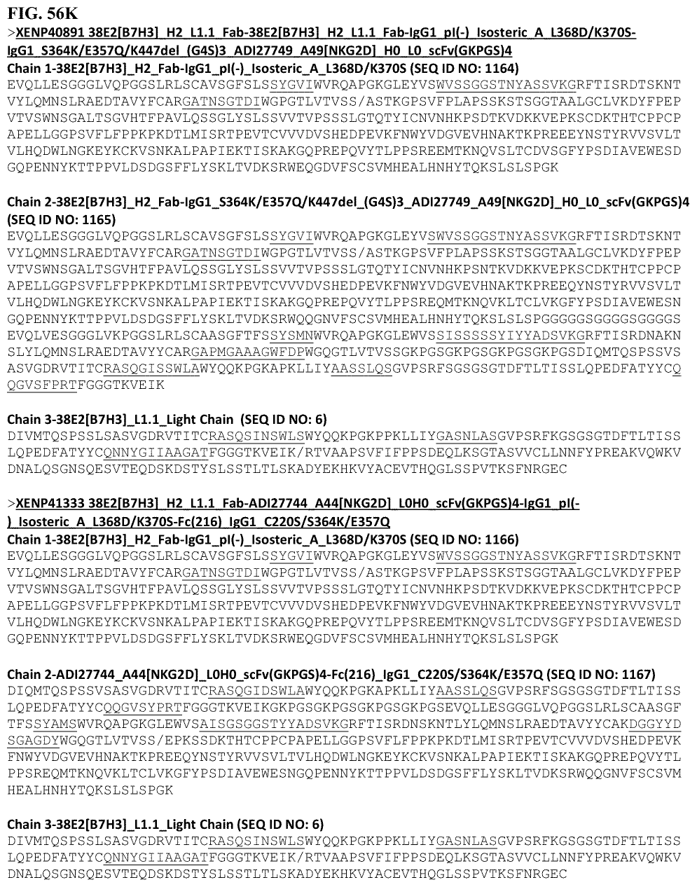

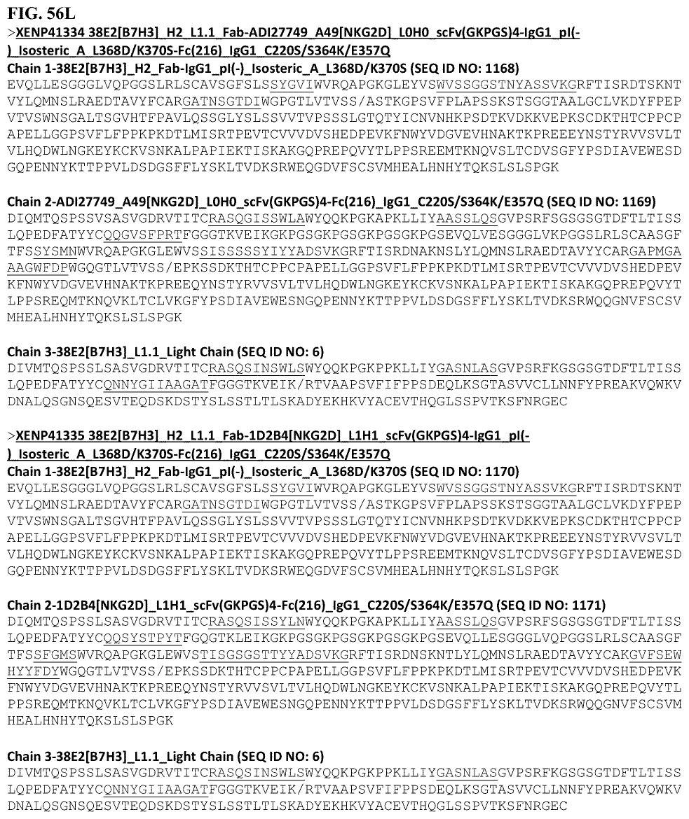

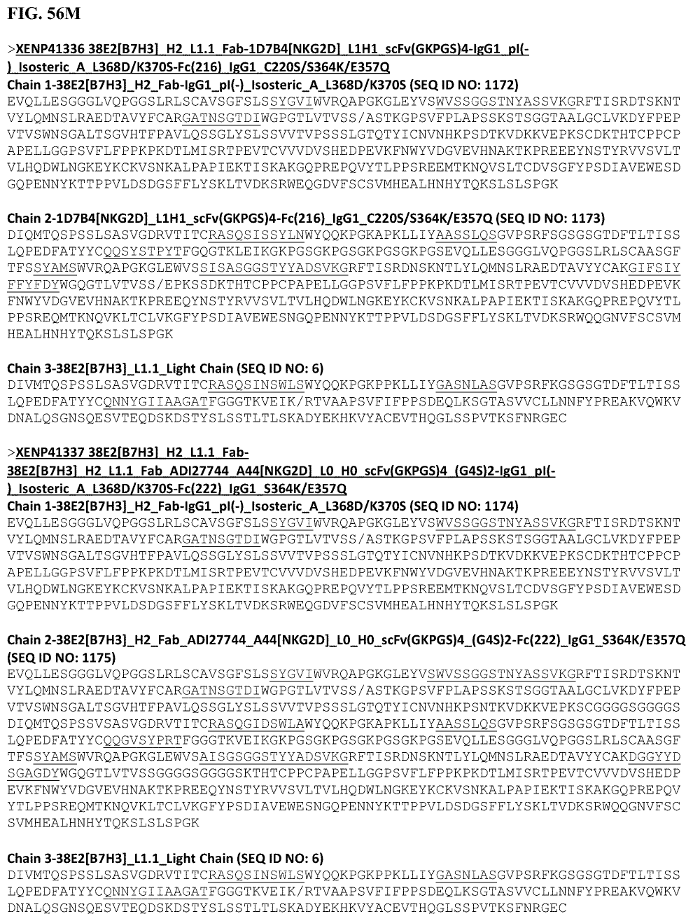

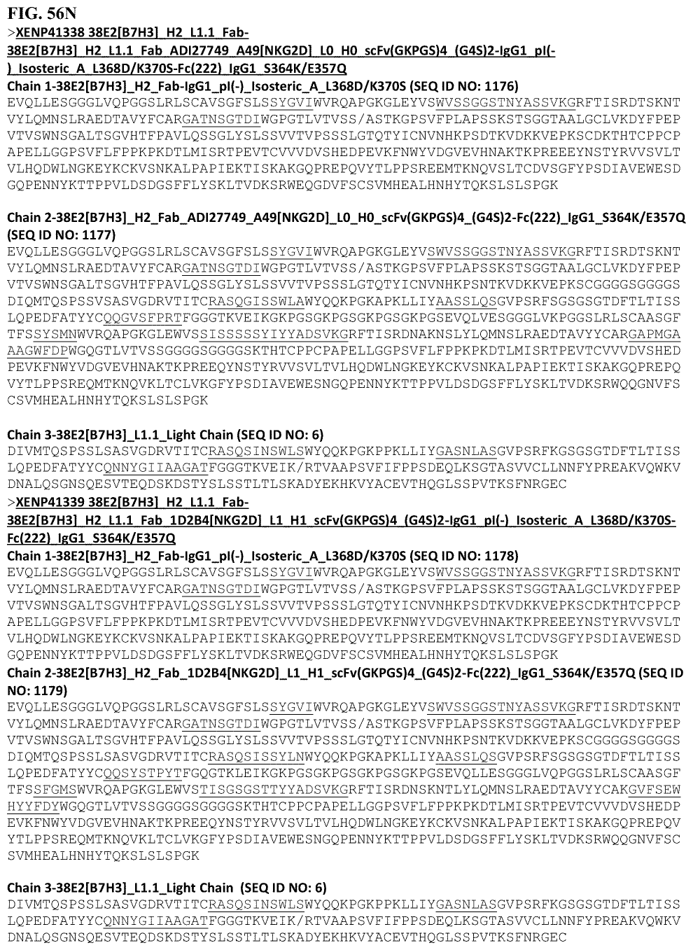

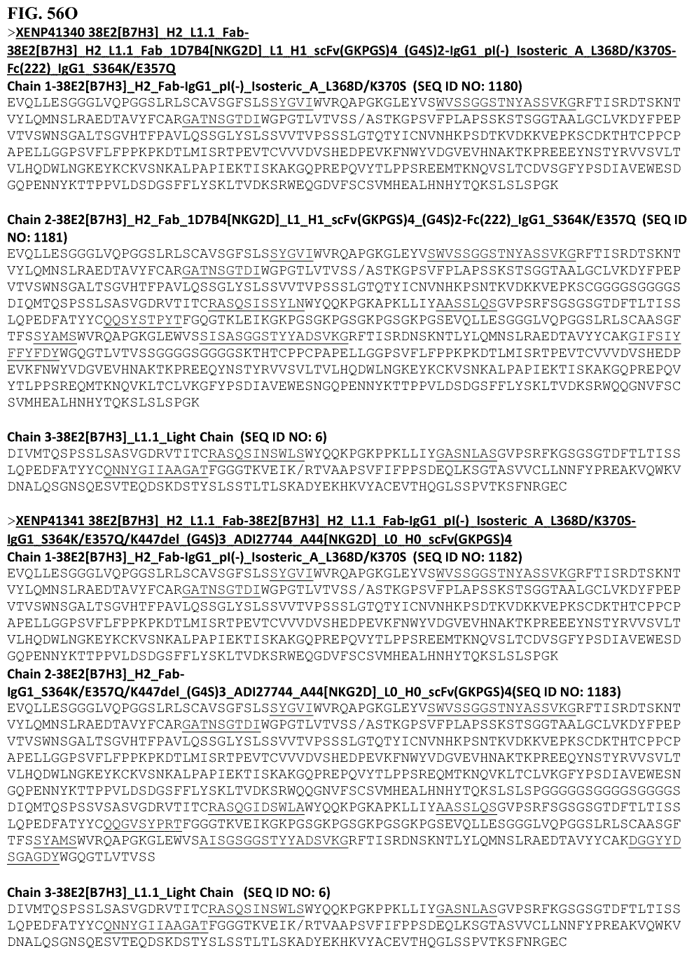

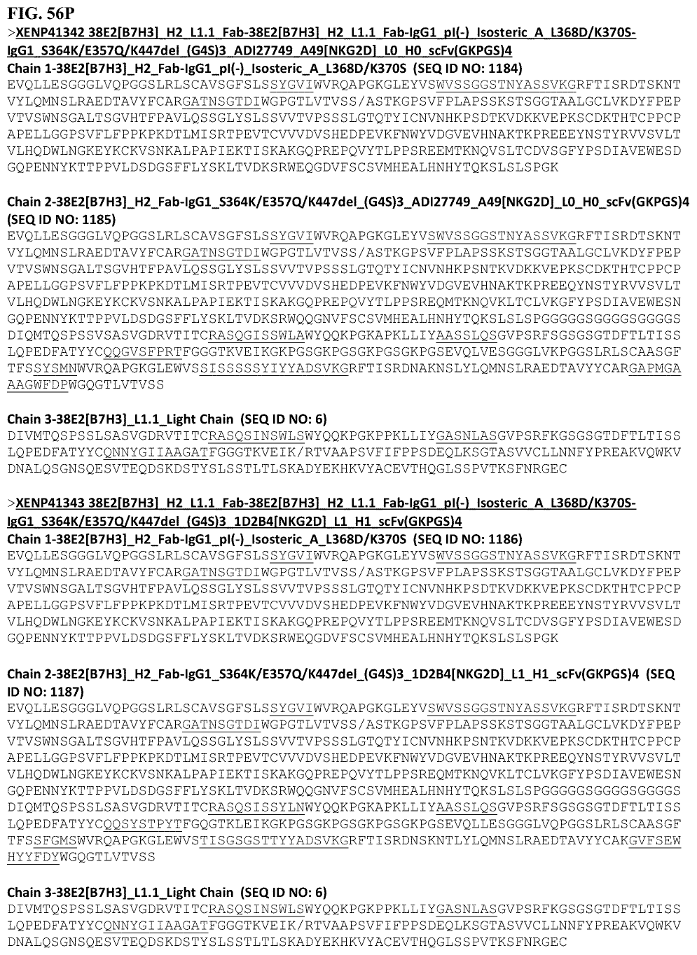

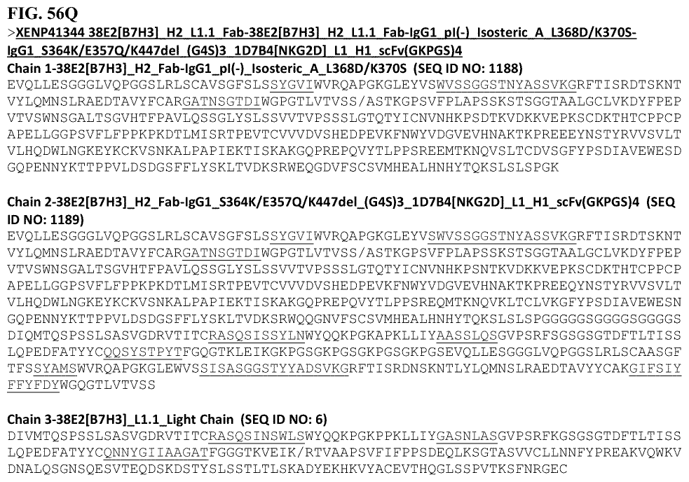

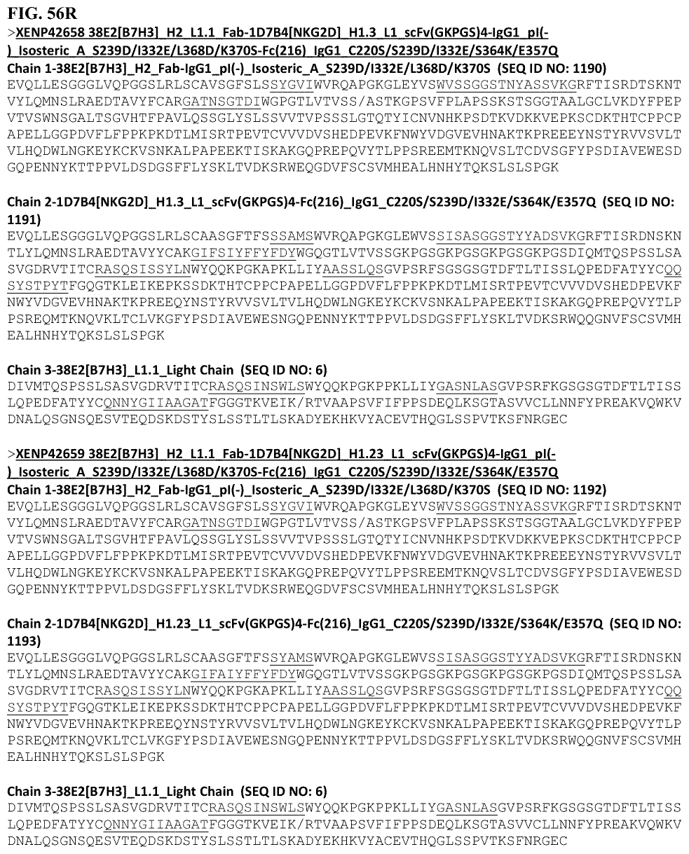

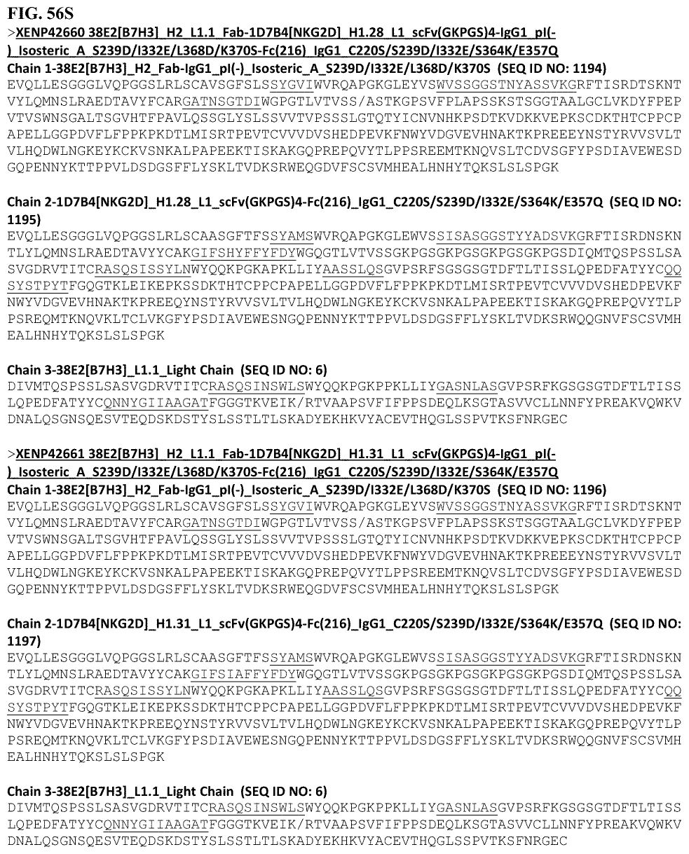

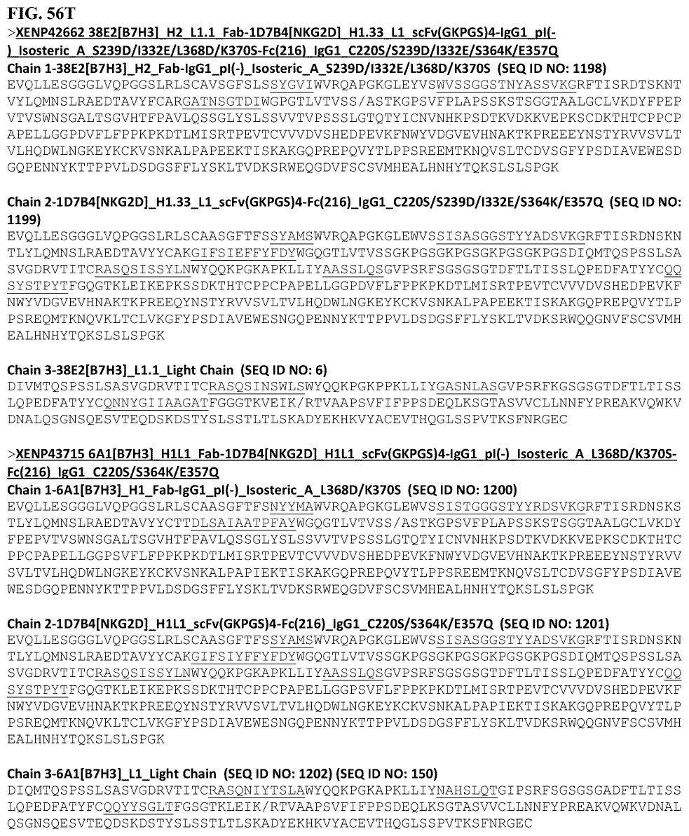

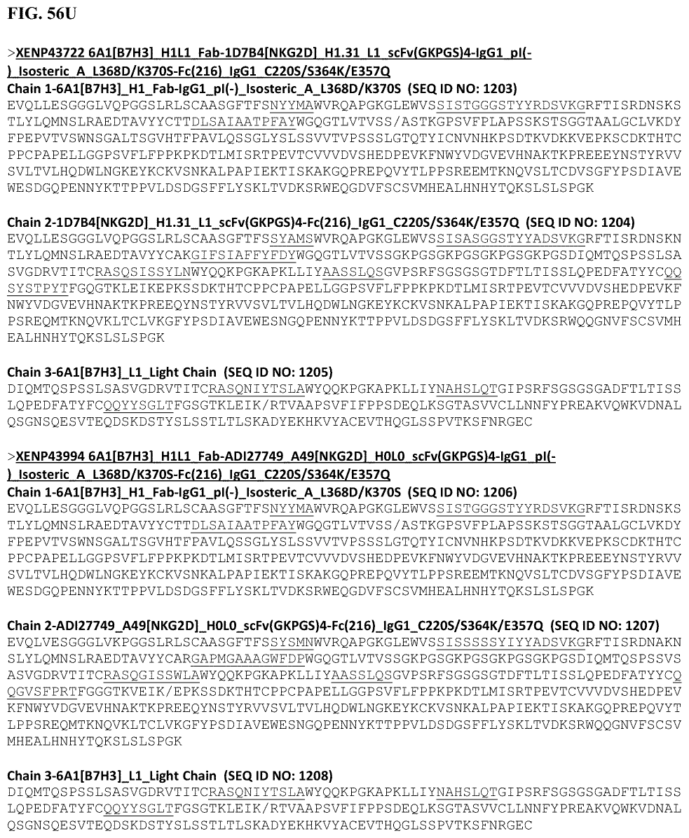

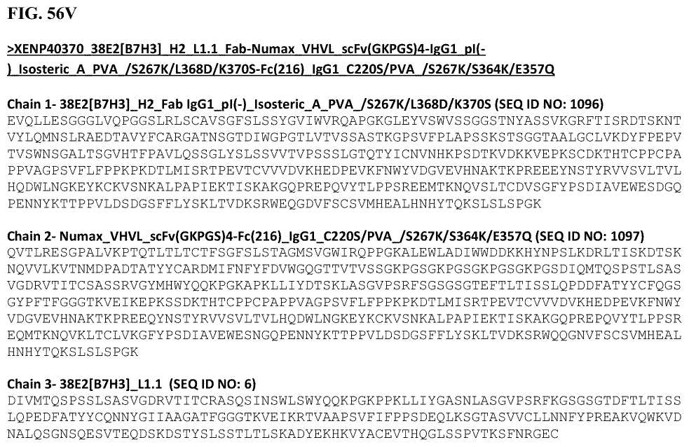

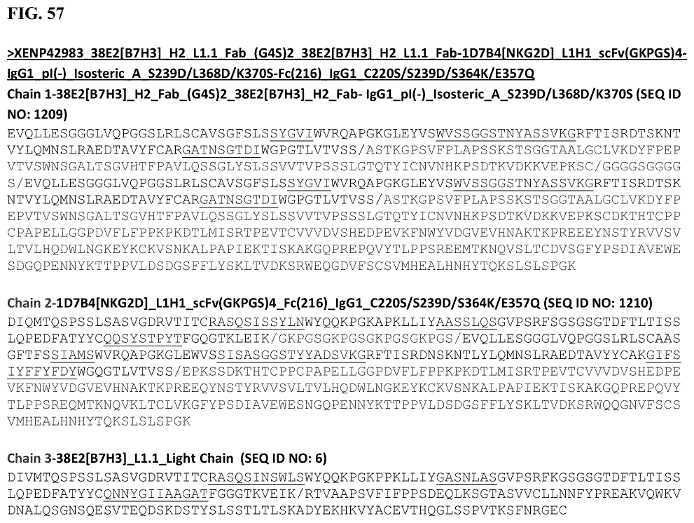

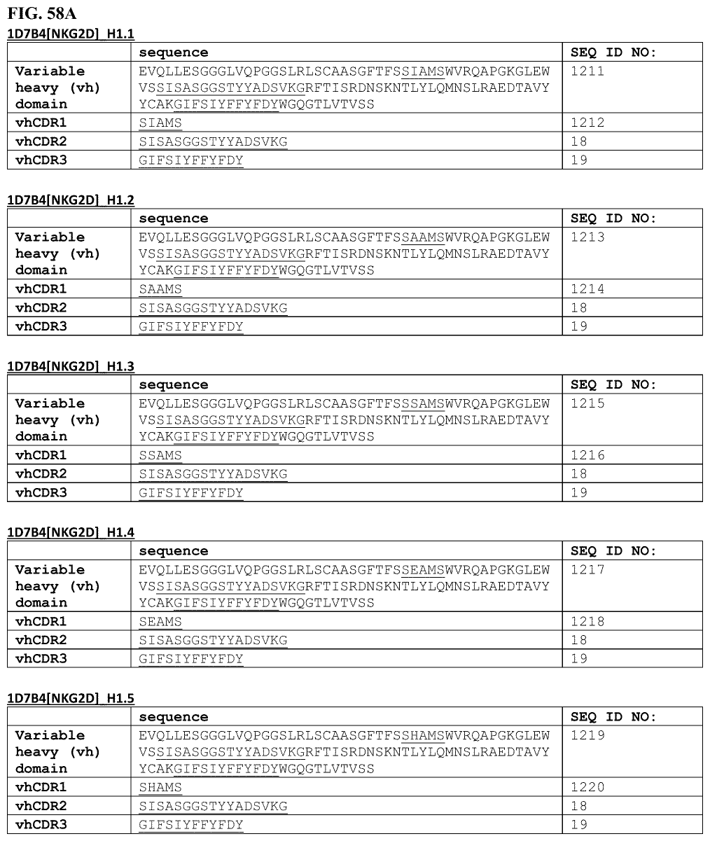

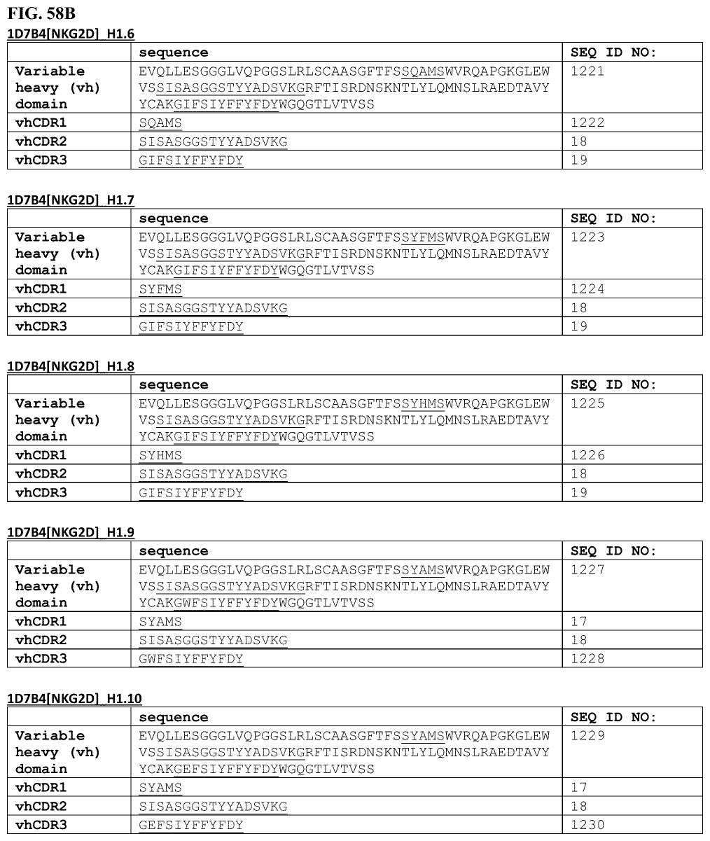

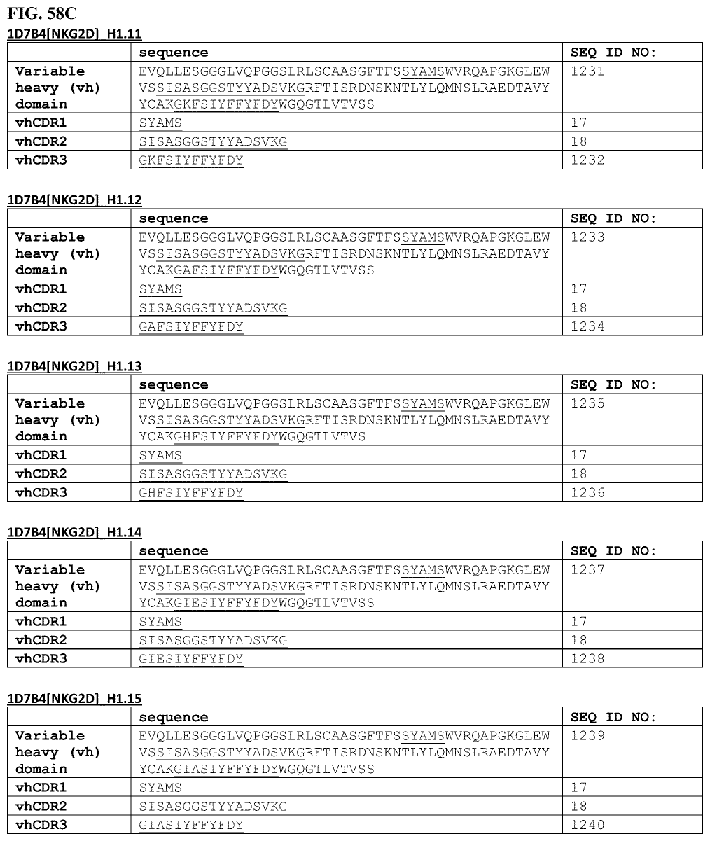

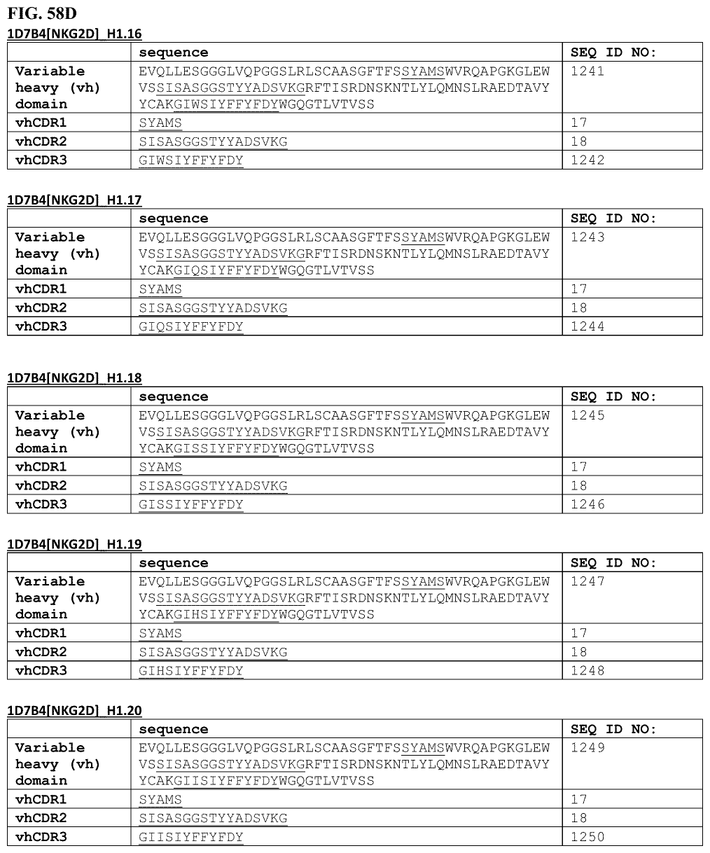

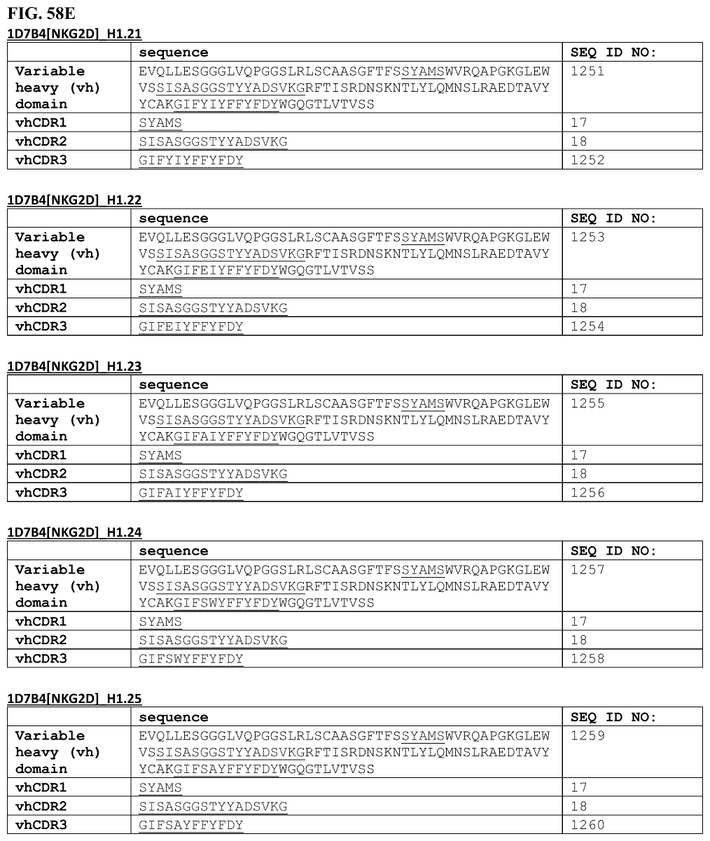

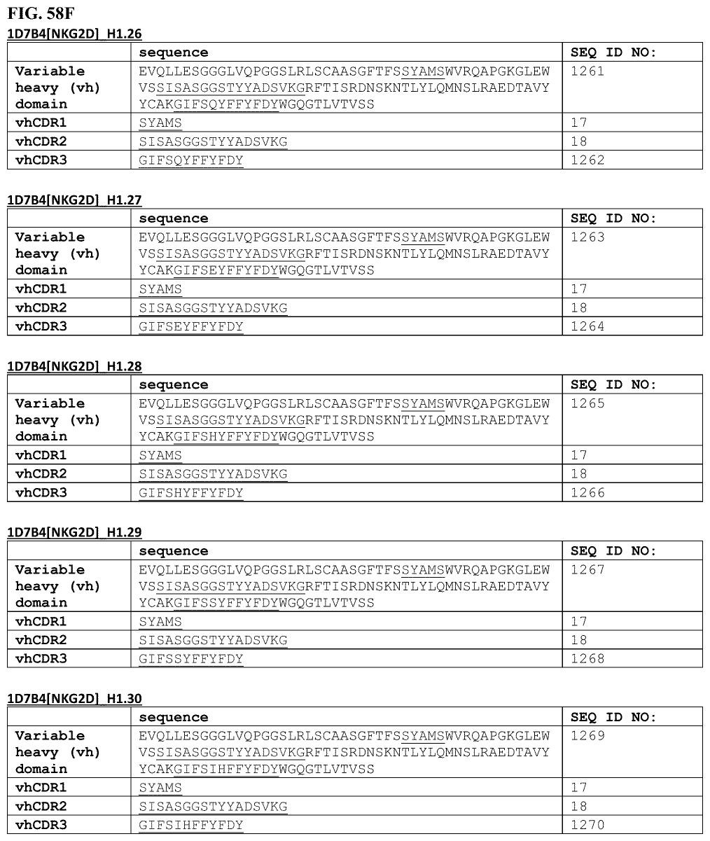

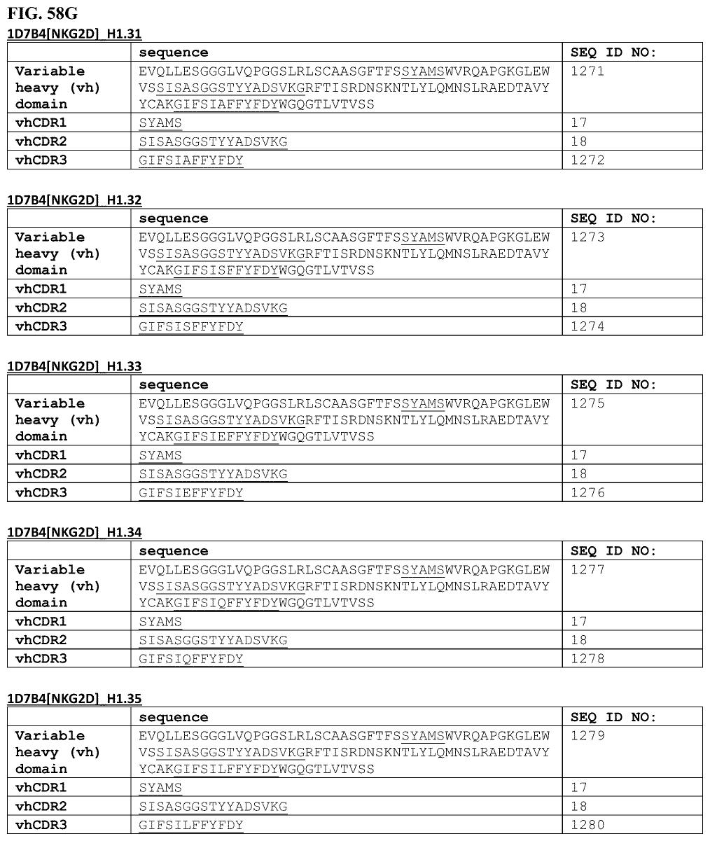

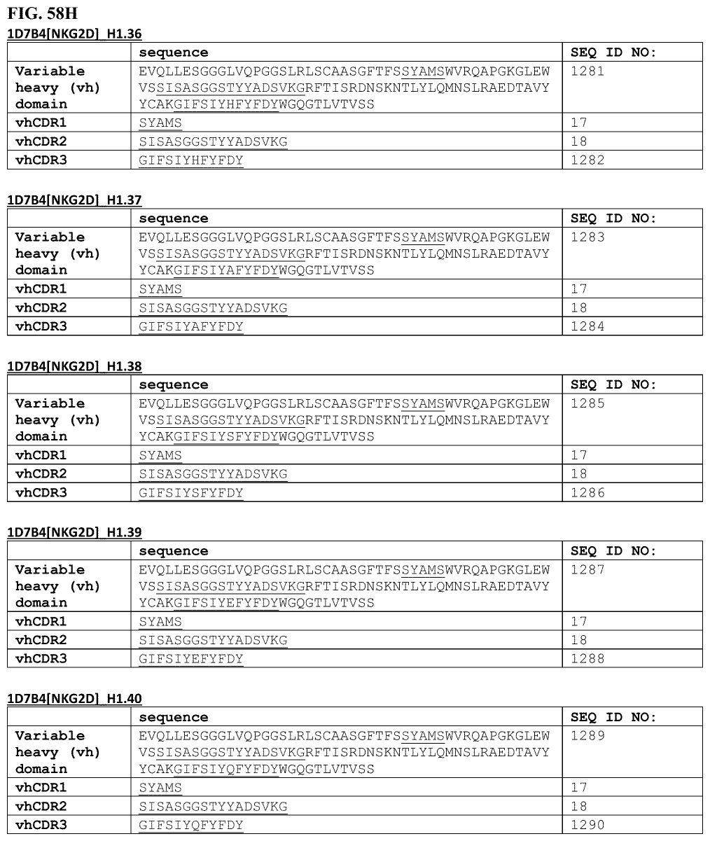

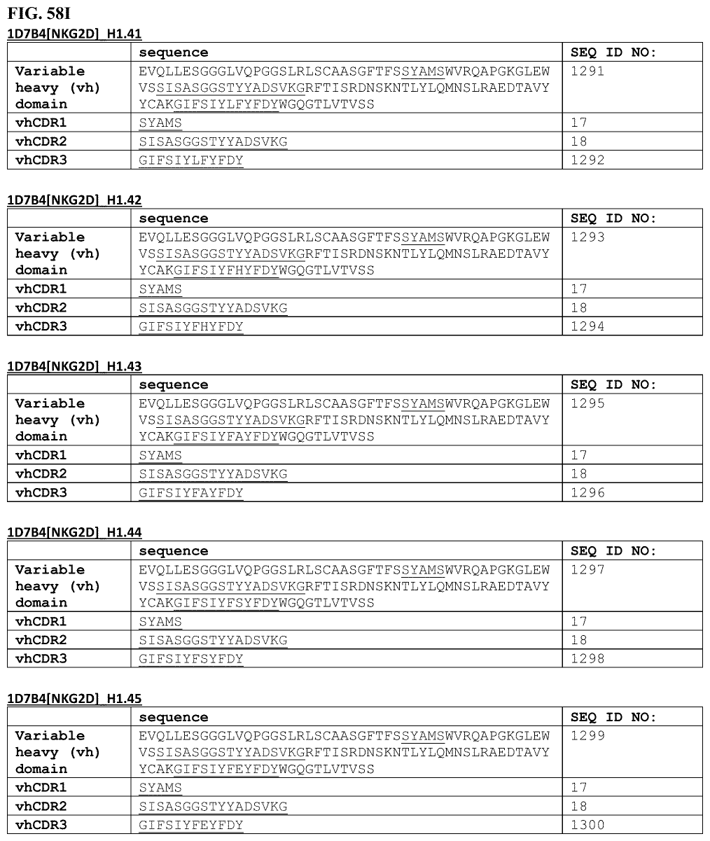

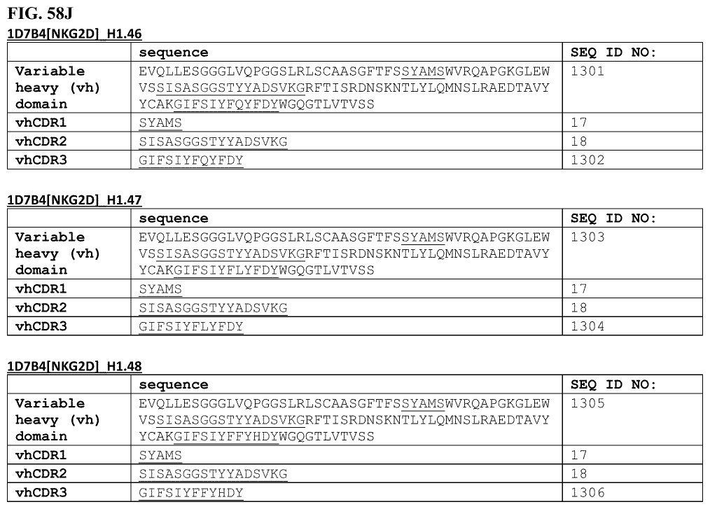

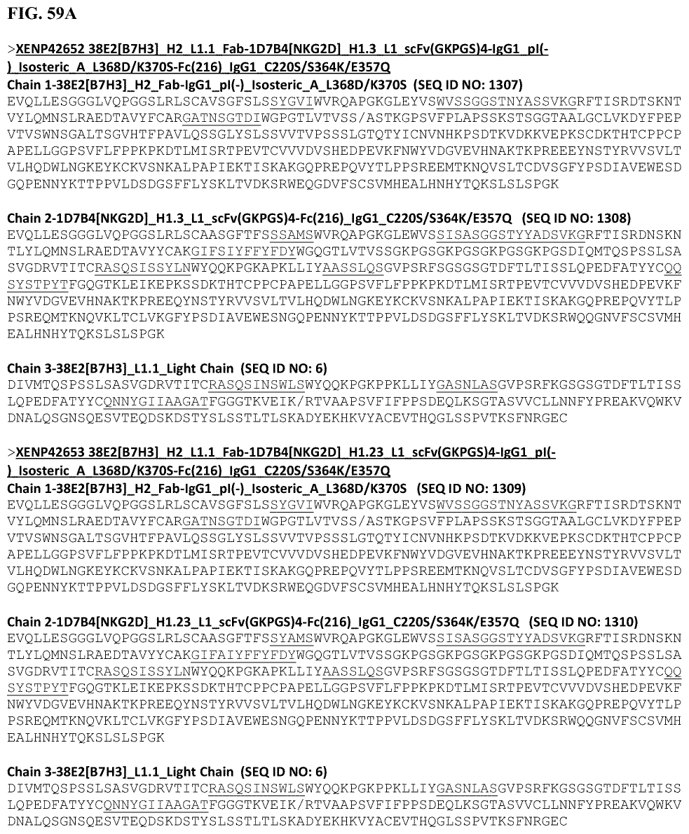

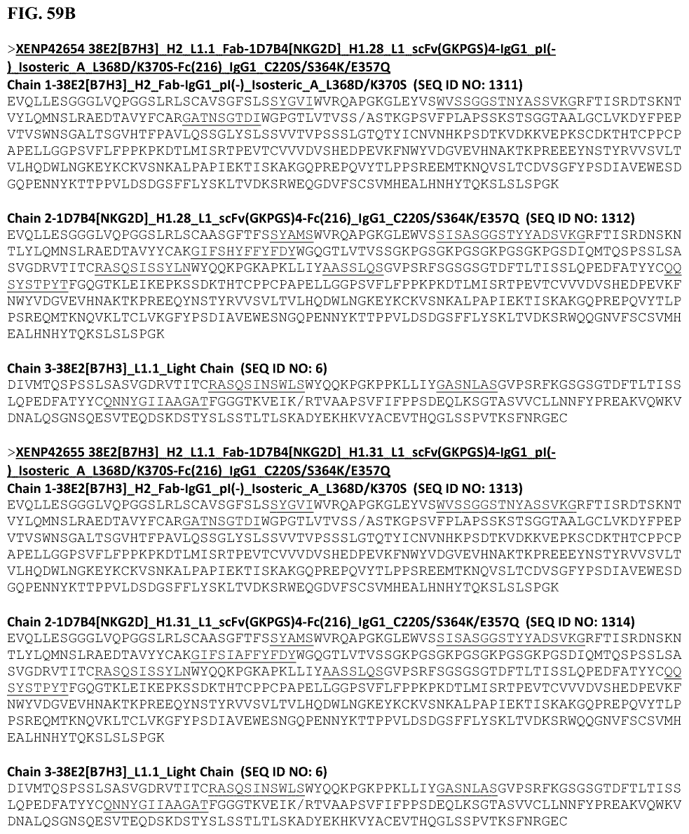

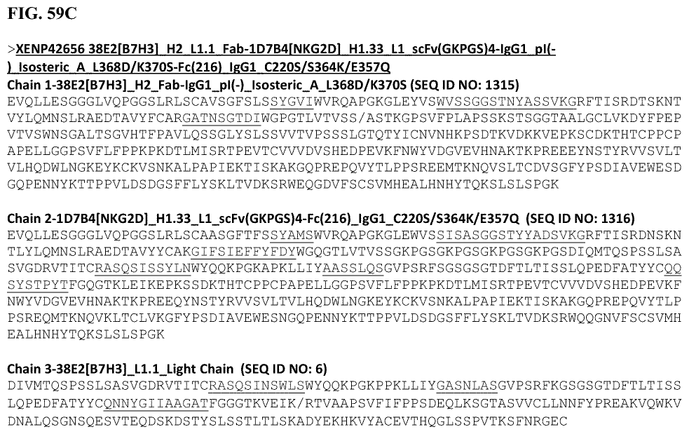

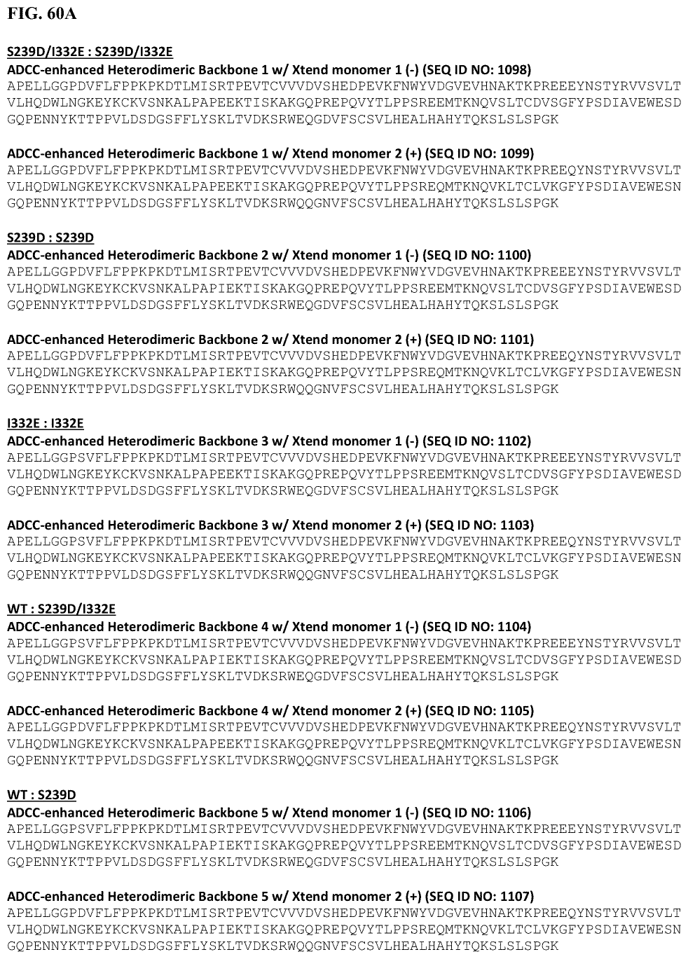

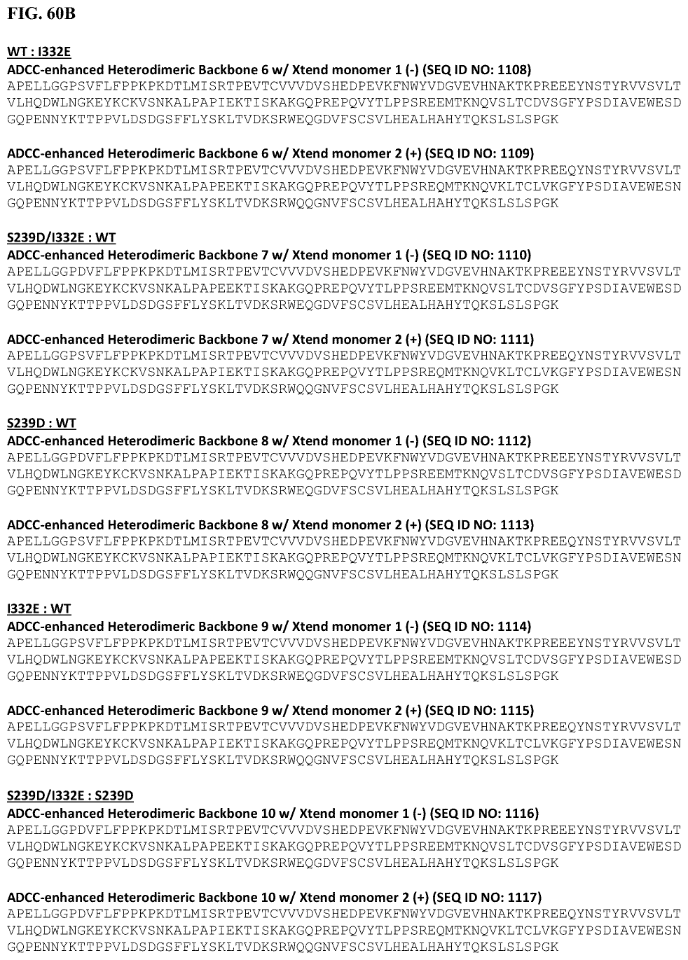

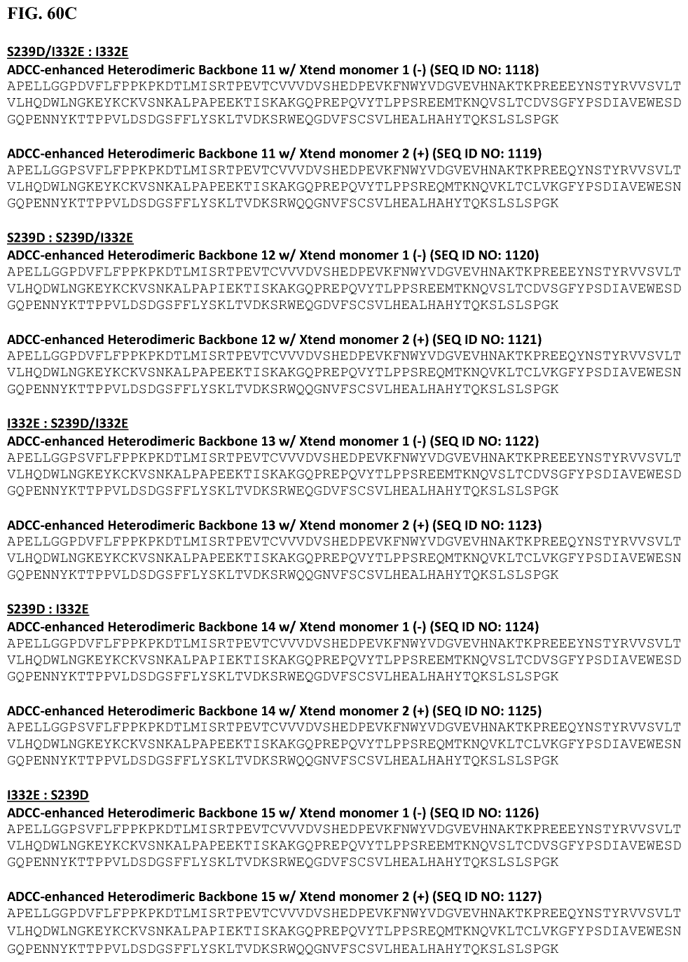

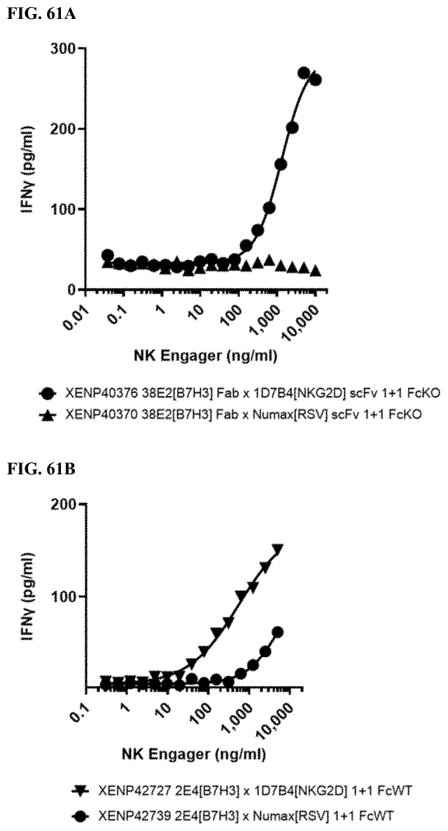

FIGS. 52A-52C show the sequences of several useful heterodimeric αB7H3×αNKG2D bsAb backbones based on human IgG1 and having enhanced ADCC function and enhanced serum half-life. The sequences here are based on heterodimeric Fc backbone 8 in FIG. 35, although the ADCC variants in FIG. 34 may also be included in any of the other heterodimeric Fc backbones in FIG. 34. ADCC-enhanced Heterodimeric Backbone 1 with Xtend includes S239D/I332E on both the first and the second heterodimeric Fc chain. ADCC-enhanced Heterodimeric Backbone 2 with Xtend includes S239D on both the first and the second heterodimeric Fc chain. ADCC-enhanced Heterodimeric Backbone 3 with Xtend includes I332E on both the first and the second heterodimeric Fc chain. ADCC-enhanced Heterodimeric Backbone 4 with Xtend includes S239D/I332E on the second heterodimeric Fc chain. ADCC-enhanced Heterodimeric Backbone 5 with Xtend includes S239D on the second heterodimeric Fc chain. ADCC-enhanced Heterodimeric Backbone 6 with Xtend includes I332E on the second heterodimeric Fc chain. ADCC-enhanced Heterodimeric Backbone 7 with Xtend includes S239D/I332E on the first heterodimeric Fc chain. ADCC-enhanced Heterodimeric Backbone 8 with Xtend includes S239D on the first heterodimeric Fc chain. ADCC-enhanced Heterodimeric Backbone 9 with Xtend includes I332E on the first heterodimeric Fc chain. ADCC-enhanced Heterodimeric Backbone 10 with Xtend includes S239D/I332E on the first heterodimeric Fc chain and S239D on the second heterodimeric Fc chain. ADCC-enhanced Heterodimeric Backbone 11 with Xtend includes S239D/I332E on the first heterodimeric Fc chain and I332E on the second heterodimeric Fc chain. ADCC-enhanced Heterodimeric Backbone 12 with Xtend includes S239D on the first heterodimeric Fc chain and S239D/I332E on the second heterodimeric Fc chain. ADCC-enhanced Heterodimeric Backbone 13 with Xtend includes I332E on the first heterodimeric Fc chain and S239D/I332E on the second heterodimeric Fc chain. ADCC-enhanced Heterodimeric Backbone 14 with Xtend includes S239D on the first heterodimeric Fc chain and I332E on the second heterodimeric Fc chain. ADCC-enhanced Heterodimeric Backbone 15 with Xtend includes I332E on the first heterodimeric Fc chain and S239D on the second heterodimeric Fc chain.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1. That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIGS. 53A-53G depict illustrative sequences of heterodimeric αB7H3×αNKG2D bsAb backbone for use in the 2+1 mAb-scFv format. The format depicted here is based on heterodimeric Fc backbone 1 as depicted in FIG. 35, except further including K447_ on monomer 2 (+). It should be noted that any of the additional backbones depicted in FIGS. 35 and 50-52 may be adapted for use in the 2+1 mAb-scFv format with or without including K447_ on one or both chains.

FIGS. 54A-54L depict the sequences of the engineered affinity detuned 1D7B4 ABDs in the format of a bivalent antibody. It should be noted that these ABDs can be used in any of the other formats of the invention.

FIGS. 55A-55H depict the set of ADCC enhanced Fc variants (or WT effector function as a control in the case of the control XENP41021) in the format of a B7H3 1+1 Fab-scFv Fc.

FIGS. 56A-56V depict additional sequences of the invention.

FIG. 57 depicts an exemplary αB7H3×αNKG2D antibody in the stackFab₂-scFv-Fc format. An exemplary embodiment of this αB7H3×αNKG2D bispecific antibody includes the amino acid sequences of chain 1 (SEQ ID NO:1209), chain 2 (SEQ ID NO:1210) and chain 3 (SEQ ID NO:6).

FIGS. 58A-58J depict the affinity detuned variable heavy domains of the anti-NKG2D 1D7B4 clone and their CDRs (as in Kabat). As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIGS. 59A-59C depict the sequences for select detuned 1D7B4 variants in the 1+1 Fab-scFv-Fc format (with a B7H3 Fab and NKG2D scFv).

FIGS. 60A-60C show the sequences of several useful heterodimeric αB7H3×αNKG2D bsAb backbones based on human IgG1 and having enhanced ADCC function and alternate enhanced serum half-life variants 428L/434A. The sequences here are based on heterodimeric Fc backbone 8 in FIG. 51, although the ADCC variants in FIG. 34 may also be included in any of the other heterodimeric Fc backbones in FIG. 51. ADCC-enhanced Heterodimeric Backbone 1 with Xtend includes S239D/I332E on both the first and the second heterodimeric Fc chain. ADCC-enhanced Heterodimeric Backbone 2 with Xtend includes S239D on both the first and the second heterodimeric Fc chain. ADCC-enhanced Heterodimeric Backbone 3 with Xtend includes I332E on both the first and the second heterodimeric Fc chain. ADCC-enhanced Heterodimeric Backbone 4 with Xtend includes S239D/I332E on the second heterodimeric Fc chain. ADCC-enhanced Heterodimeric Backbone 5 with Xtend includes S239D on the second heterodimeric Fc chain. ADCC-enhanced Heterodimeric Backbone 6 with Xtend includes I332E on the second heterodimeric Fc chain. ADCC-enhanced Heterodimeric Backbone 7 with Xtend includes S239D/I332E on the first heterodimeric Fc chain. ADCC-enhanced Heterodimeric Backbone 8 with Xtend includes S239D on the first heterodimeric Fc chain. ADCC-enhanced Heterodimeric Backbone 9 with Xtend includes I332E on the first heterodimeric Fc chain. ADCC-enhanced Heterodimeric Backbone 10 with Xtend includes S239D/I332E on the first heterodimeric Fc chain and S239D on the second heterodimeric Fc chain. ADCC-enhanced Heterodimeric Backbone 11 with Xtend includes S239D/I332E on the first heterodimeric Fc chain and I332E on the second heterodimeric Fc chain. ADCC-enhanced Heterodimeric Backbone 12 with Xtend includes S239D on the first heterodimeric Fc chain and S239D/I332E on the second heterodimeric Fc chain. ADCC-enhanced Heterodimeric Backbone 13 with Xtend includes I332E on the first heterodimeric Fc chain and S239D/I332E on the second heterodimeric Fc chain. ADCC-enhanced Heterodimeric Backbone 14 with Xtend includes S239D on the first heterodimeric Fc chain and I332E on the second heterodimeric Fc chain. ADCC-enhanced Heterodimeric Backbone 15 with Xtend includes I332E on the first heterodimeric Fc chain and S239D on the second heterodimeric Fc chain.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1. That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIGS. 61A-61B show IFNγ production by NKG2D-targeting NKEs. FIG. 61A depicts the ability of NKG2D-targeting NKEs to induce IFNγ production even in the absence of FcγR engagement. FIG. 61B further illustrates that IFNγ production is driven by the NKG2D targeting arm by comparing it with an RSV×B7H3 Fc WT isotype control.

### DETAILED DESCRIPTION

### I. Overview

The description is presented to enable one of ordinary skill in the art to make and use the invention and is provided in the context of a patent application and its requirements. The section headings used herein are for organization purposes only and are not to be construed as limiting the subject matter described. While various embodiments of the invention(s) of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the invention(s) described herein may be employed in practicing any one of the inventions(s) set forth herein.

All patents, published patent applications, other publications, and sequences from GenBank, and other databases referred to herein are incorporated by reference in their entirety with respect to the related technology.

### II. Nomenclature

The antibodies provided herein are listed in several different formats. In some instances, each monomer of a particular antibody is given a unique “XENP” number, although as will be appreciated in the art, a longer sequence might contain a shorter one. For example, a “scFv-Fc” monomer of a 1+1 Fab-scFv-Fc format antibody may have a first XENP number, while the scFv domain itself will have a different XENP number. Some molecules have three polypeptides, so the XENP number, with the components, is used as a name. Thus, the molecule XENP37630, which is in 2+1 Fab₂-scFv-Fc format, comprises three sequences (see FIG. 59A) a “Fab-Fc Heavy Chain” monomer (“Chain 1”); 2) a “Fab-scFv-Fc Heavy Chain” monomer (“Chain 2”); and 3) a “Light Chain” monomer (“Chain 3”) or equivalents, although one of skill in the art would be able to identify these easily through sequence alignment. These XENP numbers are in the sequence listing as well as identifiers, and used in the Figures. In addition, one molecule, comprising the three components, gives rise to multiple sequence identifiers. For example, the listing of the Fab includes, the full heavy chain sequence, the variable heavy domain sequence and the three CDRs of the variable heavy domain sequence, the full light chain sequence, a variable light domain sequence and the three CDRs of the variable light domain sequence. A Fab-scFv-Fc monomer includes a full-length sequence, a variable heavy domain sequence, 3 heavy CDR sequences, and an scFv sequence (include scFv variable heavy domain sequence, scFv variable light domain sequence and scFv linker). Note that some molecules herein with a scFv domain use a single charged scFv linker (+H), although others can be used. In addition, the naming nomenclature of particular antigen binding domains (e.g., NKG2D and B7H3 binding domains) use a “Hx.xx_Ly.yy” type of format, with the numbers being unique identifiers to particular variable chain sequences. Thus, an Fv domain of the antigen binding domain is “H1 L1”, which indicates that the variable heavy domain, H1, was combined with the light domain L1. In the case that these sequences are used as scFvs, the designation “H1 L1”, indicates that the variable heavy domain, H1 is combined with the light domain, L1, and is in VH-linker-V_{L} orientation, from N- to C-terminus. This molecule with the identical sequences of the heavy and light variable domains but in the reverse order (V_{L}-linker-V_{H} orientation, from N- to C-terminus) would be designated “L1_H1.1”. Similarly, different constructs may “mix and match” the heavy and light chains as will be evident from the sequence listing and the figures.

### III. Definitions

In order that the application may be more completely understood, several definitions are set forth below. Such definitions are meant to encompass grammatical equivalents.

As used herein, the singular forms “a,” “an”, and “the” include plural referents unless the context clearly dictates otherwise. Thus, for example, reference to “an antigen” includes mixtures of antigens; reference to “a pharmaceutically acceptable carrier” includes mixtures of two or more such carriers, and the like. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A (alone)”, and “B (alone)”.

As used herein, the term “about” a value (or parameter) refers to ±10% of a stated value. When referring to a range of values (or parameters), the term “about” refers to +10% of the upper limit and −10% of the lower limit of a stated range of values. When a range of values is provided, it is to be understood that each intervening value between the upper and lower limit of that range, and any other stated or intervening value in that stated range, is encompassed within the scope of the present disclosure. Where the stated range includes upper and/or lower limits, ranges excluding either of those included limits are also included in the present disclosure.

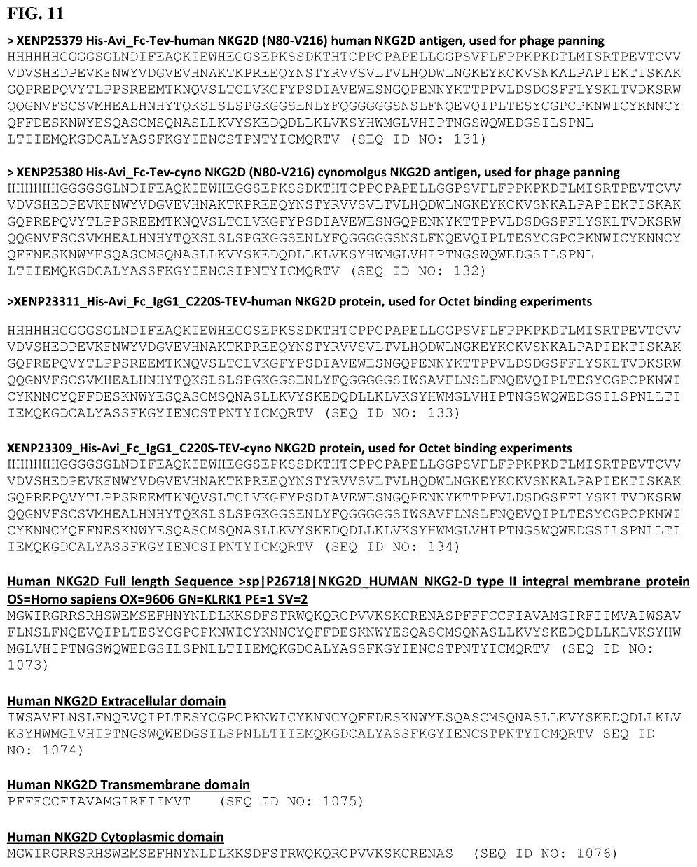

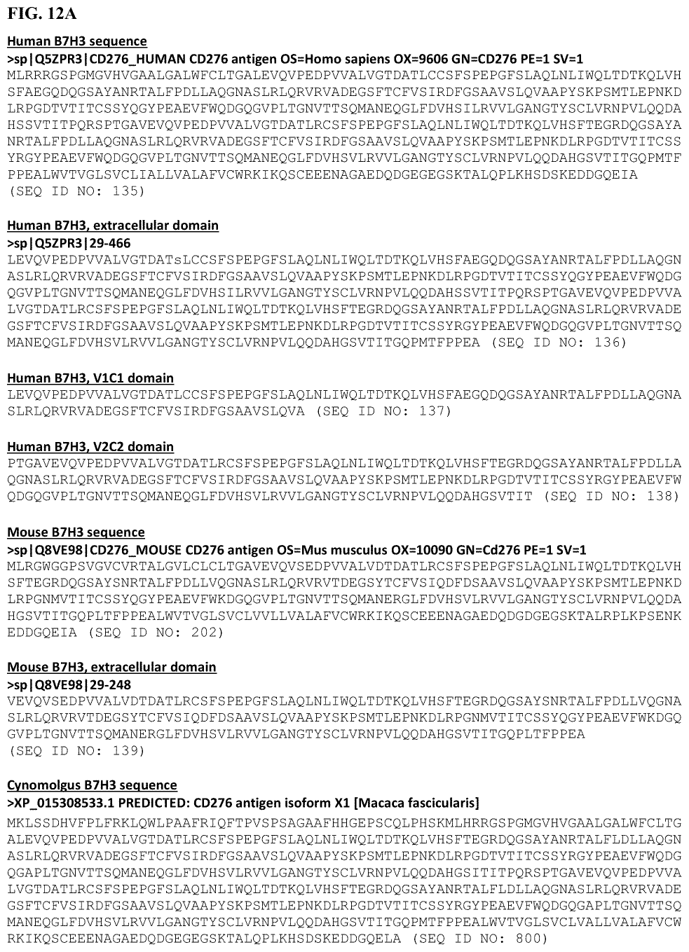

By “NKG2D,” “NKG2-D,” “natural killer group 2D,” “CD314,” (e.g., GenBank Accession Numbers NP_031386.2 (human), NP_001186734.1 (human), and NP_001076791.1 (mouse)), herein is meant a transmembrane protein belonging to the NKG2 family of C-type lectin-like receptors. NKG2D is a major recognition receptor for the detection and elimination of transformed and/or infected cells as its ligands are induced during cellular stress, either as a result of infection or genomic stress, such as in cancer. In humans, NKG2D is expressed by NK cells, γδ T cells, and CD8⁺ αβ T cells. Exemplary NKG2D sequences are depicted in FIG. 11. Unless otherwise noted, references to NKG2D are to the human NKG2D sequence.

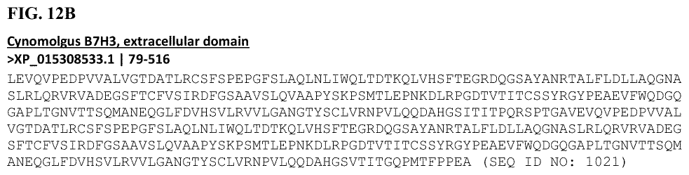

By “B7H3,” “B7-H3,” “B7RP-2,” “CD276,” “Cluster of Differentiation 276,” (e.g., GenBank Accession Numbers NP_001019907 (human), NP_001316557 (human), NP_001316558 (human), NP_079516 (human), and NP_598744 (mouse)) herein is meant a type-1 transmembrane protein that is a member of the B7 family possessing an ectodomain composed of a single IgV-IgC domain pair. B7H3 is an immune checkpoint molecule and is aberrantly overexpressed in many types of cancers. Exemplary B7H3 sequences are depicted in FIGS. 12A-12B. Unless otherwise noted, references to B7H3 are to the human B7H3 sequence.

By “ablation” herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments, and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

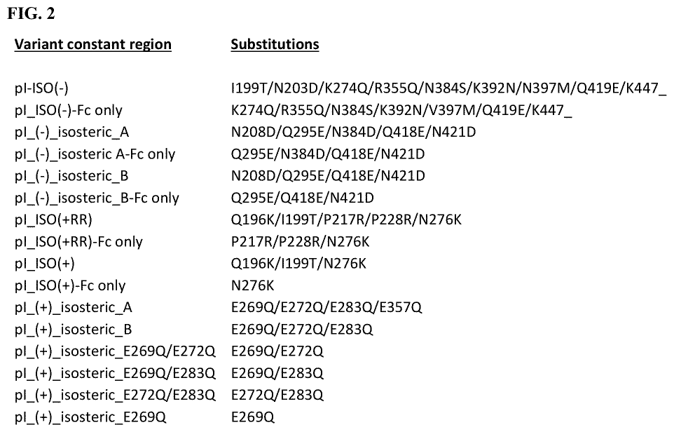

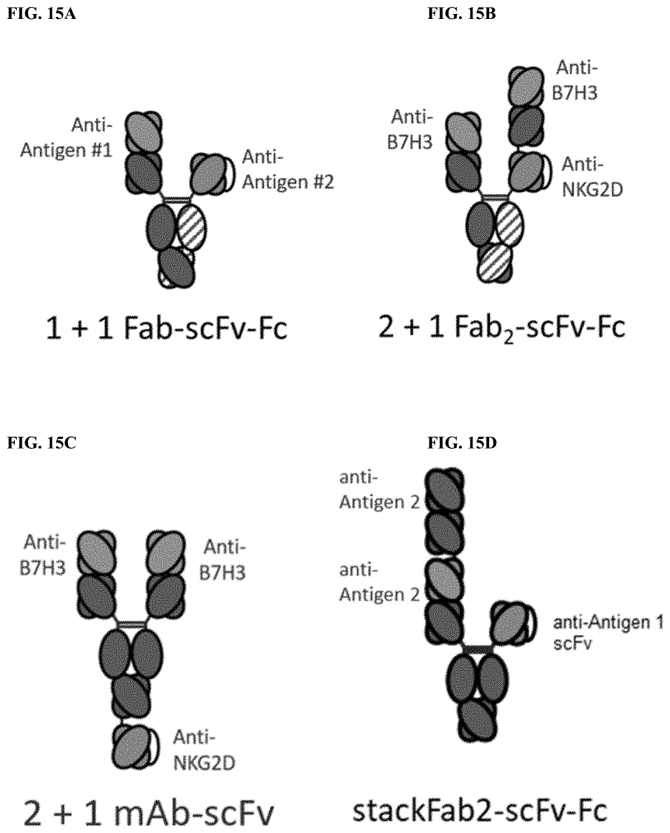

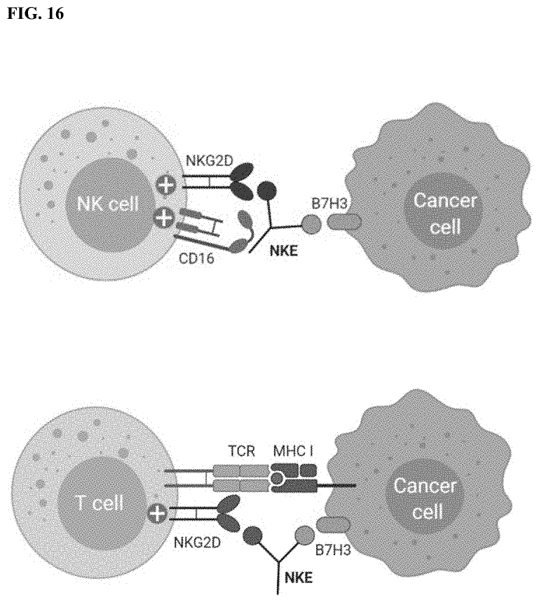

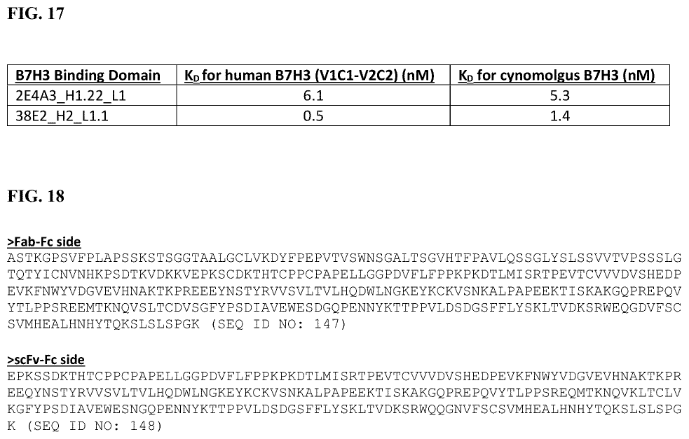

Other useful antibody formats include, but are not limited to, the “1+1 Fab-scFv-Fc,” “2+1 Fab₂-scFv-Fc,” “2+1 mAb-scFv,” and “stackFab2-scFv-Fc” formats provided herein (see, e.g., FIG. 15). Additional useful antibody formats include, but are not limited to, “mAb-Fv,” “mAb-scFv,” “central-Fv”, “one armed scFv-mAb,” “scFv-mAb,” “dual scFv,” and “trident” format antibodies, as disclosed in U.S. Pat. No. 10,793,632, which is incorporated by reference herein, particularly in pertinent part relating to antibody formats (see, e.g., FIG. 2 of U.S. Pat. No. 10,793,632).

Antibody heavy chains typically include a variable heavy (V_{H}) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminal, CH2-CH3 and hinge-CH2-CH3. In some embodiments, the Fc domain is that from IgG1, IgG2, or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus, for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (V_{L}), which includes light chain CDRs vlCDR1-3, and a constant light chain region (often referred to as C_{L} or C_{K}). The antibody light chain is typically organized from N- to C-terminus: V_{L}-C_{L}.

By “antigen binding domain” or “ABD” herein is meant a set of six Complementary Determining Regions (CDRs) that, when present as part of a polypeptide sequence, specifically binds a target antigen (e.g., B7H3 or NKG2D) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1): 55-77 (2003).

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 23-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH or V_{H}; containing the vhCDR1, vhCDR2 and vhCDR3) and the variable light domain (vl or VL or V_{L}; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the V_{κ}, V_{λ}, and/or V_{H} genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the V_{H}, CH1, V_{L}, and C_{L} immunoglobulin domains, generally on two different polypeptide chains (e.g., V_{H}-CH1 on one chain and V_{L}-C_{L} on the other). Fab may refer to this region in isolation, or this region in the context of a bispecific antibody of the invention. In the context of a Fab, the Fab comprises an Fv region in addition to the CH1 and C_{L} domains.

By “Fv” or “Fv fragment” or “Fv region” as used herein is meant the antibody region that comprises the V_{L} and V_{H} domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X L.Y means N- to C-terminal is vh-linker-vl, and L.Y H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as V_{H}-scFv linker-V_{L}, this can be reversed for any of the scFv domains (or those constructed using vh and vl sequences from Fabs), to V_{L}-scFv linker-V_{H}, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, -233E or 233E designates an insertion of glutamic acid after position 233 and before position 234. Additionally, -233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ), E233_, or E233del designates a deletion of glutamic acid at position 233. Additionally, EDA233- or EDA233 # designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild-type sequence, such as the heavy constant domain or Fc region from IgG1, IgG2, or IgG4, although human sequences with variants can also serve as “parent polypeptides”, for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2, or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides, and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcyR. Fc ligands also include Fc receptor homologs (FcRH), which are a family of Fc receptors that are homologous to the FcyRs (Davis et al., 2002, Immunological Reviews 190: 123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor,” “FcyR,” “FcγR,” or “FcgammaR” as used herein is meant any member of the family of proteins that bind the IgG antibody Fc region and is encoded by an FcyR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcyRIIb-1 and FcyRIIb-2), and FcγRIIc; and FcγRIII (CD16), including isoforms FcyRIIIa (including allotypes V158 and F158) and FcγRIIIb (including allotypes FcyRIIb-NA1 and FcyRIIb-NA2) (Jefferis et al., 2002, Immunol Lett 82:57-65, entirely incorporated by reference), as well as any undiscovered human FcyRs or FcyR isoforms or allotypes. An FcyR may be from any organism, including but not limited to humans, mice, rats, rabbits, and monkeys. Mouse FcyRs include but are not limited to FcγRI (CD64), FcγRII (CD32), FcγRIII (CD16), and FcγRIII-2 (CD16-2), as well as any undiscovered mouse FcyRs or FcyR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen binding domain comprising the variable regions of a given antibody.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pl higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild-type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a K_{D} for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹²M, or greater, where K_{D} refers to a dissociation rate of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a K_{A} or Kₐ for an antigen or epitope of at least 20-, 50-, 100-, 500-, 1000-, 5,000-, 10,000- or more times greater for the epitope relative to a control, where KA or Kₐ refers to an association rate of a particular antibody-antigen interaction. Binding affinity is generally measured using a Biacore, SPR or BLI assay.

### IV. Heterodimeric Antibodies

In another aspect, provided herein are anti-NKG2D×anti-B7H3 (also referred to herein as “αNKG2D×αB7H3,” “anti-B7H3×anti-NKG2D,” and “αB7H3×αNKG2D”) bispecific antibodies. Such antibodies include at least one NKG2D binding domain and at least one B7H3 binding domain. In some embodiments, bispecific αNKG2D×αB7H3 provided herein NK cell responses selectively in tumor sites that express B7H3.

Note that unless specified herein, the order of the antigen list in the name does not confer structure; that is an NKG2D×B7H3 1+1 Fab-scFv-Fc antibody can have the scFv bind to NKG2D or B7H3, although in some cases, the order specifies structure as indicated.

As is more fully outlined herein, these combinations of ABDs can be in a variety of formats, as outlined below, generally in combinations where one ABD is in a Fab format and the other is in an scFv format. Exemplary formats that are used in the bispecific antibodies provided herein include the 1+1 Fab-scFv-Fc and 2+1 Fab₂-scFv-Fc formats (see, e.g., FIGS. 15A and 15B). Other useful antibody formats include, but are not limited to, “mAb-scFv,” and “stackFab2-scFv-Fc” format antibodies, as depicted in FIG. 36 and more fully described below.

In addition, in general, one of the ABDs comprises a scFv as outlined herein, in an orientation from N- to C-terminus of V_{H}-scFv linker-V_{L} or V_{L}-scFv linker-V_{H}. One or both of the other ABDs, according to the format, generally is a Fab, comprising a V_{H} domain on one protein chain (generally as a component of a heavy chain) and a V_{L} on another protein chain (generally as a component of a light chain).

As will be appreciated by those in the art, any set of 6 CDRs or V_{H} and V_{L} domains can be in the scFv format or in the Fab format, which is then added to the heavy and light constant domains, where the heavy constant domains comprise variants (including within the CH1 domain as well as the Fc domain). The scFv sequences contained in the sequence listing utilize a particular charged linker, but as outlined herein, uncharged or other charged linkers can be used, including those depicted in FIG. 5.

In addition, as discussed above, the numbering used in the Sequence Listing for the identification of the CDRs is Kabat, however, different numbering can be used, which will change the amino acid sequences of the CDRs as shown in Table 2.

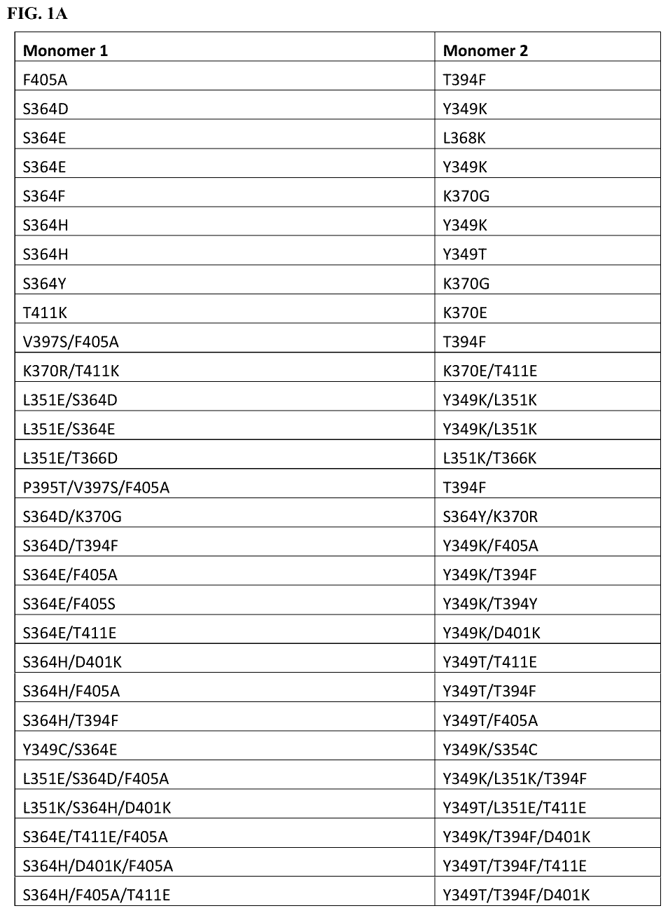

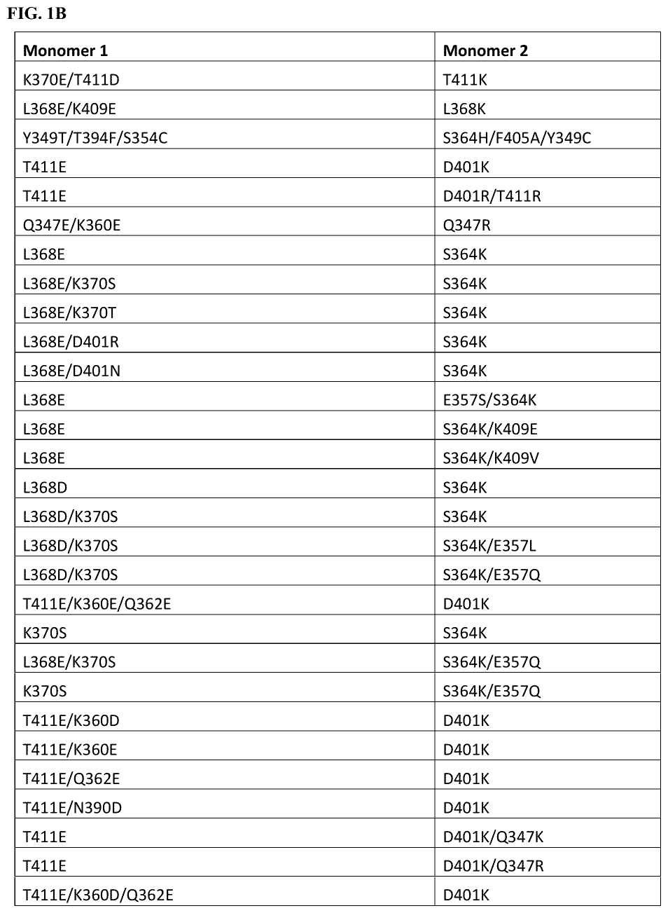

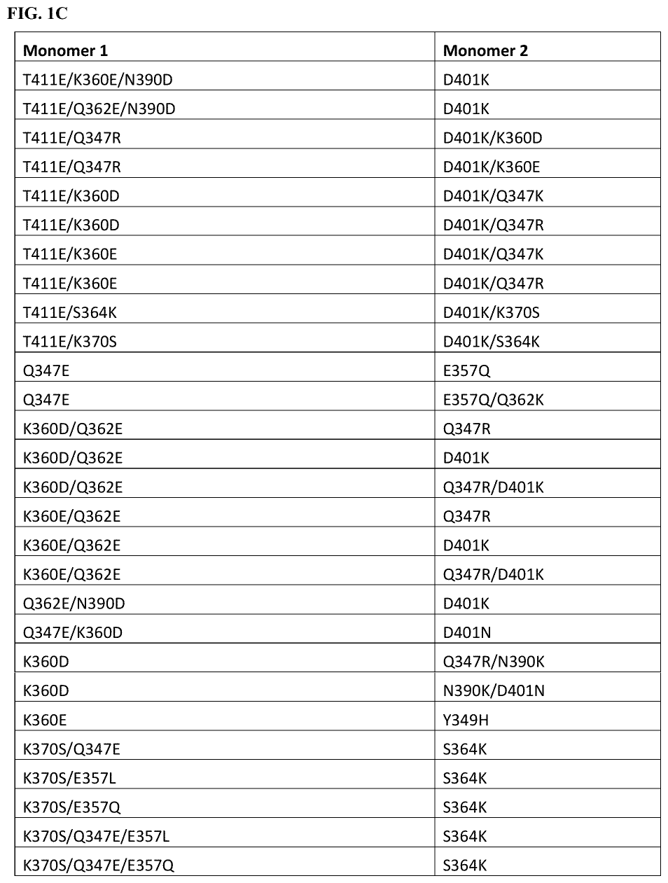

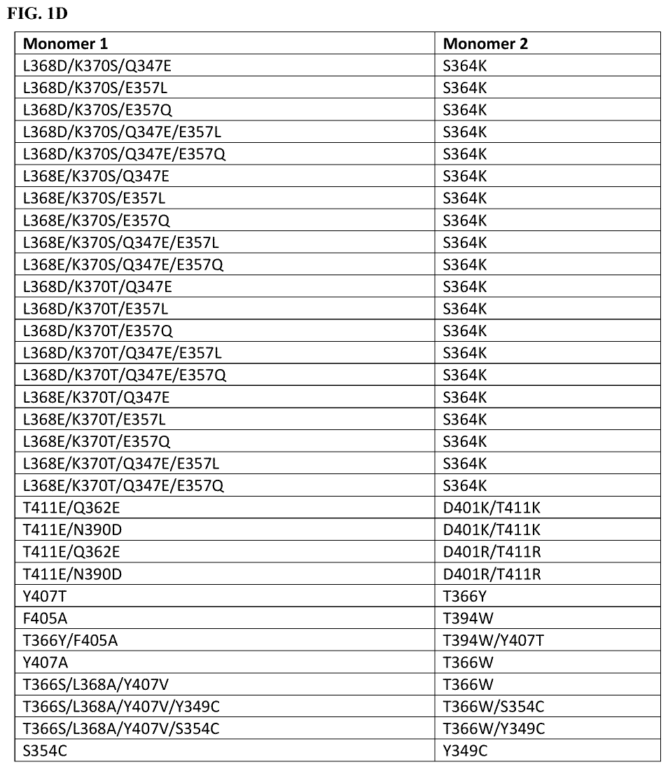

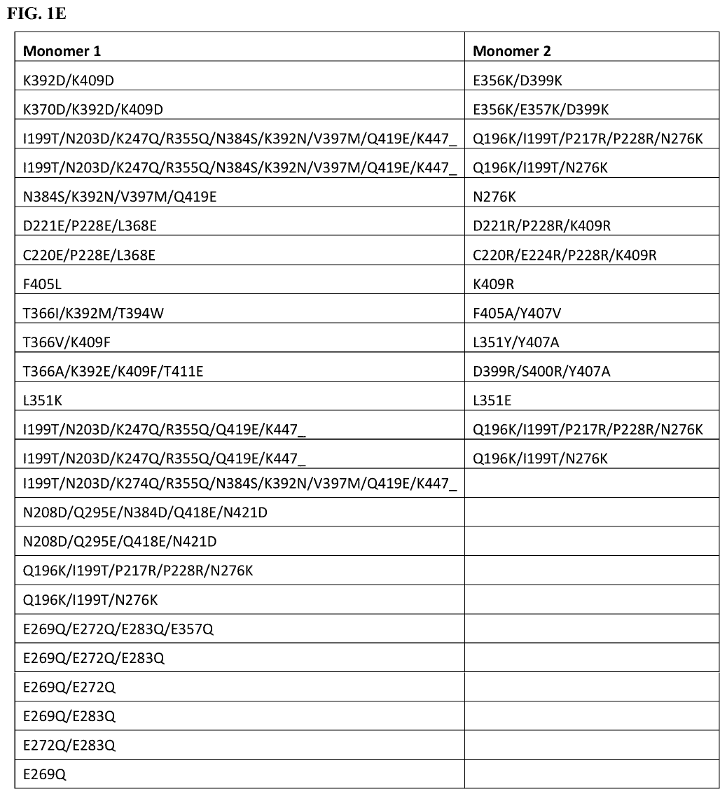

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 81, 82, 83, 84, 85, 86, 87, 88, 89, 90, 91, 92, 93, 94, 95, 96, 97, 98, or 99% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at 81, 82, 83, 84, 85, 86, 87, 88, 89, 90, 91, 92, 93, 94, 95, 96, 97, 98, or 99% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 81, 82, 83, 84, 85, 86, 87, 88, 89, 90, 91, 92, 93, 94, 95, 96, 97, 98, or 99% identity to a human germline sequence selected from those listed in FIG. 1 of U.S. Pat. No. 7,657,380.

As discussed herein, the subject heterodimeric antibodies include two antigen binding domains (ABDs), each of which bind to NKG2D or B7H3. As outlined herein, these heterodimeric antibodies can be bispecific and bivalent (each antigen is bound by a single ABD, for example, in the format depicted in FIG. 15A), or bispecific and trivalent (one antigen is bound by a single ABD and the other is bound by two ABDs, for example as depicted in FIG. 15B).

The antibodies provided herein include different antibody domains as is more fully described below. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

In particular, the formats depicted in FIG. 15 are usually referred to as “heterodimeric antibodies”, meaning that the protein has at least two associated Fc sequences self-assembled into a heterodimeric Fc domain and at least two Fv regions, whether as Fabs or as scFvs.

In certain embodiments, the antibodies described herein comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI, and ablation variants herein; that is, the number of variants is generally low, prior to the introduction of the variants described herein). In certain cases, the humanized antibody may display no more than 5, or even no more than 4, 3, 2, or 1 amino acid difference from the amino acid sequence encoded by the germline immunoglobulin gene (again, prior to the introduction of any skew, pI, and ablation variants herein; that is, the number of variants is generally low, prior to the introduction of the variants described herein). In some embodiments, the amino acid differences are in one or more of the 6 CDRs. In some embodiments, the amino acid differences are in a V_{H} and/or V_{L} framework region.

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### (i) Bispecific, Heterodimeric Antibodies as Natural Killer Cell Engagers

In one aspect, provided herein are anti-B7H3×anti-NKG2D bispecific antibodies, which are exemplary embodiments of a Natural Killer cell Engager (NKE). Generally, NKEs are multifunctional molecules that target activating or inhibitory receptors (in particular the ECD of such receptors) expressed on the surface of NK cells, bind to tumor associated antigens (in particular the ECD of such antigens) and activate Fc gamma receptors expressed on effector cells of a subject's immune system. The different domains of an NKE including the NK cell antigen binding domain, the tumor associated antigen binding domain and the Fc domains can modulate its activity and function.

As described herein and known in the art, the antibodies include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains. It should be noted that the term “Fc domain” includes both the CH2-CH3 (and optionally the hinge, hinge-CH2-CH3) of a single monomer, as well as the dimer of two Fc domains that self-assemble. That is, the heavy chain of an antibody has an Fc domain that is a single polypeptide, while the assembled bispecific antibody has an Fc domain that contains two polypeptides. Various antibody domains included in the bispecific, heterodimeric antibodies are more fully described below.

In particular, the formats schematically depicted in FIGS. 15A-15D are usually referred to as “heterodimeric antibodies,” meaning that the antibody format has at least two associated Fc sequences self-assembled into a heterodimeric Fc domain and at least two Fv regions, whether as Fabs or as scFvs.

Described below are useful variant Fc domains that include amino acid modifications (i.e., substitutions, insertions, or deletions) to enhance FcγR-mediated cytotoxicity, increase serum half-life, and facilitate the self-assembly and/or purification of the heterodimeric antibodies provided. Also, exemplary anti-B7H3×anti-NKG2D bispecific antibodies that include such variant Fc domains are described below and set forth in the Figures including FIGS. 56, 57, and 59, and the corresponding sequences in the Sequence Listing.

A. Fc Domain Variants for Increasing Antibody-Dependent Cellular Cytotoxicity (ADCC)

There are a number of useful Fc substitutions that can be made to alter binding to one or more of the FcγR receptors. Substitutions that result in increased binding (or in some cases, decreased binding) can be useful. For example, it is known that increased binding to FcγRIIIa can result in increased ADCC (antibody dependent cell-mediated cytotoxicity). In some instances, decreased binding to FcγRIIb (an inhibitory receptor) can be beneficial as well. Amino acid substitutions that find use in the present invention include those listed in U.S. Ser. No. 11/124,620 (particularly FIG. 41), U.S. Ser. Nos. 11/174,287, 11/396,495, 11/538,406, all of which are expressly incorporated herein by reference in their entirety and specifically for the variants disclosed therein.

In some embodiments, provided herein are bispecific antibodies containing Fc variants that increase antibody-dependent cellular cytotoxicity (ADCC; the cell-mediated reaction wherein nonspecific cytotoxic cells that express FcγRs recognize bound antibody on a target cell and subsequently cause lysis of the target cell) activity of the antibodies. In other words, the heterodimeric antibodies encompassed by the disclosure herein include amino acid substitutions in each or both of the Fc monomeric domains of a parental sequence, usually IgG1, that can enhance ADCC.

In some embodiments, the Fc ADCC variants (e.g., ADCC-enhanced Fc variants) comprise amino acid substitution(s) selected from the group including: S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, I332E/P247I/A339Q, S298A/E333A, S298A/E333A/K334A, V264I/I332E, S298A, S298A/I332E, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E298R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering. In some embodiments, the amino acid substitution(s) present in an Fc ADCC variants are selected from the group including: S239D, S239E, I332D, I332E, S239D/I332E, S239E/I332E, S239D/I332D, S239E/I332D, S239D/A330L/I332E, S239D/A330L, A330L/I332E, S239D/I332N, S239N/I332D, A330Y/I332E, A330L/I332E, L328V/I332E, L328T/I332E, S239Q/V264I/I332E, S239E/V264I/A330Y/I332E, L235D/S239D/A330Y/I332E, S239D/V240I/A330Y/I332E, S239D/V264T/A330Y/I332E, S239D/K326E/A330Y/I32E, S234D/K326T/A330Y/I332E, E274R, P227G, G236S, D221K, H224E, K246H, D249Y, R255Y, E258H, E258Y, T260H, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering.

In some embodiments, a first Fc domain and/or a second Fc domain of the bispecific antibody provided comprise an Fc ADCC variant selected from the group including: S239D, S239E, I332D, I332E, S239D/I332E, S239E/I332E, S239D/I332D, S239E/I332D, S239D/A330L/I332E, S239D/A330L, A330L/I332E, S239D/I332N, S239N/I332D, A330Y/I332E, A330L/I332E, L328V/I332E, L328T/I332E, S239Q/V264I/I332E, S239E/V264I/A330Y/I332E, L235D/S239D/A330Y/I332E, S239D/V240I/A330Y/I332E, S239D/V264T/A330Y/I332E, S239D/K326E/A330Y/I32E, S234D/K326T/A330Y/I332E, E274R, P227G, G236S, D221K, H224E, K246H, D249Y, R255Y, E258H, E258Y, T260H, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering.

In some embodiments, one or more of these variants can be included either in both of the Fc monomeric domains or in only one of the Fc monomeric domains of a heterodimeric antibody. In some embodiments, an anti-B7H3×anti-NKG2D bispecific antibody described includes ADCC-enhanced variants which includes one or more amino acid modifications in a first Fc domain and/or a second Fc domain, in other words, in the Fc domain of a first monomer, in the Fc domain of a second monomer, or in the Fc domains of both monomers. In some instances, a first Fc domain includes an Fc ADCC variant, and a second Fc domain does not include an Fc ADCC variant, resulting in an asymmetrical distribution of Fc ADCC variants. In other instances, a first Fc domain includes an Fc ADCC variant, and a second Fc domain includes an Fc ADCC variant. In one embodiment, the Fc ADCC variant of the first and second Fc domains can be the same amino acid substitution. Also, in one embodiment, the Fc ADCC variant of the first and second Fc domains can be different amino acid substitution.

In some embodiments, the Fc ADCC variants described bind with greater affinity to the FcγRIIIa (CD16A) human receptor. In some embodiments, the Fc variants have affinity for FcγRIIIa (CD16A) that is at least 1-fold, 5-fold, 10-fold, 100-fold, 200-fold, or 300-fold greater than that of the parental Fc domain.

In some embodiments, the Fc ADCC variants described can mediate effector function more effectively in the presence of effector cells. In some embodiments, the Fc variants mediate ADCC that is greater than that mediated by the parental Fc domain. In certain embodiments, the Fc variants mediate ADCC that is at least 5-fold, 10-fold, 20-fold, 30-fold, 40-fold, or 50-fold greater than that mediated by the parental Fc domain.

Additional detailed descriptions of Fc variants that may enhance ADCC are provided in WO2004/029207, which is expressly incorporated herein by reference in its entirety and specifically for the variants disclosed therein.

1. Fc v90 Variants

In some embodiments, an Fc domain with enhanced binding to human FcγRIIIa (CD16A) and thus increased ADCC activity (“an Fc ADCC variant”) utilizes the amino acid substitutions S239D/I332E (sometimes referred to as the “v90 variants”) in the CH2 domain of one or both of the monomeric Fc domains, according to EU numbering. In some embodiments, a bispecific antibody described herein comprises the Fc v90 variants (e.g., amino acid substitutions S239D/I332E) in both Fc domains. In some embodiments, a bispecific antibody described herein comprises the Fc v90 variants in only one of the monomeric Fc domains. In some embodiments, the antibody comprises the Fc v90 variants in one of the monomeric Fc domains and lacks the Fc v90 variants in another Fc domain. In some embodiments, the antibody comprises the Fc v90 variants in an Fc domain and an amino acid substitution S239D in the CH2 domain of another Fc domain, according to EU numbering. In certain embodiments, the antibody comprises the Fc v90 variants in an Fc domain and an amino acid substitution I332E in the CH2 domain of another Fc domain, according to EU numbering. In some embodiments, the antibody comprises the Fc v90 variants in an Fc domain and lacks an amino acid substitution selected from S239D, I332E and S239D/I332E in the CH2 domain of another Fc domain, according to EU numbering. In some embodiments, one monomeric Fc domain comprises the S239D variant and the other comprises the I332E variant. In some embodiments, one monomeric Fc domain comprises the S239D variant and the other comprises no Fc ADCC variant. In some embodiments, one monomeric Fc domain comprises the I332E variant and the other comprises no Fc ADCC variant.

As will be appreciated by those in the art, in the case of these asymmetrical Fc ADCC variants, which monomer receives which variant(s) can be based on the “strandedness” outlined herein; that is, it may be useful to calculate the pI of different combinations and utilize the Fc ADCC variants such that the pIs of the two monomers are different to facilitate purification.

In some embodiments, monomer 1 comprises a first Fc v90 variants, and monomer 2 comprises the amino acid substitution S239D or I332E. In some embodiments, monomer 1 comprises the Fc V90 variants, and monomer 2 does not comprise the amino acid substitution(s) S239D, I332E or S239D/I332E. In some embodiments, at least one of the Fc domains of the bispecific antibody comprises the Fc v90 variants. A first Fc domain may comprise the Fc v90 variants, or it may comprise a parental sequence relative to the Fc v90 variants (e.g., a wild-type Fc domain, a Fc domain with one or more amino acid modifications that improves ADCC but does not include S239D, I332E or S239D/I332E substitutions, and the like). In such instances where at least one of the Fc domains comprises a parental sequence, relative to the Fc v90 variants, for the purposes of this section, this Fc domain may be referred to as a “WT Fc domain” with respect to the S239 and I332 positions of the Fc domain. In some embodiments, the antibody described herein comprises an Fc domain having an amino acid substitution of either S239D, I332E, or S239D/I332E, and another Fc domain having an amino acid substitution of either S239D, I332E, or S239D/I332E. In some embodiments, the antibody described herein comprises an Fc domain having an amino acid substitution of either S239D, I332E, or S239D/I332E, and another Fc domain without an amino acid substitution of either S239D, I332E, or S239D/I332E.

In some embodiments, the first Fc domain and the second Fc domain contain a set of ADCC-enhanced variant substitutions (first Fc domain variant:second Fc domain variant) selected from the group including: S239:I332E; S239D:S239D; S239D:WT; S239D:S239D/I332E; S239D/I332E:WT; S239D/I332E:S239D; S239D/I332E:I332E; S239D/I332E:S239D/I332E; I332E:WT; I332E:I332E; I332E:S239D; I332E:S239D/I332E; WT:S239D; WT:I332E; WT:S239D/I332E, according to EU numbering. In some embodiments, monomer 1 and monomer 2 contain a set of ADCC-enhanced variant substitutions (monomer 1 monomer 2) selected from the group including: S239:I332E; S239D:S239D; S239D:WT; S239D:S239D/I332E; S239D/I332E:WT; S239D/I332E:S239D; S239D/I332E:I332E; S239D/I332E:S239D/I332E; I332E:WT; I332E:I332E; I332E:S239D; I332E:S239D/I332E; WT:S239D; WT:I332E; WT:S239D/I332E, according to EU numbering.

In some embodiments, Fc domains with enhanced ADCC can further comprise one or more additional modifications at one or more of the following positions, including, but not limited to, 236, 243, 298, 299, or 330 in the CH2 domain, according to EU numbering. In some embodiments, the Fc variant domains comprise an amino acid substitution including, but not limited to: 236A, 243L, 298A, 299T, or 330L in the CH2 domain, according to EU numbering.

In some embodiments, an ADCC-enhanced Fc variant further includes, but is not limited, an amino acid substitution at one or more positions of the CH2 domain, according to EU numbering selected from the group including: position 236, 243, 298, 299, and 330. In some embodiments, an ADCC-enhanced Fc variant includes an amino acid substitution selected from the group including: 236A, 243L, 298A, 299T, 330L, 239D/332E, 236A/332E, 239D/332E/330L, 332E/330L, and any combination thereof in the CH2 domain, according to EU numbering. In some embodiments, the first Fc domain and/or the second Fc domain comprises an ADCC-enhanced Fc variant including, but not limited to, an amino acid substitution selected from the group including: 236A, 243L, 298A, 299T, 330L, 239D/332E, 236A/332E, 239D/332E/330L, 332E/330L, and any combination thereof in the CH2 domain, according to EU numbering, such that the Fc ADCC variant is the same in both Fc domain. Alternatively, the Fc ADCC variant is a different variant in each of the Fc domains.

Engineered antibodies comprising such ADCC-enhanced Fc variants can also have higher-affinity FcγRIIIa binding, thus resulting in stronger ADCC activity with NK cells. Bispecific antibodies having a variant Fc domain described herein can be useful and effective for NK cell-mediated killing of tumor cells.

2. Fc Variants to Increase Binding to FcγRIIIa/CD16A

There are additional Fc substitutions that find use in enhancing FcγRIIIa binding. In some embodiments, the Fc domains of the bispecific antibodies provided include one or more Fc domains having increased binding to FcγRIIIa as compared to human IgG1 produced in standard research and production cell lines. In some embodiments, the Fc variants with improved binding affinity to at least FcγRIIIa have amino acid substitution(s) selected from the group including: V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E298R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering of the Fc domain. Additional Fc variants with enhanced binding affinity, specificity and/or avidity to FcγRIIIa are disclosed the specification and FIG. 41 of U.S. Pat. No. 8,188,231.

The described bispecific antibodies contain such Fc variants that provide enhanced effector function and substantial increases in affinity for FcγRIIIa. In some embodiments, the Fc variants improve binding to FcγRIIIa allotypes such as, for example, both V158 and F158 polymorphic forms of FcγRIIIa. The FcγR binding affinities of these Fc variants can be evaluated using assay recognized by those skilled in the art including, but not limited to, a Surface Plasmon Resonance (SPR) and/or a BLI binding assay (such as Biacore, Octet, or Carterra LSA).

B. Fc Variants for Increasing Binding to FcRn

Provided herein are additional Fc substitutions that find use in increased binding to the FcRn receptor and increased serum half-life, as specifically disclosed in U.S. Ser. No. 12/341,769, hereby incorporated by reference in its entirety, including, but not limited to, N434S, N434A, M428L, V308F, V259I, M428L/N434S, M428L/N434A, V259I/V308F, Y436I/M428L, Y436I/N434S, Y436V/N434S, Y436V/M428L, M252Y/S254T/T256E, and V259I/V308F/M428L. Such modification may be included in one or both Fc domains of the subject antibody.

In some embodiments, additional Fc variants can increase serum half-life of a bispecific antibody compared to a parental Fc domain. In some embodiments, the Fc variants have one or more amino acid modifications (i.e., substitutions, insertions or deletions) at one or more of the following amino acid residues or positions selected from the group including: 234, 235, 238, 250, 252, 254, 256, 265, 272, 286, 303, 305, 307, 311, 312, 317, 322, 340, 356, 360, 362, 376, 378, 380, 382, 413, 424, 428, and 434, according to EU numbering of the Fc region.

In some embodiments, the Fc variants have one or more amino acid substitutions selected from the group including: 234F, 235Q, 250E, 250Q, 252T, 252Y, 254T, 256E, 428L, 428F, 434S, 434A, 428L/434S, 428L/434A, 252Y/254T/256E, 234F/235Q/252T/254T/256E/322Q, 250E/428F, 250E/428L, 250Q/428F, and 250Q/428L, according to EU numbering.

In some embodiments, antibodies described can include M428L/N434S or M428L/N434A substitutions in one or both Fc domains, which can result in longer half-life in serum. In more embodiments, a first Fc domain or a second Fc domain include M428L/N434S substitutions. In more embodiments, a first Fc domain and a second Fc domain include M428L/N434S substitutions. In certain embodiments, a first Fc domain or a second Fc domain include M428L/N434A substitutions. In certain embodiments, a first Fc domain and a second Fc domain include M428L/N434A substitutions. Such substitutions can result in longer half-life in serum of molecules comprising such.

C. Fc Variants for Heterodimerization

In some embodiments, the anti-B7H3×anti-NKG2D bispecific antibodies provided herein are heterodimeric bispecific antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibodies away from homodimers are further discussed in detailed below.

There are a number of suitable pairs of sets of heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other; that is, these pairs of sets form an interface between the two monomers that encourages heterodimer formation and discourages homodimer formation, allowing the percentage of heterodimers that spontaneously form under biological conditions to be over 90%, rather than the expected 50% (25 homodimer A/A:50% heterodimer A/B25% homodimer B/B).

1. Skew Variants

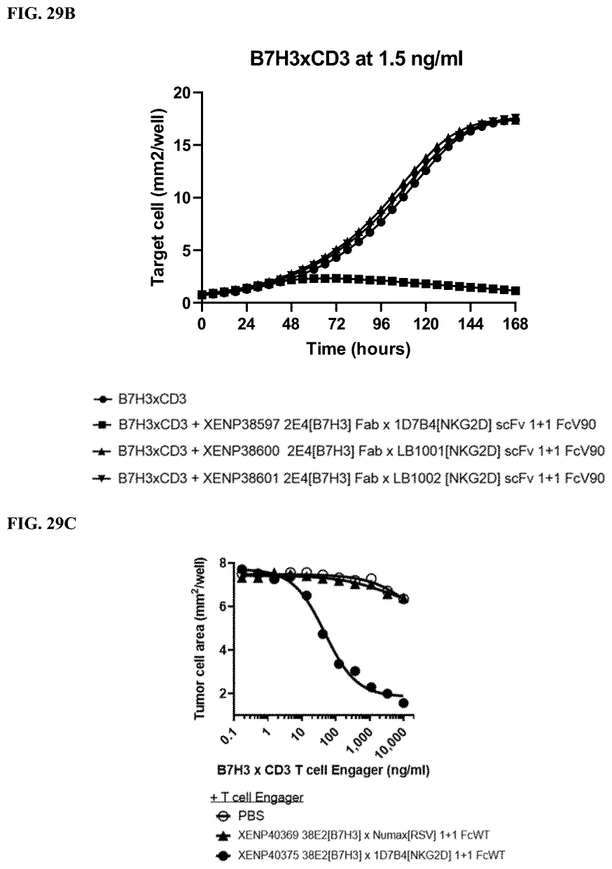

In some embodiments, the heterodimeric antibody includes skew (e.g., steric) variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 1A-1E.

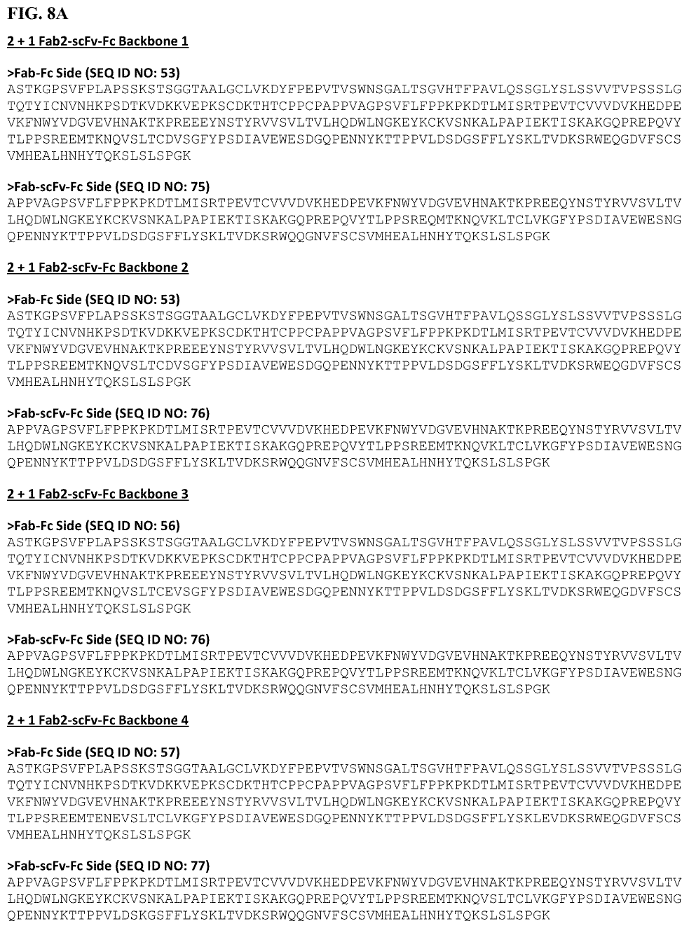

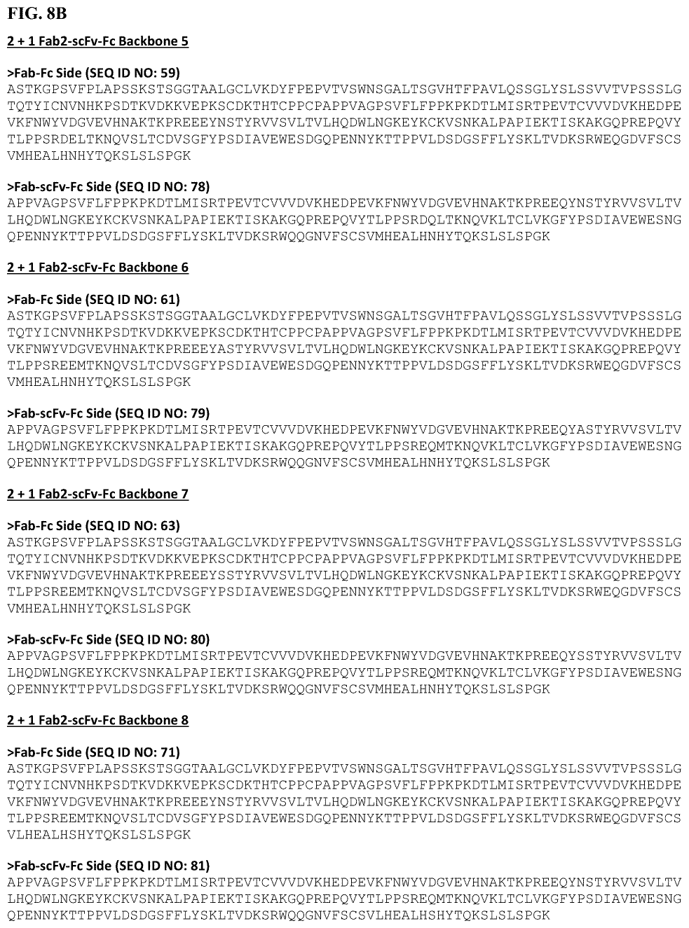

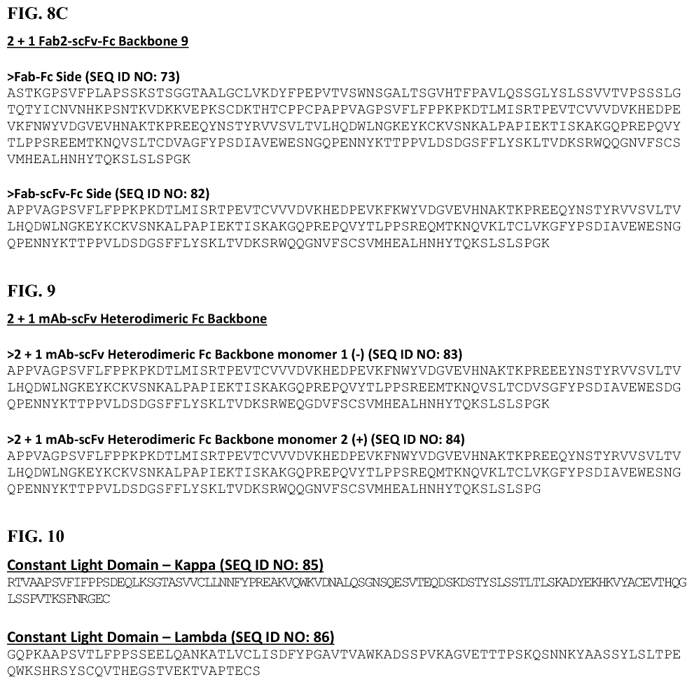

Thus, suitable Fc heterodimerization variant pairs that will permit the formation of heterodimeric Fc regions are shown in the figures including FIGS. 1A-1E, 4, 7A-7C, 8A-8C, 35A-35D, and 49A-49D. Thus, a first Fc domain has first Fc heterodimerization variants and the second Fc domain has second Fc heterodimerization variants selected from the pairs in FIGS. 1A-1E, 7A-7C, 8A-8C, 35A-35D, and 49A-49D.

One mechanism is generally referred to in the art as “knobs and holes,” referring to amino acid engineering that creates steric influences to favor heterodimeric formation and disfavor homodimeric formation can also optionally be used; this is sometimes referred to as “knobs and holes,” as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety. The Figures identify a number of “monomer A-monomer B” pairs that rely on “knobs and holes”. In addition, as described in Merchant et al., Nature Biotech. 16:677 (1998), these “knobs and hole” mutations can be combined with disulfide bonds to skew formation to heterodimerization.

An additional mechanism that finds use in the generation of heterodimers is sometimes referred to as “electrostatic steering” as described in Gunasekaran et al., J. Biol. Chem. 285(25):19637 (2010), hereby incorporated by reference in its entirety. This mechanism is also sometimes referred to herein as “charge pairs”. In this embodiment, electrostatics are used to skew the formation towards heterodimerization. As those in the art will appreciate, these variants may also have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R (e.g., these are “monomer corresponding sets) and C220E/P228E/368E paired with C220R/E224R/P228R/K409R. In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization skew variants are depicted in FIGS. 1A-1E, 4, 7A-7C, 8A-8C, 35A-35D, and 49A-49D. Such skew variants include, but are not limited to: S364K/E357Q:L368D/K370S; L368D/K370S:S364K; L368E/K370S:S364K; T411E/K360E/Q362E:D401K; L368D/K370S:S364K/E357L; K370S:S364K/E357Q (EU numbering). In terms of nomenclature, the pair “S364K/E357Q:L368D/K370S” means that one of the monomers has the double variant set S364K/E357Q and the other has the double variant set L368D/K370S. In exemplary embodiments, the heterodimeric antibody includes Fc heterodimerization variants as sets: S364K/E357Q:L368D/K370S; L368D/K370S:S364K; L368E/K370S:S364K; T411E/K360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C or T366S/L368A/Y407V/S354C:T366W/Y349C) are all skew variant amino acid substitution sets of Fc heterodimerization variants. In an exemplary embodiment, the heterodimeric antibody includes a “S364K/E357Q:L368D/K370S” amino acid substitution set. In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotypic variants, FcRn variants, ablation variants, etc. into one or both of the first and second Fc domains of the heterodimeric antibody. Further, individual modifications can also independently and optionally be included or excluded from the subject the heterodimeric antibody.

Additional monomer A and monomer B variants that can be combined with other variants, optionally and independently in any amount, such as pI variants outlined herein or other steric variants that are shown in FIG. 37 of US 2012/0149876, the figure and legend and SEQ ID NOs of which are incorporated expressly by reference herein.

In some embodiments, the steric variants outlined herein can be optionally and independently incorporated with any pI variant (or other variants such as, for example, Fc ADCC variants, FcRn variants, etc.) into one or both monomers, and can be independently and optionally included or excluded from the proteins of the antibodies described herein.

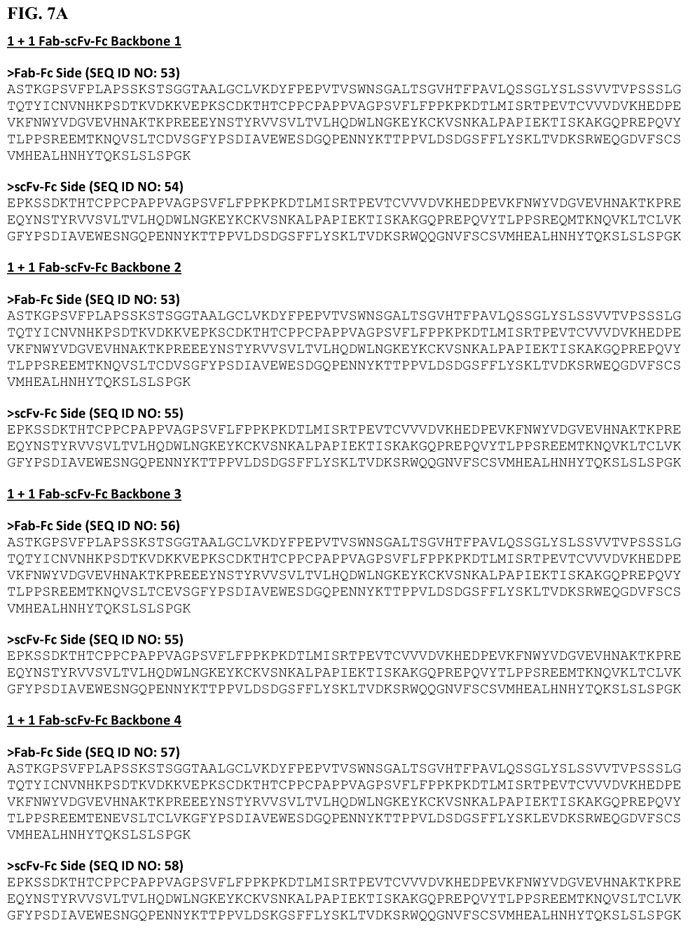

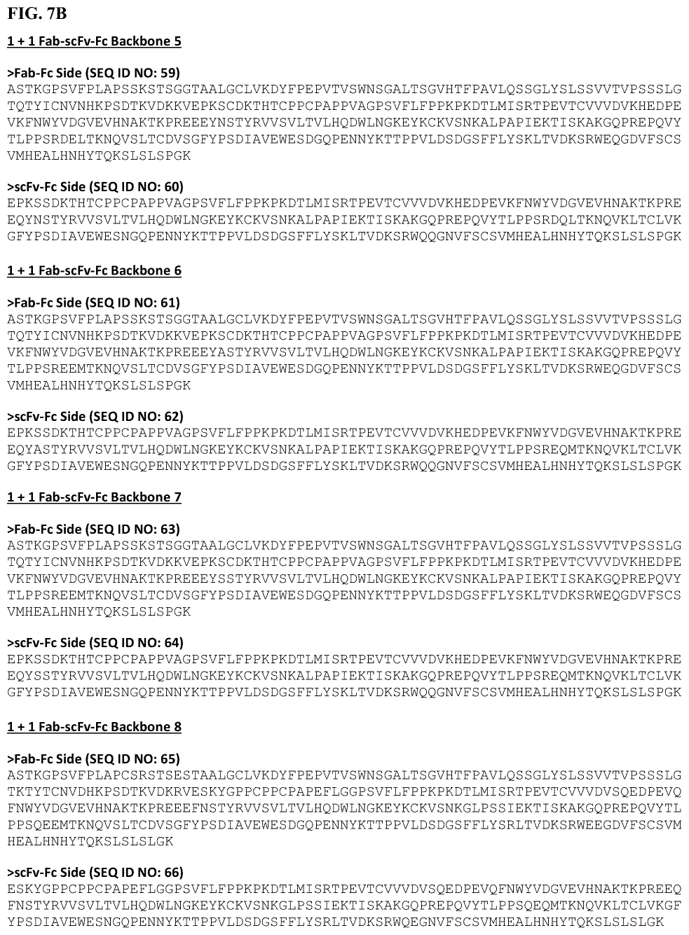

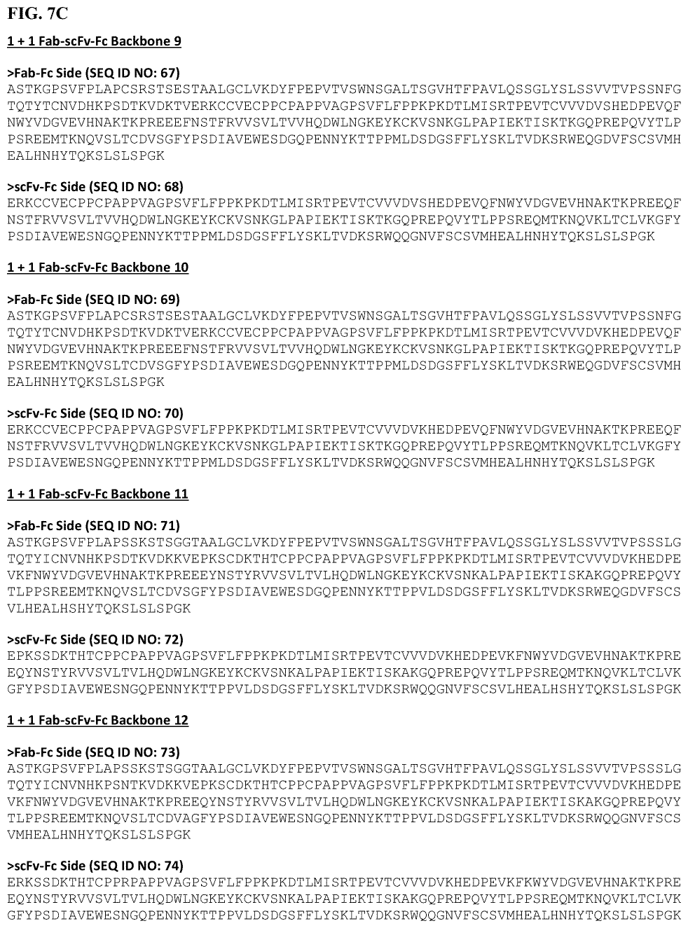

A subset of skew variants are “knobs in holes” (KIH) variants. Exemplary “knob-in-hole” variants are depicted in FIG. 7 of U.S. Pat. No. 8,216,805, which is incorporated herein by reference. Such “knob-in-hole” variants include, but are not limited to: an amino acid substitution at position 347, 349, 350, 351, 357, 366, 368, 370, 392, 394, 395, 397, 398, 399, 405, 407 and/or 409 of the CH3 constant domain of an IgG such as an IgG1, IgG2a, IgG2b, or IgG4 (Kabat numbering). In some embodiments, the “knob-in-hole” variants include, but are not limited to: an amino acid substitution at Y349, L351, E357, T366, L368, K370, N390, K392, T394, D399, S400, F405, Y407, K409, R409, T411, or any combination thereof of the CH3 domain of an IgG such as an IgG1, IgG2a, IgG2b, IgG4 (EU numbering). In some embodiments, the “knob-in-hole” variants include, but are not limited to: one or more amino acid substitutions including Y349D/E, L351D/K/Y, E357K, T366A/K/Y, L368E, K370E, N390D/K/R, K392E/F/L/M/R, T394W, D399K/R/W/Y, S400D/E/K/R, F405A/I/M/S/T/V/W, Y407A/Y, K409E/D/F, R409E/D/F, and T411D/E/K/N/Q/R/W.

In some embodiments, such variants include one or more amino acid substitutions including, but not limited to: Y349C, E357K, S354C, T366S, T366W, T366Y, L368A, K370E, T394S T394W, D399K, F405A, F405W, Y407A, Y407T, Y407V, R409D, T366Y/F405A, T394W/Y407T, T366W/F405W, T394S/Y407A, F405W/Y407A, and T366W/T394S (EU numbering). In some embodiments, these variants include knob:hole paired substitutions including, but not limited to: T366W:Y407V; S354C/T366W:Y349C/T366S/Y407V; Y349C/T366W:S354C/T366S/L368A/Y407V; Y349C/T366W/R409D/K370E:S354C/T366S/L368A/Y407V/D399K/E357K; R409D/K370E:D399K/E357K; T366W:T366S/L368A/Y407V; T366W/R409D/K370E:T366S/L368A/Y407V/D399K/E357K; T366W:T366S/L368A/Y407V; T366W/Y366Y:T366S/L368A/T394W/F405A/Y407V; Y349C/T366W:S354C/T366S/L368A/Y407V; Y349C/T366W/R409D/K370E:S354C/T366S/L368A/Y407V/D399K/E357K paired substitutions, according to EU numbering.

Additional exemplary “knob-in-hole” variants as described by the amino acid substitutions of the CH3 domains can be found in, for example, Carter et al., J. Immunol. Methods, 248(1-2):7-15 (2001), Merchant et al. Nat. Biotechnol. 16(7):677-81 (1998), Ridgway et al. Protein Eng. 9(7):617-2 (1996), and U.S. Pat. Nos. 8,216,805 and 10,287,352, the disclosures of which are herein incorporated by reference in their entireties.

2. pI (Isoelectric Point) Variants for Heterodimers

In some embodiments, the heterodimeric antibody includes purification variants that advantageously allow for the separation of heterodimeric antibody (e.g., anti-B7H3×anti-NKG2D bispecific antibody) from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format and the “2+1 Fab₂-scFv-Fc” format, also allows separation on the basis of size. As described above, it is also possible to “skew” the formation of heterodimers over homodimers using skew variants. Thus, a combination of heterodimerization skew variants and pI variants find particular use in the heterodimeric antibodies provided herein.

Additionally, as more fully outlined below, depending on the format of the heterodimeric antibody, pI variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc,” format can include charged scFv linkers (either positive or negative), that give a further pI boost for purification purposes. As will be appreciated by those in the art, some 1+1 Fab-scFv-Fc formats are useful with just charged scFv linkers and no additional pI adjustments, although the antibodies described herein do provide pI variants that are on one or both of the monomers, and/or charged domain linkers as well. In addition, additional amino acid engineering for alternative functionalities may also confer pI changes, such as Fc, FcRn and KO variants.

In subject heterodimeric antibodies for which pI is used as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B can be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (e.g., aspartic acid to lysine) or changing a charged residue to a neutral residue (e.g., loss of a charge; lysine to serine). A number of these pI variants are shown in the FIGS. 1A-1E, 2, 4, 7A-7E, 8A-8C, 35A-35D, and 49A-49D.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the antibodies described herein.

As will be appreciated by those in the art, the number of pI variants to be included on each or both monomer(s) to achieve good separation will depend in part on the starting pI of the components, for example in the 1+1 Fab-scFv-Fc and 2+1 Fab₂-scFv-Fc formats, the starting pI of the scFv and Fab(s) of interest. That is, to determine which monomer to engineer or in which “direction” (e.g., more positive, or more negative), the Fv sequences of the two target antigens are calculated and a decision is made from there. As is known in the art, different Fvs will have different starting pIs which are exploited in the antibodies described herein. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively, or in addition to isotypic substitutions, the possibility of immunogenicity resulting from the pI variants is significantly reduced by utilizing isosteric substitutions (e.g., Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize, and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

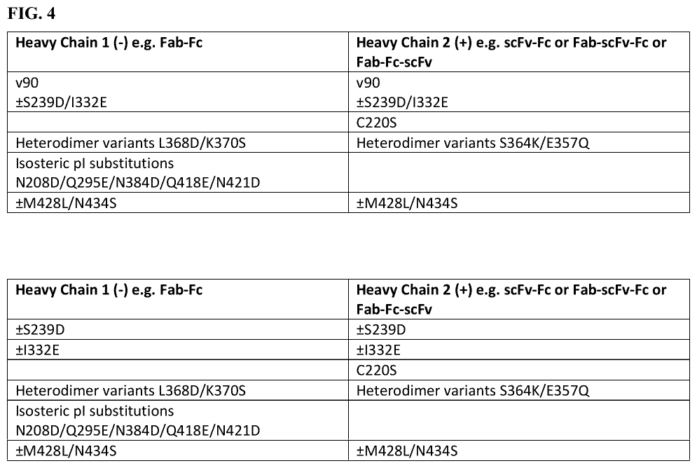

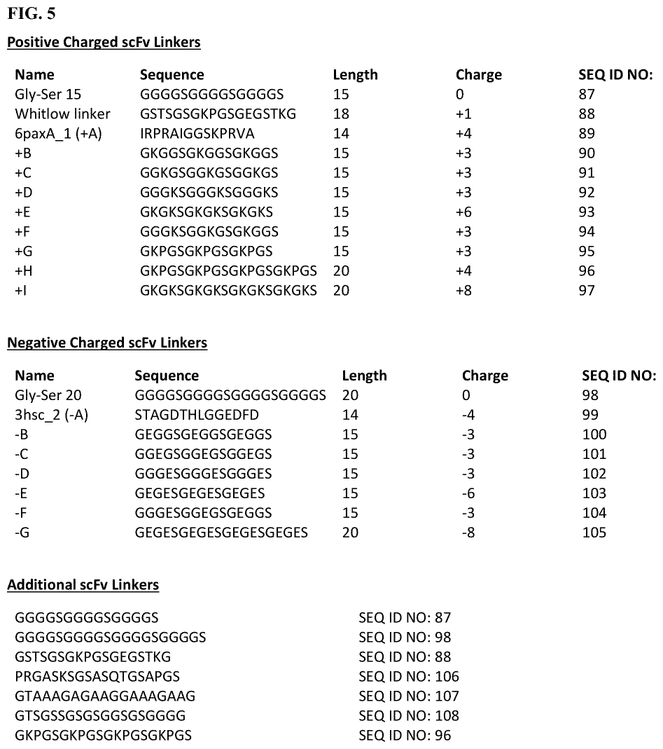

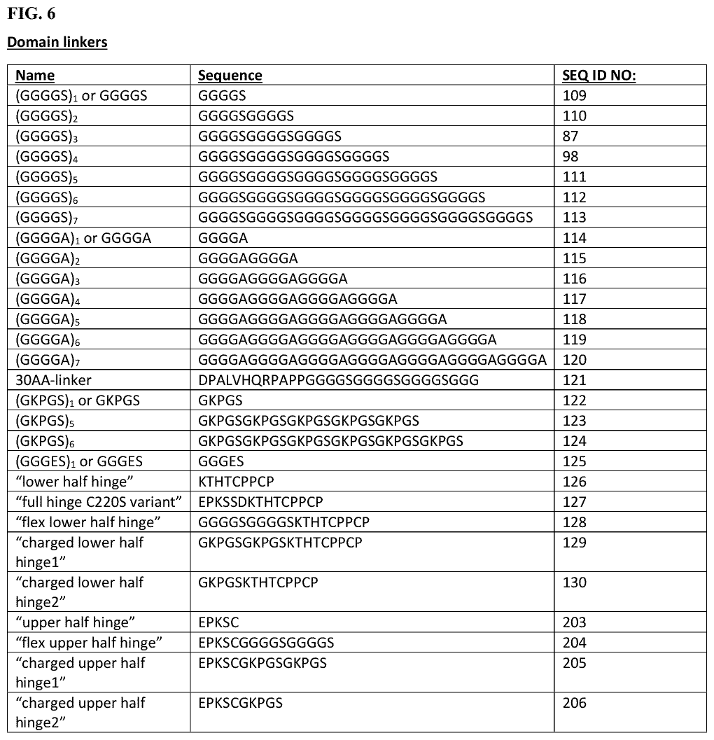

Exemplary combinations of pI variants are shown in FIGS. 4 and 5, and FIG. 30 of US Publ. App. No. 2016/0355608, all of which are herein incorporated by reference in its entirety and specifically for the disclosure of pI variants. Preferred combinations of pI variants are shown in FIGS. 1A-1E, 2 and 4. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)4 (SEQ ID NO: 1). However, as will be appreciated by those in the art, the first monomer includes a CH1 domain, including position 208. Accordingly, in constructs that do not include a CH1 domain (for example for fusion proteins that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 2 and the other monomer has a charged linker (either in the form of a charged scFv linker because that monomer comprises an scFv or a charged domain linker, as the format dictates, which can be selected from those depicted in FIG. 5).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389, 392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

3. Isotypic Variants

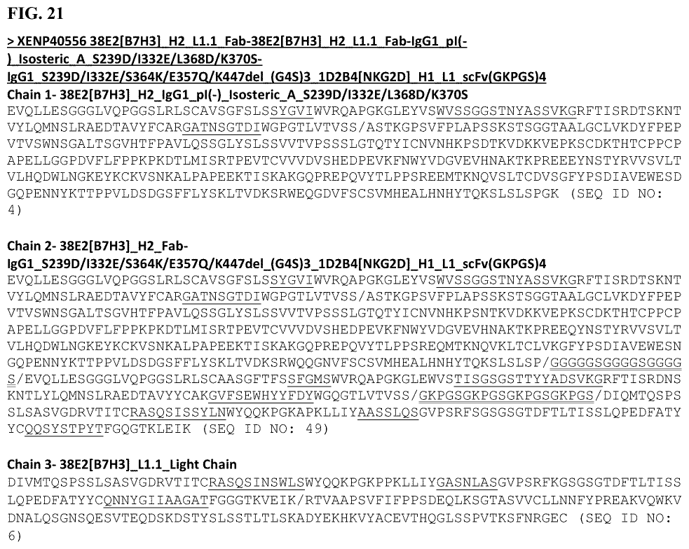

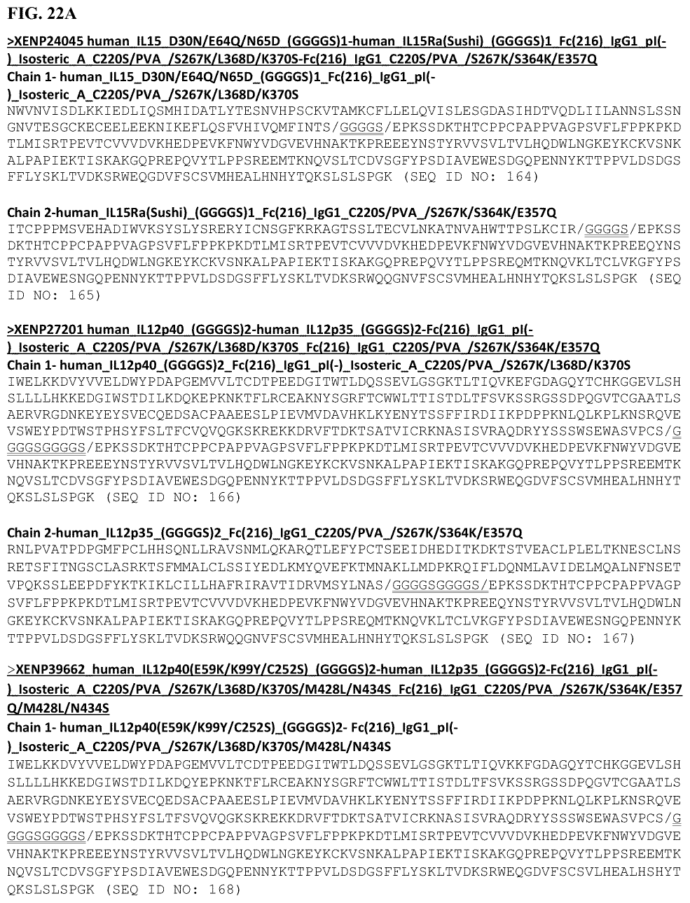

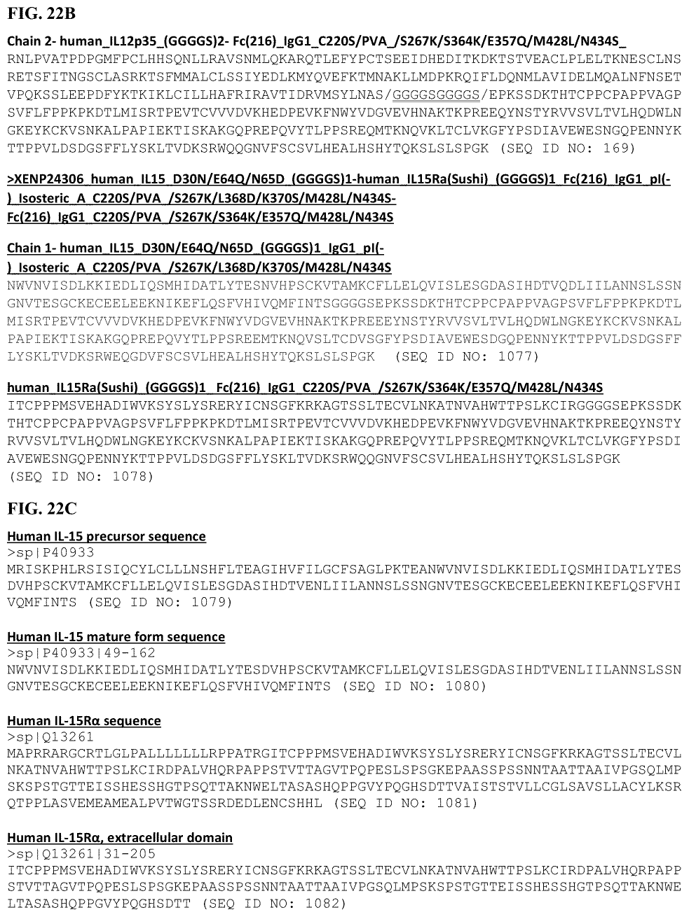

In addition, many embodiments of the antibodies described herein rely on the “importation” of pI amino acids at particular positions from one IgG isotype into another, thus reducing or eliminating the possibility of unwanted immunogenicity being introduced into the variants. A number of these are shown in FIG. 21 of U.S. Publ. App. No. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

4. Calculating pI

The pI of each monomer can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of U.S. Publ. App. No. 2014/0370013. As discussed herein, which monomer to engineer is generally decided by the inherent pI of the Fv and scaffold regions. Alternatively, the pI of each monomer can be compared.

5. pI Variants that Also Confer Better FcRn In Vivo Binding

In the case where the pI variant decreases the pI of the monomer, they can have the added benefit of improving serum retention in vivo.

Although still under examination, Fc regions are believed to have longer half-lives in vivo, because binding to FcRn at pH 6 in an endosome sequesters the Fc (Ghetie and Ward, 1997, Immunol Today, 18(12): 592-598, entirely incorporated by reference). The endosomal compartment then recycles the Fc to the cell surface. Once the compartment opens to the extracellular space, the higher pH 7.4, induces the release of Fc back into the blood. In mice, Dall'Acqua et al. showed that Fc mutants with increased FcRn binding at pH 6 and pH 7.4 actually had reduced serum concentrations and the same half-life as wild-type Fc (Dall'Acqua et al., 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010, PEDS, 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

6. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed herein.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

7. Additional Heterodimerization Variants

In some embodiments, the first Fc domain comprises one or more amino acid substitutions selected from the group including: L351Y, D399R, D399K, S400D, S400E, S400R, S400K, F405A, F405I, F405M, F405T, F405S, F405V, F405W, Y407A, Y407I, Y407L, Y407V, and any combination thereof, and the second Fc domain comprises one or more amino acid substitutions selected from the group including: T350V, T366A, T366I, T366L, T366M, T366Y, T366S, T366C, T366V, T366W, N390D, N390E, N390R, K392L, K392M, K392I, K392D, K392E, T394W, K409F, K409W, T411N, T411R, T411Q, T411K, T411D, T411E, T411W, and any combination thereof.

In some embodiments, other heterodimerization pair variants include, but are not limited to, amino acid substitutions of L234A/L235A:wildtype; L234A/L235A:L234K/L235K; L234D/L235E:L234K/L235K; E233A/L234D/L235E:E233A/L234R/L235R; L234D/L235E:E233K/L234R/L235R; E233A/L234K/L235A:E233K/L234A/L235K; E269Q/D270N:E269K/D270R; and WT:L235K/A327K of the CH2 domain, according to the EU numbering.

In some embodiments, the first and/or second Fc domains comprise one or more amino acid substitutions selected from the group including: S239D, D265S, S267D, E269K, S298A, K326E, A330L and I332E. In certain instances, the Fc paired variants include, but are not limited to, S239D/D265S/I332E/E269K:S239D/D265S/S298A; S239D/K326E/A330L/I332E:S298A or S239D/K326E/A330L/I332E/E269K:S298A of the CH2 domain, according to EU numbering.

Additional descriptions of useful heterodimeric variants are disclosed in U.S. Pat. Nos. 9,732,155; 10,457,742 and 10,875,931 and U.S. Publ. App. Nos. 2021/0277150 and 2020/0087414, the disclosures of which, including the description of Fc domain variants are herein incorporated by reference in their entireties.

D. Ablation Variants

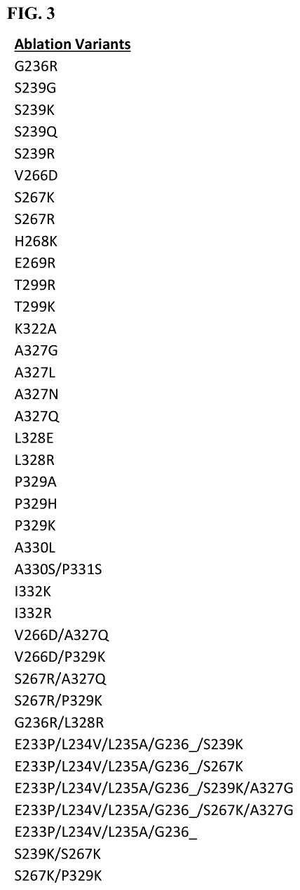

While in general NK engager multispecific antibodies retain at binding to CD16A (including “wild type” binding or increased binding to CD16A as outlined above), in some cases, surprisingly, NK engager activity can be seen even when binding to CD16A has been reduced or ablated. Accordingly, provided is another category of functional Fc variants to include are “FcγR ablation variants” or “Fc knock out (FcKO or KO)” variants. In these embodiments, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind a target antigen monovalently it is generally desirable to ablate FcγRIIIa binding to eliminate or significantly reduce ADCC activity. Wherein one of the Fc domains comprises one or more Fcγ receptor ablation variants. These ablation variants are depicted in FIG. 3, and each can be independently and optionally included or excluded, with preferred aspects utilizing ablation variants selected from the group including G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fc receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

E. Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the Figures, other combinations can be generated, following the basic rule of altering the pI difference between two monomers to facilitate purification.

In addition, any of the heterodimerization variants, skew, and pI, are also independently and optionally combined with Fc ADCC variants, Fc variants, FcRn variants, or Fc ablation variants, as generally outlined herein.

Exemplary combination of variants that are included in some embodiments of the heterodimeric 1+1 Fab-scFv-Fc and 2+1 Fab₂-scFv-Fc format antibodies are included in FIG. 4. In certain embodiments, the anti-B7H3×anti-NKG2D antibody is a heterodimeric 1+1 Fab-scFv-Fc or 2+1 Fab₂-scFv-Fc format antibody as shown in FIG. 15.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

F. Afucosylated Fc Domains

In some embodiments, the increased binding of a Fc domain to CD16A is the result of producing the NKE in a cell line that reduces or eliminates the incorporation of fucose into the glycosylation of the NKE. See, for example, Pereira et al., MAbs (2018) 10(5):693-711.

In some embodiments, antibodies comprising Fc domains described are produced in a host cell such that the Fc domains have reduced fucosylation or no fucosylation compared to a parental Fc domain. In some instances, antibodies described are produced in a genetically modified host cell, wherein the genetic modification to the host cell results in the overexpression of β(1,4)-N-acetylglucosaminyltransferase III (GnTIII), a glycosyltransferase catalyzing the formation of bisected oligosaccharides, which are generally also non-fucosylated. N-glycosylation of the Fc domain can play a role in binding to FcγR; and afucosylation of the N-glycan can increase the binding capacity of the Fc domain to FcγRIIIa. As discussed in further detail above, an increase in FcγRIIIa binding can enhance ADCC, which can be advantageous in certain antibody therapeutic applications in which cytotoxicity is desirable.

In some embodiments, an Fc domain is engineered such that it has reduced fucosylation or no fucosylation, compared to a parental Fc domain. In the context of an Fc domain, the terms “afucosylation,” “afucosylated,” “defucosylation,” and “defucosylated” are used interchangeably, and generally refer to the absence or removal of core-fucose from the N-glycan attached to the CH2 domain of an Fc domain. For instance, an afucosylated antibody lacks core fucosylation in the Fc domain. As used herein, the phrase “a low level of fucosylation” or “reduced fucosylation” generally refers to an overall fucosylation level in a specific Fc domain that is no more than about 10.0%, no more than 5.0%, no more than 2.5%, no more than 1.0%, no more than about 0.5%, no more than 0.25%, no more than about 0.1%, or no more than 0.01%, compared to the fucosylation level of parental Fc domain. The term “% fucosylation” generally refers to the level of fucosylation in a specific Fc domain compared to that of a parental Fc domain. The % fucosylation can be measured according to any suitable method known in the relevant art, such as, for example, by mass spectrometry (MS), HPLC-Chip Cube MS (Agilent), and reverse phase-HPLC.

In some embodiments, a particular level of fucosylation is desired. In some embodiments, a Fc variant is provided, wherein the Fc variant comprises a particular level of afucosylation. In some further embodiments, the fucosylation level of the Fc variant is no more than about 10.0%, no more than about 9.0%, no more than about 8.0%, no more than about 7.0%, no more than about 6.0%, no more than about 5.0%, no more than about 4.0%, no more than about 3.0%, no more than about 2.0%, no more than about 1.5%, no more than about 1.0%, no more than about 0.5%, no more than 0.25%, no more than about 0.1%, or no more than 0.01%, compared to that of a parental Fc domain.

In some embodiments, antibodies comprising afucosylated Fc domains can be enriched (to obtain a particular level of afucosylation) by affinity chromatography using resins conjugated with a fucose binding moiety, such as, for example, an antibody or lectin specific for fucose, with some embodiments finding particular utility when fucose is present in a 1-6 linkage (see, e.g., Kobayashi et al., 2012, J. Biol. Chem. 287:33973-82).

In some embodiments, the fucosylated species of the Fc domain can be separated from the afucosylated species of the Fc domain (to obtain a particular level of afucosylation) using an anti-fucose specific antibody in an affinity column. Alternatively, or in addition to, afucosylated species can be separated from fucosylated species based on the differential binding affinity to FcγRIIIa using affinity chromatography (again, to obtain a particular level of afucosylation).

### (ii) NKG2D Antigen Binding Domains

In one aspect, provided herein are NKG2D antigen binding domains (ABDs) and compositions that include such NKG2D antigen binding domains (ABDs), including anti-NKG2D×anti-B7H3 bispecific antibodies. Such NKG2D binding domains and related antibodies find use, for example, in the treatment of B7H3 associated cancers. It is recognized that the NKG2D ABDs are capable of binding to the extracellular domain (ECD) of human NKG2D.

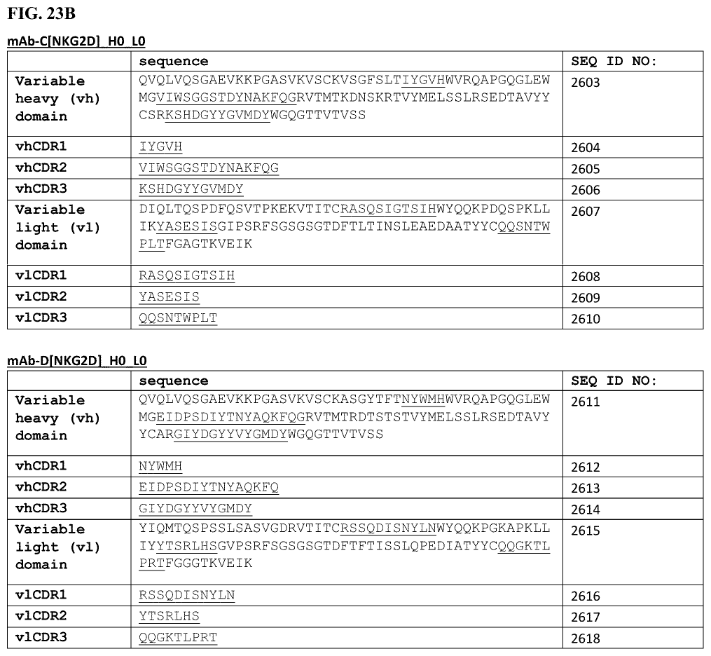

Suitable NKG2D binding domain can comprise a set of 6 CDRs (VHCDR1-3 and VLCDR1-3) or V_{H} and V_{L} domains as are depicted in the figures including FIGS. 23A, 23B and 58A-58J as well as the corresponding sequences in the sequence listing. The sequence listing also includes sequences of additional V_{H}/V_{L} pairs for binding NKG2D, as recognized by those skilled in the art. In some embodiments, the NKG2D ABD has a set of vhCDRs selected from the vhCDR1, vhCDR2 and vhCDR3 sequences from a V_{H} selected from the group including: 1D7B4[NKG2D]_H1, 1D2B4[NKG2D]_H0, mAb-C[NKG2D]_H0, and mAb-D[NKG2D]_H0, as shown in FIGS. 23A and 23B. In many embodiments, the NKG2D ABD has a set of vhCDRs selected from the set of vhCDR1, vhCDR2 and vhCDR3 sequences selected from the group including: SEQ ID NOS:17-19, SEQ ID NOS:33-35, SEQ ID NOS:2604-2606, SEQ ID NOS:2612-2614, as shown in FIGS. 23A and 23B.

In some embodiments, the NKG2D ABD has a set of vhCDRs selected from the vhCDR1, vhCDR2 and vhCDR3 sequences from a V_{H} selected from the group including: 1D7B4[NKG2D]_H1.1; 1D7B4[NKG2D]_H1.2; 1D7B4[NKG2D]_H1.3; 1D7B4[NKG2D]_H1.4; 1D7B4[NKG2D]_H1.5; 1D7B4[NKG2D]_H1.6; 1D7B4[NKG2D]_H1.7; 1D7B4[NKG2D]_H1.8; 1D7B4[NKG2D]_H1.9; 1D7B4[NKG2D]_H1.10; 1D7B4[NKG2D]_H1.11; 1D7B4[NKG2D]_H1.12; 1D7B4[NKG2D]_H1.13; 1D7B4[NKG2D]_H1.14; 1D7B4[NKG2D]_H1.15; 1D7B4[NKG2D]_H1.16; 1D7B4[NKG2D]_H1.17; 1D7B4[NKG2D]_H1.18; 1D7B4[NKG2D]_H1.19; 1D7B4[NKG2D]_H1.20; 1D7B4[NKG2D]_H1.21; 1D7B4[NKG2D]_H1.22; 1D7B4[NKG2D]_H1.23; 1D7B4[NKG2D]_H1.24; 1D7B4[NKG2D]_H1.25; 1D7B4[NKG2D]_H1.26; 1D7B4[NKG2D]_H1.27; 1D7B4[NKG2D]_H1.28; 1D7B4[NKG2D]_H1.29; 1D7B4[NKG2D]_H1.30; 1D7B4[NKG2D]_H1.31; 1D7B4[NKG2D]_H1.32; 1D7B4[NKG2D]_H1.33; 1D7B4[NKG2D]_H1.34; 1D7B4[NKG2D]_H1.35; 1D7B4[NKG2D]_H1.36; 1D7B4[NKG2D]_H1.37; 1D7B4[NKG2D]_H1.38; 1D7B4[NKG2D]_H1.39; 1D7B4[NKG2D]_H1.40; 1D7B4[NKG2D]_H1.41; 1D7B4[NKG2D]_H1.42; 1D7B4[NKG2D]_H1.43; 1D7B4[NKG2D]_H1.44; 1D7B4[NKG2D]_H1.45; 1D7B4[NKG2D]_H1.46; 1D7B4[NKG2D]_H1.47; 1D7B4[NKG2D]_H1.48; SEQ ID NOS:1212, 18 and 19; SEQ ID NOS:1214, 18 and 19; SEQ ID NOS:1216, 18 and 19; SEQ ID NOS:1218, 18 and 19; SEQ ID NOS:1219, 18 and 19; SEQ ID NOS:1220, 18 and 19; SEQ ID NOS:1222, 18 and 19; SEQ ID NOS:1224, 18 and 19; SEQ ID NOS:1226, 18 and 19; SEQ ID NOS:17, 18 and 1228; SEQ ID NOS:17, 18 and 1230; SEQ ID NOS:17, 18 and 1232; SEQ ID NOS:17, 18 and 1234; SEQ ID NOS:17, 18 and 1236; SEQ ID NOS:17, 18 and 1238; SEQ ID NOS:17, 18 and 1240; SEQ ID NOS:17, 18 and 1242; SEQ ID NOS:17, 18 and 1244; SEQ ID NOS:17, 18 and 1246; SEQ ID NOS:17, 18 and 1248; SEQ ID NOS:17, 18 and 1250; SEQ ID NOS:17, 18 and 1252; SEQ ID NOS:17, 18 and 1254; SEQ ID NOS:17, 18 and 1256; SEQ ID NOS:17, 18 and 1258; SEQ ID NOS:17, 18 and 1260; SEQ ID NOS:17, 18 and 1262; SEQ ID NOS:17, 18 and 1264; SEQ ID NOS:17, 18 and 1266; SEQ ID NOS:17, 18 and 1268; SEQ ID NOS:17, 18 and 1270; SEQ ID NOS:17, 18 and 1272; SEQ ID NOS:17, 18 and 1274; SEQ ID NOS:17, 18 and 1276; SEQ ID NOS:17, 18 and 1278; SEQ ID NOS:17, 18 and 1280; SEQ ID NOS:17, 18 and 1282; SEQ ID NOS:17, 18 and 1284; SEQ ID NOS:17, 18 and 1286; SEQ ID NOS:17, 18 and 1288; SEQ ID NOS:17, 18 and 1290; SEQ ID NOS:17, 18 and 1292; SEQ ID NOS:17, 18 and 1294; SEQ ID NOS:17, 18 and 1296; SEQ ID NOS:17, 18 and 1298; SEQ ID NOS:17, 18 and 1300; SEQ ID NOS:17, 18 and 1302; SEQ ID NOS:17, 18 and 1304; SEQ ID NOS:17, 18 and 1306, as shown in FIGS. 58A-58J.

In some embodiments, the V_{H} domain of the NKG2D ABD is selected from the group including: 1D7B4[NKG2D]_H1, 1D2B4[NKG2D]_H0, mAb-C[NKG2D]_H0, mAb-D[NKG2D]_H0, and SEQ ID NOS:50, 52, 2603 and 2611, as shown in FIGS. 23A and 23B.

In many embodiments, the V_{H} domain of the NKG2D ABD is selected from the group including: 1D7B4[NKG2D]_H1.1; 1D7B4[NKG2D]_H1.2; 1D7B4[NKG2D]_H1.3; 1D7B4[NKG2D]_H1.4; 1D7B4[NKG2D]_H1.5; 1D7B4[NKG2D]_H1.6; 1D7B4[NKG2D]_H1.7; 1D7B4[NKG2D]_H1.8; 1D7B4[NKG2D]_H1.9; 1D7B4[NKG2D]_H1.10; 1D7B4[NKG2D]_H1.11; 1D7B4[NKG2D]_H1.12; 1D7B4[NKG2D]_H1.13; 1D7B4[NKG2D]_H1.14; 1D7B4[NKG2D]_H1.15; 1D7B4[NKG2D]_H1.16; 1D7B4[NKG2D]_H1.17; 1D7B4[NKG2D]_H1.18; 1D7B4[NKG2D]_H1.19; 1D7B4[NKG2D]_H1.20; 1D7B4[NKG2D]_H1.21; 1D7B4[NKG2D]_H1.22; 1D7B4[NKG2D]_H1.23; 1D7B4[NKG2D]_H1.24; 1D7B4[NKG2D]_H1.25; 1D7B4[NKG2D]_H1.26; 1D7B4[NKG2D]_H1.27; 1D7B4[NKG2D]_H1.28; 1D7B4[NKG2D]_H1.29; 1D7B4[NKG2D]_H1.30; 1D7B4[NKG2D]_H1.31; 1D7B4[NKG2D]_H1.32; 1D7B4[NKG2D]_H1.33; 1D7B4[NKG2D]_H1.34; 1D7B4[NKG2D]_H1.35; 1D7B4[NKG2D]_H1.36; 1D7B4[NKG2D]_H1.37; 1D7B4[NKG2D]_H1.38; 1D7B4[NKG2D]_H1.39; 1D7B4[NKG2D]_H1.40; 1D7B4[NKG2D]_H1.41; 1D7B4[NKG2D]_H1.42; 1D7B4[NKG2D]_H1.43; 1D7B4[NKG2D]_H1.44; 1D7B4[NKG2D]_H1.45; 1D7B4[NKG2D]_H1.46; 1D7B4[NKG2D]_H1.47; 1D7B4[NKG2D]_H1.48; SEQ ID NOS:1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1295, 1297, 1301, 1303, and 1305, as shown in FIGS. 58A-58J.

In some embodiments, the NKG2D ABD has a set of vlCDRs selected from the vlCDR1, vlCDR2 and vlCDR3 sequences from a V_{L} selected from the group including: 1D7B4[NKG2D]_L1, 1D2B4[NKG2D]_L0, mAb-C[NKG2D]_L0, and mAb-D[NKG2D]_L0, as shown in FIGS. 23A and 23B. In some embodiments, the V_{L} domain of the NKG2D ABD is selected from the group including: 1D7B4[NKG2D]_L1, 1D2B4[NKG2D]_L0, mAb-C[NKG2D]_L0, mAb-D[NKG2D]_L0, and SEQ ID NOS:51, 2607 and 2615, as shown in FIGS. 23A and 23B.

Accordingly, included herein are NKG2D ABDs that have a set of 6 CDRs (vhCDR1, vhCDR2, vhCDR3, vlCDR1, vlCDR2 and vlCDR3) from V_{H}/V_{L} pairs selected from the group including: 1D7B4[NKG2D]_H1_L1, 1D2B4[NKG2D]_H0_L0, mAb-C[NKG2D]_H0_L0, mAb-D[NKG2D]_H0_L0, SEQ ID NOS:17-19, 23, 24, and 26; SEQ ID NOS:33-35, 23, 24, and 26; SEQ ID NOS:2604-2606 and 2608-2610; and SEQ ID NOS:2612-2614 and 2616-2618, as shown in FIGS. 23A and 23B. In many embodiments, the NKG2D ABDs have a set of 6 CDRs (vhCDR1, vhCDR2, vhCDR3, vlCDR1, vlCDR2 and vlCDR3) from V_{H}/V_{L} pairs selected from the group including: 1D7B4[NKG2D]_H1.1_L1; 1D7B4[NKG2D]_H1.2_L1; 1D7B4[NKG2D]_H1.3_L1; 1D7B4[NKG2D]_H1.4_L1; 1D7B4[NKG2D]_H1.5_L1; 1D7B4[NKG2D]_H1.6_L1; 1D7B4[NKG2D]_H1.7_L1; 1D7B4[NKG2D]_H1.8_L1; 1D7B4[NKG2D]_H1.9_L1; 1D7B4[NKG2D]_H1.10_L1; 1D7B4[NKG2D]_H1.11_L1; 1D7B4[NKG2D]_H1.12_L1; 1D7B4[NKG2D]_H1.13_L1; 1D7B4[NKG2D]_H1.14_L1; 1D7B4[NKG2D]_H1.1_L15; 1D7B4[NKG2D]_H1.16_L1; 1D7B4[NKG2D]_H1.17_L1; 1D7B4[NKG2D]_H1.18_L1; 1D7B4[NKG2D]_H1.19_L1; 1D7B4[NKG2D]_H1.20_L1; 1D7B4[NKG2D]_H1.21_L1; 1D7B4[NKG2D]_H1.22_L1; 1D7B4[NKG2D]_H1.23_L1; 1D7B4[NKG2D]_H1.24_L1; 1D7B4[NKG2D]_H1.25_L1; 1D7B4[NKG2D]_H1.26_L1; 1D7B4[NKG2D]_H1.27_L1; 1D7B4[NKG2D]_H1.28_L1; 1D7B4[NKG2D]_H1.29_L1; 1D7B4[NKG2D]_H1.30_L1; 1D7B4[NKG2D]_H1.31_L1; 1D7B4[NKG2D]_H1.32_L1; 1D7B4[NKG2D]_H1.33_L1; 1D7B4[NKG2D]_H1.34_L1; 1D7B4[NKG2D]_H1.35_L1; 1D7B4[NKG2D]_H1.36_L1; 1D7B4[NKG2D]_H1.37_L1; 1D7B4[NKG2D]_H1.38_L1; 1D7B4[NKG2D]_H1.39_L1; 1D7B4[NKG2D]_H1.40_L1; 1D7B4[NKG2D]_H1.41_L1; 1D7B4[NKG2D]_H1.42_L1; 1D7B4[NKG2D]_H1.43_L1; 1D7B4[NKG2D]_H1.44_L1; 1D7B4[NKG2D]_H1.45_L1; 1D7B4[NKG2D]_H1.46_L1; 1D7B4[NKG2D]_H1.47_L1; 1D7B4[NKG2D]_H1.48_L1; SEQ ID NOS:1212, 18, 19, 23, 24, and 26; SEQ ID NOS:1214, 18, 19, 23, 24, and 26; SEQ ID NOS:1216, 18, 19, 23, 24, and 26; SEQ ID NOS:1218, 18, 19, 23, 24, and 26; SEQ ID NOS:1220, 18, 19, 23, 24, and 26; SEQ ID NOS:1222, 18, 19, 23, 24, and 26; SEQ ID NOS:1224, 18, 19, 23, 24, and 26; SEQ ID NOS:1226, 18, 19, 23, 24, and 26; SEQ ID NOS:17, 18, 1230, 23, 24, and 26; SEQ ID NOS:17, 18, 1232, 23, 24, and 26; SEQ ID NOS:17, 18, 1234, 23, 24, and 26; SEQ ID NOS:17, 18, 1236, 23, 24, and 26; SEQ ID NOS:17, 18, 1238, 23, 24, and 26; SEQ ID NOS:17, 18, 1240, 23, 24, and 26; SEQ ID NOS:17, 18, 1242, 23, 24, and 26; SEQ ID NOS:17, 18, 1244, 23, 24, and 26; SEQ ID NOS: 17, 18, 1246, 23, 24, and 26; SEQ ID NOS:17, 18, 1248, 23, 24, and 26; SEQ ID NOS:17, 18, 1250, 23, 24, and 26; SEQ ID NOS:17, 18, 1252, 23, 24, and 26; SEQ ID NOS:17, 18, 1254, 23, 24, and 26; SEQ ID NOS:17, 18, 1256, 23, 24, and 26; SEQ ID NOS:17, 18, 1258, 23, 24, and 26; SEQ ID NOS:17, 18, 1260, 23, 24, and 26; SEQ ID NOS:17, 18, 1262, 23, 24, and 26; SEQ ID NOS:17, 18, 1264, 23, 24, and 26; SEQ ID NOS:17, 18, 1266, 23, 24, and 26; SEQ ID NOS: 17, 18, 1268, 23, 24, and 26; SEQ ID NOS:17, 18, 1270, 23, 24, and 26; SEQ ID NOS:17, 18, 1272, 23, 24, and 26; SEQ ID NOS:17, 18, 1274, 23, 24, and 26; SEQ ID NOS:17, 18, 1276, 23, 24, and 26; SEQ ID NOS:17, 18, 1278, 23, 24, and 26; SEQ ID NOS:17, 18, 1280, 23, 24, and 26; SEQ ID NOS:17, 18, 1282, 23, 24, and 26; SEQ ID NOS:17, 18, 1284, 23, 24, and 26; SEQ ID NOS:17, 18, 1286, 23, 24, and 26; SEQ ID NOS:17, 18, 1288, 23, 24, and 26; SEQ ID NOS: 17, 18, 1290, 23, 24, and 26; SEQ ID NOS:17, 18, 1292, 23, 24, and 26; SEQ ID NOS:17, 18, 1294, 23, 24, and 26; SEQ ID NOS:17, 18, 1296, 23, 24, and 26; SEQ ID NOS:17, 18, 1298, 23, 24, and 26; SEQ ID NOS:17, 18, 1300, 23, 24, and 26; SEQ ID NOS:17, 18, 1302, 23, 24, and 26; SEQ ID NOS:17, 18, 1304, 23, 24, and 26; SEQ ID NOS:17, 18, 1306, 23, 24, and 26, as shown in FIGS. 23A and 58A-58J.

Additionally, included herein are NKG2D ABDs that have V_{H}/V_{L} pairs selected from the group including: 1D7B4[NKG2D]_H1_L1, 1D2B4[NKG2D]_H0_L0, mAb-C[NKG2D]_H0_L0, mAb-D[NKG2D]_H0_L0, SEQ ID NOS:50 and 51, SEQ ID NOS:52 and 51, SEQ ID NOS:2603 and 2607, and SEQ ID NOS:2611 and 2615, as shown in FIGS. 23A and 23B. In some embodiments, the NKG2D ABDs have V_{H}/V_{L} pairs selected from the group including: 1D7B4[NKG2D]_H1.1_L1; 1D7B4[NKG2D]_H1.2_L1; 1D7B4[NKG2D]_H1.3_L1; 1D7B4[NKG2D]_H1.4_L1; 1D7B4[NKG2D]_H1.5_L1; 1D7B4[NKG2D]_H1.6_L1; 1D7B4[NKG2D]_H1.7_L1; 1D7B4[NKG2D]_H1.8_L1; 1D7B4[NKG2D]_H1.9_L1; 1D7B4[NKG2D]_H1.10_L1; 1D7B4[NKG2D]_H1.11_L1; 1D7B4[NKG2D]_H1.12_L1; 1D7B4[NKG2D]_H1.13_L1; 1D7B4[NKG2D]_H1.14_L1; 1D7B4[NKG2D]_H1.1_L15; 1D7B4[NKG2D]_H1.16_L1; 1D7B4[NKG2D]_H1.17_L1; 1D7B4[NKG2D]_H1.18_L1; 1D7B4[NKG2D]_H1.19_L1; 1D7B4[NKG2D]_H1.20_L1; 1D7B4[NKG2D]_H1.21_L1; 1D7B4[NKG2D]_H1.22_L1; 1D7B4[NKG2D]_H1.23_L1; 1D7B4[NKG2D]_H1.24_L1; 1D7B4[NKG2D]_H1.25_L1; 1D7B4[NKG2D]_H1.26_L1; 1D7B4[NKG2D]_H1.27_L1; 1D7B4[NKG2D]_H1.28_L1; 1D7B4[NKG2D]_H1.29_L1; 1D7B4[NKG2D]_H1.30_L1; 1D7B4[NKG2D]_H1.31_L1; 1D7B4[NKG2D]_H1.32_L1; 1D7B4[NKG2D]_H1.33_L1; 1D7B4[NKG2D]_H1.34_L1; 1D7B4[NKG2D]_H1.35_L1; 1D7B4[NKG2D]_H1.36_L1; 1D7B4[NKG2D]_H1.37_L1; 1D7B4[NKG2D]_H1.38_L1; 1D7B4[NKG2D]_H1.39_L1; 1D7B4[NKG2D]_H1.40_L1; 1D7B4[NKG2D]_H1.41_L1; 1D7B4[NKG2D]_H1.42_L1; 1D7B4[NKG2D]_H1.43_L1; 1D7B4[NKG2D]_H1.44_L1; 1D7B4[NKG2D]_H1.45_L1; 1D7B4[NKG2D]_H1.46_L1; 1D7B4[NKG2D]_H1.47_L1; 1D7B4[NKG2D]_H1.48_L1; SEQ ID NOS:1211 and 51; SEQ ID NOS:1213 and 51; SEQ ID NOS:1215 and 51; SEQ ID NOS:1217 and 51; SEQ ID NOS:1219 and 51; SEQ ID NOS:1221 and 51; SEQ ID NOS:1223 and 51; SEQ ID NOS:1225 and 51; SEQ ID NOS:1227 and 51; SEQ ID NOS:1229 and 51; SEQ ID NOS:1231 and 51; SEQ ID NOS:1233 and 51; SEQ ID NOS:1235 and 51; SEQ ID NOS:1237 and 51; SEQ ID NOS:1239 and 51; SEQ ID NOS:1241 and 51; SEQ ID NOS:1243 and 51; SEQ ID NOS:1245 and 51; SEQ ID NOS:1247 and 51; SEQ ID NOS:1249 and 51; SEQ ID NOS:1251 and 51; SEQ ID NOS:1253 and 51; SEQ ID NOS:1255 and 51; SEQ ID NOS:1257 and 51; SEQ ID NOS:1259 and 51; SEQ ID NOS:1261 and 51; SEQ ID NOS:1263 and 51; SEQ ID NOS:1265 and 51; SEQ ID NOS:1267 and 51; SEQ ID NOS:1269 and 51; SEQ ID NOS:1271 and 51; SEQ ID NOS:1273 and 51; SEQ ID NOS:1275 and 51; SEQ ID NOS:1277 and 51; SEQ ID NOS:1279 and 51; SEQ ID NOS:1281 and 51; SEQ ID NOS:1283 and 51; SEQ ID NOS:1285 and 51; SEQ ID NOS:1287 and 51; SEQ ID NOS:1289 and 51; SEQ ID NOS:1291 and 51; SEQ ID NOS:1293 and 51; SEQ ID NOS:1295 and 51; SEQ ID NOS:1297 and 51; SEQ ID NOS:1299 and 51; SEQ ID NOS:1301 and 51; SEQ ID NOS:1303 and 51; and SEQ ID NOS:1305 and 51, as shown in FIGS. 23A and 58A-58J.

In some embodiments, the V_{H}/V_{L} pairs of NKG2D scFvs are selected from the group including: 1D7B4_H1.3_L1, 1D7B4_H1.23_L1, 1D7B4_H1.28_L1, and 1D7B4_H1.31_L1. When the anti-NKG2D ABD is a scFv domain, the V_{H} and V_{L} domains can be in either orientation.

In some embodiments, the V_{H}/V_{L} pairs of NKG2D Fabs are selected from the group including: 1D7B4_H1.3_L1, 1D7B4_H1.23_L1, 1D7B4_H1.28_L1, and 1D7B4_H1.31_L1.

In some embodiments, the NKG2D ABDs have a V_{H}NL pair selected from the group including: SEQ ID NOS:940 and 3; SEQ ID NOS:941 and 3; SEQ ID NOS:942 and 3; SEQ ID NOS:943 and 3; SEQ ID NOS:944 and 3; SEQ ID NOS:945 and 3; SEQ ID NOS:946 and 3; SEQ ID NOS:947 and 3; SEQ ID NOS:948 and 3; SEQ ID NOS:949 and 3; SEQ ID NOS:950 and 3; SEQ ID NOS:951 and 3; SEQ ID NOS:952 and 3; SEQ ID NOS:953 and 3; SEQ ID NOS:954 and 3; SEQ ID NOS:955 and 3; SEQ ID NOS:956 and 3; SEQ ID NOS:957 and 3; SEQ ID NOS:958 and 3; SEQ ID NOS:959 and 3; SEQ ID NOS:960 and 3; SEQ ID NOS:961 and 3; SEQ ID NOS:962 and 3; SEQ ID NOS:963 and 3; SEQ ID NOS:964 and 3; SEQ ID NOS:965 and 3; SEQ ID NOS:966 and 3; SEQ ID NOS:967 and 3; SEQ ID NOS:968 and 3; SEQ ID NOS:969 and 3; SEQ ID NOS:970 and 3; SEQ ID NOS:971 and 3; SEQ ID NOS:972 and 3; SEQ ID NOS:973 and 3; SEQ ID NOS:974 and 3; SEQ ID NOS:975 and 3; SEQ ID NOS:976 and 3; SEQ ID NOS:977 and 3; SEQ ID NOS:978 and 3; SEQ ID NOS:979 and 3; SEQ ID NOS:980 and 3; SEQ ID NOS:981 and 3; SEQ ID NOS:982 and 3; SEQ ID NOS:983 and 3; SEQ ID NOS:984 and 3; SEQ ID NOS:985 and 3; SEQ ID NOS:986 and 3; SEQ ID NOS:987 and 3, as shown in FIGS. 54A-54L.

As will be appreciated by those in the art, suitable NKG2D antigen binding domains can comprise a set of 6 CDRs as depicted in the Figures, either as they are underlined or, in the case where a different numbering scheme is used as described herein and as shown in Table 2, as the CDRs that are identified using other alignments within the V_{H} and V_{L} sequences of those depicted in FIGS. 23A-23B and 58A-58J. Suitable ABDs can also include the entire V_{H} and V_{L} sequences as depicted in these sequences and Figures, used as scFvs or as Fabs. In many of the embodiments herein that contain an Fv to NKG2D, it is the Fab monomer that binds NKG2D.

In addition to the parental CDR sets disclosed in the figures and sequence listing that form an ABD to NKG2D, provided herein are variant NKG2D ABDs having CDRs that include at least one modification of the NKG2D ABD CDRs disclosed herein (e.g., FIGS. 23A-23B and 58A-58J and the sequence listing). In one embodiment, the NKG2D ABD of the subject heterodimeric antibody includes a set of 6 CDRs with 1, 2, 3, 4, 5, 6, 7, 8, 9, 10 amino acid modifications as compared to the 6 CDRs of an NKG2D binding domain V_{H}/V_{L} pair as described herein, including the figures and sequence listing. In exemplary embodiments, the NKG2D ABD of the subject heterodimeric antibody includes a set of 6 CDRs with 1, 2, 3, 4, 5, 6, 7, 8, 9, 10 amino acid modifications as compared to the 6 CDRs of one of the following NKG2D binding domain V_{H}/V_{L} pairs: 1D7B4_H1.3_L1, 1D7B4_H1.23_L1, 1D7B4_H1.28_L1, and 1D7B4_H1.31_L1. In certain embodiments, the NKG2D ABD of the subject antibody is capable of binding to NKG2D, as measured at least one of a Biacore, surface plasmon resonance (SPR), BLI (biolayer interferometry, e.g., Octet assay) assay, and/or flow cytometry, with the latter finding particular use in many embodiments. In particular embodiments, the NKG2D ABD is capable of binding human NKG2D (see, for example, FIG. 11). In some cases, each variant CDR has no more than 1 or 2 amino acid changes, with no more than 1 per CDR being particularly useful.

In some embodiments, the NKG2D ABD of the subject antibody includes 6 CDRs that are at least 90, 91, 92, 93, 94, 95, 96, 97, 98 or 99% identical to the 6 CDRs of an NKG2D ABD as described herein, including the figures and sequence listing. In exemplary embodiments, the NKG2D ABD of the subject antibody includes 6 CDRs that are at least 90, 91, 92, 93, 94, 95, 96, 97, 98 or 99% identical to the 6 CDRs of one of the following NKG2D binding domain V_{H}/V_{L} pairs: 1D7B4_H1.3_L1, 1D7B4_H1.23_L1, 1D7B4_H1.28_L1, and 1D7B4_H1.31_L1. In certain embodiments, the NKG2D ABD of the subject antibody is capable of binding to NKG2D, as measured at least one of a Biacore, surface plasmon resonance (SPR), BLI (biolayer interferometry, e.g., Octet assay) assay, and/or flow cytometry, with the latter finding particular use in many embodiments. In particular embodiments, the NKG2D ABD is capable of binding human NKG2D antigen (see, for example, FIG. 11).

In an exemplary embodiment, the NKG2D ABD of the subject antibody includes the variable heavy (V_{H}) domain and variable light (V_{L}) domain of any one of the NKG2D binding domain V_{H}/V_{L} pairs described herein, including the figures and sequence listing.

In some embodiments, the NKG2D ABD of the subject antibody includes a NKG2D antigen binding domain comprising a V_{H}/V_{L} pair present in the antibody molecules selected from the group including: SEQ ID NOS: 1320-1321, 1322-1323, 1324-1325-, 1326-1327, 1328-1329, 1330-1331, 1332-1333, 1334-1335, 1336-1337, 1338-1339, 1340-1341, 1342-1343, 1344-4345, 1346-1347, 1348-1349, 1350-1351, 1352-1353, 1354-1355, 1362-1363, 1364-1365, 1366-1367, 1368-1369, 1370-1371, 1372-1373, 1374-1375, 1376-1377, 1379-1379, 1380-1381, 1382-1383, 1384-1385, 1386-1387, 1388-1389, 1390-1391, 1392-1393, 1394-1395, 1396-1397, 1398-1399, 1400-1401, 1402-1403, 1404-1405, 1406-1407, 1408-1409, 1410-1411, 1412-1413, 1432-1433, 1434-1435, 1436-1437, 1438-1439, 1440-1441, 1442-1443, 1444-1445, 1446-1447, 1484-1485, 1495-1496, 1497-1498, 1499-1500, 1501-1502, 1503-1504, 1505-1506 and 1507-1508, 1896-1897, 1898-1899, 1900-1901, 1902-1903, 1904-1905, 1906-1907, 1908-1909, 1910-1911, 1912-1913, 1914-1915, 1916-1917, 1918-1919, 1920-1921, 1922-1923, 1924-1925, 1926-1927, 1928-1929, 1930-1931, 1932-1933, 1934-1935, 1936-1937, 1938-1939, 1940-1941, 1942-1943, 1944-1945, 1946-1947, 1948-1949, 1950-1951, 1952-1953, 1954-1955, 1956-1957, 1958-1959, 1960-1961, 1962-1963, 1964-1965, 1966-1967, 1968-1969, 1970-1971, 1972-1973, 1974-1975, 1976-1977, 1978-1979, 1980-1981, 1982-1983, 1984-1985, 1986-1987, 1988-1989, and 1990-1991, as presented in the Sequence Listing. In some embodiments, the NKG2D ABD of the subject antibody includes a NKG2D antigen binding domain comprising a V_{H}/V_{L} pair described herein, including in the figures and sequence list. Useful NKG2D ABDs are provided in the sequence listing including those recognized by those skilled in the art to bind the ECD of NKG2D.

In some embodiments, the NKG2D ABD is a variant of a parental NKG2D ABD such that the variant includes at least one amino acid substitution in the variable heavy domain. In some instances, the NKG2D ABD variant has detuned (e.g., modulated or changed such as reduced) affinity for the ECD of NKG2D compared to the parental NKG2D ABD. For instance, a detuned NKG2D ABD affords reduced fratricide while possessing potent binding activity.

Provided herein are consensus framework regions (FR) and complementarity determining regions (CDRs) (as in Kabat) for anti-NKG2D clone 1D7B4 detuned variable heavy domains and variable light domains (see, e.g., Table 3). In some embodiments, the NKG2D antigen binding domain provided herein includes one or more of the sequences depicted in Table 3.

TABLE 3

| | Amino acid | SEQ ID | |
|---|---|---|---|
| | sequence | NO: | |
| 1D7B4 | | | |
| VH | | | |
| HFR1 | EVQLLESGGGLVQPG | 1060 | |
| | GSLRLSCAASGFTFS | | |
| HCDR1 | SXX₂MS | | X₁ is selected |
| | | | from Y, I, A, |
| | | | S, H, Q; |
| | | | X₂ is selected |
| | | | from Y, N, A, H |
| HFR2 | WRQAPGKGLEWWS | 1061 | |
| HCDR2 | SISASGGSTYY | 1062 | |
| | ADSVKG | | |
| HFR3 | RFTISRDNSKNTLY | 1063 | |
| | LQMNSLRAEDTAVY | | |
| | YCAK | | |
| HCDR3 | GX₁FX₂X₃X₄X₅X₆Y | 1064 | X₁ is selected |
| | X₇DY | | from I, W, K, |
| | | | A, H |
| | | | X₂ is selected |
| | | | from S, A |
| | | | X₃ is selected |
| | | | from I, W, A, |
| | | | Q, E, H, S |
| | | | X₄ is selected |
| | | | from Y, H, A, |
| | | | S, E, Q, L |
| | | | X₅ is selected |
| | | | from F, H, A, |
| | | | S, E, Q, L |
| | | | X₆ is selected |
| | | | from F, H, E |
| | | | X₇ is selected |
| | | | from F, H |
| HFR4 | WGQGTLVTVSS | 1065 | |
| 1A7 VL | | | |
| LFR1 | DIQMTQSPSSLSAS | 1066 | |
| | VGDRVTITC | | |
| LCDR1 | RASQSISSYLN | 1067 | |
| LFR2 | WYQQKPGKAPKL | 1068 | |
| | LIY | | |
| LCDR2 | AASSLQS | 1069 | |
| LFR3 | GVPSRFSGSGSGT | 1070 | |
| | DFTLTISSLQPED | | |
| | FATYYC | | |
| LCDR3 | QQSYSTPYT | 1071 | |
| LFR4 | FGQGTKLEIK | 1072 | |

In some embodiments, the NKG2D antigen binding domain includes at least one of the HCDR1-3 and/or LCDR1-3 sequences of Table 3. In some embodiments, the NKG2D antigen binding domain includes at least one of the HFR1-4 and/or LFR1-4 sequences of Table 3.

(iii) B7H3 Antigen Binding Domains

In another aspect, provided herein are B7H3 ABDs and compositions that include such B7H3 ABDs including anti-NKG2D×anti-B7H3 antibodies. Such B7H3 binding domains and related antibodies find use, for example, in the treatment of B7H3 associated cancers. It is understood that the B7H3 ABDs described herein are capable of binding to the extracellular domain (ECD) of human B7H3.

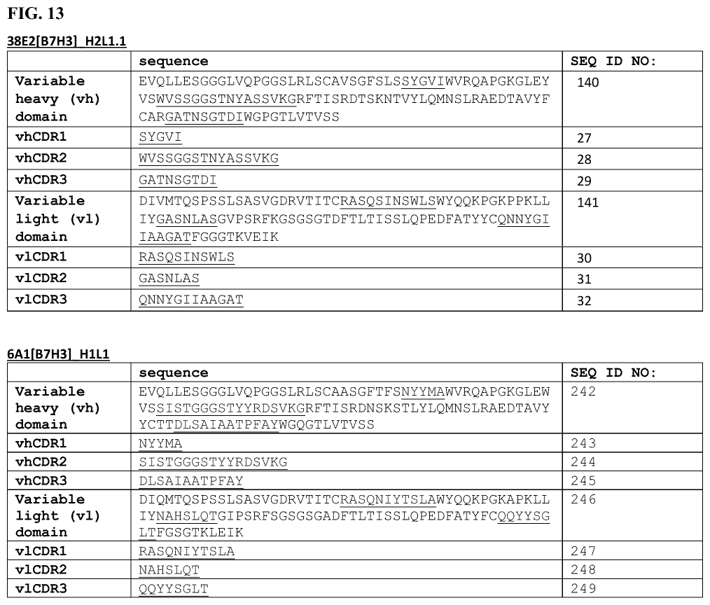

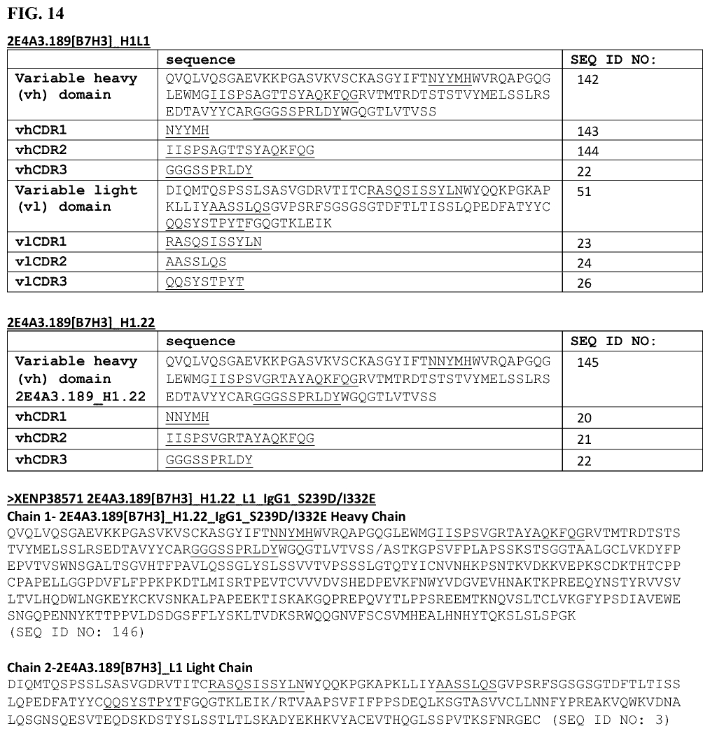

Suitable B7H3 binding domains can comprise a set of 6 CDRs (i.e., vhCDR1-3 and vlCDR1-3) or V_{H} and V_{L} domains as depicted in the figures including in FIGS. 13 and 14 as well as the corresponding sequences in the sequence listing. The sequence listing also includes sequences of additional V_{H}/V_{L} pairs for binding B7H3, as recognized by those skilled in the art.

B7H3 antigen binding domain sequences that are of particular use include, but are not limited to, 38E2_H2_L1.1, 6A1_H1_L1, 2E4A3.189_H1_L1, 2E4A3.189_H1.22_L1, as depicted in FIGS. 13 and 14. When the anti-B7H3 ABD is a scFv domain, the V_{H} and V_{L} domains can be in either orientation.

As will be appreciated by those in the art, suitable B7H3 antigen binding domains can comprise a set of 6 CDRs as depicted in the Figures, either as they are underlined or, in the case where a different numbering scheme is used as described herein and as shown in Table 2, as the CDRs that are identified using other alignments within the V_{H} and V_{L} sequences of those depicted in FIGS. 13 and 14. Suitable ABDs can also include the entire V_{H} and V_{L} sequences as depicted in these sequences and Figures, used as scFvs or as Fabs. In many of the embodiments herein that contain an Fv to B7H3, it is the Fab monomer that binds B7H3.

In addition to the parental CDR sets disclosed in the figures and sequence listing that form an ABD to B7H3, provided herein are variant B7H3 ABDS having CDRs that include at least one modification of the B7H3 ABD CDRs disclosed herein (e.g., FIGS. 13 and 14 and the sequence listing). In one embodiment, the B7H3 ABD of the subject heterodimeric antibody (e.g., anti-B7H3×anti-NKG2D antibody) includes a set of 6 CDRs with 1, 2, 3, 4, 5, 6, 7, 8, 9, 10 amino acid modifications as compared to the 6 CDRs of a B7H3 ABD as described herein, including the figures and sequence listing. In exemplary embodiments, the B7H3 ABD of the subject heterodimeric antibody includes a set of 6 CDRs with 1, 2, 3, 4, 5, 6, 7, 8, 9, 10 amino acid modifications as compared to the 6 CDRs of one of the following CD3 binding domains: 38E2_H2_L1.1, 6A1_H1_L1, 2E4A3.189_H1_L1, 2E4A3.189_H1.22_L1, as depicted in FIGS. 13 and 14.

In certain embodiments, the B7H3 ABD of the subject antibody is capable of binding CD3 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the B7H3 ABD is capable of binding human B7H3 antigen (see FIG. 12).

In some embodiments, the B7H3 ABD of the subject antibody includes 6 CDRs that are at least 90, 91, 92, 93, 94, 95, 96, 97, 98 or 99% identical to the 6 CDRs of a B7H3 ABD as described herein, including the figures and sequence listing. In exemplary embodiments, the B7H3 ABD of the subject antibody includes 6 CDRs that are at least 90, 91, 92, 93, 94, 95, 96, 97, 98 or 99% identical to the 6 CDRs of one of the following B7H3 binding domains: 38E2_H2_L1.1, 6A1_H1_L1, 2E4A3.189_H1_L1, 2E4A3.189_H1.22_L1, as depicted in FIGS. 13 and 14. In certain embodiments, the B7H3 ABD is capable of binding to the B7H3, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the B7H3 ABD is capable of binding human B7H3 antigen (see FIG. 12).

In another exemplary embodiment, the B7H3 ABD of the subject antibody includes the variable heavy (V_{H}) domain and variable light (V_{L}) domain of any one of the B7H3 binding domains described herein, including the figures and sequence listing.

Additionally, included herein are B7H3 ABDs that have the variable heavy chain depicted in SEQ ID NO: 11 or SEQ ID NO: 13, and variable light chain depicted in SEQ ID NO: 12 or SEQ ID NO: 14 from U.S. Pat. No. 10,501,544, incorporated by reference in its entirety, with particularity for relevant disclosure pertaining to B7H3 ABDs and the accompanying sequences described therein. Further, included herein are B7H3 antigen binding domain with: a) the V_{H} CDRs depicted in SEQ ID NO: 1, SEQ ID NO: 25, and SEQ ID NO: 33 in combination with the V_{L} CDRs depicted in SEQ ID NO: 34, SEQ ID NO: 36, and SEQ ID NO: 6; or b) the V_{H} CDRs depicted in SEQ ID NO: 1, SEQ ID NO: 25, and SEQ ID NO: 10 in combination with the V_{L} CDRs depicted in SEQ ID NO: 38, SEQ ID NO: 30, and SEQ ID NO: 6; both from U.S. Pat. No. 9,963,509, incorporated by reference in its entirety, with particularity for relevant disclosure pertaining to B7H3 ABDs and the accompanying sequences described therein. Further, included herein are anti-B7H3 antigen binding domains with the V_{H} CDRs depicted in SEQ ID NO: 1, SEQ ID NO: 9, and SEQ ID NO: 10 in combination with the V_{L} CDRs depicted in SEQ ID NO: 11, SEQ ID NO: 312, and SEQ ID NO: 6; from U.S. Pat. No. 10,865,245, incorporated by reference in its entirety, with particularity for relevant disclosure pertaining to B7H3 ABDs and the accompanying sequences described therein. Still further, included herein are anti-B7H3 antigen binding domain with: a) vhCDR1 with the sequence depicted in SEQ ID NO: 110; b) vhCDR2 with the sequence depicted in SEQ ID NO: 111; c) vhCDR3 with the sequence depicted in SEQ ID NO: 113; d) vlCDR1 with the sequence depicted in SEQ ID NO: 114; e) vlCDR2 with the sequence depicted in SEQ ID NO: 115; and f) vlCDR3 with the sequence depicted in SEQ ID NO: 116, from WO2020/033702, incorporated by reference in its entirety, with particularity for relevant disclosure pertaining to B7H3 ABDs and the accompanying sequences described therein. Yet further, included herein is an anti-B7H3 antigen binding domain with: a) vhCDR1 with the sequence depicted in SEQ ID NO: 118; b) vhCDR2 with the sequence depicted in SEQ ID NO: 119; c) vhCDR3 with the sequence depicted in SEQ ID NO: 120; d) vlCDR1 with the sequence depicted in SEQ ID NO: 121; e) vlCDR2 with the sequence depicted in SEQ ID NO: 122; and f) vlCDR3 with the sequence depicted in SEQ ID NO: 123, from WO2020/033702, incorporated by reference in its entirety, with particularity for relevant disclosure pertaining to B7H3 ABDs and the accompanying sequences described therein. Still even further, included herein is an anti-B7H3 antigen binding domain with: a) vhCDR1 with the sequence depicted in SEQ ID NO: 371; b) vhCDR2 with the sequence depicted in SEQ ID NO: 372; c) vhCDR3 with the sequence depicted in SEQ ID NO: 373; d) vlCDR1 with the sequence depicted in SEQ ID NO: 374; e) vlCDR2 with the sequence depicted in SEQ ID NO: 375; and f) vlCDR3 with the sequence depicted in SEQ ID NO: 376, from WO2020/033702, incorporated by reference in its entirety, with particularity for relevant disclosure pertaining to B7H3 ABDs and the accompanying sequences described therein.

In some embodiments, the subject antibody includes a B7H3 ABD that includes a variable heavy domain and/or a variable light domain that are variants of another B7H3 ABD V_{H} and V_{L} domain disclosed herein. In one embodiment, the variant V_{H} domain and/or V_{L} domain has from 1, 2, 3, 4, 5, 6, 7, 8, 9 or 10 amino acid changes from a V_{H} and/or V_{L} domain of a B7H3 ABD described herein, including the figures and sequence listing. In exemplary embodiments, the variant V_{H} domain and/or V_{L} domain has from 1, 2, 3, 4, 5, 6, 7, 8, 9 or 10 amino acid changes from a V_{H} and/or V_{L} domain of one of the following B7H3 binding domains: 38E2_H2_L1.1, 6A1_H1_L1, 2E4A3.189_H1_L1, 2E4A3.189_H1.22_L1, as shown in FIGS. 13-14. In some embodiments, one or more amino acid changes are in the V_{H} and/or V_{L} framework regions (FR1, FR2, FR3, and/or FR4). In some embodiments, one or more amino acid changes are in one or more CDRs. In certain embodiments, the B7H3 ABD of the subject antibody is capable of binding to B7H3, as measured at least one of a Biacore, surface plasmon resonance (SPR), flow cytometry, and/or BLI (biolayer interferometry, e.g., Octet assay) assay, with the latter finding particular use in many embodiments. In particular embodiments, the B7H3 ABD is capable of binding human B7H3 antigen (see FIG. 12).

### (iv) Linkers

As shown herein, there are a number of suitable linkers (for use as either domain linkers or scFv linkers) that can be used to covalently attach the recited domains (e.g., scFvs, Fabs, Fc domains, etc.), including traditional peptide bonds, generated by recombinant techniques. Exemplary linkers to attach domains of the subject antibody to each other are depicted in FIG. 6. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example, (GS)n, (GSGGS)n (SEQ ID NO: 2619), (GGGGS)n (SEQ ID NO: 2620), and (GGGS)n (SEQ ID NO: 2621), where n is an integer of at least one (and generally from 3 to 4), glycine-alanine polymers, alanine-serine polymers, and other flexible linkers, some of which are shown in FIGS. 5 and 6. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of C_{L}/CH1 domain but not all residues of C_{L}/CH1 domain; for example, the first 5-12 amino acid residues of the C_{L}/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

In some embodiments, the linker is a “domain linker”, used to link any two domains as outlined herein together. For example, in FIG. 15, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example, those in FIG. 6 as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used. Exemplary useful domain linkers are depicted in FIG. 6.

With particular reference to the domain linker used to attach the scFv domain to the Fc domain in the “2+1” format, there are several domain linkers that find particular use, including “full hinge C220S variant,” “flex half hinge,” “charged half hinge 1,” and “charged half hinge 2” as shown in FIG. 6.

In some embodiments, the linker is a “scFv linker”, used to covalently attach the V_{H} and V_{L} domains as discussed herein. In many cases, the scFv linker is a charged scFv linker, a number of which are shown in FIG. 5. Accordingly, in some embodiments, the antibodies described herein further provide charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen.

Charged domain linkers can also be used to increase the pI separation of the monomers of the antibodies described herein as well, and thus those included in FIG. 5 can be used in any embodiment herein where a linker is utilized.

### VI. Useful Formats of the Invention

As will be appreciated by those in the art and discussed more fully below, the heterodimeric bispecific antibodies provided herein can take on a wide variety of configurations, as are generally depicted in FIG. 15. The heterodimeric formats of the antibodies described herein can have different valences as well as be bispecific. That is, heterodimeric antibodies of the antibodies described herein can be bivalent and bispecific, wherein one target NK cell antigen (e.g., NKG2D) is bound by one binding domain and the one target tumor antigen (e.g., B7H3) is bound by a second binding domain. In some embodiments, heterodimeric antibodies described herein can be bivalent and bispecific, wherein one target tumor antigen is bound by one binding domain and another target tumor antigen is bound by a second binding domain. The heterodimeric antibodies can also be trivalent and bispecific, wherein the first antigen is bound by two binding domains and the second antigen by a second binding domain. As is outlined herein, when NKG2D is one of the target antigens, in some embodiments, the NKG2D is bound only monovalently, to reduce potential side effects.

The antibodies described herein utilize anti-NKG2D antigen binding domains in combination with anti-B7H3 binding domains. As will be appreciated by those in the art, any collection of anti-NKG2D CDRs, anti-NKG2D variable light and variable heavy domains, Fabs and scFvs as described herein, and depicted in any of the Figures can be used. Similarly, any of the anti-B7H3 antigen binding domains can be used, whether CDRs, variable light and variable heavy domains, Fabs and scFvs as described herein, and depicted in any of the Figures can be used, optionally and independently combined in any combination.

1. 1+1 Fab-scFv-Fc Format

One heterodimeric scaffold that finds particular use in the antibodies described herein is the “1+1 Fab-scFv-Fc” or “bottle-opener” format as shown in FIG. 15A with an exemplary combination of an NKG2 antigen binding domain and a B7H3 antigen binding domain. In this embodiment, one heavy chain monomer of the antibody contains a single chain Fv (“scFv”, as defined below) and an Fc domain. The scFv includes a variable heavy domain (VH1) and a variable light domain (VL1), wherein the VH1 is attached to the VL1 using an scFv linker that can be charged (see, e.g., FIG. 5). The scFv is attached to the heavy chain using a domain linker (see, e.g., FIG. 6). The other heavy chain monomer is a “regular” heavy chain (VH-CH1-hinge-CH2-CH3). The 1+1 Fab-scFv-Fc also includes a light chain that interacts with the V_{H}-CH1 to form a Fab. This structure is sometimes referred to herein as the “bottle-opener” format, due to a rough visual similarity to a bottle-opener. The two heavy chain monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below.

There are several distinct advantages to the present “1+1 Fab-scFv-Fc” format. As is known in the art, antibody analogs relying on two scFv constructs often have stability and aggregation problems, which can be alleviated in the antibodies described herein by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

Many of the embodiments outlined herein rely in general on the 1+1 Fab-scFv-Fc or “bottle opener” format antibody that comprises a first monomer comprising an scFv, comprising a variable heavy and a variable light domain, covalently attached using an scFv linker (charged, in many but not all instances), where the scFv is covalently attached to the N-terminus of a first Fc domain usually through a domain linker The domain linker can be either charged or uncharged and exogenous or endogenous (e.g., all or part of the native hinge domain). Any suitable linker can be used to attach the scFv to the N-terminus of the first Fc domain. In some embodiments, the domain linker is chosen from the domain linkers in FIG. 6. The second monomer of the 1+1 Fab-scFv-Fc format or “bottle opener” format is a heavy chain, and the composition further comprises a light chain.

In some embodiments, the scFv is the domain that binds to NKG2D, and the Fab forms a B7H3 binding domain. In other embodiments, the scFv is the domain that binds B7H3, and the Fab forms a NKG2D binding domain. An exemplary anti-B7H3×anti-NKG2D bispecific antibody in the 1+1 Fab-scFv-Fc format is depicted in FIGS. 19 and 59.

In some embodiments, one or both Fc domains of this format can include one or more of: (i) FcγRIIIa variants, (ii) pI variants, (iii) skew variants, and (iv) FcRn variants, as well as any combination thereof, as desired and described herein. In certain embodiments, the Fc domains of the 1+1 Fab-scFv-Fc format contain FcγRIIIa variants, skew variants, pI variants, and optionally FcRn variants. In many embodiments, the Fc domains of the 1+1 Fab-scFv-Fc format contain FcγRIIIa variants, skew variants, pI variants, and FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S, M428L/N434A or M252Y/S254T/T256E), optionally charged scFv linkers (including those shown in FIG. 5), optionally ablation variants (including those shown in FIG. 3), and the heavy chain comprises pI variants (including those shown in FIG. 2).

In certain embodiments, the 1+1 Fab-scFv-Fc scaffold format includes a first monomer that includes a scFv-domain linker-CH2-CH3 monomer, a second monomer that includes a first variable heavy domain-CH1-hinge-CH2-CH3 monomer and a third monomer that includes a first variable light domain. In some embodiments, the CH2-CH3 of the first monomer is a first variant Fc domain and the CH2-CH3 of the second monomer is a second variant Fc domain. In some embodiments, the scFv includes a scFv variable heavy domain and a scFv variable light domain that form an NKG2D antigen binding domain. In certain embodiments, the scFv variable heavy domain and scFv variable light domain are covalently attached using an scFv linker (charged, in many but not all instances. See, e.g., FIG. 5). In some embodiments, the first variable heavy domain and first variable light domain form a B7H3 binding domain. In certain embodiments, the scFv includes a scFv variable heavy domain and a scFv variable light domain that form a B7H3 antigen binding domain. In certain embodiments, the scFv variable heavy domain and scFv variable light domain are covalently attached using an scFv linker (charged, in many but not all instances. See, e.g., FIG. 5). In some embodiments, the first variable heavy domain and first variable light domain form an NKG2D antigen binding domain.

Some embodiments include 1+1 Fab-scFv-Fc formats that comprise: a) a first monomer (the “scFv monomer”) that comprises a charged scFv linker (with the +H sequence of FIG. 5 being preferred in some embodiments), the skew variants S364K/E357Q, and an scFv that binds to NKG2D as outlined herein; b) a second monomer (the “Fab monomer”) that comprises the skew variants L368D/K370S, the pI variants N208D/Q295E/N384D/Q418E/N421D, and a variable heavy domain; and c) a light chain that includes a variable light domain light domain (V_{L}) and a constant light domain (C_{L}), wherein numbering is according to EU numbering. The variable heavy domain and variable light domain form a B7H3 antigen binding domain. In some embodiments, the scFv monomer further includes the v90 variants S239D/I332E. In other embodiments, the Fab monomer further includes the v90 variants S239D/I332E. In some embodiments, the scFv and Fab monomers both further include the M428L/N434S, M428L/N434A or M252Y/S254T/T256E variants.

FIGS. 7-9, 35, 50-53 and 60 show some exemplary Fc domain sequences or variants thereof that are useful in the 1+1 Fab-scFv-Fc format antibodies. The “monomer 1” sequences depicted in such figures typically refer to the Fc domain of the “Fab-Fc heavy chain” and the “monomer 2” sequences refer to the Fc domain of the “scFv-Fc heavy chain.” FIG. 4 depicts exemplary Fc variants that can be present in Fc domains of 1+1 Fab-scFv-Fc format antibodies. Further, FIG. 10 provides useful C_{L} sequences that can be used with this format.

Any suitable NKG2D ABD can be included in the 1+1 Fab-scFv-Fc format antibody, included those provided herein. NKG2D antigen binding domain sequences that are of particular use in these embodiments include, but are not limited to, V_{H} and V_{L} domains selected from have V_{H}/V_{L} pairs selected from the group including: 1D7B4_H1_L1; 1D2B4_H0_L0; mAb-C_H0_L0; mAb-D_H0_L0; 1D7B4_L1_H1; 1D2B4_L0_H0; mAb-C_L0_H0; mAb-D_L0_H0; 1D7B4_H1.1_L1; 1D7B4_H1.2_L1; 1D7B4_H1.3_L1; 1D7B4_H1.4_L1; 1D7B4_H1.5_L1; 1D7B4_H1.6_L1; 1D7B4_H1.7_L1; 1D7B4_H1.8_L1; 1D7B4_H1.9_L1; 1D7B4_H1.10_L1; 1D7B4_H1.11_L1; 1D7B4_H1.12_L1; 1D7B4_H1.13_L1; 1D7B4_H1.14_L1; 1D7B4_H1.15_L1; 1D7B4_H1.16_L1; 1D7B4_H1.17_L1; 1D7B4_H1.18_L1; 1D7B4_H1.19_L1; 1D7B4_H1.20_L1; 1D7B4_H1.21_L1; 1D7B4_H1.22_L1; 1D7B4_H1.23_L1; 1D7B4_H1.24_L1; 1D7B4_H1.25_L1; 1D7B4_H1.26_L1; 1D7B4_H1.27_L1; 1D7B4_H1.28_L1; 1D7B4_H1.29_L1; 1D7B4_H1.30_L1; 1D7B4_H1.31_L1; 1D7B4_H1.32_L1; 1D7B4_H1.3_L13; 1D7B4_H1.34_L1; 1D7B4_H1.35_L1; 1D7B4_H1.36_L1; 1D7B4_H1.37_L1; 1D7B4_H1.38_L1; 1D7B4_H1.39_L1; 1D7B4_H1.40_L1; 1D7B4_H1.41_L1; 1D7B4_H1.42_L1; 1D7B4_H1.43_L1; 1D7B4_H1.44_L1; 1D7B4_H1.45_L1; 1D7B4_H1.46_L1; 1D7B4_H1.47_L1; 1D7B4_H1.48_L1; 1D7B4_L1_H1.1; 1D7B4_L1_H1.2; 1D7B4_L1_H1.3; 1D7B4_L1_H1.4; 1D7B4_L1_H1.5; 1D7B4_L1_H1.6; 1D7B4_L1_H1.7; 1D7B4_L1_H1.8; 1D7B4_L1_H1.9; 1D7B4_L1_H1.10; 1D7B4_L1_H1.11; 1D7B4_L1_H1.12; 1D7B4_L1_H1.13; 1D7B4_L1_H1.14; 1D7B4_L1_H1.15; 1D7B4_L1_H1.16; 1D7B4_L1_H1.17; 1D7B4_L1_H1.18; 1D7B4_L1_H1.19; 1D7B4_L1_H1.20; 1D7B4_L1_H1.21; 1D7B4_L1_H1.22; 1D7B4_L1_H1.23; 1D7B4_L1_H1.24; 1D7B4_L1_H1.25; 1D7B4_L1_H1.26; 1D7B4_L1_H1.27; 1D7B4_L1_H1.28; 1D7B4_L1_H1.29; 1D7B4_L1_H1.30; 1D7B4_L1_H1.31; 1D7B4_L1_H1.32; 1D7B4_L1_H1.33; 1D7B4_L1_H1.34; 1D7B4_L1_H1.35; 1D7B4_L1_H1.36; 1D7B4_L1_H1.37; 1D7B4_L1_H1.38; 1D7B4_L1_H1.39; 1D7B4_L1_H1.40; 1D7B4_L1_H1.41; 1D7B4_L1_H1.42; 1D7B4_L1_H1.43; 1D7B4_L1_H1.44; 1D7B4_L1_H1.45; 1D7B4_L1_H1.46; 1D7B4_L1_H1.47; 1D7B4_L1_H1.48; 1D2B4_H1_L1; 1D2B4_L1_H1; ADI27744_A44_H0_L0; ADI27744_A44_L0_H0; ADI27749_A49_H0_L0; ADI27749_A49_L0_H0; or variants thereof, as well as those depicted in FIGS. 19, 56, 58 and 59.

In some embodiments, the αNKG2D ABD V_{H}/V_{L} pairs are found in, for example, SEQ ID NOS:1020 and 3; SEQ ID NOS:1022 and 3; SEQ ID NOS:1023 and 1024; SEQ ID NOS:1025 and 1026; SEQ ID NOS:1211 and 51; SEQ ID NOS:1213 and 51; SEQ ID NOS:1215 and 51; SEQ ID NOS:1217 and 51; SEQ ID NOS:1219 and 51; SEQ ID NOS:1221 and 51; SEQ ID NOS:1223 and 51; SEQ ID NOS:1225 and 51; SEQ ID NOS:1227 and 51; SEQ ID NOS:1229 and 51; SEQ ID NOS:1231 and 51; SEQ ID NOS:1233 and 51; SEQ ID NOS:1235 and 51; SEQ ID NOS:1237 and 51; SEQ ID NOS:1239 and 51; SEQ ID NOS:1241 and 51; SEQ ID NOS:1243 and 51; SEQ ID NOS:1245 and 51; SEQ ID NOS:1247 and 51; SEQ ID NOS:1249 and 51; SEQ ID NOS:1251 and 51; SEQ ID NOS:1253 and 51; SEQ ID NOS:1255 and 51; SEQ ID NOS:1257 and 51; SEQ ID NOS:1259 and 51; SEQ ID NOS:1261 and 51; SEQ ID NOS:1263 and 51; SEQ ID NOS:1265 and 51; SEQ ID NOS:1267 and 51; SEQ ID NOS:1269 and 51; SEQ ID NOS:1271 and 51; SEQ ID NOS:1273 and 51; SEQ ID NOS:1275 and 51; SEQ ID NOS:1277 and 51; SEQ ID NOS:1279 and 51; SEQ ID NOS:1281 and 51; SEQ ID NOS:1283 and 51; SEQ ID NOS:1285 and 51; SEQ ID NOS:1287 and 51; SEQ ID NOS:1289 and 51; SEQ ID NOS:1291 and 51; SEQ ID NOS:1293 and 51; SEQ ID NOS:1295 and 51; SEQ ID NOS:1297 and 51; SEQ ID NOS:1299 and 51; SEQ ID NOS:1301 and 51; SEQ ID NOS:1303 and 51; and SEQ ID NOS:1305 and 51, or variants thereof, as well as those depicted in the FIGS. 56 and 58.

NKG2D antigen binding domain sequences or NKG2D ABD V_{H}/V_{L} pairs finding particular use in these embodiments include, but are not limited to, 1D2B4_H1_L1; 1D2B4_L1_H1; ADI27744_A44_H0_L0; ADI27744_A44_L0_H0; ADI27749_A49_H0_L0; ADI27749_A49_L0_H0; 1D7B4_H1_L1; 1D7B4_L1_H1; 1D7B4_H1.3_L1; 1D7B4_L1_H1.3; 1D7B4_H1.23_L1; 1D7B4_L1_H1.23; 1D7B4_H1.28_L1; 1D7B4_L1_H1.28; 1D7B4_H1.31_L1; 1D7B4_L1_H1.31; 1D7B4_H1.33_L1; 1D7B4_L1_H1.33, or a variant thereof. In certain embodiments, the αNKG2D ABD V_{H}/V_{L} pairs are selected from the group including: ADI27744_A44_H0_L0; ADI27749_A44_H0_L0; 1D7B4_H1.3_L1; 1D7B4_H1.23_L1; 1D7B4_H1.28_L1; 1D7B4_H1.31_L1; 1D7B4_H1.33_L1, as well as V_{H}/V_{L} sequence pairs provided in SEQ ID NOS:1308, 1310, 1312, 1314 and 1316. In particular embodiments, the NKG2D scFv is any one selected from the group including those in SEQ ID NOS:1308, 1310, 1312, 1314 and 1316.

Any suitable B7H3 ABD can be included in the 1+1 Fab-scFv-Fc format antibody, including those provided herein. B7H3 ABDs that are of particular use in these embodiments include, but are not limited to, V_{H} and V_{L} domains selected from have V_{H}/V_{L} pairs selected from the group including: 38E2_H2_L1.1; 38E2_L1.1_H2; 6A1_H1_L1; 6A1_L1_H1; 2E4A3.189_H1_L1; 2E4A3.189_L1_H1; 2E4A3.189_H1.22_L1; 2E4A3.189_L1_H1.22, or a variant thereof, as well as those depicted in FIGS. 13 and 14. In certain embodiments, the αB7H3 ABD V_{H}/V_{L} pairs are selected from the group including: 2E4A3.189_H1.22_L1; 38E2_H2_L1.1, 6A1_H1_L1; SEQ ID NOS:140 and 141; SEQ ID NOS:242 and 246; SEQ ID NOS:145 and 51; or variants thereof, as well as those depicted in the FIGS. 13 and 14.

Exemplary 1+1 Fab-scFv format antibodies are depicted in FIG. 56, such as XENP40106, XENP40372, XENP40375, XENP40376, XENP40377, XENP40381, XENP40457, XENP41333, XENP41334, XENP41335, XENP41336, XENP42658, XENP42659, XENP42660, XENP42661, XENP42662, XENP43715, XENP43722, and XENP43994, as well as in the sequence listing.

2. 2+1 Fab₂-scFv-Fc Format

One heterodimeric scaffold that finds particular use in the antibodies described herein is the “2+1 Fab₂-scFv-Fc” format (also referred to in previous related filings as “central-scFv format”) shown in FIG. 15B with an exemplary combination of an NKG2D binding domain and two tumor target antigen (B7H3) binding domains. In this embodiment, the format relies on the use of an inserted scFv domain thus forming a third antigen binding domain, wherein the Fab portions of the two monomers bind B7H3 and the “extra” scFv domain binds NKG2D. The scFv domain is inserted between the Fc domain and the CH1-Fv region of one of the monomers, thus providing a third antigen binding domain. As described, B7H3×NKG2D bispecific antibodies having the 2+1 Fab₂-scFv-Fc format are potent in inducing redirected T cell cytotoxicity in cellular environments that express low levels of B7H3. Moreover, as shown in the examples, B7H3×NKG2D bispecific antibodies having the 2+1 Fab₂-scFv-Fc format allow for the “fine tuning” of immune responses as such antibodies exhibit a wide variety of different properties, depending on the B7H3 and/or NKG2D binding domains used. For example, such antibodies exhibit differences in selectivity for cells with different B7H3 expression, potencies for B7H3 expressing cells, ability to elicit cytokine release, and sensitivity to soluble B7H3. These B7H3 antibodies find use, for example, in the treatment of B7H3-associated cancers.

In this embodiment, one monomer comprises a first heavy chain comprising a first variable heavy domain, a CH1 domain (and optional hinge) and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using optional domain linkers (VH1-CH1-[optional linker]-VH2-scFv linker-VL2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in FIG. 6. In some embodiments, the optional linker is a hinge or a fragment thereof. The other monomer is a standard Fab side (i.e., VH1-CH1-hinge-CH2-CH3). This embodiment further utilizes a common light chain comprising a variable light domain and a constant light domain, that associates with the heavy chains to form two identical Fabs that bind B7H3.

In some embodiments, one or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the 2+1 Fab₂-scFv-Fc format contain FcγRIIIa variants, skew variants, pI variants, and optionally FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S, M428L/N434A or M252Y/S254T/T256E), optionally charged scFv linkers (including those shown in FIG. 5), optionally ablation variants (including those shown in FIG. 3), and the heavy chain comprises pI variants (including those shown in FIG. 2).

FIG. 52 shows some exemplary Fc domain sequences that are useful with the 2+1 Fab₂-scFv-Fc format. The “monomer 1” sequences typically refer to the Fc domain of the “Fab-Fc heavy chain” and the “monomer 2” sequences refer to the Fc domain of the “Fab-scFv-Fc heavy chain” as shown in FIG. 52. Other useful Fc domain sequences or variants thereof can be found in FIGS. 7-9, 35, 50-53, and 60. In some embodiments, the Fc domain pairs include the monomer 1 and monomer 2 sequences set forth in SEQ ID NOS:815-816, 817-818, 819-820, 821-822, 823-824, 825-826, 827-828, 829-830, 821-832, 833-834, 835-836, 837-838, 839-840, 841-842. 843-844, 845-846, 847-848, 849-850, 851-852, 853-854, 855-856, 857-858, 859-860, 861-862, 863-864, 865-866, 867-868, 869-870, 871-872, or 873-874. Further, FIG. 9 provides useful C_{L} sequences that can be used with this format.

Any suitable NKG2D ABD can be included in the 2+1 Fab₂-scFv-Fc format antibody, included those provided herein. NKG2D antigen binding domain sequences that are of particular use in these embodiments include, but are not limited to, V_{H} and V_{L} domains selected from have V_{H}/V_{L} pairs selected from the group including: 1D7B4_H1_L1; 1D2B4_H0_L0; mAb-C_H0_L0; mAb-D_H0_L0; 1D7B4_L1_H1; 1D2B4_L0_H0; mAb-C_L0_H0; mAb-D_L0_H0; 1D7B4_H1.1_L1; 1D7B4_H1.2_L1; 1D7B4_H1.3_L1; 1D7B4_H1.4_L1; 1D7B4_H1.5_L1; 1D7B4_H1.6_L1; 1D7B4_H1.7_L1; 1D7B4_H1.8_L1; 1D7B4_H1.9_L1; 1D7B4_H1.10_L1; 1D7B4_H1.11_L1; 1D7B4_H1.12_L1; 1D7B4_H1.13_L1; 1D7B4_H1.14_L1; 1D7B4_H1.15_L1; 1D7B4_H1.16_L1; 1D7B4_H1.17_L1; 1D7B4_H1.18_L1; 1D7B4_H1.19_L1; 1D7B4_H1.20_L1; 1D7B4_H1.21_L1; 1D7B4_H1.22_L1; 1D7B4_H1.23_L1; 1D7B4_H1.24_L1; 1D7B4_H1.25_L1; 1D7B4_H1.26_L1; 1D7B4_H1.27_L1; 1D7B4_H1.28_L1; 1D7B4_H1.29_L1; 1D7B4_H1.30_L1; 1D7B4_H1.31_L1; 1D7B4_H1.32_L1; 1D7B4_H1.3_L13; 1D7B4_H1.34_L1; 1D7B4_H1.35_L1; 1D7B4_H1.36_L1; 1D7B4_H1.37_L1; 1D7B4_H1.38_L1; 1D7B4_H1.39_L1; 1D7B4_H1.40_L1; 1D7B4_H1.41_L1; 1D7B4_H1.42_L1; 1D7B4_H1.43_L1; 1D7B4_H1.44_L1; 1D7B4_H1.45_L1; 1D7B4_H1.46_L1; 1D7B4_H1.47_L1; 1D7B4_H1.48_L1; 1D7B4_L1_H1.1; 1D7B4_L1_H1.2; 1D7B4_L1_H1.3; 1D7B4_L1_H1.4; 1D7B4_L1_H1.5; 1D7B4_L1_H1.6; 1D7B4_L1_H1.7; 1D7B4_L1_H1.8; 1D7B4_L1_H1.9; 1D7B4_L1_H1.10; 1D7B4_L1_H1.11; 1D7B4_L1_H1.12; 1D7B4_L1_H1.13; 1D7B4_L1_H1.14; 1D7B4_L1_H1.15; 1D7B4_L1_H1.16; 1D7B4_L1_H1.17; 1D7B4_L1_H1.18; 1D7B4_L1_H1.19; 1D7B4_L1_H1.20; 1D7B4_L1_H1.21; 1D7B4_L1_H1.22; 1D7B4_L1_H1.23; 1D7B4_L1_H1.24; 1D7B4_L1_H1.25; 1D7B4_L1_H1.26; 1D7B4_L1_H1.27; 1D7B4_L1_H1.28; 1D7B4_L1_H1.29; 1D7B4_L1_H1.30; 1D7B4_L1_H1.31; 1D7B4_L1_H1.32; 1D7B4_L1_H1.33; 1D7B4_L1_H1.34; 1D7B4_L1_H1.35; 1D7B4_L1_H1.36; 1D7B4_L1_H1.37; 1D7B4_L1_H1.38; 1D7B4_L1_H1.39; 1D7B4_L1_H1.40; 1D7B4_L1_H1.41; 1D7B4_L1_H1.42; 1D7B4_L1_H1.43; 1D7B4_L1_H1.44; 1D7B4_L1_H1.45; 1D7B4_L1_H1.46; 1D7B4_L1_H1.47; 1D7B4_L1_H1.48; 1D2B4_H1_L1; 1D2B4_L1_H1; ADI27744_A44_H0_L0; ADI27744_A44_L0_H0; ADI27749_A49_H0_L0; ADI27749_A49_L0_H0; or variants thereof, as well as those depicted in FIGS. 19, 56, 58 and 59.

In some embodiments, the αNKG2D ABD V_{H}/V_{L} pairs are found in, for example, SEQ ID NOS:1020 and 3; SEQ ID NOS:1022 and 3; SEQ ID NOS:1023 and 1024; SEQ ID NOS:1025 and 1026; SEQ ID NOS:1211 and 51; SEQ ID NOS:1213 and 51; SEQ ID NOS:1215 and 51; SEQ ID NOS:1217 and 51; SEQ ID NOS:1219 and 51; SEQ ID NOS:1221 and 51; SEQ ID NOS:1223 and 51; SEQ ID NOS:1225 and 51; SEQ ID NOS:1227 and 51; SEQ ID NOS:1229 and 51; SEQ ID NOS:1231 and 51; SEQ ID NOS:1233 and 51; SEQ ID NOS:1235 and 51; SEQ ID NOS:1237 and 51; SEQ ID NOS:1239 and 51; SEQ ID NOS:1241 and 51; SEQ ID NOS:1243 and 51; SEQ ID NOS:1245 and 51; SEQ ID NOS:1247 and 51; SEQ ID NOS:1249 and 51; SEQ ID NOS:1251 and 51; SEQ ID NOS:1253 and 51; SEQ ID NOS:1255 and 51; SEQ ID NOS:1257 and 51; SEQ ID NOS:1259 and 51; SEQ ID NOS:1261 and 51; SEQ ID NOS:1263 and 51; SEQ ID NOS:1265 and 51; SEQ ID NOS:1267 and 51; SEQ ID NOS:1269 and 51; SEQ ID NOS:1271 and 51; SEQ ID NOS:1273 and 51; SEQ ID NOS:1275 and 51; SEQ ID NOS:1277 and 51; SEQ ID NOS:1279 and 51; SEQ ID NOS:1281 and 51; SEQ ID NOS:1283 and 51; SEQ ID NOS:1285 and 51; SEQ ID NOS:1287 and 51; SEQ ID NOS:1289 and 51; SEQ ID NOS:1291 and 51; SEQ ID NOS:1293 and 51; SEQ ID NOS:1295 and 51; SEQ ID NOS:1297 and 51; SEQ ID NOS:1299 and 51; SEQ ID NOS:1301 and 51; SEQ ID NOS:1303 and 51; and SEQ ID NOS:1305 and 51, or a variant thereof, as well as those depicted in the FIGS. 56 and 58.

NKG2D antigen binding domain sequences or NKG2D ABD V_{H}/V_{L} pairs finding particular use in these embodiments include, but are not limited to, 1D2B4_H1_L1; 1D2B4_L1_H1; ADI27744_A44_H0_L0; ADI27744_A44_L0_H0; ADI27749_A49_H0_L0; ADI27749_A49_L0_H0; 1D7B4_H1_L1; 1D7B4_L1_H1; 1D7B4_H1.3_L1; 1D7B4_L1_H1.3; 1D7B4_H1.23_L1; 1D7B4_L1_H1.23; 1D7B4_H1.28_L1; 1D7B4_L1_H1.28; 1D7B4_H1.31_L1; 1D7B4_L1_H1.31; 1D7B4_H1.33_L1; 1D7B4_L1_H1.33, or a variant thereof. In certain embodiments, the αNKG2D ABD V_{H}/V_{L} pairs are selected from the group including: ADI27744_A44_H0_L0; ADI27749_A44_H0_L0; 1D7B4_H1.3_L1; 1D7B4_H1.23_L1; 1D7B4_H1.28_L1; 1D7B4_H1.31_L1; 1D7B4_H1.33_L1, as well as V_{H}/V_{L} sequence pairs provided in SEQ ID NOS:1308, 1310, 1312, 1314 and 1316. In particular embodiments, the NKG2D scFv is any one selected from the group including those in SEQ ID NOS:1308, 1310, 1312, 1314 and 1316.

Any suitable B7H3 ABD can be included in the 2+1 Fab₂-scFv-Fc format antibody, including those provided herein. B7H3 ABDs that are of particular use in these embodiments include, but are not limited to, V_{H} and V_{L} domains selected from have V_{H}/V_{L} pairs selected from the group including: 38E2_H2_L1.1; 38E2_L1.1_H2; 6A1_H1_L1; 6A1_L1_H1; 2E4A3.189_H1_L1; 2E4A3.189_L1_H1; 2E4A3.189_H1.22_L1; 2E4A3.189_L1_H1.22, or a variant thereof, as well as those depicted in FIGS. 13 and 14. In certain embodiments, the αB7H3 ABD V_{H}/V_{L} pairs are selected from the group including: 2E4A3.189_H1.22_L1; 38E2_H2_L1.1, 6A1_H1_L1; SEQ ID NOS:140 and 141; SEQ ID NOS:242 and 246; SEQ ID NOS:145 and 51; or variants thereof, as well as those depicted in the FIGS. 13 and 14.

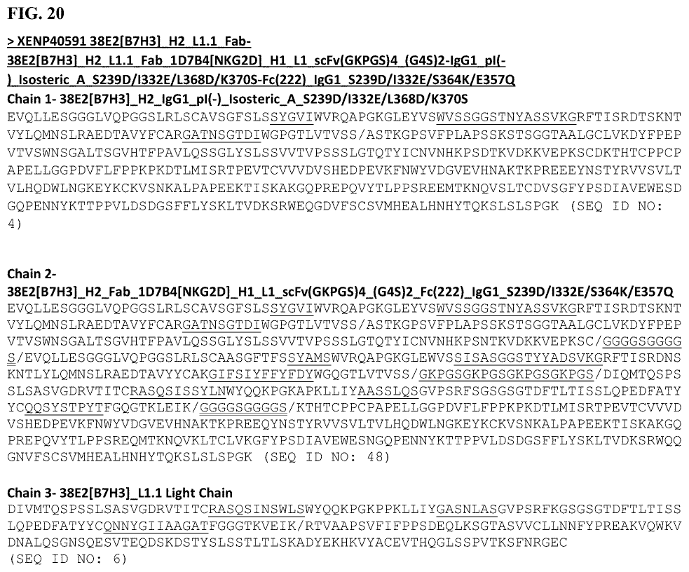

An exemplary anti-B7H3×anti-NKG2D 2+1 Fab₂-scFv-Fc format antibody comprising B7H3 Fab portions and an NKG2D scFv is presented in FIG. 20 as well as SEQ ID NOS:4, 48 and 6. Other exemplary 2+1 Fab₂-scFv-Fc format antibodies specific for B7H3 and NKG2D are shown in FIG. 56, such as XENP40584, XENP40587, XENP40590, XENP40888, XENP41337, XENP41338, XENP41339, and XENP41340, as well as in the sequence listing.

3. 2+1 mAb-scFv Format

One heterodimeric scaffold that finds particular use in the antibodies described herein is the mAb-scFv format (FIG. 15C). In this embodiment, the format relies on the use of a C-terminal attachment of a scFv to one of the monomers, thus forming a third antigen binding domain, wherein the Fab portions of the two monomers bind, for example, B7H3 and the “extra” scFv domain, for example, binds NKG2D. Thus, the first monomer comprises a first heavy chain (comprising a variable heavy domain and a constant domain), with a C-terminally covalently attached scFv comprising a scFv variable light domain, an scFv linker and a scFv variable heavy domain in either orientation (VH1-CH1-hinge-CH2-CH3-[optional linker]-VH2-scFv linker-VL2 or VH1-CH1-hinge-CH2-CH3-[optional linker]-VL2-scFv linker-VH2). This embodiment further utilizes a common light chain comprising a variable light domain and a constant light domain, that associates with the heavy chains to form two identical Fabs that bind B7H3. In some embodiments, an exemplary mAb-scFv format includes a first Fc comprising an N-terminal Fab arm that binds B7H3 and a second Fc comprising an N-terminal Fab arm that binds B7H3 and a C-terminal scFv that binds NKG2D. Such as format can include a first monomer comprising, from the N-terminus to the C-terminus, VH1-CH1-hinge-CH2-CH3, a second monomer comprising, from the N-terminus to the C-terminus, VH1-CH1-hinge-CH2-CH3-scFv, and a third monomer comprising, from the N-terminus to the C-terminus, V_{L}-C_{L}, wherein the first VH1-V_{L} pair bind B7H3, the second VH1-V_{L} pair bind B7H3, and the scFv binds NKG2D. In another embodiment of the mAb-scFv, the first and second VH1-V_{L} pairs bind NKG2D and the scFv binds B7H3.

In some embodiments, one or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the 2+1 mAb-scFv format contain FcγRIIIa variants, skew variants, pI variants, and optionally FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S, M428L/N434A or M252Y/S254T/T256E), optionally charged scFv linkers (including those shown in FIG. 5), optionally ablation variants (including those shown in FIG. 3), and the heavy chain comprises pI variants (including those shown in FIG. 2).

Some exemplary Fc domain sequences that are useful in the 2+1 mAb-scFv format antibodies are shown in the figures, such as FIGS. 9, 35, 50-53, and 60. The “monomer 1” sequences depicted in such figures typically refer to the Fc domain of the “Fab-Fc heavy chain” and the “monomer 2” sequences refer to the Fc domain of the “scFv-Fc heavy chain.” Further, FIG. 10 provides useful C_{L} sequences that can be used with this format.

In some embodiments, the heterodimeric Fc backbones of the first and second monomers of the mAb-scFv format include a backbone pair set forth in FIG. 53 including those pairs of SEQ ID NOS:875-876, 877-878, 879-880, 881-882, 883-884, 885-886, 887 and 889, 890-891, 892-893, 894-895, 896-897, 898-899, 900-901, 902-903, 904-905, 906-907, 908-909, 910-911, 912-913, 914-915, 916-917, 918-919, 920-921, 922-923, 924-925, 926-927, 928-929, 930-931, 932-933, 934-935, 936-937, and 938-939. In some embodiments, the heterodimeric Fc backbones include amino acid substitutions such that the Fc domains have increased ADCC activity. In certain embodiments, the heterodimeric Fc backbone also includes a variant for increasing serum half-life, include, but not limited to, the M428L/N434S, M428L/N434A or M252Y/S254T/T256E variants.

The antibodies described herein provide mAb-scFv formats, where the NKG2D binding domain sequences are shown in FIGS. 23 and 58 or a variant thereof and the B7H3 binding domain sequences are shown in FIGS. 13 and 14 or a variant thereof.

Any suitable NKG2D ABD can be included in the 2+1 mAb-scFv format antibody, included those provided herein. NKG2D antigen binding domain sequences that are of particular use in these embodiments include, but are not limited to, V_{H} and V_{L} domains selected from have V_{H}/V_{L} pairs selected from the group including: 1D7B4_H1_L1; 1D2B4_H0_L0; mAb-C_H0_L0; mAb-D_H0_L0; 1D7B4_L1_H1; 1D2B4_L0_H0; mAb-C_L0_H0; mAb-D_L0_H0; 1D7B4_H1.1_L1; 1D7B4_H1.2_L1; 1D7B4_H1.3_L1; 1D7B4_H1.4_L1; 1D7B4_H1.5_L1; 1D7B4_H1.6_L1; 1D7B4_H1.7_L1; 1D7B4_H1.8_L1; 1D7B4_H1.9_L1; 1D7B4_H1.10_L1; 1D7B4_H1.11_L1; 1D7B4_H1.12_L1; 1D7B4_H1.13_L1; 1D7B4_H1.14_L1; 1D7B4_H1.15_L1; 1D7B4_H1.16_L1; 1D7B4_H1.17_L1; 1D7B4_H1.18_L1; 1D7B4_H1.19_L1; 1D7B4_H1.20_L1; 1D7B4_H1.21_L1; 1D7B4_H1.22_L1; 1D7B4_H1.23_L1; 1D7B4_H1.24_L1; 1D7B4_H1.25_L1; 1D7B4_H1.26_L1; 1D7B4_H1.27_L1; 1D7B4_H1.28_L1; 1D7B4_H1.29_L1; 1D7B4_H1.30_L1; 1D7B4_H1.31_L1; 1D7B4_H1.32_L1; 1D7B4_H1.3_L13; 1D7B4_H1.34_L1; 1D7B4_H1.35_L1; 1D7B4_H1.36_L1; 1D7B4_H1.37_L1; 1D7B4_H1.38_L1; 1D7B4_H1.39_L1; 1D7B4_H1.40_L1; 1D7B4_H1.41_L1; 1D7B4_H1.42_L1; 1D7B4_H1.43_L1; 1D7B4_H1.44_L1; 1D7B4_H1.45_L1; 1D7B4_H1.46_L1; 1D7B4_H1.47_L1; 1D7B4_H1.48_L1; 1D7B4_L1_H1.1; 1D7B4_L1_H1.2; 1D7B4_L1_H1.3; 1D7B4_L1_H1.4; 1D7B4_L1_H1.5; 1D7B4_L1_H1.6; 1D7B4_L1_H1.7; 1D7B4_L1_H1.8; 1D7B4_L1_H1.9; 1D7B4_L1_H1.10; 1D7B4_L1_H1.11; 1D7B4_L1_H1.12; 1D7B4_L1_H1.13; 1D7B4_L1_H1.14; 1D7B4_L1_H1.15; 1D7B4_L1_H1.16; 1D7B4_L1_H1.17; 1D7B4_L1_H1.18; 1D7B4_L1_H1.19; 1D7B4_L1_H1.20; 1D7B4_L1_H1.21; 1D7B4_L1_H1.22; 1D7B4_L1_H1.23; 1D7B4_L1_H1.24; 1D7B4_L1_H1.25; 1D7B4_L1_H1.26; 1D7B4_L1_H1.27; 1D7B4_L1_H1.28; 1D7B4_L1_H1.29; 1D7B4_L1_H1.30; 1D7B4_L1_H1.31; 1D7B4_L1_H1.32; 1D7B4_L1_H1.33; 1D7B4_L1_H1.34; 1D7B4_L1_H1.35; 1D7B4_L1_H1.36; 1D7B4_L1_H1.37; 1D7B4_L1_H1.38; 1D7B4_L1_H1.39; 1D7B4_L1_H1.40; 1D7B4_L1_H1.41; 1D7B4_L1_H1.42; 1D7B4_L1_H1.43; 1D7B4_L1_H1.44; 1D7B4_L1_H1.45; 1D7B4_L1_H1.46; 1D7B4_L1_H1.47; 1D7B4_L1_H1.48; 1D2B4_H1_L1; 1D2B4_L1_H1; ADI27744_A44_H0_L0; ADI27744_A44_L0_H0; ADI27749_A49_H0_L0; ADI27749_A49_L0_H0; or variants thereof, as well as those depicted in FIGS. 19, 56, 58 and 59.

In some embodiments, the αNKG2D ABD V_{H}/V_{L} pairs are found in, for example, SEQ ID NOS:1020 and 3; SEQ ID NOS:1022 and 3; SEQ ID NOS:1023 and 1024; SEQ ID NOS:1025 and 1026; SEQ ID NOS:1211 and 51; SEQ ID NOS:1213 and 51; SEQ ID NOS:1215 and 51; SEQ ID NOS:1217 and 51; SEQ ID NOS:1219 and 51; SEQ ID NOS:1221 and 51; SEQ ID NOS:1223 and 51; SEQ ID NOS:1225 and 51; SEQ ID NOS:1227 and 51; SEQ ID NOS:1229 and 51; SEQ ID NOS:1231 and 51; SEQ ID NOS:1233 and 51; SEQ ID NOS:1235 and 51; SEQ ID NOS:1237 and 51; SEQ ID NOS:1239 and 51; SEQ ID NOS:1241 and 51; SEQ ID NOS:1243 and 51; SEQ ID NOS:1245 and 51; SEQ ID NOS:1247 and 51; SEQ ID NOS:1249 and 51; SEQ ID NOS:1251 and 51; SEQ ID NOS:1253 and 51; SEQ ID NOS:1255 and 51; SEQ ID NOS:1257 and 51; SEQ ID NOS:1259 and 51; SEQ ID NOS:1261 and 51; SEQ ID NOS:1263 and 51; SEQ ID NOS:1265 and 51; SEQ ID NOS:1267 and 51; SEQ ID NOS:1269 and 51; SEQ ID NOS:1271 and 51; SEQ ID NOS:1273 and 51; SEQ ID NOS:1275 and 51; SEQ ID NOS:1277 and 51; SEQ ID NOS:1279 and 51; SEQ ID NOS:1281 and 51; SEQ ID NOS:1283 and 51; SEQ ID NOS:1285 and 51; SEQ ID NOS:1287 and 51; SEQ ID NOS:1289 and 51; SEQ ID NOS:1291 and 51; SEQ ID NOS:1293 and 51; SEQ ID NOS:1295 and 51; SEQ ID NOS:1297 and 51; SEQ ID NOS:1299 and 51; SEQ ID NOS:1301 and 51; SEQ ID NOS:1303 and 51; and SEQ ID NOS:1305 and 51, or a variant thereof, as well as those depicted in the FIGS. 56 and 58.

NKG2D antigen binding domain sequences or NKG2D ABD V_{H}/V_{L} pairs finding particular use in these embodiments include, but are not limited to, 1D2B4_H1_L1; 1D2B4_L1_H1; ADI27744_A44_H0_L0; ADI27744_A44_L0_H0; ADI27749_A49_H0_L0; ADI27749_A49_L0_H0; 1D7B4_H1_L1; 1D7B4_L1_H1; 1D7B4_H1.3_L1; 1D7B4_L1_H1.3; 1D7B4_H1.23_L1; 1D7B4_L1_H1.23; 1D7B4_H1.28_L1; 1D7B4_L1_H1.28; 1D7B4_H1.31_L1; 1D7B4_L1_H1.31; 1D7B4_H1.33_L1; 1D7B4_L1_H1.33, or a variant thereof. In certain embodiments, the αNKG2D ABD V_{H}/V_{L} pairs are selected from the group including: ADI27744_A44_H0_L0; ADI27749_A44_H0_L0; 1D7B4_H1.3_L1; 1D7B4_H1.23_L1; 1D7B4_H1.28_L1; 1D7B4_H1.31_L1; 1D7B4_H1.33_L1, as well as V_{H}/V_{L} sequence pairs provided in SEQ ID NOS:1308, 1310, 1312, 1314 and 1316. In particular embodiments, the NKG2D scFv is any one selected from the group including those in SEQ ID NOS:1308, 1310, 1312, 1314 and 1316.

Any suitable B7H3 ABD can be included in the 2+1 mAb-scFv format antibody, including those provided herein. B7H3 ABDs that are of particular use in these embodiments include, but are not limited to, V_{H} and V_{L} domains selected from have V_{H}/V_{L} pairs selected from the group including: 38E2_H2_L1.1; 38E2_L1.1_H2; 6A1_H1_L1; 6A1_L1_H1; 2E4A3.189_H1_L1; 2E4A3.189_L1_H1; 2E4A3.189_H1.22_L1; 2E4A3.189_L1_H1.22, or a variant thereof, as well as those depicted in FIGS. 13 and 14. In certain embodiments, the αB7H3 ABD V_{H}/V_{L} pairs are selected from the group including: 2E4A3.189_H1.22_L1; 38E2_H2_L1.1, 6A1_H1_L1; SEQ ID NOS:140 and 141; SEQ ID NOS:242 and 246; SEQ ID NOS:145 and 51; or variants thereof, as well as those depicted in the FIGS. 13 and 14.

Exemplary anti-B7H3×anti-NKG2D 2+1 mAb-scFv format antibody is depicted in FIG. 21, as well as SEQ ID NOS:4, 49 and 6. Other exemplary 2+1 mAb-scFv format antibodies are depicted in FIG. 56, such as XENP40552, XENP40555, XENP40557, XENP40558, XENP40559, XENP40891, XENP41341, XENP41342, XENP41343, and XENP41344, as well as in the sequence listing.

4. 2+1 stackFab₂-scFv-Fc Format

Another heterodimeric scaffold that finds particular use in the antibodies described herein is the stackFab₂-scFv-Fc format (FIG. 15D).

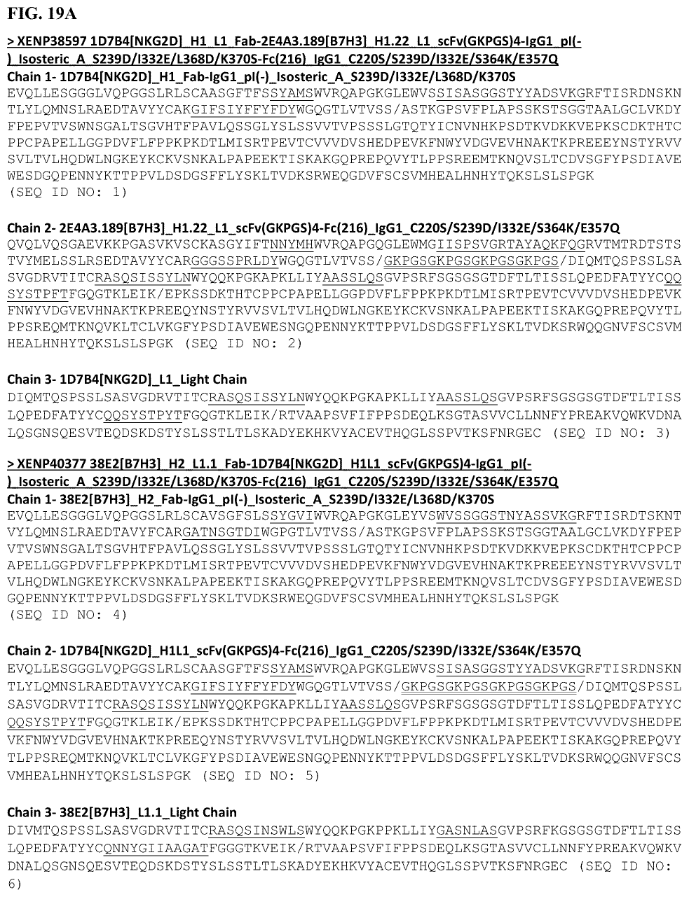

In this embodiment, the format relies on the use of a stacked Fab portions that bind, for example B7H3, and an scFv domain that binds, for example, NKG2D. In this format, a first monomer comprises, from N-terminus to C-terminus, a first heavy chain (comprising a variable heavy domain and a constant domain), a domain linker, and a second heavy chain (comprising a variable heavy domain and a constant domain) and a first Fc domain; a second monomer comprises, from N-terminus to C-terminus, scFv comprising a scFv variable light domain, an scFv linker and a scFv variable heavy domain in either orientation (VH1-CH1-hinge-CH2-CH3-[optional linker]-VH2-scFv linker-VL2 or VH1-CH1-hinge-CH2-CH3-[optional linker]-VL2-scFv linker-VH2), and a second Fc domain; and a third monomer comprising a common light chain including a variable light domain and a constant light domain. This embodiment further utilizes a common light chain comprising a variable light domain and a constant light domain, that associates with the heavy chains to form two identical Fabs that bind B7H3. In some embodiments, the first Fc domain of the Fab monomer comprises the sequence of SEQ ID NO:147 and the second Fc domain of the scFv monomer comprises the sequence of SEQ ID NO:148, also shown in FIG. 18.

In some embodiments, one or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the stackFab₂-scFv-Fc format contain FcγRIIIa variants, skew variants, pI variants, and optionally FcRn variants. In many embodiments, the Fc domains of the stackFab₂-scFv-Fc format contain FcγRIIIa variants, skew variants, pI variants, and FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S, M428L/N434A or M252Y/S254T/T256E), optionally charged scFv linkers (including those shown in FIG. 5), optionally ablation variants (including those shown in FIG. 3), and the heavy chain comprises pI variants (including those shown in FIG. 2).

FIGS. 7-9, 35, 50-53 and 60 show some exemplary Fc domain sequences or variants thereof that are useful in the stackFab₂-scFv-Fc format antibodies.

Any suitable NKG2D ABD can be included in the stackFab₂-scFv-Fc format antibody, included those provided herein. NKG2D antigen binding domain sequences that are of particular use in these embodiments include, but are not limited to, V_{H} and V_{L} domains selected from have V_{H}/V_{L} pairs selected from the group including: 1D7B4_H1_L1; 1D2B4_H0_L0; mAb-C_H0_L0; mAb-D_H0_L0; 1D7B4_L1_H1; 1D2B4_L0_H0; mAb-C_L0_H0; mAb-D_L0_H0; 1D7B4_H1.1_L1; 1D7B4_H1.2_L1; 1D7B4_H1.3_L1; 1D7B4_H1.4_L1; 1D7B4_H1.5_L1; 1D7B4_H1.6_L1; 1D7B4_H1.7_L1; 1D7B4_H1.8_L1; 1D7B4_H1.9_L1; 1D7B4_H1.10_L1; 1D7B4_H1.11_L1; 1D7B4_H1.12_L1; 1D7B4_H1.13_L1; 1D7B4_H1.14_L1; 1D7B4_H1.15_L1; 1D7B4_H1.16_L1; 1D7B4_H1.17_L1; 1D7B4_H1.18_L1; 1D7B4_H1.19_L1; 1D7B4_H1.20_L1; 1D7B4_H1.21_L1; 1D7B4_H1.22_L1; 1D7B4_H1.23_L1; 1D7B4_H1.24_L1; 1D7B4_H1.25_L1; 1D7B4_H1.26_L1; 1D7B4_H1.27_L1; 1D7B4_H1.28_L1; 1D7B4_H1.29_L1; 1D7B4_H1.30_L1; 1D7B4_H1.31_L1; 1D7B4_H1.32_L1; 1D7B4_H1.3_L13; 1D7B4_H1.34_L1; 1D7B4_H1.35_L1; 1D7B4_H1.36_L1; 1D7B4_H1.37_L1; 1D7B4_H1.38_L1; 1D7B4_H1.39_L1; 1D7B4_H1.40_L1; 1D7B4_H1.41_L1; 1D7B4_H1.42_L1; 1D7B4_H1.43_L1; 1D7B4_H1.44_L1; 1D7B4_H1.45_L1; 1D7B4_H1.46_L1; 1D7B4_H1.47_L1; 1D7B4_H1.48_L1; 1D7B4_L1_H1.1; 1D7B4_L1_H1.2; 1D7B4_L1_H1.3; 1D7B4_L1_H1.4; 1D7B4_L1_H1.5; 1D7B4_L1_H1.6; 1D7B4_L1_H1.7; 1D7B4_L1_H1.8; 1D7B4_L1_H1.9; 1D7B4_L1_H1.10; 1D7B4_L1_H1.11; 1D7B4_L1_H1.12; 1D7B4_L1_H1.13; 1D7B4_L1_H1.14; 1D7B4_L1_H1.15; 1D7B4_L1_H1.16; 1D7B4_L1_H1.17; 1D7B4_L1_H1.18; 1D7B4_L1_H1.19; 1D7B4_L1_H1.20; 1D7B4_L1_H1.21; 1D7B4_L1_H1.22; 1D7B4_L1_H1.23; 1D7B4_L1_H1.24; 1D7B4_L1_H1.25; 1D7B4_L1_H1.26; 1D7B4_L1_H1.27; 1D7B4_L1_H1.28; 1D7B4_L1_H1.29; 1D7B4_L1_H1.30; 1D7B4_L1_H1.31; 1D7B4_L1_H1.32; 1D7B4_L1_H1.33; 1D7B4_L1_H1.34; 1D7B4_L1_H1.35; 1D7B4_L1_H1.36; 1D7B4_L1_H1.37; 1D7B4_L1_H1.38; 1D7B4_L1_H1.39; 1D7B4_L1_H1.40; 1D7B4_L1_H1.41; 1D7B4_L1_H1.42; 1D7B4_L1_H1.43; 1D7B4_L1_H1.44; 1D7B4_L1_H1.45; 1D7B4_L1_H1.46; 1D7B4_L1_H1.47; 1D7B4_L1_H1.48; 1D2B4_H1_L1; 1D2B4_L1_H1; ADI27744_A44_H0_L0; ADI27744_A44_L0_H0; ADI27749_A49_H0_L0; ADI27749_A49_L0_H0; or variants thereof, as well as those depicted in FIGS. 19, 56, 58 and 59.

In some embodiments, the αNKG2D ABD V_{H}/V_{L} pairs are found in, for example, SEQ ID NOS:1020 and 3; SEQ ID NOS:1022 and 3; SEQ ID NOS:1023 and 1024; SEQ ID NOS:1025 and 1026; SEQ ID NOS:1211 and 51; SEQ ID NOS:1213 and 51; SEQ ID NOS:1215 and 51; SEQ ID NOS:1217 and 51; SEQ ID NOS:1219 and 51; SEQ ID NOS:1221 and 51; SEQ ID NOS:1223 and 51; SEQ ID NOS:1225 and 51; SEQ ID NOS:1227 and 51; SEQ ID NOS:1229 and 51; SEQ ID NOS:1231 and 51; SEQ ID NOS:1233 and 51; SEQ ID NOS:1235 and 51; SEQ ID NOS:1237 and 51; SEQ ID NOS:1239 and 51; SEQ ID NOS:1241 and 51; SEQ ID NOS:1243 and 51; SEQ ID NOS:1245 and 51; SEQ ID NOS:1247 and 51; SEQ ID NOS:1249 and 51; SEQ ID NOS:1251 and 51; SEQ ID NOS:1253 and 51; SEQ ID NOS:1255 and 51; SEQ ID NOS:1257 and 51; SEQ ID NOS:1259 and 51; SEQ ID NOS:1261 and 51; SEQ ID NOS:1263 and 51; SEQ ID NOS:1265 and 51; SEQ ID NOS:1267 and 51; SEQ ID NOS:1269 and 51; SEQ ID NOS:1271 and 51; SEQ ID NOS:1273 and 51; SEQ ID NOS:1275 and 51; SEQ ID NOS:1277 and 51; SEQ ID NOS:1279 and 51; SEQ ID NOS:1281 and 51; SEQ ID NOS:1283 and 51; SEQ ID NOS:1285 and 51; SEQ ID NOS:1287 and 51; SEQ ID NOS:1289 and 51; SEQ ID NOS:1291 and 51; SEQ ID NOS:1293 and 51; SEQ ID NOS:1295 and 51; SEQ ID NOS:1297 and 51; SEQ ID NOS:1299 and 51; SEQ ID NOS:1301 and 51; SEQ ID NOS:1303 and 51; and SEQ ID NOS:1305 and 51, or a variant thereof, as well as those depicted in the FIGS. 56 and 58.

NKG2D antigen binding domain sequences or NKG2D ABD V_{H}/V_{L} pairs finding particular use in these embodiments include, but are not limited to, 1D2B4_H1_L1; 1D2B4_L1_H1; ADI27744_A44_H0_L0; ADI27744_A44_L0_H0; ADI27749_A49_H0_L0; ADI27749_A49_L0_H0; 1D7B4_H1_L1; 1D7B4_L1_H1; 1D7B4_H1.3_L1; 1D7B4_L1_H1.3; 1D7B4_H1.23_L1; 1D7B4_L1_H1.23; 1D7B4_H1.28_L1; 1D7B4_L1_H1.28; 1D7B4_H1.31_L1; 1D7B4_L1_H1.31; 1D7B4_H1.33_L1; 1D7B4_L1_H1.33, or a variant thereof. In certain embodiments, the αNKG2D ABD V_{H}/V_{L} pairs are selected from the group including: ADI27744_A44_H0_L0; ADI27749_A44_H0_L0; 1D7B4_H1.3_L1; 1D7B4_H1.23_L1; 1D7B4_H1.28_L1; 1D7B4_H1.31_L1; 1D7B4_H1.33_L1, as well as V_{H}/V_{L} sequence pairs provided in SEQ ID NOS:1308, 1310, 1312, 1314 and 1316. In particular embodiments, the NKG2D scFv is any one selected from the group including those in SEQ ID NOS:1308, 1310, 1312, 1314 and 1316.

Any suitable B7H3 ABD can be included in the 2+1 mAb-scFv format antibody, including those provided herein. B7H3 ABDs that are of particular use in these embodiments include, but are not limited to, V_{H} and V_{L} domains selected from have V_{H}/V_{L} pairs selected from the group including: 38E2_H2_L1.1; 38E2_L1.1_H2; 6A1_H1_L1; 6A1_L1_H1; 2E4A3.189_H1_L1; 2E4A3.189_L1_H1; 2E4A3.189_H1.22_L1; 2E4A3.189_L1_H1.22, or a variant thereof, as well as those depicted in FIGS. 13 and 14. In certain embodiments, the αB7H3 ABD V_{H}/V_{L} pairs are selected from the group including: 2E4A3.189_H1.22_L1; 38E2_H2_L1.1, 6A1_H1_L1; SEQ ID NOS:140 and 141; SEQ ID NOS:242 and 246; SEQ ID NOS:145 and 51; or variants thereof, as well as those depicted in the FIGS. 13 and 14.

An exemplary anti-B7H3×anti-NKG2D 2+1 stackFab₂-scFv format antibody is depicted in FIG. 57, as well as SEQ ID NOS: 1209, 1210 and 6.

5. 1+1 CLC Format

One heterodimeric scaffold that finds particular use in the antibodies described herein is the “1+1 common light chain” (or “1+1 CLC”) format, as shown in FIG. 2 of U.S. Pat. No. 10,793,632, hereby incorporated by reference in its entirety including the figures and legends. The 1+1 CLC format antibody includes: a first monomer that includes a VH1-CH1-hinge-CH2-CH3, wherein VH1 is a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer “common light chain” comprising V_{L}-C_{L}, wherein V_{L} is a common variable light domain and C_{L} is a constant light domain. In such embodiments, the V_{L} pairs with the VH1 to form a first binding domain with a first antigen binding specificity; and the V_{L} pairs with the VH2 to form a second binding domain with a second antigen binding specificity. In some embodiments, the 1+1 CLC format antibody is a bivalent antibody. In some embodiments, one or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the 1+1 Fab-scFv-Fc format contain FcγRIIIa variants, skew variants, pI variants, and optionally FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S, M428L/N434A or M252Y/S254T/T256E), optionally charged scFv linkers (including those shown in FIG. 5), optionally ablation variants (including those shown in FIG. 3), and the heavy chain comprises pI variants (including those shown in FIG. 2).

In some embodiments, one of the first or second antigen binding domains is an NK cell antigen binding domain. Any suitable NKG2D antigen binding domain (as described herein and in the Figures and sequence listing) or a variant thereof can be included in the subject 1+1 CLC format antibody.

In some embodiments, one of the first or second antigen binding domains is a B7H3 antigen binding domain. Any suitable B7H3 binding domain (as described herein and in the Figures and sequence listing) or a variant thereof can be included in the subject 1+1 CLC format antibody.

6. 2+1 CLC Format

One heterodimeric scaffold that finds particular use in the antibodies described herein is the “2+1 common light chain” (or (“2+1 CLC”) format, as shown in FIG. 2 of U.S. Pat. No. 10,793,632, hereby incorporated by reference in its entirety including the figures and legends. The 2+1 CLC format includes: a first monomer that includes a VH1-CH1-linker-VH1-CH1-hinge-CH2-CH3, wherein the first and second VH1 are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” V_{L}-C_{L}, wherein V_{L} is a common variable light domain and C_{L} is a constant light domain. The V_{L} domain pairs with each of the VH1 domains of the first monomer to form two first binding domains, each with a first antigen binding specificity; and the V_{L} pairs with the VH2 to form a second binding domain with a second antigen binding specificity. The linker of the first monomer can be any suitable linker, including, but not limited to, any one of the domain linkers described in FIG. 6 (see, e.g., SEQ ID NOS: 87, 98, 109-130, and 203-206). In some embodiments, the linker is EPKSCGKPGSGKPGS (SEQ ID NO: 205). In some embodiments, the 2+1 CLC format antibody is a trivalent antibody. In some embodiments, the first antigen binding specificity is to NKG2D such as the ECD of NKG2D and the second antigen binding specificity is for B7H3 such as the ECD of B7H3. In other embodiments, the first antigen binding specificity is for B7H3 such as the ECD of B7H3 and the second antigen binding specificity is for NKG2D such as the ECD of NKG2D. In some embodiments, one or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the 1+1 Fab-scFv-Fc format contain FcγRIIIa variants, skew variants, pI variants, and optionally FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S, M428L/N434A or M252Y/S254T/T256E), optionally charged scFv linkers (including those shown in FIG. 5), optionally ablation variants (including those shown in FIG. 3), and the heavy chain comprises pI variants (including those shown in FIG. 2).

In some embodiments, each of the first antigen binding domains or the second antigen binding domain is an NKG2D antigen binding domain. Any suitable NKG2D antigen binding domain (as described herein and in the figures and sequence listing) or a variant thereof can be included in the subject 2+1 CLC format antibody.

In some embodiments, each of the first antigen binding domains or the second antigen binding domain is a B7H3 binding domain. Any suitable B7H3 antigen binding domain (as described herein and in the figures and sequence listing) or a variant thereof can be included in the subject 2+1 CLC format antibody.

7. mAb-Fv Format

One heterodimeric scaffold that finds particular use in the antibodies described herein is the “mAb-Fv” format, as shown in FIG. 2 of U.S. Pat. No. 10,793,632, hereby incorporated by reference in its entirety including the figures and legends. In one embodiment, the format relies on the use of a C-terminal attachment of an “extra” variable heavy domain to one monomer and the C-terminal attachment of an “extra” variable light domain to the other monomer, thus forming a third ABD (i.e., an “extra” Fv domain), wherein the Fab portions of the two monomers bind the ECD of B7H3 and the “extra” scFv domain binds the ECD of NKG2D. In another embodiment, the format relies on the use of a C-terminal attachment of an “extra” variable heavy domain to one monomer and the C-terminal attachment of an “extra” variable light domain to the other monomer, thus forming a third ABD (i.e., an “extra” Fv domain), wherein the Fab portions of the two monomers bind the ECD of NKG2D and the “extra” scFv domain binds the ECD of B7H3. In some embodiments, one or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the 1+1 Fab-scFv-Fc format contain FcγRIIIa variants, skew variants, pI variants, and optionally FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S or M428L/N434A), optionally charged scFv linkers (including those shown in FIG. 5), optionally ablation variants (including those shown in FIG. 3), and the heavy chain comprises pI variants (including those shown in FIG. 2).

In this embodiment, the first monomer comprises: a first heavy chain, comprising a first variable heavy domain and a first constant heavy domain comprising a first Fc domain, with a first variable light domain covalently attached to the C-terminus of the first Fc domain using a domain linker (VH1-CH1-hinge-CH2-CH3-[optional linker]-VL2). The second monomer comprises: a second variable heavy domain, a second constant heavy domain comprising a second Fc domain, and a third variable heavy domain covalently attached to the C-terminus of the second Fc domain using a domain linker (VH1-CH1-hinge-CH2-CH3-[optional linker]-VH2). This embodiment further utilizes a common light chain comprising a variable light domain and a constant light domain, which associates with the heavy chains to form two identical Fabs that include two identical Fvs. The two C-terminally attached variable domains (VL2 and VH2) make up the “extra” third Fv.

As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NKG2D antigen binding domain (as described herein and in the Figures and sequence listing, or a variant thereof) can be included in the subject mAb-Fv format antibody.

Any suitable B7H3 binding domain (as described herein and in the Figures and sequence listing, or a variant thereof) can be included in the subject mAb-Fv format antibody.

8. Dual scFv Format

One heterodimeric scaffold that finds particular use in the antibodies described herein is the “dual scFv” format, as shown in FIG. 2 of U.S. Pat. No. 10,793,632, hereby incorporated by reference in its entirety including the figures and legends.

In some embodiments, the format relies on the use of two scFv-Fc monomers (both in either the V_{H}-scFv linker-V_{L}-[optional domain linker]-CH2-CH3 format, the V_{L}-scFv linker-V_{H}-[optional domain linker]-CH2-CH3 format, or with one monomer in one orientation and the other monomer in the other orientation). In some instances, one of the scFv-Fc monomers binds NKG2D such as the ECD thereof and the other scFv-Fc monomers binds B7H3 such as the ECD thereof. In the format, both ABDs are in an scFv format. Any suitable NKG2D ABD and B7H3 ABD can be included in the subject bispecific antibodies in the dual scFv format, including any of the NKG2D ABDs and B7H3 ABDs described herein, in the Figures, and sequence listing, as well as variants thereof. In some embodiments, the first scFv or the second scFv is the domain that binds to NKG2D. In some embodiments, one Fc domain (CH2-CH3 of one monomer) or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the 1+1 Fab-scFv-Fc format contain FcγRIIIa variants, skew variants, pI variants, and optionally FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S or M428L/N434A), optionally charged scFv linkers (including those shown in FIG. 5), optionally ablation variants (including those shown in FIG. 3), and the heavy chain comprises pI variants (including those shown in FIG. 2).

Any suitable NKG2D ABD V_{H}/V_{L} pairs described herein (and in the Figures and sequence listing), or a variant thereof, can be included in the subject dual scFv format antibody. In some embodiments, the first scFv or the second scFv is the domain that binds to B7H3. Any suitable B7H3 ABD V_{H}/V_{L} pairs described herein (and in the Figures and sequence listing), or a variant thereof, can be included in the subject dual scFv format antibody.

9. One-Armed scFv-mAb Format

One heterodimeric scaffold that finds particular use in the antibodies described herein is the “one-armed scFv-mAb” format, as shown in FIG. 2 of U.S. Pat. No. 10,793,632, hereby incorporated by reference in its entirety including the figures and legends. This format includes: 1) a first monomer that comprises an “empty” Fc domain; 2) a second monomer that includes a first variable heavy domain (V_{H}), a scFv domain (a second ABD), an Fc domain, where the scFv domain is attached to the N-terminus of the first variably heavy domain; and 3) a light chain that includes a first variable light domain and a constant light domain. The first variable heavy domain and the first variable light domain form a first antigen binding domain and the scFv is a second antigen binding domain. In some embodiments of the format, one of the first ABDs and the second ABDs binds NKG2D such as the ECD of NKG2D, and the other ABD binds B7H3 such as the ECD of NKG2D. In some embodiments, one or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the 1+1 Fab-scFv-Fc format contain FcγRIIIa variants, skew variants, pI variants, and optionally FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S or M428L/N434A), optionally charged scFv linkers (including those shown in FIG. 5), optionally ablation variants (including those shown in FIG. 3), and the heavy chain comprises pI variants (including those shown in FIG. 2).

Any suitable NKG2D antigen binding domain (as described herein and in the Figures and sequence listing) or a variant thereof can be included in the subject one-armed scFv-mAb format antibody.

Any suitable B7H3 antigen binding domain (as described herein and in Figures and sequence listing) or a variant thereof can be included in the subject one-armed scFv-mAb format antibody.

10. One-Armed Central-scFv Format

One heterodimeric scaffold that finds particular use in the antibodies described herein is the “one-armed central-scFv” format, as shown in FIG. 2 of U.S. Pat. No. 10,793,632, hereby incorporated by reference in its entirety including the figures and legends. In the format, one monomer comprises solely an Fc domain (i.e., a first Fc domain), while the other monomer includes a Fab domain (a first ABD), a scFv (a second ABD), and an Fc domain (i.e., a second Fc domain), where the scFv domain is inserted between the first Fc domain and the second Fc domain. In this format, the Fab portion binds a first antigen and the scFv binds a second antigen. In other words, one monomer comprises a first heavy chain comprising a first variable heavy domain, a CH1 domain, and a Fc domain, with a scFv comprising a scFv variable light domain, an scFv linker, and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). The embodiment further utilizes a light chain comprising a variable light domain and a constant light domain that associates with the heavy chain to form a Fab. In some embodiments, the Fab portion binds B7H3 such as the ECD thereof and the scFv binds NKG2D such as the ECD thereof. In some embodiments, the Fab portion binds NKG2D such as the ECD thereof and the scFv binds B7H3 such as the ECD thereof.

In some embodiments, one or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the 1+1 Fab-scFv-Fc format contain FcγRIIIa variants, skew variants, pI variants, and optionally FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S or M428L/N434A), optionally charged scFv linkers (including those shown in FIG. 5), optionally ablation variants (including those shown in FIG. 3), and the heavy chain comprises pI variants (including those shown in FIG. 2).

Any suitable NKG2D antigen binding domain (as described herein and in the and sequence listing) or a variant thereof can be included in the subject one-armed central-scFv format antibody.

Any suitable B7H3 binding domain (as described herein and in the Figures and sequence listing) or a variant thereof can be included in the subject one-armed central-scFv format antibody.

11. Central-Fv Format

One heterodimeric scaffold that finds particular use in the antibodies described herein is the “central-Fv” format, as shown in FIG. 2 of U.S. Pat. No. 10,793,632, hereby incorporated by reference in its entirety including the figures and legends. In one embodiment, the format relies on the use of an inserted Fv domain (i.e., the central Fv domain) thus forming an “extra” third ABD, wherein the Fab portions of the two monomers bind B7H3 and the “extra” central Fv domain binds NKG2D. The Fv domain is inserted between the Fc domain and the CH1-Fv region of the monomers, thus providing a third ABD, wherein each monomer contains a component of the Fv (e.g., one monomer comprises a variable heavy domain and the other comprises a variable light domain of the “extra” central Fv domain). In another embodiment, the format relies on the use of an inserted Fv domain (i.e., the central Fv domain) thus forming an “extra” third ABD, wherein the Fab portions of the two monomers bind NKG2D and the “extra” central Fv domain binds B7H3. The Fv domain is inserted between the Fc domain and the CH1-Fv region of the monomers, thus providing a third ABD, wherein each monomer contains a component of the Fv (e.g., one monomer comprises a variable heavy domain and the other comprises a variable light domain of the “extra” central Fv domain).

In this embodiment, one monomer comprises a first heavy chain comprising a first variable heavy domain, a CH1 domain, and Fc domain, and an additional variable light domain. The additional variable light domain is covalently attached between the C-terminus of the CH1 domain of the heavy constant domain and the N-terminus of the first Fc domain using domain linkers (VH1-CH1-[optional linker]-VL2-hinge-CH2-CH3). The other monomer comprises a first heavy chain comprising a first variable heavy domain, a CH1 domain and Fc domain, and an additional variable heavy domain (VH1-CH1-[optional linker]-VH2-hinge-CH2-CH3). The additional variable heavy domain is covalently attached between the C-terminus of the CH1 domain of the heavy constant domain and the N-terminus of the first Fc domain using domain linkers. In some embodiments, the format further utilizes a common light chain comprising a variable light domain and a constant light domain, that associates with the heavy chains to form two identical Fabs that each bind B7H3. The additional variable heavy domain and additional variable light domain form an “extra” central Fc that binds NKG2D. In other embodiments, the format further utilizes a common light chain comprising a variable light domain and a constant light domain, that associates with the heavy chains to form two identical Fabs that each bind NKG2D. The additional variable heavy domain and additional variable light domain form an “extra” central Fc that binds B7H3.

In some embodiments, one or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the 1+1 Fab-scFv-Fc format contain FcγRIIIa variants, skew variants, pI variants, and optionally FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group consisting of S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S or M428L/N434A), optionally charged scFv linkers (including those shown in FIG. 5), optionally ablation variants (including those shown in FIG. 3), and the heavy chain comprises pI variants (including those shown in FIG. 2).

Any suitable NK cell antigen binding domain (as described herein and in the Figures and sequence listing) or a variant thereof can be included in the subject central-Fv format antibody.

Any suitable B7H3 antigen binding domain (as described herein and in the Figures and sequence listing) or a variant thereof can be included in the subject central-Fv format antibody.

12. scFv-mAb Format

One heterodimeric scaffold that finds particular use in the antibodies described herein is the “scFv-mAb” format, as shown in FIG. 2 of U.S. Pat. No. 10,793,632, hereby incorporated by reference in its entirety including the figures and legends. In one embodiment, the format relies on the use of a N-terminal attachment of a scFv to one of the monomers, thus forming a third ABD, wherein the Fab portions of the two monomers bind B7H3 and the “extra” scFv domain binds NKG2D. In another embodiment, the format relies on the use of a N-terminal attachment of a scFv to one of the monomers, thus forming a third ABD, wherein the Fab portions of the two monomers bind NKG2D and the “extra” scFv domain binds B7H3.

In one embodiment, the first monomer comprises a first heavy chain (comprising a variable heavy domain and a constant domain), with a N-terminally covalently attached scFv comprising a scFv variable light domain, an scFv linker, and a scFv variable heavy domain in either orientation ((VH1-scFv linker-VL1-[optional domain linker]-VH2-CH1-hinge-CH2-CH3) or (with the scFv in the opposite orientation (VL1-scFv linker-VH1-[optional domain linker]-VH2-CH1-hinge-CH2-CH3))). This embodiment further utilizes a common light chain comprising a variable light domain and a constant light domain that associates with the heavy chains to form two identical Fabs that bind B7H3. As such, the scFv binds NKG2D.

In some embodiments, one or both Fc domains of these constructs can include one or more of: (i) FcγRIIIa variants, (ii) pI variants, (iii) skew variants, and (iv) FcRn variants, as well as any combination thereof, as desired and described herein. In certain embodiments, the Fc domains of the 1+1 Fab-scFv-Fc format contain FcγRIIIa variants, skew variants, pI variants, and optionally FcRn variants. In some embodiments, such Fc domains include asymmetric FcγRIIIa variants such that one Fc domain includes S239D/I332E substitutions, and the other Fc domain includes no FcγRIIIa variants, amino acid substitution(s) selected from the group including S239D, I332E, S239D/I332E, G236A, S239E, I332D, G236A/I332E, S239D/I332E/A330L, I332E/A330L, F243L, S298A, E333A, K334A, S298A/E333A, and S298A/E333A/K334A. In many embodiments, the Fc domains generally include skew variants (e.g., a set of amino acid substitutions as shown in FIG. 1, with particularly useful skew variants being selected from the group including: S364K/E357Q:L368D/K370S; L368D/K370S:S364K; L368E/K370S:S364K; T411E/K360E/Q362E:D401K; L368D/K370S:S364K/E357L; K370S:S364K/E357Q; T366S/L368A/Y407V:T366W and T366S/L368A/Y407V/Y349C:T366W/S354C), and optionally FcRn variants (including M428L/N434S or M428L/N434A), optionally charged scFv linkers (including those shown in FIG. 5), optionally ablation variants (including those shown in FIG. 3), and the heavy chain comprises pI variants (including those shown in FIG. 2).

Any suitable NKG2D antigen binding domain (as described herein and in the Figures and sequence listing) or a variant thereof can be included in the subject scFv-mAb format antibody.

Any suitable B7H3 antigen binding domain (as described herein and in the Figures and sequence listing) or a variant thereof can be included in the subject scFv-mAb format antibody.

13. Non-Heterodimeric Bispecific Antibodies

As will be appreciated by those in the art, the anti-NKG2D×anti-B7H3 antibodies provided herein can also be included in non-heterodimeric bispecific formats, as shown in FIG. 2 of U.S. Pat. No. 10,793,632, hereby incorporated by reference in its entirety including the figures and legends. In this format, the anti-NKG2D×anti-B7H3 antibody includes: (i) a first monomer comprising a VH1-CH1-hinge-CH2-CH3, (ii) a second monomer comprising a VH2-CH1-hinge-CH2-CH3, (iii) a first light chain comprising a VL1-C_{L}, and (iv) a second light chain comprising a VL2-C_{L}. In such embodiments, the VH1 and VL1 form a first ABD, and VH2 and VL2 form a second ABD. In some embodiments, one of the first or second antigen binding domains binds B7H3 and the other antigen binding domain binds NKG2D. In certain embodiments, one of the first or second antigen binding domains binds NKG2D and the other antigen binding domain binds B7H3.

Any suitable NKG2D antigen binding domain (as described herein and in the Figures and sequence listing) or a variant thereof can be included in the subject non-heterodimeric bispecific format antibody.

Any suitable B7H3 antigen binding domain (as described herein and in the Figures and sequence listing) or a variant thereof can be included in the subject non-heterodimeric bispecific format antibody.

14. Trident Format

One heterodimeric scaffold that finds particular use in the antibodies described herein is the “Trident” format, as shown in FIG. 2 of U.S. Pat. No. 10,793,632, hereby incorporated by reference in its entirety including the figures and legends. Such a “Trident” format is generally described in WO2015/184203, hereby expressly incorporated by reference in its entirety and in particular for the figures, legends, definitions, and sequences of “Heterodimer-Promoting Domains” or “HPDs”, including “K-coil” and “E-coil” sequences. Tridents rely on using two different HPDs that associate to form a heterodimeric structure as a component of the structure. In this embodiment, the Trident format includes a “traditional” heavy and light chain (e.g., VH1-CH1-hinge-CH2-CH3 and VL1-C_{L}), a third chain comprising a first “diabody-type binding domain” or “DART©,” VH2-(linker)-VL3-HPD1, and a fourth chain comprising a second DART®, VH3-(linker)-(linker)-VL2-HPD2. The VH1 and VL1 form a first ABD, the VH2 and VL2 form a second ABD, and the VH3 and VL3 form a third ABD. In some cases, the second and third ABDs bind the same antigen, in this instance generally a tumor target antigen (TTA) such as B7H3, e.g., bivalently, with the first ABD binding NKG2D (such as the ECD of NKG2D) monovalently.

Any suitable NKG2D antigen binding domain (as described herein and in the Figures and sequence listing) or a variant thereof can be included in the subject Trident format antibody.

Any suitable B7H3 antigen binding domain (as described herein and in the Figures and sequence listing) or a variant thereof can be included in the subject Trident format antibody.

15. Monospecific Monoclonal Antibodies

As will be appreciated by those in the art, the novel NKG2D ABD sequences outlined herein can also be used in both monospecific antibodies (e.g., “traditional monoclonal antibodies”) or non-heterodimeric bispecific formats. Accordingly, in some embodiments, the antibodies described herein provide monoclonal (monospecific) antibodies comprising the 6 CDRs and/or the vh and vl sequences from the figures, generally with IgG1, IgG2, or IgG4 constant regions, with IgG1, IgG2 and IgG4 (including IgG4 constant regions comprising a S228P amino acid substitution) finding particular use in some embodiments. That is, any sequence herein with a “H_L” designation can be linked to the constant region of a human IgG1 antibody.

Any suitable NKG2D ABD can be included in the monospecific antibody, including any of the NKG2D ABDs described herein.

In other embodiments, the monospecific antibody is an NKG2D monospecific antibody that has a V_{H}/V_{L} pair selected from the group including: 1D7B4_H1_L1; 1D2B4_H0_L0; mAb-C_H0_L0; mAb-D_H0_L0; 1D7B4_L1_H1; 1D2B4_L0_H0; mAb-C_L0_H0; mAb-D_L0_H0; 1D7B4_H1.1_L1; 1D7B4_H1.2_L1; 1D7B4_H1.3_L1; 1D7B4_H1.4_L1; 1D7B4_H1.5_L1; 1D7B4_H1.6_L1; 1D7B4_H1.7_L1; 1D7B4_H1.8_L1; 1D7B4_H1.9_L1; 1D7B4_H1.10_L1; 1D7B4_H1.11_L1; 1D7B4_H1.12_L1; 1D7B4_H1.13_L1; 1D7B4_H1.14_L1; 1D7B4_H1.15_L1; 1D7B4_H1.16_L1; 1D7B4_H1.17_L1; 1D7B4_H1.18_L1; 1D7B4_H1.19_L1; 1D7B4_H1.20_L1; 1D7B4_H1.21_L1; 1D7B4_H1.22_L1; 1D7B4_H1.23_L1; 1D7B4_H1.24_L1; 1D7B4_H1.25_L1; 1D7B4_H1.26_L1; 1D7B4_H1.27_L1; 1D7B4_H1.28_L1; 1D7B4_H1.29_L1; 1D7B4_H1.30_L1; 1D7B4_H1.31_L1; 1D7B4_H1.32_L1; 1D7B4_H1.3_L13; 1D7B4_H1.34_L1; 1D7B4_H1.35_L1; 1D7B4_H1.36_L1; 1D7B4_H1.37_L1; 1D7B4_H1.38_L1; 1D7B4_H1.39_L1; 1D7B4_H1.40_L1; 1D7B4_H1.41_L1; 1D7B4_H1.42_L1; 1D7B4_H1.43_L1; 1D7B4_H1.44_L1; 1D7B4_H1.45_L1; 1D7B4_H1.46_L1; 1D7B4_H1.47_L1; 1D7B4_H1.48_L1; 1D7B4_L1_H1.1; 1D7B4_L1_H1.2; 1D7B4_L1_H1.3; 1D7B4_L1_H1.4; 1D7B4_L1_H1.5; 1D7B4_L1_H1.6; 1D7B4_L1_H1.7; 1D7B4_L1_H1.8; 1D7B4_L1_H1.9; 1D7B4_L1_H1.10; 1D7B4_L1_H1.11; 1D7B4_L1_H1.12; 1D7B4_L1_H1.13; 1D7B4_L1_H1.14; 1D7B4_L1_H1.15; 1D7B4_L1_H1.16; 1D7B4_L1_H1.17; 1D7B4_L1_H1.18; 1D7B4_L1_H1.19; 1D7B4_L1_H1.20; 1D7B4_L1_H1.21; 1D7B4_L1_H1.22; 1D7B4_L1_H1.23; 1D7B4_L1_H1.24; 1D7B4_L1_H1.25; 1D7B4_L1_H1.26; 1D7B4_L1_H1.27; 1D7B4_L1_H1.28; 1D7B4_L1_H1.29; 1D7B4_L1_H1.30; 1D7B4_L1_H1.31; 1D7B4_L1_H1.32; 1D7B4_L1_H1.33; 1D7B4_L1_H1.34; 1D7B4_L1_H1.35; 1D7B4_L1_H1.36; 1D7B4_L1_H1.37; 1D7B4_L1_H1.38; 1D7B4_L1_H1.39; 1D7B4_L1_H1.40; 1D7B4_L1_H1.41; 1D7B4_L1_H1.42; 1D7B4_L1_H1.43; 1D7B4_L1_H1.44; 1D7B4_L1_H1.45; 1D7B4_L1_H1.46; 1D7B4_L1_H1.47; 1D7B4_L1_H1.48; 1D2B4_H1_L1; 1D2B4_L1_H1; ADI27744_A44_H0_L0; ADI27744_A44_L0_H0; ADI27749_A49_H0_L0; ADI27749_A49_L0_H0; or variants thereof, as well as those depicted in FIGS. 19, 56, 58 and 59.

In some embodiments, the αNKG2D ABD V_{H}/V_{L} pairs are found in, for example, SEQ ID NOS:1020 and 3; SEQ ID NOS:1022 and 3; SEQ ID NOS:1023 and 1024; SEQ ID NOS:1025 and 1026; SEQ ID NOS:1211 and 51; SEQ ID NOS:1213 and 51; SEQ ID NOS:1215 and 51; SEQ ID NOS:1217 and 51; SEQ ID NOS:1219 and 51; SEQ ID NOS:1221 and 51; SEQ ID NOS:1223 and 51; SEQ ID NOS:1225 and 51; SEQ ID NOS:1227 and 51; SEQ ID NOS:1229 and 51; SEQ ID NOS:1231 and 51; SEQ ID NOS:1233 and 51; SEQ ID NOS:1235 and 51; SEQ ID NOS:1237 and 51; SEQ ID NOS:1239 and 51; SEQ ID NOS:1241 and 51; SEQ ID NOS:1243 and 51; SEQ ID NOS:1245 and 51; SEQ ID NOS:1247 and 51; SEQ ID NOS:1249 and 51; SEQ ID NOS:1251 and 51; SEQ ID NOS:1253 and 51; SEQ ID NOS:1255 and 51; SEQ ID NOS:1257 and 51; SEQ ID NOS:1259 and 51; SEQ ID NOS:1261 and 51; SEQ ID NOS:1263 and 51; SEQ ID NOS:1265 and 51; SEQ ID NOS:1267 and 51; SEQ ID NOS:1269 and 51; SEQ ID NOS:1271 and 51; SEQ ID NOS:1273 and 51; SEQ ID NOS:1275 and 51; SEQ ID NOS:1277 and 51; SEQ ID NOS:1279 and 51; SEQ ID NOS:1281 and 51; SEQ ID NOS:1283 and 51; SEQ ID NOS:1285 and 51; SEQ ID NOS:1287 and 51; SEQ ID NOS:1289 and 51; SEQ ID NOS:1291 and 51; SEQ ID NOS:1293 and 51; SEQ ID NOS:1295 and 51; SEQ ID NOS:1297 and 51; SEQ ID NOS:1299 and 51; SEQ ID NOS:1301 and 51; SEQ ID NOS:1303 and 51; and SEQ ID NOS:1305 and 51, or variants thereof, as well as those depicted in the FIGS. 56 and 58.

NKG2D antigen binding domain sequences or NKG2D ABD V_{H}/V_{L} pairs finding particular use in these embodiments include, but are not limited to, 1D2B4_H1_L1; 1D2B4_L1_H1; ADI27744_A44_H0_L0; ADI27744_A44_L0_H0; ADI27749_A49_H0_L0; ADI27749_A49_L0_H0; 1D7B4_H1_L1; 1D7B4_L1_H1; 1D7B4_H1.3_L1; 1D7B4_L1_H1.3; 1D7B4_H1.23_L1; 1D7B4_L1_H1.23; 1D7B4_H1.28_L1; 1D7B4_L1_H1.28; 1D7B4_H1.31_L1; 1D7B4_L1_H1.31; 1D7B4_H1.33_L1; 1D7B4_L1_H1.33, or a variant thereof. In certain embodiments, the αNKG2D ABD V_{H}/V_{L} pairs are selected from the group including: ADI27744_A44_H0_L0; ADI27749_A44_H0_L0; 1D7B4_H1.3_L1; 1D7B4_H1.23_L1; 1D7B4_H1.28_L1; 1D7B4_H1.31_L1; 1D7B4_H1.33_L1, as well as V_{H}/V_{L} sequence pairs provided in SEQ ID NOS:1308, 1310, 1312, 1314 and 1316. In particular embodiments, the NKG2D scFv is any one selected from the group including those in SEQ ID NOS:1308, 1310, 1312, 1314 and 1316.

### VII. Nucleic Acids

The disclosure further provides nucleic acid compositions encoding the anti-NKG2D antibodies provided herein, including, but not limited to, NKG2D×B7H3 bispecific antibodies and NKG2D monospecific antibodies.

As will be appreciated by those in the art, the nucleic acid compositions will depend on the format and scaffold of the heterodimeric protein. Thus, for example, when the format requires three amino acid sequences, such as for the 1+1 Fab-scFv-Fc format (e.g., a first amino acid monomer comprising an Fc domain and a scFv, a second amino acid monomer comprising a heavy chain and a light chain), three nucleic acid sequences can be incorporated into one or more expression vectors for expression. Similarly, some formats (e.g., dual scFv formats such as disclosed in FIG. FIG. 2 of U.S. Pat. No. 10,793,632) only two nucleic acids are needed; again, they can be put into one or two expression vectors.

As is known in the art, the nucleic acids encoding the components of the antibodies described herein can be incorporated into expression vectors as is known in the art, and depending on the host cells used to produce the heterodimeric antibodies described herein. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the antibodies described herein are then transformed into any number of different types of host cells as is well known in the art, including mammalian, bacterial, yeast, insect and/or fungal cells, with mammalian cells (e.g., CHO cells), finding use in many embodiments.

In some embodiments, nucleic acids encoding each monomer and the optional nucleic acid encoding a light chain, as applicable depending on the format, are each contained within a single expression vector, generally under different or the same promoter controls. In embodiments of particular use in the antibodies described herein, each of these two or three nucleic acids are contained on a different expression vector. As shown herein and in U.S. Pat. No. 9,822,186, hereby incorporated by reference, different vector ratios can be used to drive heterodimer formation. That is, surprisingly, while the proteins comprise first monomer:second monomer:light chains (in the case of many of the embodiments herein that have three polypeptides comprising the heterodimeric antibody) in a 1:1:2 ratio, these are not the ratios that give the best results.

The heterodimeric antibodies described herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI, and the heterodimer also has a distinct pI, thus facilitating isoelectric purification of the “1+1 Fab-scFv-Fc” and “2+1” heterodimers (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### VIII. Biological and Biochemical Functionality of the Heterodimeric Bispecific Antibodies

Generally, the bispecific NKG2D×B7H3 antibodies described herein are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### IX. Treatments

Once made, the compositions of the invention find use in a number of oncology applications, by treating cancer, generally by enhancing immune responses, including, activating NK cells, enhancing NK cell mediated lysis of tumor cells and providing co-stimulation to T cells in the tumor environment. Such compositions can be combined with proinflammatory cytokines for increased cytotoxicity against tumor cells.

A. Combination with Cytokine of Other Therapies

In some embodiments, a bispecific anti-B7H3×anti-NKG2D antibody described herein can be used in combination with a cytokine therapy. In some embodiments, a cytokine therapy includes, but is not limited, to an IL-15 therapy, an IL-12 therapy or a combination thereof.

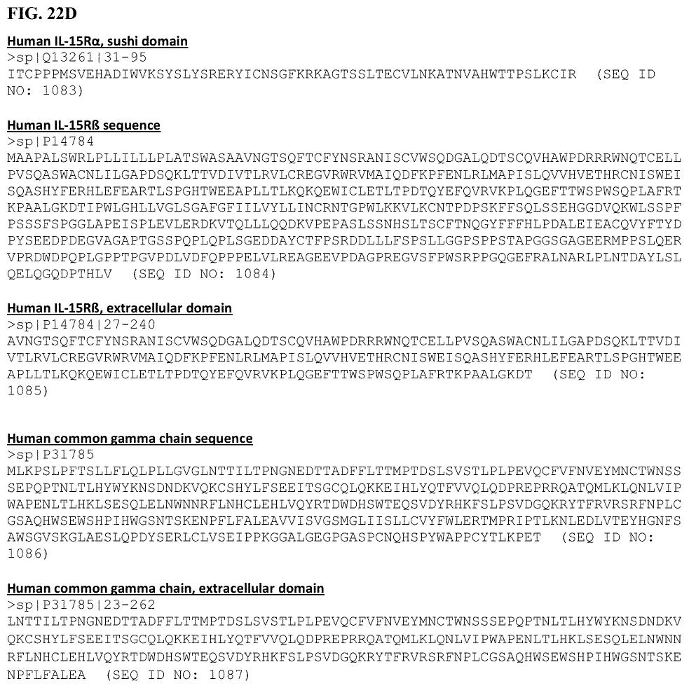

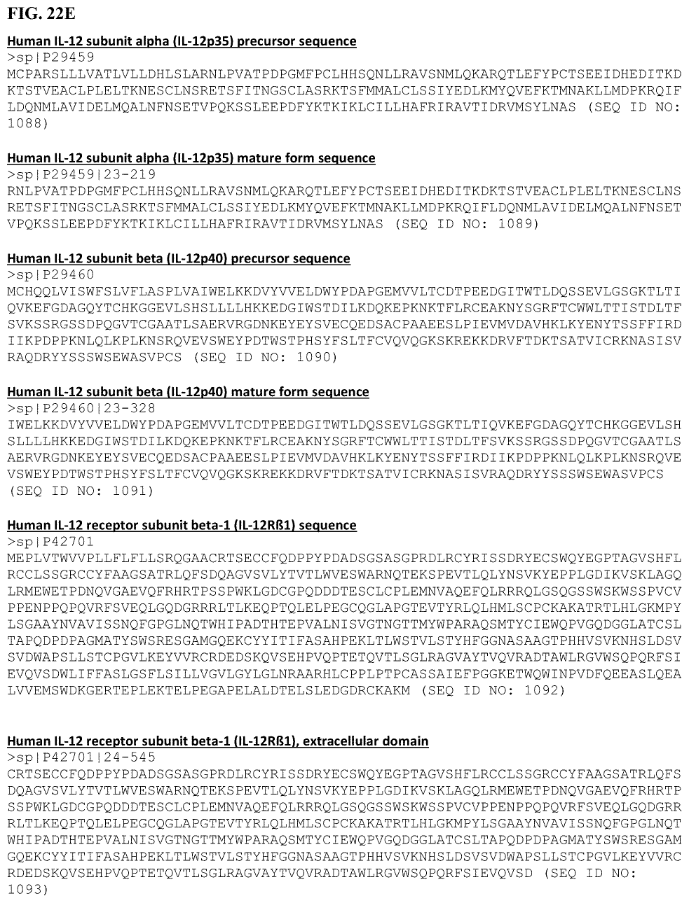

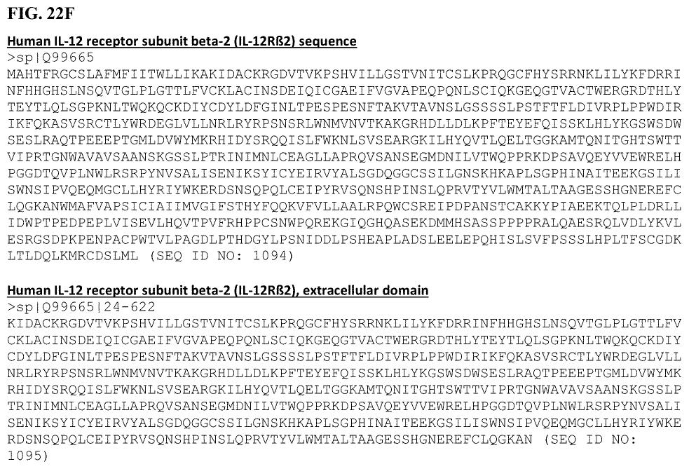

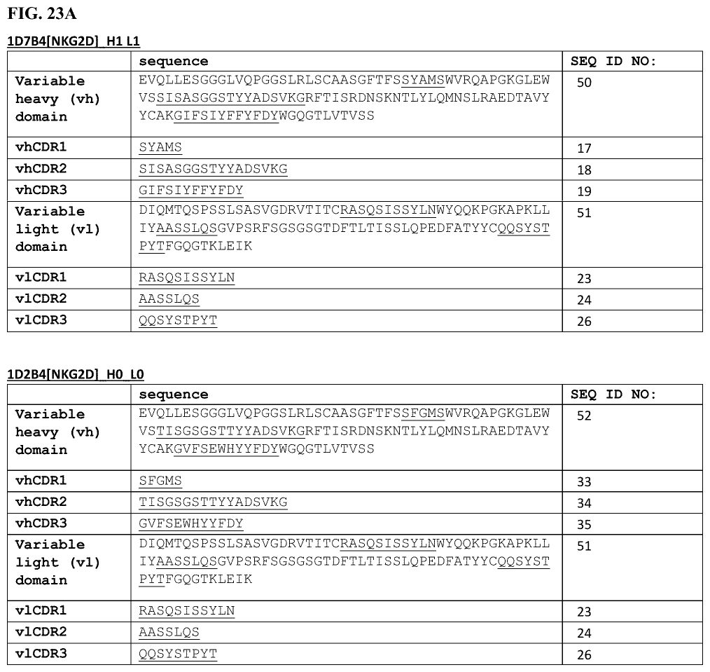

Non-limiting examples of an IL-15 therapy include administration to a subject an IL-15 protein or a fragment thereof, an IL-15 variant protein or a fragment thereof, an IL-15 fusion protein, and an IL-15 agonist. Useful IL-15 fusion proteins include, but are not limited to those shown in FIGS. 22A-22B, such as XENP24045 and XENP24306. Sequences of exemplary IL-15 fusion proteins are provided in the sequence listing such as for SEQ ID NOS:164-165 and SEQ ID NOS:1077-1078.

IL-15 agonists are well-known in the art. Non-limiting examples of IL-15 agonists include PF-07209960 (Pfizer), KD033 (Kadmon), SOT201/SO-C108 (SOTIO/Cytune), SOT101/SO C101/RLI-15 (SOTIO/Cytune), XmAb24306 (Genentech/Xencor), NKTR-255 (Nektar), ALT-803/N-803/Anktiva (Altor Bioscience), and NIZ985 (Novartis). In some embodiments, the IL-15 agonist is an IL-15 protein. The wild-type human IL-15 protein comprises the amino acid sequence shown in FIG. 22C. In some embodiments, the IL-15 agonist is an IL-15 variant protein. IL-15 variant proteins are well-known in the art. See, e.g., U.S. Pat. Nos. 5,552,303; 5,574,138; 6,001,973; 6,013,480; 6,548,065; 6,764,836; 6,998,476; 7,858,081; 8,163,879; 8,178,660; 8,415,456; 8,507,222; 8,940,288; 9,303,080; 9,365,630; 9,371,368; 9,428,563; 9,428,573; 9,493,533; 9,725,492; 9,790,261; 9,932,387; and 9,975,937; U.S. Publication Nos. 2004/009149, 2006/0057680, 2006/0236411, 2006/0257361, 2007/0106066, 2007/0134718, 2009/0105455, 2009/0238791, 2015/0359853, 2016/0130318, 2016/0175459, 2016/0184399, 2016/0275236, 2017/0020963, 2017/0202924, 2017/0246253, 2018/0044424, 2018/0126003, and 2018/0200366; and PCT Publication Nos. WO9527722, WO2016095642, WO2017081082, WO2017046200, WO2017136818, WO2018013855 WO2018023093, WO2018071918, and WO2018071919, each of which is incorporated herein by reference in its entirety. Sequences of human IL-15 proteins, such as the precursor protein and the mature protein are set for in FIG. 22C as SEQ ID NOS:1079 and 1080, respectively and in the sequence listing.

Non-limiting examples of an IL-12 therapy include administration to a subject an IL-12 protein or a fragment thereof, an IL-12 variant protein or a fragment thereof, an IL-12 fusion protein, and an IL-12 agonist. Useful IL-12 fusion proteins include, but are not limited to those shown in FIGS. 22A-22B, such as XENP27201 and XENP39662. Sequences of exemplary IL-12 fusion proteins are provided in the sequence listing such as for SEQ ID NOS:166-167 and SEQ ID NOS:168-169. Other IL-12 fusion proteins include DF6002 (BMS-946415) and those described in PCT Publication No. WO2020086758, which is incorporated herein by reference in its entirety.

IL-12 agonists are also well-known in the art. In some embodiments, the IL-12 agonist is an IL-12 protein. The wild-type IL-12p35 and IL-12p40 subunits comprise the amino acid sequences shown in FIG. 22E. Sequences of human IL-12 proteins, such as the IL-12p35 precursor and mature proteins and the IL-12p40 precursor and mature proteins are set for in FIG. 22E as SEQ ID NOS:1088-1091 and in the sequence listing.

In some embodiments, a bispecific anti-B7H3×anti-NKG2D antibody described herein can be used in combination with a bispecific T-cell engager antibody that binds to the ECDs of B7H3 and CD3 or an antigen binding fragment thereof to the subject. In some embodiments, a useful bispecific T-cell engager antibody can be any one described in Ma et al., Invest New Drugs, 2019 October, 37(5):1036-1043 and PCT Publication Nos. WO2021/027674 and WO2017/030926, which are herein incorporated by reference in their entireties.

In some instances, administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

B. Antibody Compositions for In Vivo Administration

Formulations of the antibodies used in accordance with the antibodies described herein are prepared for storage by mixing an antibody having the desired degree of purity with optional pharmaceutically acceptable carriers, excipients, or stabilizers (Remington's Pharmaceutical Sciences 16th edition, Osol, A. Ed. [1980]), in the form of lyophilized formulations or aqueous solutions.

C. Administration Modalities

The antibodies provided herein administered to a subject, in accord with known methods, such as intravenous administration as a bolus or by continuous infusion over a period of time.

D. Treatment Modalities

In the methods described herein, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MM) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the disclosure includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for cancer therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specifications for the dosage unit forms of the disclosure are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies described herein depend on the disease or condition to be treated and may be determined by the persons skilled in the art.

### IX. Other Embodiments

Provided herein are sequences of exemplary embodiments of the NKG2D×B7H3 bispecific antibodies described herein as well as useful antibodies for use as controls.

In some embodiments, a NKG2D binding domain V_{H}/V_{L} pair or a B7H3 binding domain V_{H}/V_{L} is shown in the sequence listing as, for example, SEQ ID NOS: 1318-1319; 1356-1358; 1359-1361; 1414-1416; 1417-1419; 1420-1422; 1423-1425; 1426-1428; 1429-1431; 1451-1453; 1454-1453; 1457-1459; 1460-1462; 1463-1465; 1466-1468; 1469-1471; 1472-1474; 1475-1477; 1475-1480; 1481-1483; 1489-1488; 1489-1491; 1492-1494; 1507-1509; 1510-1512; 1513-1515; 1516-1518; 1519-1521; 1522-1524; 1525-1527; 1528-1530; 1531-1533; 1534-1536; 1537-1539; 1540-1542; 1543-1545; 1546-1548; 1549-1551; 1552-1554; 1555-1557; 1558-1560; 1561-1563; 1564-1566; 1567-1569; 1570-1572; 1573-1575; 1576-1578; 1579-1581; 1582-1584; 0585-1587; 1588-1590; 1591-1593; 1594-1596; 1597-1599; 1600-1602; 1603-1605; 1606-1608; 1609-1611; 1612-1614; 1615-1617; 1618-1620; 1621-1623; 1624-1626; 1627-1629; 1630-1632; 1633-1635; 1636-1638; 1639-1641; 1642-1644; 1645-1647; 1648-1650; 1651-1653; 1654-1656; 1657-1659; 1660-1662; 1663-1665; 1666-1671; 1672-1674; 1675-1677; 1678-1680; 1681-1683; 1684-1686; 1687-1689; 1690-1692; 1693-1695; 1696-1698; 1699-1701; 1702-1704; 1705-1707; 1708-1710; 1711-1713; 1714-1716; 1717-1719; 1720-1722; 1723-1725; 1726-1728; 1729-1731; 1732-1734; 1732-1734; 1735-1737; 1738-1740; 1741-1743; 1744-1746; 1747-1749; 1750-1752; 1753-1755; 1756-1758; 1759-1761; 1762-1764; 1765-1767; 1768-1770; 1771-1773; 1774-1776; 1777-1779; 1780-1782; 1783-1785; 1786-1788; 1789-1791; 1800-1802; 1803-1805; 1806-1808; 1809-1811; 1812-1814; 1815-1817; 1818-1820; 1821-1823; 1824-1826; 1827-1829; 1830-1832; 1833-1835; 1836-1838; 1839-1841; 1842-1844; 1845-1847; 1848-1850; 1851-1853; 1854-1856; 1857-1859; 1860-1862; 1863-1865; 1866-1868; 1869-1871; 1872-1874; 1875-1877; 1878-1880; 1881-1883; 1884-1886; 1887-1889; 1890-1892; 1893-1895; 1995-1997; 1998-2000; 2001-2003; 2004-2006; 2007-2009; 2010-2012; 2013-2015; 2016-2018; 2019-2021; 2022-2024; 2025-2027; 2028-2230; 2031-2033; 2034-2036; 2037-2039; 2040-2043; 2043-2045; 2046-2048; 2049-2051; 2052-2054; 2055-2057; 2058-2060; 2061-2063; 2064-2066; 2067-2069; 2070-2072; 2073-2075; 2076-2078; 2079-2081; 2082-2084; 2085-2087; 2088-2090; 2091-2093; 2094-2096; 2097-2099; 2100-2102; 2103-2105; 2106-2108; 2109-2111; 2112-2114; 2115-2117; 2118-2120; 2121-2123; 2124-2126; 2127-2129; 2130-2132; 2133-2135; 2136-2138; 2139-2141; 2142-2144; 2145-2147; 2148-2150; 2151-2153; 2154-2156; 2157-2159; 2160-2162; 2163-2165; 2166-2168; 2169-2171; 2172-2174; 2175-2177; 2178-2180; 2182-2183; 2184-2186; 2187-2189; 2190-2192; 2193-2195; 2196-2198; 2299-2201; 2202-2204; 2205-2207; 2208-2210; 2211-2213; 2214-2216; 2217-2219; 2220-2222; 2223-2225; 2226-2228; 2229-2231; 2232-2234; 2235-2237; 2238-2240; 2241-2243; 2244-2246; 2247-2249; 2250-2252; 2253-2255; 2256-2258; 2259-2261; 2262-226; 2265-2267; 2268-2270; 2271-2273; 2274-2276; 2277-2279; 2280-2282; 2283-2285; 2286-2288; 2289-2291; 2292-2294; 2295-2297; 2298-2300; 2301-2303; 2304-2306; 2307-2309; 2310-2312; 2313-2315; 2316-2318; 2319-2321; 2322-2324; 2325-2537; 2327-2330; 2331-2333; 2334-2336; 2337-2339; 2340-2342; 2343-2346; 2349-2351; 2352-2354; 2355-2357; 2358-2360; 2361-2363; 2364-2366; 2367-2369; 2370-2372; 2373-2375; 2376-2378; 2379-2381; 2382-2384; 2385-2387; 2388-2390; 2391-2393; 2394-2396; 2397-2399; 2400-2402; 2403-2405; 2406-2408; 2409-2411; 2412-2413; 2415-2417; 2418-2420; 2421-2423; 2424-2426; 2427-2429; 2430-2432; 2433-2435; 2439-2441; 2442-2444; 2447-2449; 2450-2452; 2453-2455; 2453-2458; 2459-2461-2462-2464; and 2465-2467. In some embodiments, the NKG2D ABD of the subject antibody includes a V_{H} that is at least 90, 95, 97, 98 or 99% identical to a V_{H} domain in the sequence listing and a V_{L} that is at least 90, 95, 97, 98 or 99% identical to the V_{L} domain in the sequence listing. In some embodiments, the B7H3 ABD of the subject antibody includes a V_{H} that is at least 90, 95, 97, 98 or 99% identical to a V_{H} domain in the sequence listing and a V_{L} that is at least 90, 95, 97, 98 or 99% identical to the V_{L} domain in the sequence listing. Any NKG2D ABDs and B7H3 ABDs described in U.S. Provisional Patent Application No. 63/278,999 which is hereby incorporated by reference in its entirety such as the figures, figure legends, and sequences provided therein, can be used in the heterodimeric antibodies described herein.

In some embodiments, a NKG2D binding domain V_{H}/V_{L} pair that is useful for an scFv or Fab portion of any bispecific antibodies described is shown in the sequence listing as, for example, SEQ ID NOS: 2468-2469, 2470-2471, 2472-2473, 2474 and 2478, 2479-2480, 2481-2482, 2483-2484, 2485-2486, 2487-2488, 2489-2490, 2491-2492, 2493-2494, 2495-2496, 2497-2498, 2499-2500, 2501-2502, 2503-2504, 2505-2506, 2507-2508, 2509-2510, 2511-2512, 2513-2514, 2515-2516, 2517-2518, 2519-2520, 2521-2522, 2523-2524, 2525-2526, 2527-2528, 2529-2530, 2531-2532, 2533-2534, 2535-2536, 2537-2538, 2539-2540, 2541-2542, 2543-2544, 2545-2546, 2547-2548, 2549-2550, 2553-2554, 2555-2556, 2557-2558, 2559-2560, 2561-2562, 2563-2564, 2565-2566, 2567-2568, 2569-2570, 2571-2572, 2573-2574, 2589-2590, 2591-2592, 2593-2594, 2595-2596, 2597-2598, 2599-2600, 2601-2602, 2575 and 2576; 2577 and 2576, 2577 and 2578, 2579 and 2582, 2579 and 2583, 2579 and 2584, 2580 and 2582, 2580 and 2583, 2580 and 2584, 2581 and 2582, 2581 and 2583, 2581 and 2584, 2585 and 2587, 2585 and 2588, 2586 and 2587, 2586 and 2588, as well as 5C5, 13C6, 001, 013, 014, 018, 230, 296, 320, 395, P1A34972, P1AE4973, P1A34975, P1AE4977, P1AE4978, P1AE4979, P1AE4980, P1AE4981, ADI-27705, ADI-27724, ADI-27740, ADI-27741, ADI-27743, ADI-28153, ADI-28226, ADI-28154, ADI-29399, ADI-29401, ADI-29403, ADI-29405, ADI-29407, ADI-29419, ADI-29421, ADI-29424, ADI-29425, ADI-29426, ADI-29429, ADI-29447, ADI-29404, ADI-28200, mAb E, MS, 21F2, MS, 21F2, 16F31, KYK-2.0, 1D7B4, KYK-1.0, 11B2D10, 6E5A7, 6H7E7, mAb D, ADI-27727, ADI-27729, ADI-27749, ADI-27744, ADI-27743, ADI-27378, ADI-27379, ADI-29463, mAb A_H1_L1, mAb A_H1_L2, mAb A_H2_L1, mAb A_H2_L2, mAb B_H1_L1, mAb B_H1_L1.1, mAb B_H1_L2, mAb B_H2_L, mAb B_H2_L1.1, mAb B_H2_L2, mAb B_H3_L1, mAb B_H3_L1.1, mAb B_H3_L2, mAb C_H1_L1, mAb C_H1_L2, mAb C_H2_L1, and mAb C_H2_L2.

All cited references are herein expressly incorporated by reference in their entirety.

Whereas particular embodiments of the disclosure have been described above for purposes of illustration, it will be appreciated by those skilled in the art that numerous variations of the details may be made without departing from the invention as described in the appended claims.

### EXAMPLES

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### Example 1: NKG2D Binding Domains

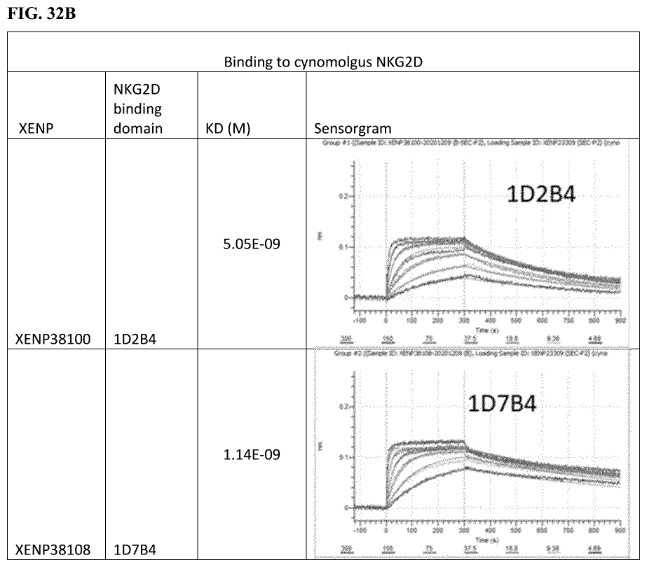

Several NKG2D antibody binding domains (ABDs) that may find use in the invention were discovered by phage display library screening as previously described in U.S. patent application Ser. No. 16/724,118, hereby incorporated by reference. Recombinant human NKG2D (XENP25379; sequences depicted in FIG. 11) and cynomolgus NKG2D (XENP25380; sequences depicted in FIG. 11) were generated in-house for phage panning. In-house de novo phage libraries were built displaying Fab and scFv variants (respectively referred to hereon as “Fab library” and “scFv library”) on phage coat protein pIII. Both the Fab library and the scFv library were panned in five rounds as follows: 1) human NKG2D, 2) cynomolgus NKG2D, 3) human NKG2D, 4) cynomolgus NKG2D, and 5) human NKG2D with increasing levels of stringency (both in terms of antigen concentration as well as wash stringency). The binding of the phage clones to NKG2D was then analyzed by ELISA. 1D7B4 and 1D2B4 were two of the NKG2D binding clones produced from this phage display campaign. Variable heavy and variable light chain sequences for 1D7B4 and 1D2B4 are depicted in FIG. 23. KD values and sensorgrams for 1D7B4 and 1D2B4 in the format of a bispecific antibody binding to human NKG2D or cynomolgus NKG2D are depicted in FIG. 32. Additional NKG2D ABDs disclosed in U.S. patent application Ser. No. 16/724,118 may also find use in the invention, including mAb C and mAb D. The sequence of the variable heavy and variable light chains for mAb C and mAb D may be found in FIG. 23. mAb C and mAb D do not compete for the same NKG2D epitope as 1D7B4 nor 1D2B4, but do compete with one another (data not shown), and therefore may have unique advantages.

### Example 2: B7113 Binding Domains

The B7H3 binding domains used herein, and methods of their discovery have been previously described in U.S. patent application Ser. No. 17/407,135, hereby incorporated by reference. B7H3 binding domains designated 38E2 and 6A1 of the B7H3 binding domains utilized herein were obtained from rabbit hybridoma and humanized using string content optimization (see, e.g., U.S. Pat. No. 7,657,380, issued Feb. 2, 2010). The variable heavy and variable light amino acid sequences for exemplary humanized rat hybridoma-derived 38E2 and 6A1 are depicted respectively in FIG. 13.

An additional B7H3 binding domain, 2E4A3.189, was discovered as previously described in U.S. patent application Ser. No. 17/407,135 through a phage display campaign. It then underwent affinity maturation, with substitutions engineered into the V_{H} only. This effort resulted in clone 2E4A3.189_H1.22. Sequences for 2E4A3.189_H1_L1, and affinity matured 2E4A3.189 H1.22, as well as XENP38571, an exemplary bivalent antibody comprising the 2E4A3.189_H1.22_L1 binding domain, are depicted in FIG. 14.

### Example 3: Engineering B7H3×NKG2D NK Cell Engagers

A number of formats for B7H3×NKG2D bsAbs were conceived, illustrative formats for which are outlined below and depicted in FIG. 15.

3A: 1+1 Fab-scFv-Fc Format

One format utilizing a Fab domain and an scFv is the 1+1 Fab-scFv-Fc format (depicted schematically in FIG. 15A) which comprises a first monomer comprising a single-chain Fv (“scFv”) with a first antigen binding specificity covalently attached to a first heterodimeric Fc domain, a second monomer comprising a heavy chain variable region (V_{H}) covalently attached to a complementary second heterodimeric Fc domain, and a light chain (LC) transfected separately so that a Fab domain having a second antigen binding specificity is formed with the variable heavy domain. Sequences for illustrative αB7H3×αNKG2D bsAbs (based on binding domains as described in Examples 1 and 2) in the 1+1 Fab-scFv-Fc format are depicted in FIG. 19.

3B: 2+1 Fab2-scFv-Fc Format

Another such format is the 2+1 Fab2-scFv-Fc format (depicted schematically in FIG. 15B) which comprises a first monomer comprising a V_{H} domain covalently attached to an scFv (having a first antigen binding specificity) covalently attached to a first heterodimeric Fc domain, a second monomer comprising a V_{H} domain covalently attached to a complementary second heterodimeric Fc domain, and a LC transfected separately so that Fab domains having a second antigen binding specificity are formed with the V_{H} domains. Sequences for illustrative αB7H3×αNKG2D bsAbs (based on binding domains as described in Examples 1 and 2) in the 2+1 Fab2-scFv-Fc format are depicted in FIG. 20.

3C: 2+1 mAb-scFv Format

An additional format utilizing Fab domains and scFv is the 2+1 mAb-scFv format (depicted schematically in FIG. 15-C) which comprises a first monomer comprising a V_{H} domain covalently attached to a first heterodimeric Fc domain covalently attached to an scFv (having a first antigen binding specificity), a second monomer comprising a V_{H} domain covalently attached to a complementary second heterodimeric Fc domain, and a LC transfected separately so that Fab domains having a second antigen specificity are formed with the V_{H} domains. Sequences for illustrative αB7H3×αNKG2D bsAbs (based on binding domains as described in Examples 1 and 2) in the 2+1 mAb-scFv format are depicted in FIG. 21.

### Example 4: Fc Effector Function Engineering

### Example 4A: Fc Effector Function Engineering Enhances NK Cell Activation

Since NK cells rely on multiple signaling events for full activation, NKEs were designed for synergistic simultaneous engagement of FcγRIIIa (also known as CD16) and NKG2D. In order to determine the effect of Fc engagement with CD16 on the ability of B7H3×NKG2D bsAbs to activate NK cells, an activation assay was performed comparing NKG2D×B7H3 bsAbs with varying Fc domains. Test articles XENP38597, XENP38108, and XENP38101 were all produced in the 1+1 Fab-scFv-Fc format, each having a 1D7B4 NKG2D binding domain and a 2E4A3.189 B7H3 binding domain. In regard to the Fc domain, XENP38101 has an Fc comprising ablation variant S267K (numbering according to Kabat), which ablates FcγR binding. Ablation variants, also known as Fc knock-out or “FcKO” variants, are depicted in FIG. 3. XENP38597 has an Fc domain comprising substitutions S239D/I332E (numbering according to Kabat) that increase binding to FcγRIIIa. These S239D/I332E substitutions in an Fc monomeric domain may also be referred to herein as a “V90” variant or an ADCC enhancing Fc variant. The amino acid sequence for an exemplary antibody backbone comprising the V90 variant (S239D/I332E substitutions) is depicted in FIG. 18. Lastly, XENP38108 comprises an Fc domain containing neither ablation variants nor V90 variants. Sequences for XENP38597, XENP38108, and XENP38101 are depicted in FIG. 19.

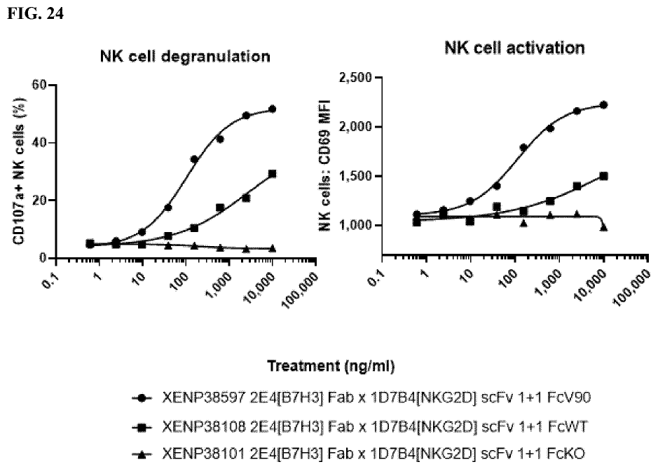

In this assay, PBMCs were mixed with MCF7 cancer cells at a 40:1 ratio, which corresponds to approximately a 1:1 NK cell to MCF7 ratio. Cells were then treated with the one of the three XENPs described above at a range of concentrations as indicated in FIG. 24, and incubated for 4 hours. NK cell activation was then measured by degranulation marker CD107a and activation marker CD69 using flow cytometry. As seen in FIG. 24, XENP38101, having the FcKO ablation variants, was not able to activate NK cells. Meanwhile XENP38108, having a WT Fc domain in respect to FcR binding, was able to moderately activate NK cells, and XENP38597, having the V90 Fc variants allowing for enhanced binding to CD16a, were able to activate NK cells at a significantly higher level.

### Example 4B: Further Tuning of the Fc Effector Function

Inclusion of the ADCC enhancing S239D/I332E (V90) Fc variants in antibody constructs can result in decreased stability and a lower yield. To address this, an experimental set of B7H3 antibodies in the 1+1 Fab-scFv-Fc format (sequences for which are depicted in FIG. 55) were designed and produced with Fc regions having different combinations of S239D and/or I332E substitutions on either one or both Fc monomers, either symmetrically or asymmetrically. B7H3 was used as the ABD in both the Fab and the scFv arm for these test articles so that the binding and activity assays would be solely assessing CD16 engagement. FIGS. 51-52 depict the sequences of the various symmetric & asymmetric ADCC-enhanced backbone sequences, both without and with the addition of the Xtend M428L/N434S variants. In order to investigate the ADCC activity, MCF7 cells were seeded in a 96 well plate and incubated overnight. The next day, the ADCC Reporter Bioassay kit from Promega (Catalog #G7018) was used according to the manufacturer's protocol. Test articles and effector cells were added to the MCF7 cells, and after a 6 hour incubation, Bio-Glo luciferase reagent was added and plates were analyzed with an EnVision plate reader. This revealed a ladder of ADCC activity depicted numerically in FIG. 40 and graphically in FIG. 41. As demonstrated by the values in FIG. 40, this Fc engineering was successful in improving both the production yields and the stability as measured by the melting temperature (Tm). In general, the more similar the Fc region is to the wild-type IgG1 Fc at positions 239 and 332 the better the stability, but the lower the affinity for CD16 and the lower the ADCC and target cell killing activity. However, test articles with only the S239D mutations, such as XENP41023, have significant higher stability as compared to V90 and also provide a slightly higher affinity for CD16 as compared to XENP41024 which only has the I332E mutation. Additionally, S239D imposes a smaller decrease in stability and production than does the I332E mutation. The range of properties and affinities conferred by the different symmetric and asymmetric Fc variants disclosed may each provide unique advantages in certain contexts.

### Example 5: NKEs Augment Activation of NK Cells

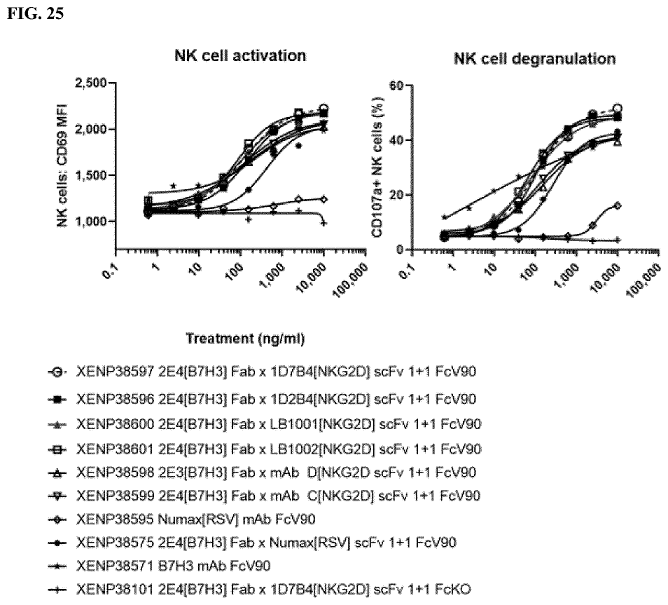

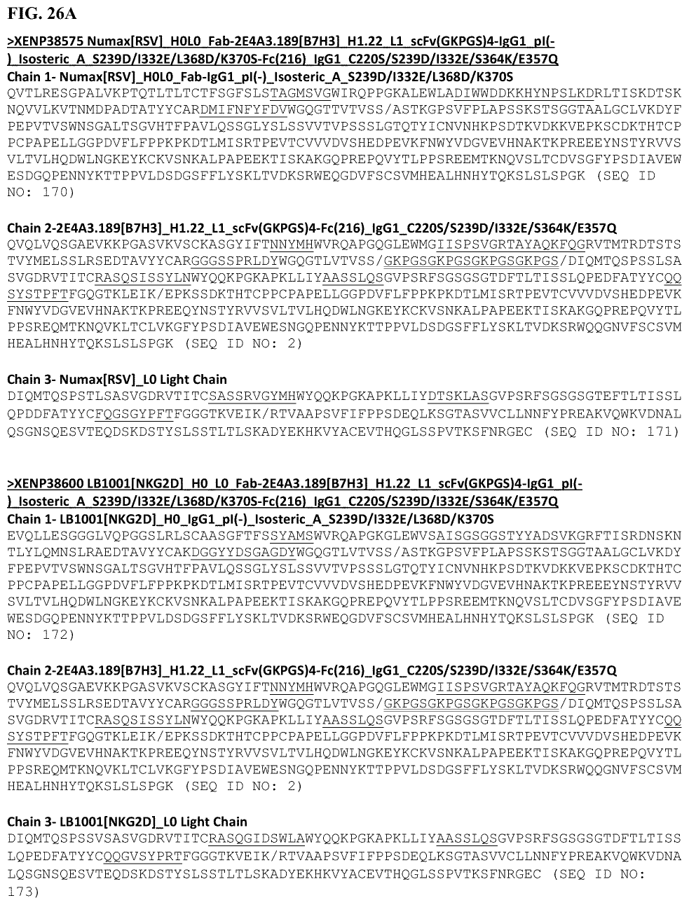

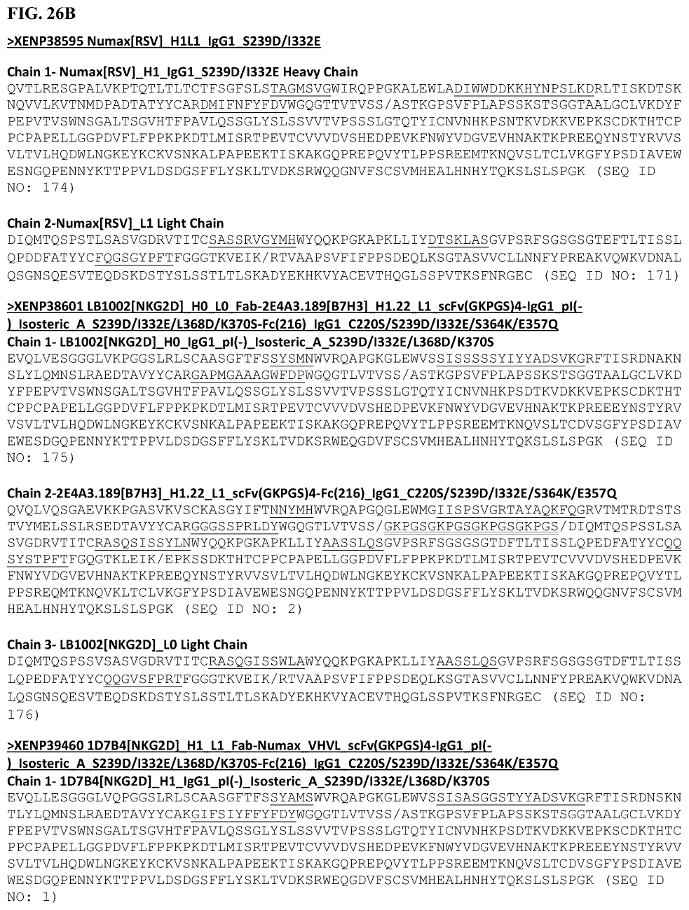

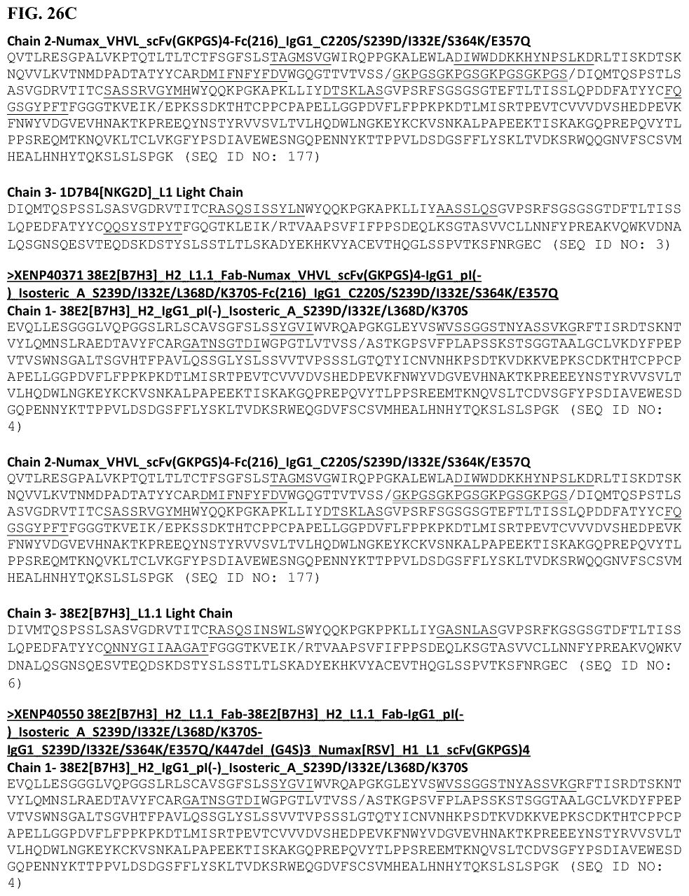

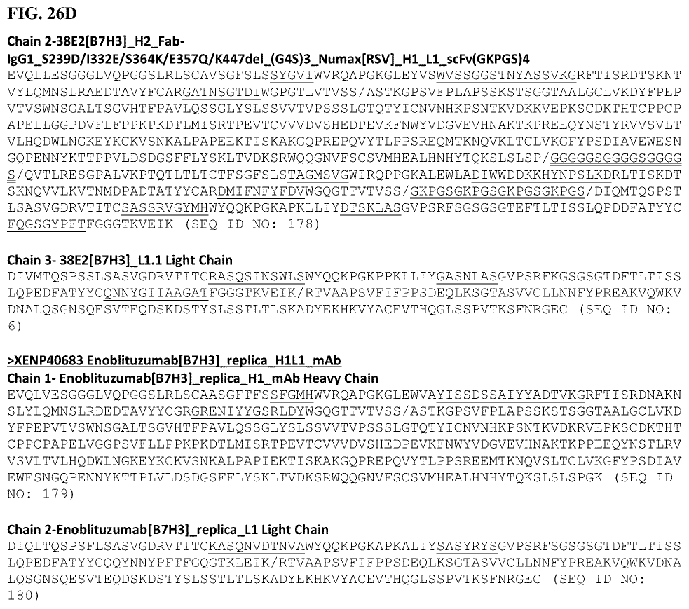

After establishing the key role that Fc engagement of FcγRIIIa plays in NK cell activation, additional 1+1 Fab-scFv-Fc format constructs were produced, all comprising the V90 substitutions but with varying NKG2D binding domains. In an assay, PBMCs were co-cultured with MCF7 cancer cells at a 40:1 ratio, which corresponds to approximately a 1:1 NK cell to MCF7 ratio, and then cells were treated with test articles including XENP38597, having the 1D7B4 binding domain and XENP38596, having the 1D2B4 binding domain, alongside other comparator molecules and controls. After a 4-hour incubation, flow cytometry was used to measure degranulation marker CD107a. The results seen in FIG. 25 showed 1D7B4 and 1D2B4 to be two of the most active NK engagers. Control test articles such as XENP38575 (sequences depicted in FIG. 26), having a B7H3 binding domain but no NKG2D binding domain, resulted in decreased NK cell activation compared to NKG2D×B7H3 bsAbs.

### Example 6: NKEs Enhance NK Cell Mediated Cytotoxicity

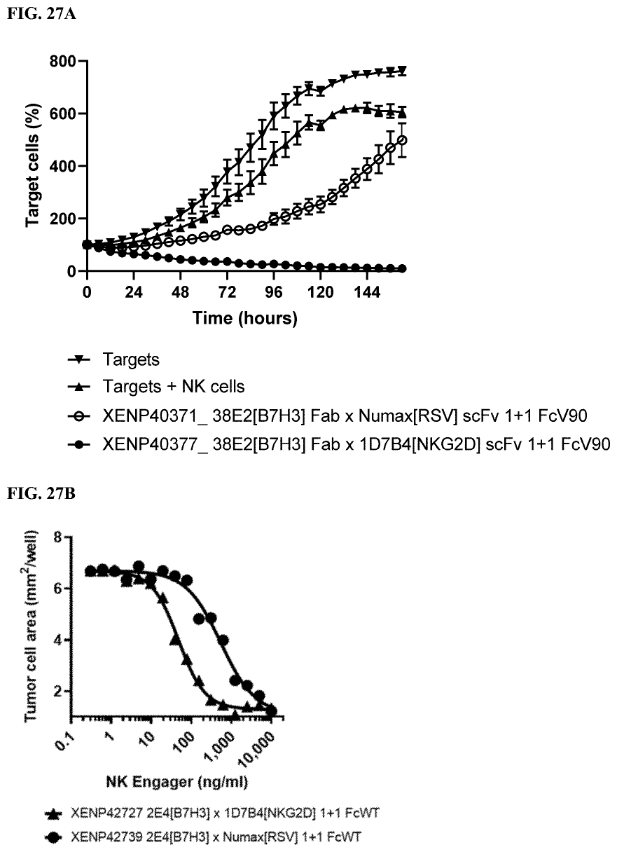

In order to assess the ability of NKEs to kill target cells, an experiment was performed in which resting NK cells were co-cultured with MCF7-RFP tumor cells at an E:T ratio of 5:1. Treatments of either XENP40371 or XENP40377 were added at a concentration of 4.6 ng/ml. The growth of the MCF7 tumor cells was assessed over time using Incucyte. As depicted in FIG. 27, XENP40371, having a B7H3 binding domain but no NKG2D binding domain, has the ability to kill some percentage of target cells. However, XENP40377, having both the 1D7B4 NKG2D binding domain and the 38E2 B7H3 binding domain, showed a significantly improved ability to kill target cells compared with XENP40371. The sequence for XENP40371 is depicted in FIG. 26 and the sequence for XENP40377 is depicted in FIG. 19.

### Example 7: MHC Downregulation Increases Target Cells Sensitivity to NKE Driven Cell Lysis

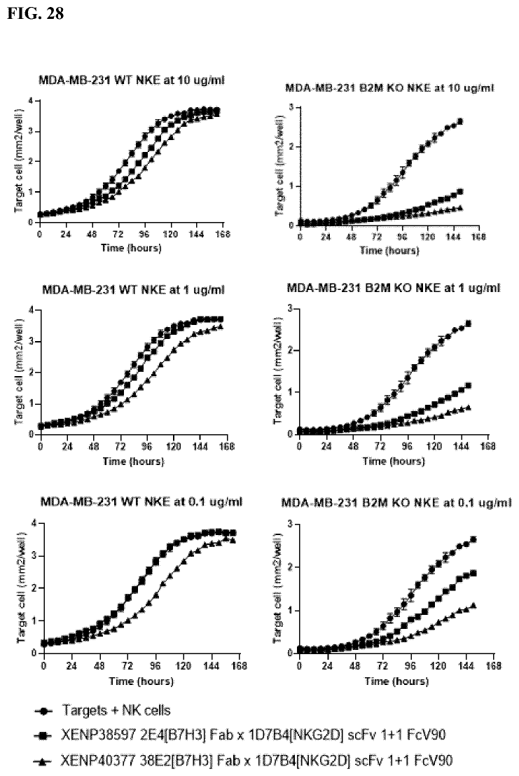

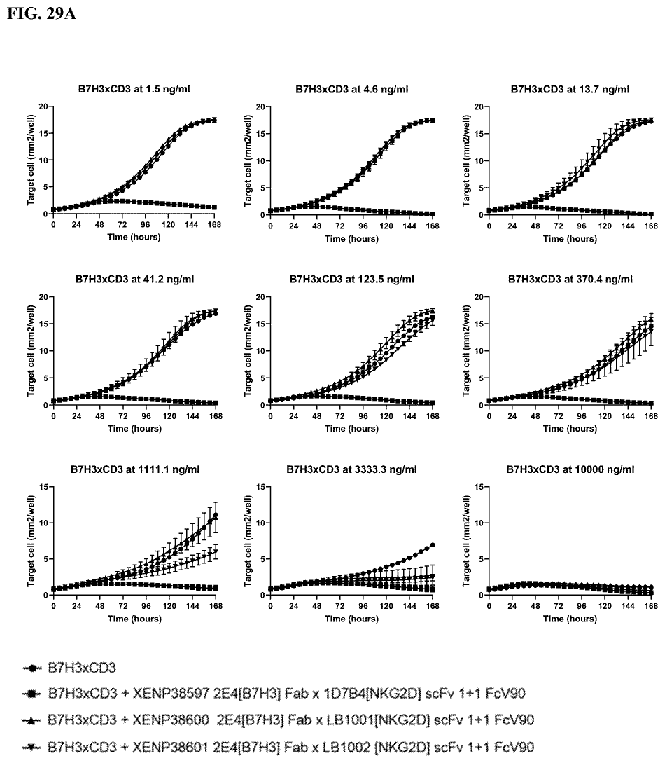

As mentioned previously, one potential benefit of NKE cancer therapies over existing T cell engager cancer therapies is the ability of NKEs to target cancer cells that have reduced MHC expression and are therefore less responsive to therapies targeting the adaptive immune system. In order to investigate this concept with NKG2D×B7H3 NKEs, an assay was performed comparing the effects of NKEs on wild-type MDA-MB-231 cells with the effects of NKEs on MDA-MB-231 cells in which a component of the MHC, beta-2 microglobulin (B2M), has been knocked out. NK cells were added to MDA-MB-231 WT or MDA-MB-231 B2M knockout cells at a 5:1 E:T ratio. XENP38597 or XENP40377 were added at a range of 0.1 μg/ml to 10 μg/ml, and the target cell count was recorded over time by Incucyte. As apparent in FIG. 28, NKEs delivered as a single agent showed significantly improved cell killing on MDA-MB-231 B2M knockout cells compared to MDA-MB-231 WT cells.

In addition, as seen most clearly when the NKE concentration is 0.1 μg/ml, XENP40377, having higher affinity B7H3 binding domain 38E2, killed target cells more effectively than XENP38597, having the comparatively lower affinity B7H3 binding domain 2E4A3.189. This demonstrates that tumor antigen domain affinity is important for functional activity.

### Example 8: NKEs Provide Co-Stimulation Signal to T-Cells in Presence of TCR-Mediated Signaling Resulting in Tumor Lysis

In order to assess the ability of NKEs to provide co-stimulation to T-cells and thereby enhance tumor cell killing, an experiment was performed in which T cells were added to MCF7 target cells at a 5:1 E:T ratio. All cells were dosed at a constant concentration of 10 μg/ml of NKEs. One NKE used in this experiment was XENP38597, having a 1D7B4 NKG2D binding domain. The other two NKEs used were XENP38600 and XENP38601, which have the same format and B7H3 binding domain as XENP38597, but which use comparator NKG2D binding domains instead. Sequences for XENP38600 and XENP38601 are depicted in FIG. 26. The cells were also treated with a B7H3×CD3 T-cell engager (e.g., an anti-B7H3×anti-CD3 bispecific antibody) at doses ranging from 1.52 ng/ml to 10 μg/ml. The cells were incubated in the Incucyte, where target cell viability was measured every 6 hours over a span of 160 hours. As can be seen in FIG. 29A, XENP38597 was able to activate T cells starting even at the lowest dose of TCR stimulation. On the other hand, the comparator molecules do not begin to demonstrate any lysis of tumor cells until much higher doses of B7H3×CD3 XENP31346; doses at which the T cell engager begins to effectively lyse tumor cells independently. Only XENP38597, and not the comparator molecules, were able to co-stimulate T cells to kill tumor cells.

### Example 9: NKEs Synergize with Pro-Inflammatory Cytokines in Killing Target Cells

9A: NKEs Show Synergistic Activity when Combined with IL-15

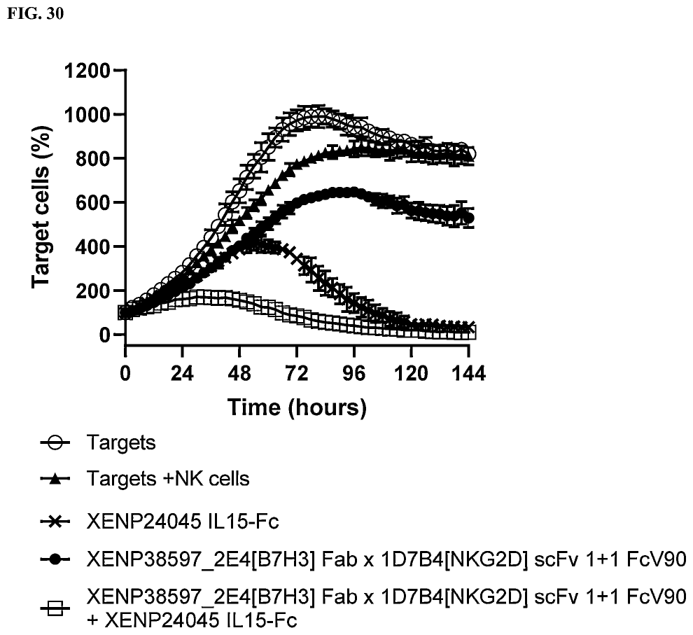

In order to determine the effect of IL-15 on NKEs ability to kill target cells, an experiment was conducted in which NK cells were co-cultured with OVCAR8-NIR target cells at a 5:1 E:T ratio. A control group of cells was then left alone, while other groups were dosed with either 10 μg/ml IL15-Fc (XENP24045, the sequence for which is depicted in FIG. 22), 10 μg/ml NKG2D×B7H3 bsAb (XENP38597), or both. Target cell counts were then measured over time using Incucyte. As seen in FIG. 30, the combination of both IL15-Fc and NKG2D×B7H3 bsAb was significantly more efficacious in killing target cells than either of the two treatments alone.

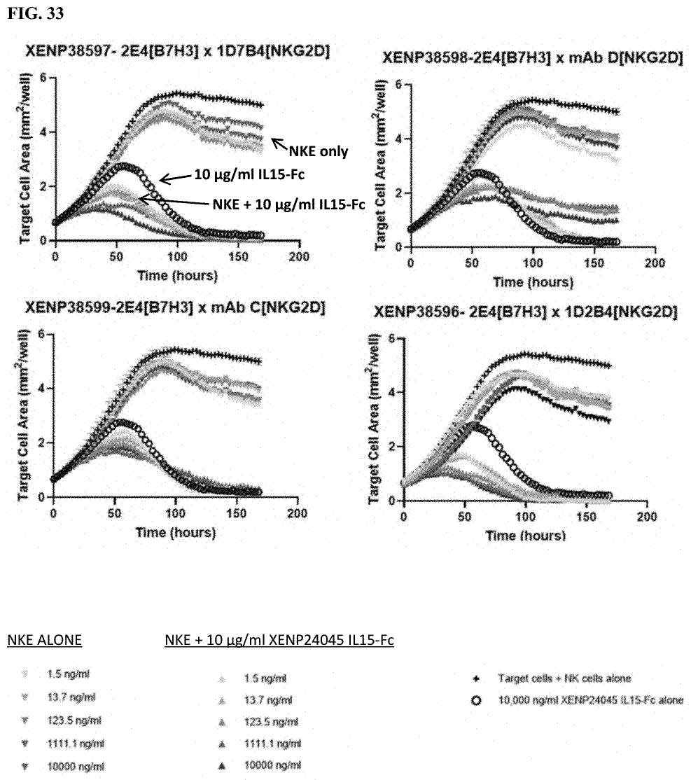

In an additional experiment depicted in FIG. 33, XENP38597 was compared with other XENPs in the 1+1 Fab-scFv-Fc format having different NKG2D binding domains. NK cells were co-cultured with OVCAR8 target cells at a 5:1 E:T ratio. Cells were dosed with 10 μg/ml IL-15 Fc (XENP24045) and NKEs were titrated in at a dose range of 0.2 ng/ml to 10 μg/ml. Incucyte was used to quantify target cells over time. As seen in FIG. 33, XENP38597 and XENP38596, having ID7B4 and ID2B4 NKG2D binding domains respectively, demonstrated better dose response and synergy with IL-15 compared to XENP38599 (having the mAb C NKG2D binding domain) or XENP38598 (having the mAb D NKG2D binding domain).

9B: NKEs Show Synergistic Activity when Combined with IL-12

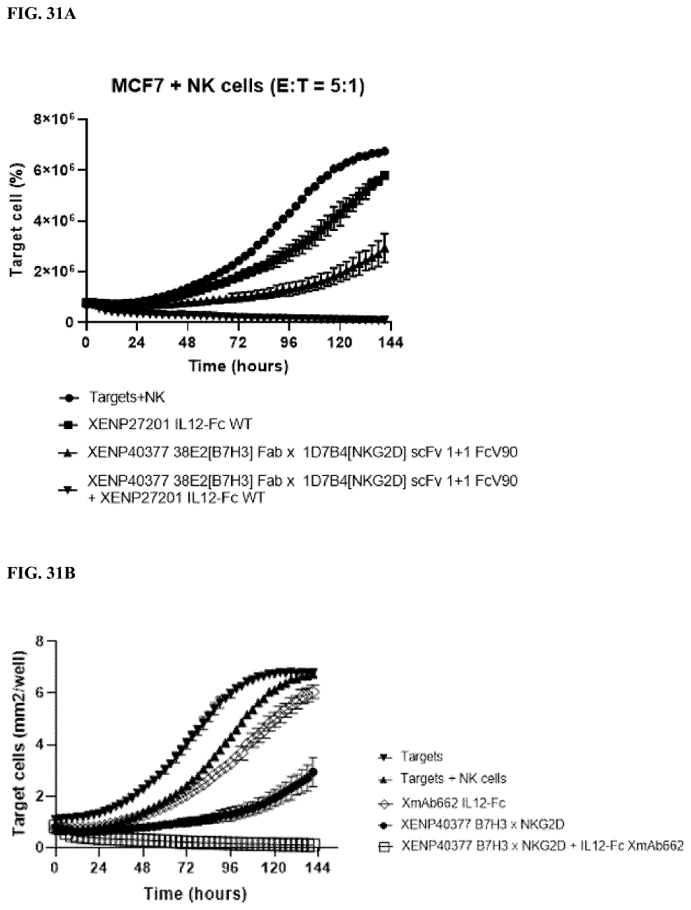

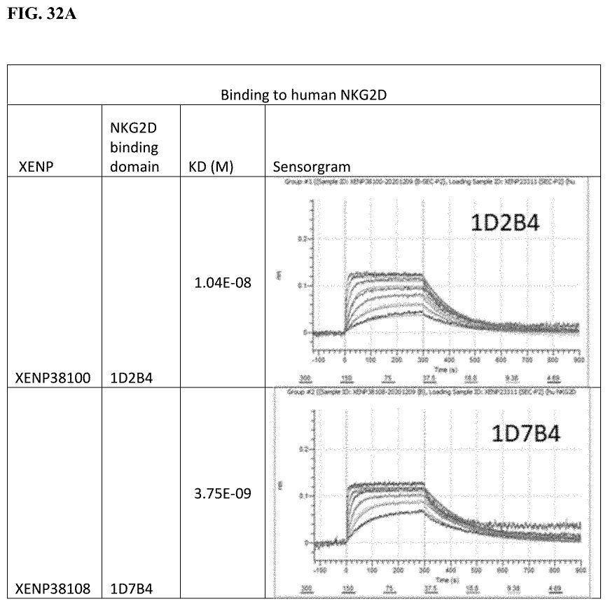

In order to investigate whether NKEs might have synergy with other cytokines in addition to IL-15, another experiment was performed in which NK cells were co-cultured with MCF7 target cells at a 5:1 E:T ratio. A control group of cells was then left alone, while other groups were dosed with either 10 μg/ml IL12-Fc (XENP27201, sequence for which is depicted in FIG. 22), 4 μg/ml NKG2D×B7H3 bsAb (XENP40377), or both. Target cell counts were then measured over time using Incucyte. As seen in FIG. 31A, the combination of both IL12-Fc and XENP40377 also showed significantly better target cell killing than either of the two treatments alone. In a second similar Incucyte experiment, cells were again dosed with 4 μg/ml XENP40377 alone or in combination with IL-12-Fc, but using 2 μg/ml of IL-12-Fc XmAb662 (XENP39662, the sequence for which is depicted in FIG. 22) rather than XENP27201. Again, as depicted in FIG. 31B, the combination of IL-12-Fc with XENP40377 demonstrates much better killing than either treatment alone.

### Example 10: Additional Engineering of Anti-NKG2D Binding Domain ID7B4

Because NKEs can engage NK cells via more than one domain (e.g., the anti-NKG2D binding domain and the Fc domain), trans engagement between NK cells can lead to fratricide. It was hypothesized that fratricide could be minimized through decreasing NKG2D affinity to a point where there is no fratricide but there is still on-target potency. Toward this end, a library of anti-NKG2D clones with detuned affinity was designed based on the 1D7B4 clone. The detuned anti-NKG2D variants were incorporated into NKEs and tested for their ability to provide on-target toxicity and avoid fratricide.

10A: Establishing a 1D7B4 Affinity Ladder

The detuned 1D7B4 variants were produced as bivalent antibodies (sequences for which are depicted in FIG. 54) and as His-tagged Fabs in order to facilitate biophysical analysis on Octet and Biacore. The detuned 1D7B4 variable heavy chains and their respective CDRs are depicted in FIG. 58. In an initial Octet HTX screen of all detuned 1D7B4 variants, NKG2D antigen was captured at 20 nM for 5 min using AHC sensors, and then dipped into ˜300, 100, 33.3 and 0 nM of each Fab in supernatant. A table summarizing the test article mutations and affinity values is depicted in FIG. 42. From this screening, a smaller set was selected as an affinity ladder to move forward for further analysis. Selected detuned 1D7B4 variants were then produced in 1+1 Fab-scFv-Fc format (with a B7H3 Fab and NKG2D scFv), sequences for which are depicted in FIG. 59. These constructs were then analyzed using a Biacore T200. A CM5 chip was amine coupled to anti-His capture mAb with XENP23311 (His-tagged NKG2D-Fc, sequence for which is depicted in FIG. 11) and Acro Bio's commercial human NKG2D ligands. Then the detuned 1D7B4 analytes in the B7H3×NKG2D 1+1 Fab-scFv-Fc format were flowed at concentrations of 15000, 5000, 1666.7, 555.6, 185.2, 61.7, 20.6, 6.9, 2.3, 0.76, and 0.25 nM with a 5 minute association and 10 minute dissociation time. The experiment was run at 25° C. FIG. 43 depicts the affinity data of these constructs for NKG2D-Fc. As shown, XENP42652, XENP42653, XENP42654, XENP42655, and XENP42656 formed a decreased affinity ladder when compared to XENP40375.

10B: Detuned 1D7B4 Variant B7H3×NKG2D NKEs Decrease Fratricide

In order to investigate whether the detuned 1D7B4 variants were able to decrease fratricide levels, an experiment was performed to assess cell killing in the presence of NK cells only. In this experiment, 100,000 NK cells were added to each well of a plate, and serial dilutions of detuned 1D7B4 NKE test articles were added. A CD107a staining antibody was also added, and cells were incubated overnight for −12 hours. The next day, cells were stained with Zombie Aqua and analyzed via flow cytometry. One of the 1D7B4 detuned variant NKEs, the H1.28 variant, showed no decrease in the percentage of NK cells killed compared to the parental clone, XENP40377. However, the rest of the clones in this experiment all showed a range of significant reductions in fratricide, as measured by percentage of dead NK cells and depicted in FIG. 44. XENP42659 (having the 1D7B4_H1.23 variant), XENP42661 (having the 1D7B4_H1.31 variant) and XENP42662 (having the 1D7B4_H1.33 variant) showed the greatest reductions in fratricide. Additionally, there was a decrease in NK cell degranulation, measured by the percentage of CD107a+ NK cells as seen in FIG. 44. It should be noted that all the test articles in this experiment all utilize the “v90” S239D/I332E Fc variants, and when the experiment is repeated using test articles having WT Fc, fratricide is not well detected in vitro (data not shown).

10C: Detuned 1D7B4 Variant B7H3×NKG2D NKEs Retain Activity Both Independently and Synergistically with IL-15

In order to explore the impact of detuning 1D7B4 affinity on the ability of NKEs to lyse target cells, an experiment was conducted in which A375 target cells were plated and cultured with effector cells at 5:1 E:T ratio. Treatments were added at a range of concentrations as indicated in FIG. 45, with or without the addition of 5 μg/ml IL-15-Fc. Target cell death was assessed using Incucyte. As seen in FIG. 45, detuned NKEs retained the ability to kill target cells and induce IFNγ production, and these activities are enhanced when combined with IL-15. In summary, H1.3 and H1.28 were potent but had high fratricide, H1.33 showed no fratricide but also no additive activity on top of ADCC, while H1.31 and H1.23 have reduced fratricide and retained NKG2D agonistic activity. Similar results confirming this activity ranking were attained by repeating the experiment using five different huPBMC donors, and the results graphing the EC50 values from the target cell lysis curves with each different donor are depicted in FIG. 45B.

### Example 11: Further Tuning of NKE Function Through Format Modification

### Example 11A: Effects of NKG2D Fv Format and Position

NKEs in the 1+1 Fab-scFv-Fc format can be designed with either an anti-B7113 Fab arm and anti-NKG2D scFv arm, or an anti-NKG2D Fab arm and anti-B7113 scFv arm. An experiment was performed to assess the impact of these alternative formats on the levels of fratricide. NK cells were plated alone (without any target cells present), and then test articles were added at a range of doses as shown in FIG. 46 and incubated for 12 hours before assessing cell viability. As illustrated in FIG. 46, there is a reduction in the potency of NK cell fratricide when the anti-NKG2D Fv is in the scFv format. However, the reduction is less pronounced when there is a higher affinity variant of CD16 is expressed on the NK cells, highlighting the influence that V90-enhanced Fc binding to CD16 maintains in contributing to fratricide. An additional experiment was performed in which the binding to NK cells was assessed for an NKE in the 1+1 Fab-scFv-Fc format having a B7H3 Fab and NKG2D scFv (XENP38101), an NKE in the 1+1 Fab-scFv-Fc format having an NKG2D Fab and B7H3 scFv (XENP40376), and an NKE in the mAb-scFv format having B7H3 Fab arms and an NKG2D scFv (XENP40557). All 3 constructs were made in the FcKO format so that the NK cell binding being measured would not be affected by the binding of the Fc region to CD16. As depicted in FIG. 47, XENP38101 had the highest efficacy and affinity, followed by XENP40376 and XENP40557. Taken together, these experiments show that each format may provide unique advantages in certain contexts.

### Example 11B: Impact of NKG2D scFv Domain Orientation on Affinity

Whether an scFv is in the VHVL or VLVH orientation may have an impact on the affinity of the scFv for its target. NKEs having a B7H3 Fab and an NKG2D scFv were tested for their affinity in both scFv orientations in the 1+1 Fab-scFv-Fc, 2+1 Fab₂-scFv-Fc, and 2+1 mAb-scFv formats. This experiment was performed using Octet HTX with an HIS1K chip. His-tagged NKG2D antigen (XENP23311) was captured at 20 nM for 3 min and dipped into NKE test articles at concentrations of 300, 100, 33.3, 11.1, 3.7, 1.23, 0.41 and 0 nM with a 3 min association and 5 min dissociation time. Each sample was run in duplicate. As illustrated by the KD values in FIG. 48, the affinity of the 1D2B4 and 1D7B4 clones were less affected by the changes in orientation than comparator clones LB1001 or LB1002. This unexpected ability to retain a more similar affinity in either the VHVL or VLVH contexts may provide unique advantages in certain contexts.

### Example 12: NKEs Activate NK Cells and CD8+ T Cells In Vivo

In order to test the ability of NKEs to activate NK cells in vivo, a mouse study was initiated. In this study, female huCD34+ NSG mice were inoculated intradermally with 3×10⁶ ppMCF7-GFP cells per mouse on Day −16. Then on Day 0, they were dosed with a 5 mg/kg NKE treatment and a 0.2 mg/kg IL-15-Fc (XENP24045) treatment intraperitoneally. Dosing was repeated weekly for 4 weeks, and blood was drawn weekly for peripheral lymphocyte cell counts. As depicted in FIG. 49A and FIG. 49B, from a peripheral blood analysis on Day 21, CD69 is upregulated in NK cells and CD8+ T cells in groups treated with NKG2D×B7H3 NKEs but not in groups treated with RSV×B7H3 controls. This demonstrates that NKEs successfully induce NK cell and CD8+ T cell activation in vivo via NKG2D engagement. Additionally, as depicted in FIG. 49C, NKG2D-targeting NKEs also increased the percentage of IFNγ positive NK cells. Lastly, as shown in FIG. 49D, NKG2D-targeting NKEs induce proliferation as measured by the percentage of Ki-67+ cells on Day 21.

### Example 13: NKEs Induce NK-Mediated IFNγ Production

The hallmark of NKG2D-targeting NKEs is a potent induction of IFNγ production via the NKG2D pathway agonism, independent of FcγR engagement. This can be illustrated in an experiment in which NK cells were co-cultured with A375-B2M-KO-RFP tumor cell line and treated with either an NKG2D×B7H3 NKE or an RSV×B7H3 isotype control, both having an FcKO Fc region with ablated FcγR binding. Tumor cell growth was assessed with Incucyte. As depicted in FIG. 61, independent of FcγR engagement, the NKG2D targeting XENP40367 was able to induce IFNγ production.

1. A heterodimeric antibody comprising:
a) a first monomer comprising:
i) an anti-NKG2D scFv comprising a first variable heavy VH1 domain, an scFv linker and a first variable light VL1 domain; and
ii) a first Fc domain, wherein the scFv is covalently attached to the N-terminus of the first Fc domain using a domain linker;
b) a second monomer comprising a VH2-CH1-hinge-CH2-CH3 monomer, wherein VH2 is a second variable heavy domain and CH2-CH3 is a second Fc domain; and
c) a light chain comprising a second variable light VL2 domain,
wherein the second variable heavy VH2 domain and the second variable light VL2 domain form an B7H3 antigen binding domain, and
wherein the first Fc domain and/or the second Fc domain comprise an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering and have enhanced FcγRIIIA (CD16a) binding compared to first and second Fc domains lacking such substitution(s).

2. The heterodimeric antibody according to claim 1, wherein the B7H3 antigen binding domain comprises a set of vhCDR1-3 and vlCDR1-3 from a variable heavy domain and variable light domain pair selected from the group consisting of SEQ ID NOS: 27, 28, and 29 for vhCDR1-3 and SEQ ID NOS: 30, 31, and 32 for vlCDR1-3 of 38E2[B7H3]_H2_L1.1; and SEQ ID NOS: 20, 21, and 22 for vhCDR1-3 and SEQ ID NOS: 23, 24, and 26 for vlCDR1-3 of 2E43.189[B7H3]_H1.22_L1, as depicted in FIGS. 13 and 14.

3. (canceled)

4. The heterodimeric antibody according to claim 1, wherein the anti-NKG2D scFv comprises a set of vhCDR1-3 and the vlCDR1-3 from a variable heavy domain and variable light domain pair selected from the group consisting of SEQ ID NOS: 2612-2614 for vhCDR1-3 and SEQ ID NOS: 2616-2618 for vlCDR1-3 of mAb-D[NKG2D]; SEQ ID NOS: 17-18 and 1256 for vhCDR1-3 of 1D7B4[NKG2D]_H1.23 and SEQ ID NOS: 23, 24, and 26 for vlCDR1-3 of 1D7B4[NKG2D]_L1; SEQ ID NOS: 17-18 and 1272 for vhCDR1-3 of 1D7B4[NKG2D]_H1.31 and SEQ ID NOS: 23, 24, and 26 for vlCDR1-3 of 1D7B4[NKG2D]_L1; and SEQ ID NOS: 17-19 for vhCDR1-3 and SEQ ID NOS: 23, 24, and 26 for vlCDR1-3 of 1D7B4[NKG2D]_H1_L1, as depicted in FIGS. 23 and 58.

5. (canceled)

6. The heterodimeric antibody according to claim 1, wherein the first variable light domain of the anti-NKG2D scFv is covalently attached to the N-terminus of the first Fc domain using a domain linker or the first variable heavy domain of the anti-NKG2D scFv is covalently attached to the N-terminus of the first Fc domain using a domain linker.

7. (canceled)

8. The heterodimeric antibody according to claim 1, wherein the scFv linker is a charged scFv linker.

9. (canceled)

10. The heterodimeric antibody according to claim 1, wherein the first domain and/or second domain comprise an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering.

11. (canceled)

12. (canceled)

13. The heterodimeric antibody according to claim 1, wherein the first or second Fc domain comprises the amino acid substitutions S239D/I332E, wherein numbering is according to EU numbering.

14. The heterodimeric antibody according to claim 1, wherein the first and second Fc domains further comprise a set of heterodimerization variants selected from the group consisting of those depicted in FIGS. 1A-1E, wherein numbering is according to EU numbering.

15. (canceled)

16. The heterodimeric antibody according to claim 1, wherein the first or second Fc domain further comprises one or more pI variants.

17. (canceled)

18. The heterodimeric antibody according to claim 1, wherein the first and second monomers each further comprise amino acid substitutions selected from the group consisting of M428L/N434S, M428L/N434A, and M252Y/S254T/T256E, wherein numbering is according to EU numbering.

19. The heterodimeric antibody according to claim 1, selected from the group consisting of: the amino acid sequences of SEQ ID NOS: 4, 5 and 6 of XENP40377; the amino acid sequences of SEQ ID NOS: 1309-1310 and 6 of XENP42653; and the amino acid sequences of SEQ ID NOS: 1313-1314 and 6 of XENP42655, as depicted in FIGS. 19 and 59.

20. A nucleic acid composition comprising nucleic acids encoding the first and second monomers and the light chain of the antibody according to claim 1.

21. An expression vector comprising the nucleic acids according to claim 20.

22. A host cell transformed with an expression vector according to claim 21.

23. A method of making a heterodimeric antibody comprising culturing the host cell according to claim 22 under conditions wherein the heterodimeric antibody is expressed, and recovering the heterodimeric antibody.

24. A heterodimeric antibody comprising:
a) a first monomer comprising:
i) an anti-B7H3 scFv comprising a first variable heavy VH1 domain, an scFv linker and a first variable light VL1 domain; and
ii) a first Fc domain, wherein the scFv is covalently attached to the N-terminus of the first Fc domain using a domain linker;
b) a second monomer comprising a VH2-CH1-hinge-CH2-CH3 monomer, wherein VH2 is a second variable heavy domain and CH2-CH3 is a second Fc domain; and
c) a light chain comprising a second variable light VL2 domain,
wherein the second variable heavy domain and the second variable light domain form an NKG2D antigen binding domain, and
wherein the first Fc domain and/or the second Fc domain comprise an amino acid substitution(s) selected from the group consisting of S239D, I332E, S239D/I332E, G236A, S239E, I332D, G236A/I332E, S239D/I332E/A330L, I332E/A330L, F243L, and S298A, wherein numbering is according to EU numbering and have enhanced FcγRIIIA (CD16a) binding compared to first and second Fc domains lacking such substitution(s).

25.-41. (canceled)

42. The heterodimeric antibody according to claim 24, selected from the group consisting of the amino acid sequences of SEQ ID NOS: 1, 2 and 3 of XENP38597; the amino acid sequences of SEQ ID NOS:7, 8 and 3 of XENP38101; the amino acid sequences of SEQ ID NOS: 9, 10 and 3 of XENP38108; and the amino acid sequences of SEQ ID NOS: 12, 2, and 13 of XENP38598, as depicted in FIGS. 19 and 59.

43.-116. (canceled)

117. A method of treating cancer or reducing tumor growth or inhibiting cancer cell proliferation in a subject in need thereof comprising administering to the subject a therapeutically effective amount of the heterodimeric antibody of claim 1 or antigen binding fragment thereof to the subject.

118. The method of claim 117, further comprising administering an IL-12-Fc fusion protein and/or an IL-15-Fc fusion protein to the subject.

119. The method of claim 117, further comprising administering a bispecific T-cell engager antibody or an antigen binding fragment thereof to the subject.

120.-125. (canceled)
